(19) United States
(12) Patent Application Publication
(10) Pub. No.: US 2012/0022056 A1
(43) Pub. Date: Jan. 26, 2012

(54) FUSED TRICYCLIC DERIVATIVES FOR THE TREATMENT OF PSYCHOTIC DISORDERS

(76) Inventors: Jonathan Bentley, Verona (IT); Markus Bergauer, Verona (IT); Babara Bertani, Verona (IT); Matteo Biagetti, Verona (IT); Manuela Borriello, Verona (IT); Steven Mark Bromidge, Verona (IT); Massimo Gianotti, Verona (IT); Enrica Granci, Verona (IT); Colin Philip Leslie, Verona (IT); Alessandra Pasquarello, Verona (IT); Valeria Zucchelli, Verona (IT)

(21) Appl. No.: 11/574,450

(22) PCT Filed: Aug. 29, 2005
(86) PCT No.: PCT/EP05/09379

§ 371 Date: Aug. 10, 2009

(51) Int. Cl. A61K 31/5383; C07D 471/04; A61K 31/496; A61P 25/00; A61P 25/18; A61P 25/24; A61P 25/22; A61P 15/00; C07D 498/04; A61K 31/4545

(52) U.S. Cl. 514/230.2; 544/101; 544/73; 544/363; 546/84; 546/82; 514/253.03; 514/318

(57) ABSTRACT

Compounds of formula (I) wherein R¹, R², X, A, Y, B, Z¹, Q, p, r and s are defined in the specification for treating inter alia psychotic disorders, depressive disorders, anxiety disorders and sexual dysfunctions.

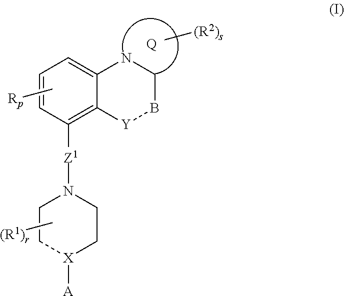

The present invention relates to novel fused tricyclic derivatives, processes for their preparation, pharmaceutical compositions containing them and their use as medicaments, inter alia for the treatment of psychotic disorders, depressive disorders, anxiety disorders and sexual dysfunctions.

WO2004/046124 discloses a series of benzoxazinone compounds having affinity for 5-HT₁ type receptors and/or possess serotonin re-uptake inhibition activity.

We have identified a novel series of compounds that possess high affinity for 5-HT₁ type receptors and/or are serotonin re-uptake inhibitors.

In a first aspect, the invention provides a compound of formula (I), a salt or prodrug thereof

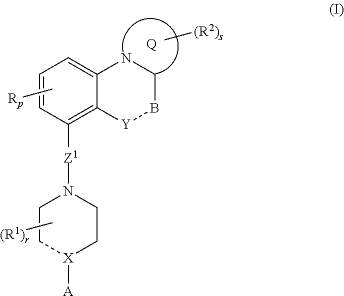

wherein
— represents independently a single or double bond;
ring Q is a 5-membered heteroaromatic ring or a 5-membered heterocyclic ring either of which contains at least one ring-nitrogen atom as shown in formula (I) and optionally 1 to 3 additional ring-heteroatoms independently selected from oxygen, nitrogen and sulphur,
B is C(R⁷)(R⁸) or C(R⁷), wherein where the bond connecting B and Y is a single bond B is C(R⁷)(R⁸) and when the bond connecting B and Y is a double bond B is C(R⁷);
Y is C(R⁷), C(R⁷)(R⁸), O or S(O)ₜ, wherein where the bond connecting B and Y is a single bond, Y is C(R⁷)(R⁸), O or S(O)ₜ and when the bond connecting B and Y is a double bond, B is C(R⁷);
Z¹ is a linking group of formula (A)

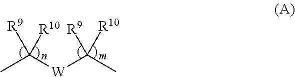

wherein
W is —(CH═CH)—; —C(═O)—; —C(═CH₂)—; —C(R⁷)(R⁸)—; —C(R⁷)(R⁸)—S(O)ₜ—; —C(R⁷)(R⁸)—O—; or a 3 to 7-membered cycloalkylene group or 3 to 7-membered cycloalkenylene group either of which groups are optionally substituted by 1 to 3 substituents which may be the same or different, selected from halogen hydroxy, cyano, C₁₋₆alkyl, haloC₁₋₆alkyl, C₁₋₆alkanoyl and C₁₋₆alkoxy; and
n and m are independently 0, 1 or 2;
X is C(R¹), N or C; wherein where the dotted bond attached to X is a single bond, X is C(R¹) or N, and when the dotted bond attached to X is a double bond, X is C;
A is indolyl, quinolyl, quinazolinyl, benzofuranyl or benzothienyl, any of which are optionally substituted by 1-4 substituents, which substituents may be the same or different, and are selected from the group consisting of halogen, hydroxy, cyano, nitro, C₁₋₆alkyl, haloC₁₋₆alkyl, C₃₋₇cycloalkyl, arylC₁₋₆alkoxy, C₁₋₆alkylthio, C₁₋₆alkoxy, haloC₁₋₆alkoxy, C₁₋₆alkoxyC₁₋₆alkyl, C₁₋₆alkoxyC₁₋₄alkoxy, C₂₋₆alkenyl, C₃₋₆alkynyl, haloC₂₋₆alkenyl, —C(O)N(R³)(R⁴), —C(O)N(R³)C₁₋₆alkoxy, —S(O)₂N(R³)(R⁴), —N(R³)(R⁴), —C(NOR⁵)R⁶, —N(R³)C(O)(R⁶), —N(R³)S(O)₂(R⁶), —C(O)R⁶, C(O)OR⁷, a heterocyclic ring, aroyl and aryl; wherein the heterocyclic ring, aroyl and aryl moieties are optionally substituted by one, two or three substituents independently selected from the group consisting of halogen, hydroxy, cyano, nitro, amino, C₁₋₆alkyl, haloC₁₋₆alkyl, C₁₋₆alkoxy and haloC₁₋₆alkoxy;
where present, each R is independently halogen, C₁₋₆alkyl, cyano, haloC₁₋₆alkyl, C₁₋₆alkanoyl, C₁₋₆alkoxy, hydroxy or trifluoromethoxy;
each R¹ is hydrogen, halogen, C₁₋₆alkyl, cyano, haloC₁₋₆alkyl, C₁₋₆alkanoyl, C₁₋₆alkoxy, hydroxy or C₁₋₆alkoxyC₁₋₆alkyl;
each R² is hydrogen, halogen, hydroxy, cyano, nitro, C₁₋₆alkyl, haloC₁₋₆alkyl, C₃₋₇cycloalkyl, arylC₁₋₆alkoxy, C₁₋₆alkylthio, C₁₋₆alkoxy, haloC₁₋₆alkoxy, C₁₋₆alkoxyC₁₋₆alkyl, C₁₋₆alkoxyC₁₋₄alkoxy, C₂₋₆alkenyl, C₃₋₆alkynyl, haloC₂₋₆alkenyl, ═O, —C(O)N(R³)(R⁴), —C(O)N(R³)C₁₋₆alkoxy, —S(O)₂N(R³)(R⁴), —N(R³)(R⁴), —C(NOR⁵)R⁶, —N(R³)C(O)(R⁶), —N(R³)S(O)₂(R⁶), —C(O)R⁶, —C(O)OR⁷, —C(O)NHNHC(O)R⁶, a heterocyclic ring, aroyl or aryl; wherein the heterocyclic ring, aroyl or aryl moieties are optionally substituted by one, two or three substituents independently selected from the group consisting of halogen, hydroxy, cyano, nitro, amino, C₁₋₆alkyl, haloC₁₋₆alkyl, C₁₋₆alkoxy and haloC₁₋₆alkoxy;
R³ and R⁴ are independently hydrogen; C₁₋₆alkyl; aryl; C₃₋₇cycloalkyl; C₃₋₇cycloalkylC₁₋₆alkyl; or where R³ and R⁴ are connected to the same nitrogen atom, together with the nitrogen, they form a 4-, 5-, 6- or 7-membered ring optionally containing one additional O, N or S ring-atom;
R⁵ is C₁₋₄alkyl, C₃₋₇cycloalkylC₁₋₆alkyl or C₃₋₇cycloalkyl;
R⁶ is hydrogen, halogen, cyano, C₃₋₇cycloalkylC₁₋₆alkyl, C₃₋₇cycloalkyl or C₁₋₆alkyl;
R⁷ and R⁸ are independently hydrogen, C₁₋₆alkyl, C₃₋₇cycloalkyl, C₃₋₇cycloalkylC₁₋₆alkyl or haloC₁₋₆alkyl;
R⁹ and R¹⁰ are independently hydrogen, C₁₋₆alkyl, cyano, haloC₁₋₆alkyl, C₁₋₆alkanoyl, C₁₋₆alkoxy, hydroxyl, halogen or C₁₋₆alkoxyC₁₋₆alkyl;
p is 0, 1 or 2;
r is 0, 1, 2 or 3;
s is 0, 1, 2 or 3; and
t is 0, 1 or 2.

The term “5-membered heteroaromatic ring” means a 5-membered aromatic ring containing at least one ring-nitrogen atom and optionally containing 1 to 3 additional ring-heteroatoms independently selected from oxygen, nitrogen and sulphur. Examples of 5-membered heteroaromatic rings are pyrrole, imidazole, pyrazole, triazole, oxadadiazole and tetrazole.

The term “5-membered heterocyclic ring” means a 5-membered heterocyclic ring which is partially or fully saturated, i.e. not aromatic, containing at least one ring-nitrogen atom and optionally containing 1 to 3 additional ring-heteroatoms selected from oxygen, nitrogen and sulphur. Examples of such rings that are partially saturated include oxazoline, isoxazoline, imidazoline, pyrroline and pyrazoline. Examples of such rings that are fully saturated include pyrrolidine, imidazolidine and oxadiazoline.

The term “heterocyclic ring” means a 5 or 6-membered monocyclic ring which is partially or fully saturated, wherein up to 5 of the carbon atoms are replaced by a heteroatom independently selected from O, S and N. Examples of “heterocyclic rings” which are fully saturated 5 or 6-membered monocyclic rings are pyrrolidine, imidazolidine, pyrazolidine, tetrahydrofuran, dioxolan, piperidine, piperazine, morpholine, thiomorpholine, tetrahydrothiophene, dioxan, tetrahydro-2H-pyran and dithiane. Examples of “heterocyclic rings” which are partially saturated 5 or 6-membered monocyclic rings are oxazoline, isoxazoline, imidazoline, pyrazoline, 1,2,3,6-tetrahydropyridine and 3,6-dihydro-2H-pyran.

The term “aryl”, whether alone or as part of another group, is intended, unless otherwise stated, to denote an aromatic carbocyclic ring or a heteroaromatic ring. Examples of aryl groups are phenyl, pyrrolyl, imidazolyl, pyrazolyl, oxadiazolyl, isothiazolyl, oxazolyl, thiazolyl, thiazinyl, furyl, thienyl, pyridyl, pyridazinyl, pyrimidinyl, azepinyl and naphthyl.

The term “C₁₋₆alkyl”, whether alone or part of another group, means an alkyl group having from one to six carbon atoms, in all isomeric forms, including methyl, ethyl, propyl, isopropyl, butyl, isobutyl, sec-butyl, tert-butyl, pentyl, neopentyl, sec-pentyl, n-pentyl, isopentyl, tert-pentyl and hexyl.

The term “halogen” and its abbreviation “halo” is used herein to describe, unless otherwise stated, a group selected from fluorine; chlorine, bromine and iodine.

The term “haloC₁₋₆alkyl” means an C₁₋₆alkyl groups with one or more halo substituents, for example CF₃.

The term “C₁₋₆alkanoyl” means an alkanoyl group having from 1 to 6 carbon atoms, such as methanoyl (or “formyl”), ethanoyl (or “acetyl”), propanoyl, butanoyl, pentanoyl and hexanoyl.

The term “C₁₋₆alkoxy” means a straight chain or branched chain alkoxy (or “alkyloxy”) group having from one to six carbon atoms, such as methoxy, ethoxy, propoxy, isopropoxy, butoxy, isobutoxy, sec-butoxy, tert-butoxy, pentoxy, neopentoxy, sec-pentoxy, n-pentoxy, isopentoxy, tert-pentoxy and hexyloxy.

The term “3 to 7 membered cycloalkylene group” refers to cycloalkylene groups having from 3 to 7 carbons, such as cyclohexylene.

The term “3 to 7 membered cycloalkenylene group” means a cycloalkenylene groups having from 3 to 7 carbons, such as cyclohexenylene.

The term “C₁₋₆alkylthio” means a straight chain or branched chain alkylthio group having from one to six carbon atoms, such as methylthio, ethylthio, propylthio, isopropylthio, butylthio, isobutylthio, sec-butylthio, tert-butylthio, pentylthio, neopentylthio, sec-pentylthio, n-pentylthio, isopentylthio, tert-pentylthio and hexylthio.

The term “arylC₁₋₆alkoxy” refers to an aryl group which is linked by a C₁₋₆alkoxy group. Examples include phenylmethoxy, phenylethoxy, naphthymethoxy, naphthylethoxy, phenylpropoxy, naphthylpropoxy, phenylbutoxy and naphthylpentoxy.

The term “C₃₋₇cycloalkyl” refers to a cycloalkyl group consisting of from 3 to 7 carbon atoms, for example cyclopropane, cyclobutane, cyclopentane, cyclohexane and cycloheptane.

The term “aroyl” refers to a group having the formula “aryl-CO” wherein “aryl” is as defined above.

The term “C₂₋₆alkenyl” refers to an unsaturated hydrocarbon group containing one or more carbon-carbon double bonds and having from two to six carbon atoms, in all isomeric forms, such as ethenyl, propenyl, butenyl, pentenyl and hexenyl.

The term “C₂₋₆alkynyl” refers to an unsaturated hydrocarbon group containing one or more carbon-carbon triple bonds, having from two to six carbon atoms, in all isomeric forms, such as propynyl, butylidyne, pentenynyl and pentylidyne.

In an embodiment, ring Q is an imidazole, triazole (e.g. 1,2,3 triazole or 1,3,4-triazole) or tetrazole. In a further embodiment, ring Q is imidazole.

In an embodiment Y is C(R⁷), C(R⁷)(R⁸) or O wherein where the bond connecting B and Y is a single bond, Y is C(R⁷)(R⁸) or O and where the bond connecting B and Y is a double bond, B is C(R⁷). In a further embodiment, when Y is C(R⁷), C(R⁷)(R⁸) or O, R⁷ and R⁸ are independently hydrogen or C₁₋₆alkyl (particularly methyl or ethyl). In a still further embodiment, R⁷ and R⁸ are hydrogen.

In an embodiment Z¹ is —CH₂—, —(CH₂)₂—, —CH₂CH(CH₃)— (wherein the left hand side on linkage Z¹ is attached to the nitrogen atom) or —(CH₂)₃—. In a further embodiment Z¹ is —(CH₂)₂.

In an embodiment X is C(R¹) or N. In a further embodiment, when X is C(R¹), R¹ is hydrogen, halogen or C₁₋₆alkyl (such as methyl or ethyl). In a further embodiment, R¹ is hydrogen or methyl. In a further embodiment X is N or CH.

In an embodiment A is quinolyl or quinazolinyl, either of which are optionally substituted by 1-4 substituents, which substituents may be the same or different, and are selected from the group consisting of halogen, hydroxy, cyano, nitro, C₁₋₆alkyl, haloC₁₋₆alkyl, C₃₋₇cycloalkyl, arylC₁₋₆alkoxy, C₁₋₆alkylthio, C₁₋₆alkoxy, haloC₁₋₆alkoxy, C₁₋₆alkoxyC₁₋₆alkyl, C₁₋₆alkoxyC₁₋₄alkoxy, C₂₋₆alkenyl, C₃₋₆alkynyl, haloC₂₋₆alkenyl, —C(O)N(R³)(R⁴), —C(O)N(R³)C₁₋₆alkoxy, —S(O)₂N(R³)(R⁴), —N(R³)(R⁴), —C(NOR⁵)R⁶, —N(R³)C(O)(R⁶), —N(R³)S(O)₂(R⁶), —C(O)R⁶, C(O)OR⁷, a heterocyclic ring, aroyl and aryl; wherein the heterocyclic ring, aroyl and aryl moieties are optionally substituted by one, two or three substituents independently selected from the group consisting of halogen, hydroxy, cyano, nitro, amino, C₁₋₆alkyl, haloC₁₋₆alkyl, C₁₋₆alkoxy and haloC₁₋₆alkoxy. In a further embodiment, the substituents on A are selected from the group consisting of halogen, cyano, C₁₋₆alkyl, haloC₁₋₆alkyl, C₁₋₆alkoxy, haloC₁₋₆alkoxy, —C(O)N(R³)(R⁴) and —C(O)R⁶. In a further embodiment, the substituents on A are selected from the group consisting of halogen (such as fluoro or chloro), C₁₋₆alkyl (such as methyl, ethyl and propyl), cyano, trifluoromethyl, C₁₋₆alkoxy (such as methoxy, ethoxy or isopropoxy) and —C(O)R⁶. In a further embodiment, A is quinolyl and the substituents on A are selected from the group consisting of halogen, cyano, C₁₋₆alkyl, haloC₁₋₆alkyl, C₁₋₆alkoxy, haloC₁₋₆alkoxy and —C(O)N(R³)(R⁴). In a further embodiment, A is 5-quinolyl and the substituents on A are selected from the group consisting of halogen, cyano, haloC₁₋₆alkyl, C₁₋₆alkoxy, haloC₁₋₆alkoxy and —C(O)N(R³)(R⁴). In a further embodiment, A is 2-methyl-5-quinolyl.

In an embodiment, when present R is halogen (such as fluoro or chloro) or C₁₋₆alkyl (such as methyl or ethyl).

In an embodiment, when present R¹ is hydrogen or C₁₋₆alkyl (such as methyl or ethyl).

In an embodiment, each R² is hydrogen, cyano, C₁₋₆alkoxy, haloC₁₋₆alkyl, ═O, —C(O)N(R³)(R⁴), —C(O)N(R³)C₁₋₆alkoxy, —S(O)₂N(R³)(R⁴), —N(R³)(R⁴), —C(NOR⁵)R⁶, —N(R³)C(O)(R⁶), —N(R³)S(O)₂(R⁶), —C(O)R⁶, —C(O)OR⁷, —C(O)NHNHC(O)R⁶, a heterocyclic ring or aryl; wherein the heterocyclic ring or aryl moieties are optionally substituted by one, two or three substituents independently selected from the group consisting of halogen, cyano, nitro, amino, C₁₋₆alkyl, haloC₁₋₆alkyl, C₁₋₆alkoxy and haloC₁₋₆alkoxy. In a further embodiment, each R² is hydrogen, cyano, C₁₋₆alkyl, ═O, —C(O)N(R³)(R⁴), —C(O)N(R³)C₁₋₆alkoxy, —C(NOR⁵)R⁶, —N(R³)C(O)(R⁶), —C(O)R⁶, —C(O)OR⁷, —C(O)NHNHC(O)R⁶, a heterocyclic ring or aryl; wherein the heterocyclic ring or aryl moieties are optionally substituted by one, two or three substituents independently selected from the group consisting of halogen, cyano, nitro, amino, C₁₋₆alkyl, haloC₁₋₆alkyl, C₁₋₆alkoxy and haloC₁₋₆alkoxy. In a further embodiment, each R² is C₁₋₆alkyl, —C(O)N(R³)(R⁴) or —C(O)R⁶.

In an embodiment s is 1 or 2.

In an embodiment the invention provides a compound of formula (I), a salt or prodrug thereof wherein
— represents independently a single or double bond;
ring Q is an imidazole, triazole (e.g. 1,2,3 triazole or 1,3,4-triazole) or tetrazole;
B is C(R⁷)(R⁸) or C(R⁷), wherein where the bond connecting B and Y is a single bond B is C(R⁷)(R⁸) and when the bond connecting B and Y is a double bond B is C(R⁷);
Y is C(R⁷), C(R⁷)(R⁸) or O wherein where the bond connecting B and Y is a single bond, Y is C(R⁷)(R⁸) or O and where the bond connecting B and Y is a double bond, B is C(R⁷);
Z¹ is —CH₂—, —(CH₂)₂—, —CH₂ CH(CH₃)— (wherein the left hand side of linkage Z¹ is attached to the nitrogen atom) or —(CH₂)₃—;
X is C(R¹) or N;
A is quinolyl or quinazolinyl, either of which are optionally substituted by 1-4 substituents, which substituents may be the same or different, and are selected from the group consisting of halogen, hydroxy, cyano, nitro, C₁₋₆alkyl, haloC₁₋₆alkyl, C₃₋₇cycloalkyl, arylC₁₋₆alkoxy, C₁₋₆alkylthio, C₁₋₆alkoxy, haloC₁₋₆alkoxy, C₁₋₆alkoxyC₁₋₆alkyl, C₁₋₆alkoxyC₁₋₄alkoxy, C₂₋₆alkenyl, C₃₋₆alkynyl, haloC₂₋₆alkenyl, —C(O)N(R³)(R⁴), —C(O)N(R³)C₁₋₆alkoxy, —S(O)₂N(R³)(R⁴), —N(R³)(R⁴), —C(NOR⁵)R⁶, —N(R³)C(O)(R⁶), —N(R³)S(O)₂(R⁶), —C(O)R⁶, C(O)OR⁷, a heterocyclic ring, aroyl and aryl; wherein the heterocyclic ring, aroyl and aryl moieties are optionally substituted by one, two or three substituents independently selected from the group consisting of halogen, hydroxy, cyano, nitro, amino, C₁₋₆alkyl, haloC₁₋₆alkyl, C₁₋₆alkoxy and haloC₁₋₆alkoxy;
when present R is halogen (such as fluoro or chloro) or C₁₋₆alkyl (such as methyl or ethyl).
when present R¹ is hydrogen or C₁₋₆alkyl (such as methyl or ethyl);
each R² is hydrogen, cyano, C₁₋₆alkyl, C₁₋₆alkoxy, haloC₁₋₆alkyl, ═O, —C(O)N(R³)(R⁴), —C(O)N(R³)C₁₋₆alkoxy, —S(O)₂N(R³)(R⁴), —N(R³)(R⁴), —C(NOR⁵)R⁶, —N(R³)C(O)(R⁶), —N(R³)S(O)₂(R⁶), —C(O)R⁶, —C(O)OR⁷, —C(O)NHNHC(O)R⁶, a heterocyclic ring or aryl; wherein the heterocyclic ring or aryl moieties are optionally substituted by one, two or three substituents independently selected from the group consisting of halogen, cyano, nitro, amino, C₁₋₆alkyl, haloC₁₋₆alkyl, C₁₋₆alkoxy and haloC₁₋₆alkoxy;
R³ and R⁴ are independently hydrogen; C₁₋₆alkyl; aryl; C₃₋₇cycloalkyl; C₃₋₇cycloalkylC₁₋₆alkyl; or where R³ and R⁴ are connected to the same nitrogen atom, together with the nitrogen, they form a 4-, 5-, 6- or 7-membered ring optionally containing one additional O, N or S ring-atom;
R⁵ is C₁₋₄alkyl, C₃₋₇cycloalkylC₁₋₆alkyl or C₃₋₇cycloalkyl;
R⁶ is hydrogen, halogen, cyano, C₃₋₇cycloalkylC₁₋₆alkyl, C₃₋₇cycloalkyl or C₁₋₆alkyl;
R⁷ and R⁸ are independently hydrogen, C₁₋₆alkyl, C₃₋₇cycloalkyl, C₃₋₇cycloalkylC₁₋₆alkyl or haloC₁₋₆alkyl;
R⁹ and R¹⁰ are independently hydrogen, C₁₋₆alkyl, cyano, haloC₁₋₆alkyl, C₁₋₆alkanoyl, C₁₋₆alkoxy, hydroxyl, halogen or C₁₋₆alkoxyC₁₋₆alkyl;
p is 0, 1 or 2;
r is 0, 1, 2 or 3;
s is 0, 1 or 2; and
t is 0, 1 or 2.

In an embodiment the invention provides a compound of formula (I), a salt or prodrug thereof wherein
— represents independently a single or double bond;
ring Q is an imidazole, triazole (e.g. 1,2,3 triazole or 1,3,4-triazole) or tetrazole;
B is C(R⁷)(R⁸) or C(R⁷), wherein where the bond connecting B and Y is a single bond B is C(R⁷)(R⁸) and when the bond connecting B and Y is a double bond B is C(R⁷);
Y is C(R⁷), C(R⁷)(R⁸) or O wherein where the bond connecting B and Y is a single bond, Y is C(R⁷)(R⁸) or O and where the bond connecting B and Y is a double bond, B is C(R⁷);
Z¹ is —(CH₂)₂—;
X is CH or N;
A is quinolyl or quinazolinyl, either of which are optionally substituted by 1-4 substituents, which substituents may be the same or different, and are selected from the group consisting of halogen, cyano, C₁₋₆alkyl, haloC₁₋₆alkyl, C₁₋₆alkoxy, haloC₁₋₆alkoxy, —C(O)N(R³)(R⁴) and —C(O)R⁶;
when present R is halogen (such as fluoro or chloro) or C₁₋₆alkyl (such as methyl or ethyl).
when present R¹ is hydrogen or C₁₋₆alkyl (such as methyl or ethyl);
each R² is hydrogen, cyano, C₁₋₆alkyl, ═O, —C(O)N(R³)(R⁴), —C(O)N(R³)C₁₋₆alkoxy, —C(NOR⁵)R⁶, —N(R³)C(O)(R⁶), —C(O)R⁶, —C(O)OR⁷, —C(O)NHNHC(O)R⁶, a heterocyclic ring or aryl; wherein the heterocyclic ring or aryl moieties are optionally substituted by one, two or three substituents independently selected from the group consisting of halogen, cyano, nitro, amino, C₁₋₆alkyl, haloC₁₋₆alkyl, C₁₋₆alkoxy and haloC₁₋₆alkoxy;
R³ and R⁴ are independently hydrogen; C₁₋₆alkyl; aryl; C₃₋₇cycloalkyl; C₃₋₇cycloalkylC₁₋₆alkyl; or where R³ and R⁴ are connected to the same nitrogen atom, together with the nitrogen, they form a 4-, 5-, 6- or 7-membered ring optionally containing one additional O, N or S ring-atom;
R⁵ is C₁₋₄alkyl, C₃₋₇cycloalkylC₁₋₆alkyl or C₃₋₇cycloalkyl;
R⁶ is hydrogen, halogen, cyano, C₃₋₇cycloalkylC₁₋₆alkyl, C₃₋₇cycloalkyl or C₁₋₆alkyl;
R⁷ and R⁸ are independently hydrogen, C₁₋₆alkyl, C₃₋₇cycloalkyl, C₃₋₇cycloalkylC₁₋₆alkyl or haloC₁₋₆alkyl;
p is 0, 1 or 2;
r is 0, 1, 2 or 3; and
s is 0, 1 or 2.

In an embodiment, the compounds of formula (I) are selected from the list consisting of:
6-{2-[4-(2-methyl-5-quinolinyl)-1-piperazinyl]ethyl}-4H-imidazo[5,1-c][1,4]benzoxazine-3-carboxamide dihydrochloride (Example 14);
N-methyl-6-{2-[4-(2-methyl-5-quinolinyl)-1-piperazinyl]ethyl}-4H-imidazo[5,1-c][1,4]benzoxazine-3-carboxamide dihydrochloride (Example 15);
6-{2-[4-(2-methyl-5-quinolinyl)-1-piperazinyl]ethyl}-3-(4-morpholinyl carbonyl)-4H-Imidazo[5,1-c][1,4]benzoxazine dihydrochloride (Example 24);
N-methyl-6-{2-[4-(2-methyl-5-quinolinyl)-1-piperidinyl]ethyl}-4,5-dihydroimidazo[1,5-a]quinoline-3-carboxamide dihydrochloride (Example 83);
N-methyl-6-{2-[4-(2-methyl-5-quinolinyl)-1-piperidinyl]ethyl}imidazo[1,5-a]quinoline-3-carboxamide dihydrochloride (Example 89);
6-{2-[4-(2-methyl-5-quinolinyl)-1-piperidinyl]ethyl}imidazo[1,5-a]quinoline-3-carboxamide dihydrochloride (Example 91);
6-{2-[(2R)-2-methyl-4-(2-methyl-5-quinolinyl)-1-piperazinyl]ethyl}tetrazolo[1,5-a]quinoline dihydrochloride (Example 94);
ethyl 6-{2-[4-(2-methyl-5-quinolinyl)-1-piperazinyl]ethyl}[1,2,3]triazolo[1,5-a]quinoline-3-carboxylate dihydrochloride (Example 97);
6-{2-[(2R)-2-methyl-4-(2-methyl-5-quinolinyl)-1-piperazinyl]ethyl}-3-(3-methyl-1,2,4-oxadiazol-5-yl)imidazo[1,5-a]quinoline dihydrochloride (Example 105);
6-{2-[4-(2-methyl-5-quinolinyl)-1-piperidinyl]ethyl}[1,2,3]triazolo[1,5-a]quinoline-3-carboxamide dihydrochloride (Example 109);
6-{2-[4-(2-methyl-5-quinolinyl)-1-piperidinyl]ethyl}-4,5-dihydroimidazo[1,5-a]quinoline-3-carboxamide dihydrochloride (Example 122);
N,7-dimethyl-6-{2-[4-(2-methyl-5-quinolinyl)-1-piperidinyl]ethyl}-4H-imidazo[5,1-c][1,4]benzoxazine-3-carboxamide dihydrochloride (Example 124); and
7-methyl-6-{2-[4-(2-methyl-5-quinolinyl)-1-piperidinyl]ethyl}-4H-imidazo[5,1-c][1,4]benzoxazine-3-carboxamide dihydrochloride (Example 125);
   or other pharmaceutical acceptable salts or free bases thereof.

In a further embodiment the compound is 6-{2-[4-(2-methyl-5-quinolinyl)-1-piperazinyl]ethyl}-4H-imidazo[5,1-c][1,4]benzoxazine-3-carboxamide dihydrochloride (Example 14).

In a further embodiment the compound is 6-{2-[4-(2-methyl-5-quinolinyl)-1-piperazinyl]ethyl}-4H-imidazo[5,1-c][1,4]benzoxazine-3-carboxamide (Example 14 free base).

In a further embodiment the compound is 6-{2-[4-(2-methyl-5-quinolinyl)-1-piperidinyl]ethyl}imidazo[1,5-a]quinoline-3-carboxamide dihydrochloride (Example 91).

In a further embodiment the compound is 6-{2-[4-(2-methyl-5-quinolinyl)-1-piperidinyl]ethyl}[1,2,3]triazolo[1,5-a]quinoline-3-carboxamide dihydrochloride (Example 109).

According to a further aspect, the invention provides a compound of formula (I) or a pharmaceutically acceptable salt thereof:

wherein
A is indolyl, quinolyl, quinazolinyl, benzofuranyl or benzothienyl, which groups are optionally substituted by 1-4 substituents, which substituents may be the same or different, and which are selected from the group consisting of halogen, hydroxy, cyano, nitro, C₁₋₆alkyl, haloC₁₋₆alkyl, C₃₋₇cycloalkyl, arylC₁₋₆alkoxy, C₁₋₆alkanoyl, C₁₋₆alkylthio, C₁₋₆alkoxy, haloC₁₋₆alkoxy, C₁₋₆alkoxyC₁₋₆alkyl, C₁₋₆alkoxyC₁₋₄alkoxy, C₁₋₆alkenyl, C₃₋₆alkynyl, haloC₁₋₆alkenyl, C(O)N(R³)(R⁴), C(O)N(R³)C₁₋₆alkoxy, S(O)₂N(R³)(R⁴), N(R³)(R⁴); C(NOR⁵)R⁶, N(R³)C(O)(R⁴), N(R³)S(O)₂(R⁴), C(O)R⁷, C(O)OR⁷, heterocyclic, aroyl or aryl wherein the aroyl or heterocyclic moiety is optionally substituted by one, two or three substituents selected from the group consisting of halogen, hydroxy, cyano, nitro, amino, C₁₋₆alkyl, haloC₁₋₆alkyl, C₁₋₆alkoxy, haloC₁₋₆alkoxy;
B is C(R⁷)(R⁸) or C(R⁷);
X is C(R¹), N or C;
Y is C(R⁷), C(R⁷)(R⁸), O or S(O)ₜ;
Z¹ is a group

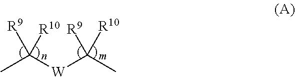

wherein n and m are independently 0, 1 or 2 and W is 3 to 7 membered cycloalkylene group or 3 to 7 membered cycloalkenylene group which groups are optionally substituted by 1 to 3 substituents which may be the same or different, and which are selected from halogen hydroxy, cyano, C₁₋₆alkyl, haloC₁₋₆alkyl, C₁₋₆alkanoyl, or C₁₋₆alkoxy or W is —(CH═CH)—, —C(═O)—, —C(═CH₂)—, —C(R⁷)(R⁸)—, C(R⁷)(R⁸)—S(O)ₜ— or —C(R⁷)(R⁸)—O—;
ring Q is a 5 membered heteroaromatic ring or a 5 membered heterocyclic ring containing at least one nitrogen and optionally containing 1 to 3 additional heteroatoms selected from oxygen, nitrogen and sulphur;
— represents independently a single or double bond provided that when:
(a) X is N or C(R¹), the bond — linked to X is a single bond
(b) Y is C(R²)(R³), N(R²), O or S(O)ₜ, the bond — linked to Y is a single bond;
R is independently halogen, C₁₋₆alkyl, cyano, haloC₁₋₆alkyl, C₁₋₆alkanoyl, C₁₋₆alkoxy, hydroxy or trifluoromethoxy;
R¹ (a) is hydrogen, C₁₋₆alkyl, cyano, haloC₁₋₆alkyl, C₁₋₆alkanoyl, C₁₋₆alkoxy hydroxy or C₁₋₆alkoxyC₁₋₆alkyl;
R² is hydrogen, halogen, hydroxy, cyano, nitro, C₁₋₆alkyl, haloC₁₋₆alkyl, C₃₋₇cycloalkyl, arylC₁₋₆alkoxy, C₁₋₆alkanoyl, C₁₋₆alkylthio, C₁₋₆alkoxy, haloC₁₋₆alkoxy, C₁₋₆alkoxyC₁₋₆alkyl, C₁₋₆alkoxyC₁₋₄alkoxy, C₁₋₆alkenyl, C₃₋₆alkynyl, haloC₁₋₆alkenyl, —O, C(O)N(R³)(R⁴), C(O)N(R³)C₁₋₆alkoxy, S(O)₂N(R³)(R⁴), N(R³)(O); C(NOR⁵)R⁶, N(R³)C(O)(R⁴), N(R³)S(O)₂(R⁴), C(O)R⁷, C(O)OR⁷, heterocyclic, aroyl or aryl wherein the aroyl or heterocyclic moiety is optionally substituted by one, two or three substituents selected from the group consisting of halogen, hydroxy, cyano, nitro, amino, C₁₋₆alkyl, haloC₁₋₆alkyl, C₁₋₆alkoxy, haloC₁₋₆alkoxy;
R³ and R⁴ are independently hydrogen, C₁₋₆alkyl, aryl, C₃₋₇cycloalkyl, C₃₋₇cycloalkylC₁₋₆alkyl or NR³R⁴ together N or form a 4-, 5-, 6- or 7-membered azacyclic group optionally containing one additional O, N or S atom in the azacycle and having 3-8 carbon atoms (including the carbon atoms contained in any optional substituent(s) of the azacycle);
R⁵ is C₁₋₄alkyl, C₃₋₇cycloalkylC₁₋₆alkyl or C₃₋₇cycloalkyl;
R⁶ is hydrogen, halogen, cyano, C₃₋₇cycloalkylC₁₋₆alkyl or C₃₋₇cycloalkyl or C₁₋₆alkyl;
R⁷ and R⁸ are independently hydrogen, C₁₋₆alkyl, or haloC₁₋₆alkyl;
R⁹ and R¹⁰ are independently hydrogen, C₁₋₆alkyl, cyano, haloC₁₋₆alkyl, C₁₋₆alkanoyl, C₁₋₆alkoxy, hydroxyl, halogen or C₁₋₆alkoxyC₁₋₆alkyl;
p is 0, 1 or 2;
r is 1, 2 or 3;
s is 1, 2 or 3;
t is 0, 1 or 2.

For the avoidance of doubt, unless otherwise indicated, the term substituted means substituted by one or more defined groups. In the case where groups may be selected from a number of alternative groups, the selected groups may be the same or different.

For the avoidance of doubt, the term independently means that where more than one substituent is selected from a number of possible substituents, those substituents may be the same or different.

The compounds of formula (I) may form acid or base addition salts. It will be appreciated that for use in medicine the salts should be pharmaceutically acceptable. Suitable pharmaceutically acceptable salts will be apparent to those skilled in the art and include those described in Berge et al, J. Pharm. Sci., 1977, 66, 1-19. Where the compounds of formula (I) contain a basic centre, they may form acid addition salts with suitable inorganic or organic acids. Examples of suitable inorganic acids are hydrochloric, hydrobromic, sulfuric, nitric, hydroiodic, metaphosphoric, or phosphoric acid. Examples of suitable organic acids are succinic, maleic, acetic, fumaric, citric, tartaric, benzoic, trifluoroacetic, malic, lactic, formic, propionic, glycolic, gluconic, camphorsulfuric, isothionic, mucic, gentisic, isonicotinic, saccharic, glucuronic, furoic, glutamic, ascorbic, anthranilic, salicylic, phenylacetic, mandelic, embonic (pamoic), ethanesulfonic, pantothenic, stearic, sulfinilic, alginic, galacturonic and arylsulfonic acids (for example benzenesulfonic, p-toluenesulfonic, methanesulfonic or naphthalenesulfonic acid). Where the compounds of formula (I) contains an acidic centre, they can form base addition salts with alkali metals, alkaline earth metals and suitable organic bases. Examples of suitable organic bases are N,N-dibenzylethylenediamine, chloroprocaine, choline, diethanolamine, ethylenediamine, meglumine (N-methylglucamine), lysine and procaine. Certain compounds of formula (I) may form acid addition salts with less than one equivalent, one equivalent or more than one equivalent of an acid (eg a dihydrochloride salt). The salts of compounds of formula (I) include all possible stoichiometric and non-stoichiometric forms. Salts having a non-physiologically acceptable anion or cation are within the scope of the invention, as they may be useful intermediates for the preparation of physiologically acceptable salts and/or for use in non-therapeutic, for example, in vitro, situations. In an embodiment, the salt is a physiologically acceptable salt.

It will be appreciated by those skilled in the art that certain protected derivatives of compounds of formula (I), which may be made prior to a final deprotection stage, may not possess pharmacological activity as such, but may, in certain instances, be administered and thereafter metabolised in the body to form compounds of the invention which are pharmacologically active. Such derivatives may therefore be described as “prodrugs”. Further, certain compounds of formula (I) may act as prodrugs of other compounds of formula (I). All protected derivatives and prodrugs of compounds of formula (I) are included within the scope of the invention. Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within compounds of the invention. In an embodiment, prodrugs for compounds of the invention include: esters, carbonate esters, hemi-esters, phosphate esters, nitro esters, sulfate esters, sulfoxides, amides, carbamates, azo-compounds, phosphamides, glycosides, ethers, acetals and ketals.

Hereinafter, the compounds of formula (I), their salts and prodrugs defined in any aspect of the invention (except intermediate compounds in chemical processes) are referred to as “compounds of the invention”.

The compounds of the invention may be prepared in crystalline or non-crystalline form. If crystalline, they may be hydrated or solvated. This invention includes within its scope stoichiometric hydrates or solvates as well as compounds containing variable amounts of water and/or solvent.

The compounds of the invention, may also exist in various polymorphic forms.

Certain compounds of the invention are capable of existing in stereoisomeric forms (e.g. geometric or (“cis-trans”) isomers, diastereomers and enantiomers) and the invention extends to each of these stereoisomeric forms and to mixtures thereof including racemates. The different stereoisomeric forms may be separated by methods known to the skilled chemist, or any given isomer may be obtained by stereospecific or asymmetric synthesis.

The invention also extends to any tautomeric forms and mixtures thereof. The present invention includes within its scope all such isomers, including mixtures.

The invention also includes all suitable isotopic variations of a compound of the invention. An isotopic variation of a compound of the invention is defined as one in which at least one atom is replaced by an atom having the same atomic number but an atomic mass different from the atomic mass usually found in nature. Examples of isotopes that can be incorporated into compounds of the invention include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the invention, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are suitable for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be suitable in some circumstances. Isotopic variations of the compounds of the invention can generally be prepared by conventional procedures such as by the illustrative methods or by the preparations described in the Examples and Descriptions hereafter using appropriate isotopic variations of suitable reagents.

Throughout the specification, general formulae are designated by Roman numerals (I), (II), (III), (IV) etc. Subsets of these general formulae are defined as (Ia), (Ib), (Ic) etc. . . . (IVa), (IVb), (IVc) etc.

Compounds of formula (Ia), i.e. compounds of general formula (I) where Z¹ is —CH(R⁷)—[CH(R⁹)]ₘ— (wherein m is 0, 1 or 2), may be prepared according to reaction scheme 1. Compounds of formula (II) are reacted with compounds of formula (III) in the presence of a suitable reducing agent, such as sodium triacetoxyborohydride. The reaction is typically carried out in an aprotic solvent (such as an ether, e.g. tetrahydrofuran, a halohydrocarbon, e.g. 1,2 dichloroethane, N,N-dimethylformamide or acetonitrile) at room temperature.

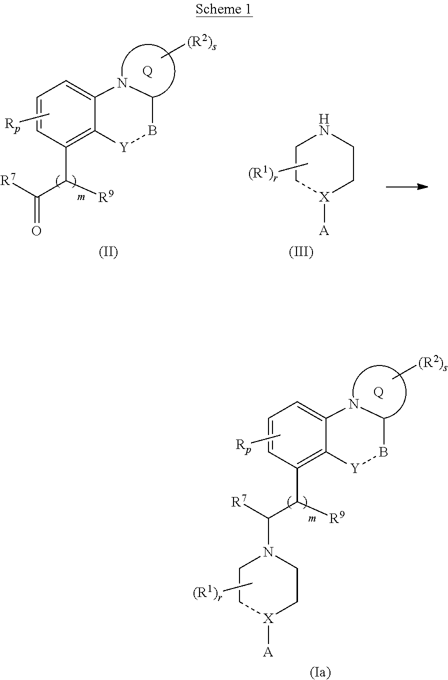

A number of compounds of formula (III) are known (see Published International patent application WO2004/046124) or may be prepared by similar procedures to those described in WO2004/046124. Other compounds of formula (III) may be prepared using similar procedures to those described in Descriptions 47, 116, 142, 143, 146, 150, 151 and 163.

Compounds of formula (II) may be prepared by oxidative cleavage of compounds of formula (IV) according to reaction scheme 2. Oxidative cleavage may be carried out using catalytic osmium tetroxide in aqueous tetrahydrofuran followed by addition of sodium periodate at room temperature. Alternative methods include ozonolysis using ozone followed by treatment with a suitable reducing agent (such as dimethyl sulphide).

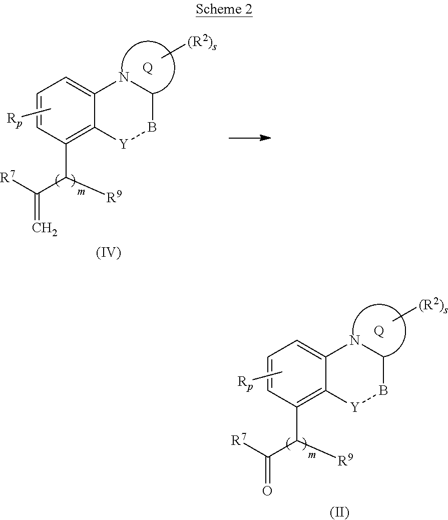

Compounds of formula (IVa) may be prepared from compounds of formula (V) by a reaction with diethyl chlorophosphate in the presence of a base (e.g potassium t-butoxide) at low temperature (such as 120 degC) followed by addition of C₁₋₆alkylisocyanoacetate and a base (e.g potassium t-butoxide) (see reaction scheme 3). The reaction is conveniently carried out in an aprotic solvent such as N,N-dimethylformamide.

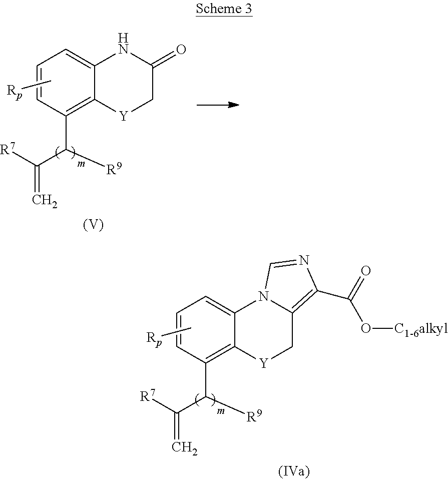

Compounds of formula (IVb) may be prepared according to reaction scheme 4. Compounds of formula (V) are treated with a suitable base (eg sodium hydride) in a suitable solvent (eg DMF) followed by treatment with R²C(O)CHR²Cl with cooling (eg at 0 degC) to give compounds of formula (VI). Treatment of compounds of formula (VI) with ammonium acetate in acetic acid under microwave radiation gives compounds of formula (IVb).

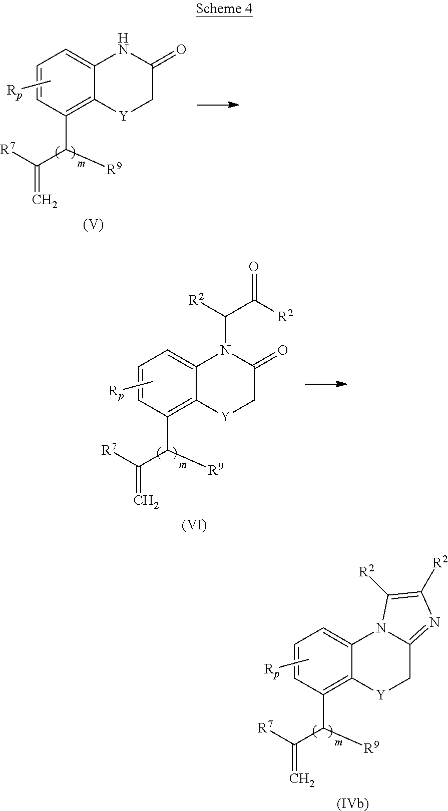

Compounds of formula (IVc) may be prepared according to reaction scheme 5. Compounds of formula (V) are reacted with Lawesson's reagent in toluene at elevated temperature (eg toluene/reflux) to give compounds of formula (VIIa). Treatment of (VIIa) with methyl iodide in acetone in the presence of a suitable base (eg potassium hydroxide), followed by treatment with aminoacetaldehyde dimethyl acetal in dry ethanol gives compounds of formula (VIII). Cyclisation under acidic conditions (eg concentrate hydrochloric acid in methanol) gives compounds of formula (IVc).

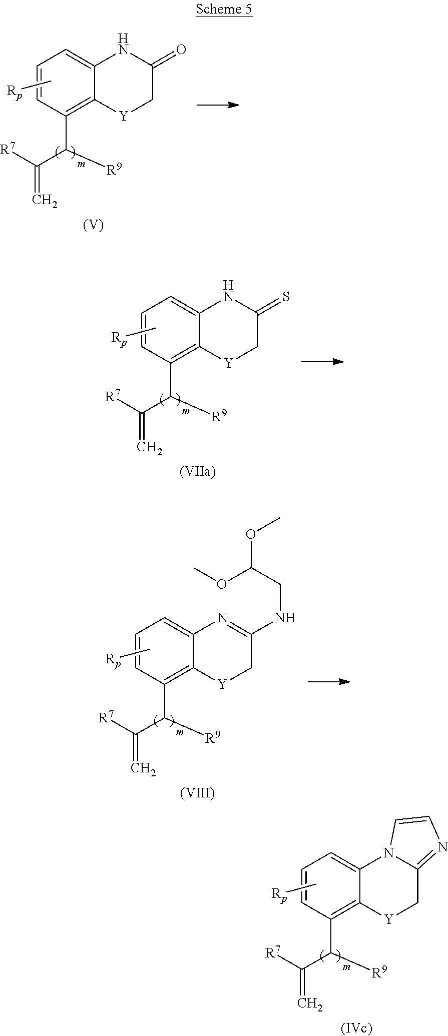

Compounds of formula (IVd) may be prepared from compounds of formula (VII) according to reaction scheme 6. Treatment of (VII) with hydrazine monohydrate in ethanol at elevated temperature (eg 80 degC) followed by treatment with (MeO)₃R² under microwave irradiation gives compounds of formula (IVd).

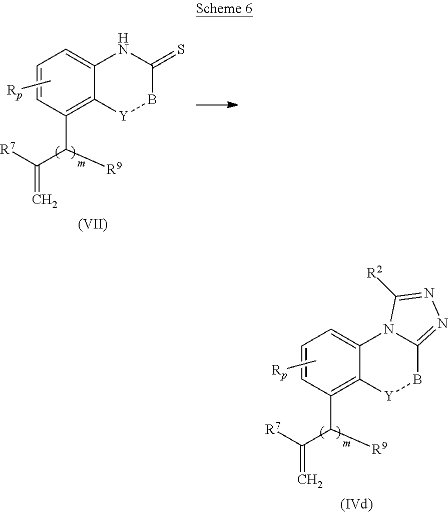

Compounds of formula (IVe) may be prepared according to reaction scheme 7. Compounds of formula (VII) are treated with hydroxylamine hydrochloride and sodium acetate in dry ethanol to give compounds of formula (IX). Compounds of formula (IX) are then treated with carbonyldiimidazole in dry tetrahydrofuran to give compounds of formula (IVe).

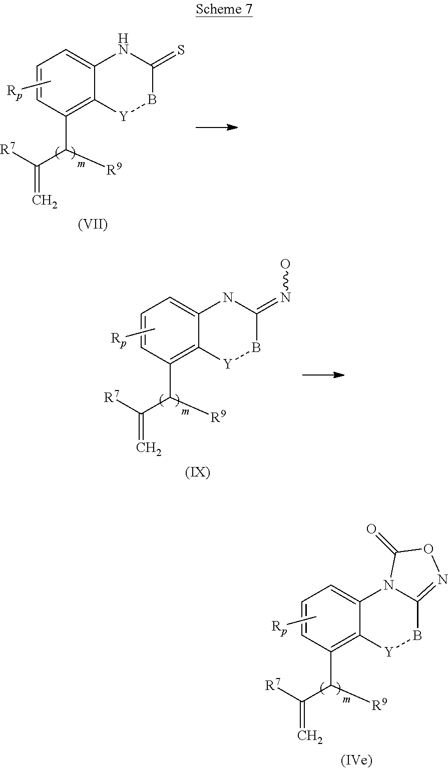

Compounds of formula (IVf) may be prepared by intramolecular 1,3-dipolar cycloaddition of compounds of formula (X) according to reaction scheme 8. The reaction is conveniently carried out in an organic solvent (e.g toluene).

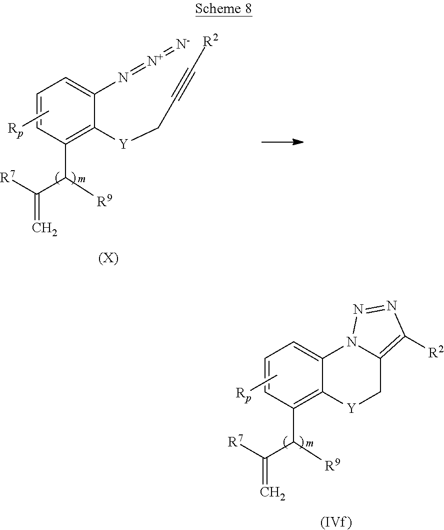

Compounds of formula (X) may be prepared by similar procedures to those described in Descriptions 2, 37, 38 and 39.

Compounds of formula (IVg) may be prepared according to reaction scheme 9 from compounds of formula (VIIa). Typical reaction conditions are described in Descriptions 19, 131, 133 and 135 hereinafter.

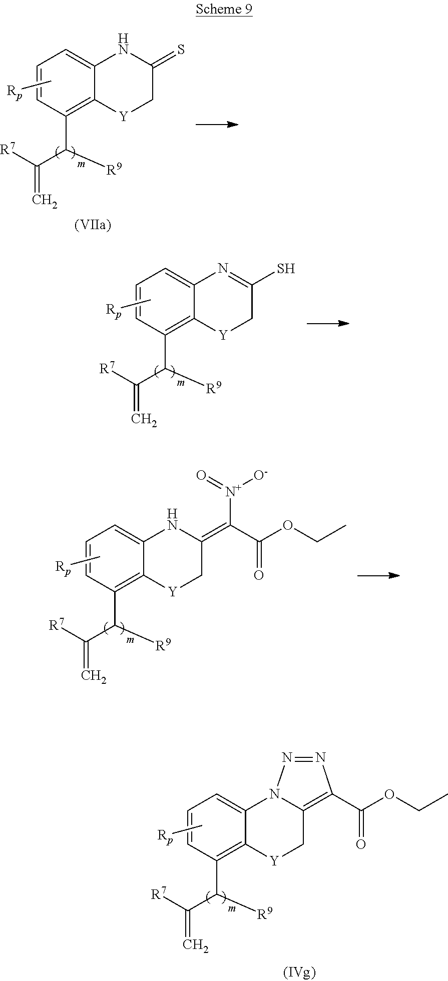

Compounds of formula (IVh) may be prepared according to reaction scheme 10 from compounds of formula (XI). Typical reaction conditions are described in Descriptions 87, 88, 89, 90, 107 and 108.

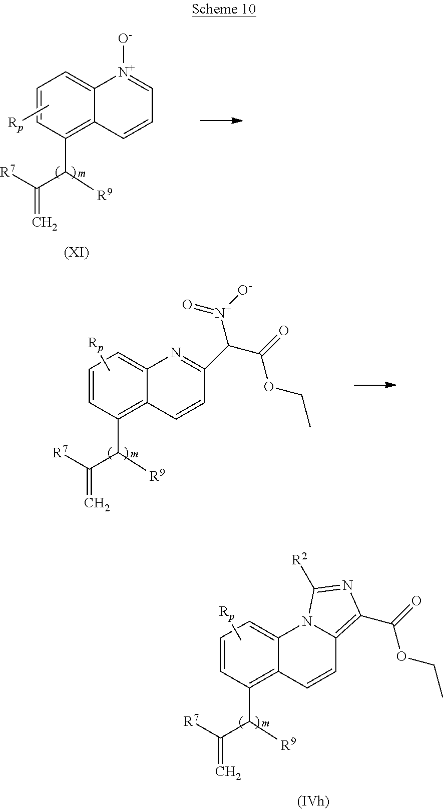

Compounds of formula (IVi) may be prepared according to reaction scheme 11 from compounds of formula (XI). Typical reaction conditions are described in Descriptions 88 and 92.

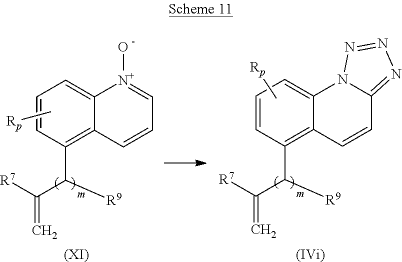

Compounds of formula (IV) may be prepared according to reaction scheme 12 from compounds of formula (XI). Typical reaction conditions are described in Descriptions 88, 94, 95 and 96.

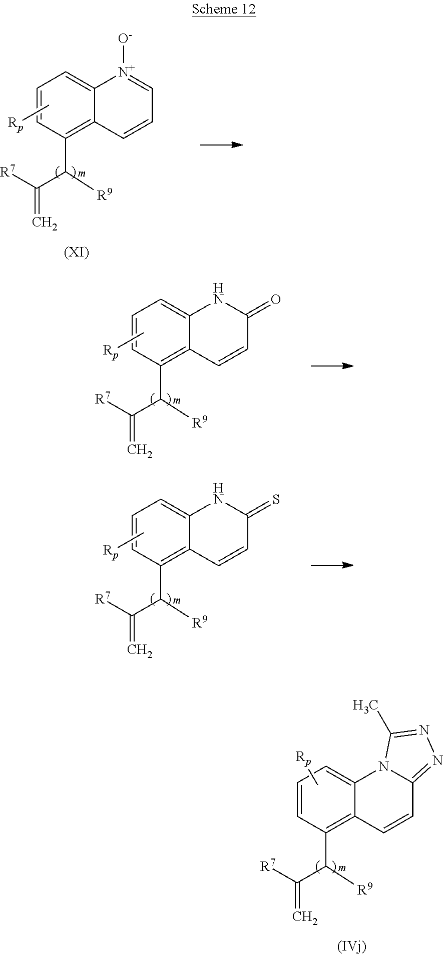

Compounds of formula (IVk) may be prepared according to reaction scheme 13 from compounds of formula (XI). Typical reaction conditions are as described in Descriptions 101, 102 and 103.

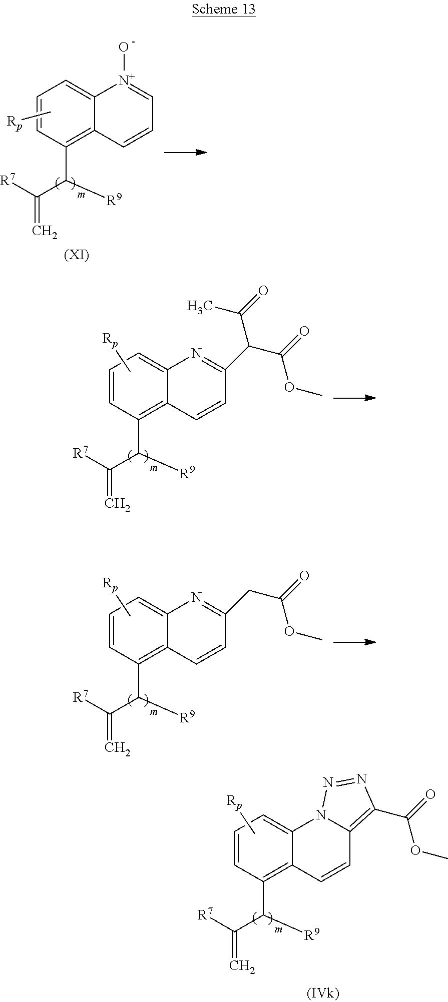

Compounds of formula (XI) may be prepared according to similar procedures to the one described for Description 88.

Compounds of formula (V) (see reaction schemes 3, 4 and 5) may be prepared according to reaction scheme 14 from compounds of formula (XII). Typical reaction conditions comprise heating (XII) in the presence of iron powder and ammonium chloride. The reaction is typically carried out in a solvent or a mixture of solvents at a temperature within the range 60-100° C. Suitable solvents include a mixture of water and alcohol (e.g methanol or ethanol).

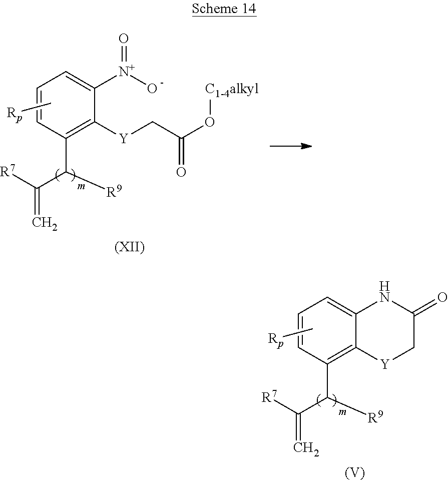

Compounds of formula (Va) may be prepared according to reaction scheme 15. Typical reaction conditions for the first step are reaction with BrCH(R⁷)C(═O)NH₂ in the presence of a suitable base (such as potassium carbonate) in a suitable solvent (such as acetone). Typical conditions for the second step are reaction in the presence of copper iodide and N,N-dimethylethylenediamine in a suitable base (such as potassium carbonate) in a suitable solvent (such as NMP) at elevated temperatures (such as 150 degC).

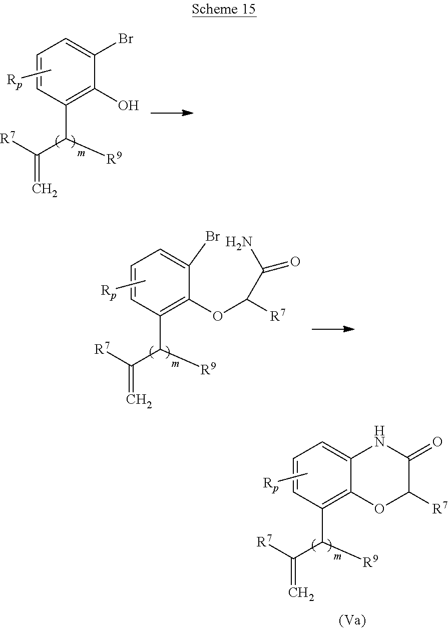

Compounds of formula (Vb) may be prepared according to reaction scheme 16 from compounds of formula XIII. Typical reaction conditions are described in Descriptions 71 and 72.

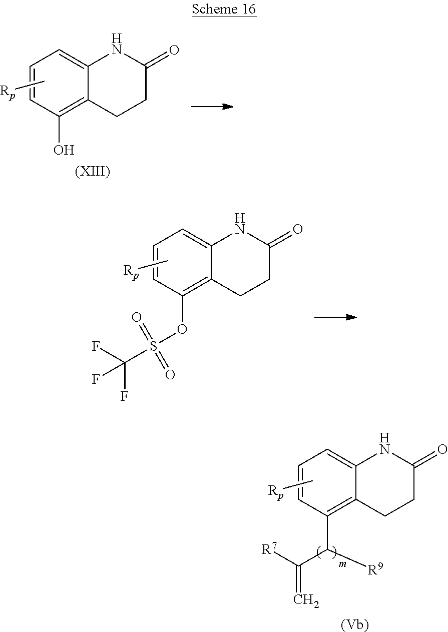

Compounds of formula (XIII) are commercially available or may be prepared using procedures known to the skilled chemist from readily available starting materials.

Compounds of formula (XIIa) may be prepared according to reaction scheme 17 by reacting compounds of formula (XIV) with a suitable base (such as sodium hydride) followed by treatment with a compound of formula (Hal)-CH₂—C(O)O—C₁₋₄alkyl where Hal is halogen (such as bromine).

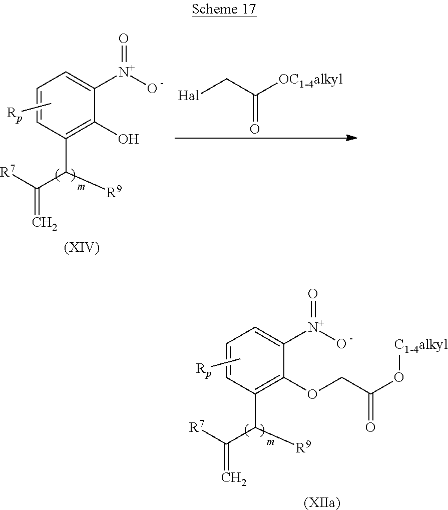

Compounds of formula (XIV) where m is 1, may be prepared by similar procedures described for Descriptions 1 and 2.

Compounds of general formula (I) may be prepared according to reaction scheme 18, wherein compounds of formula (XV) (where Hal is halogen such as chloro, bromo) are reacted with compounds of formula (III). Reaction conditions comprise heating in the presence of a base (e.g sodium carbonate or potassium carbonate) and optionally with a catalyst such as sodium iodide, in a suitable solvent (such as 1-methyl 2-pyrrolidone or methyl isobutyl ketone).

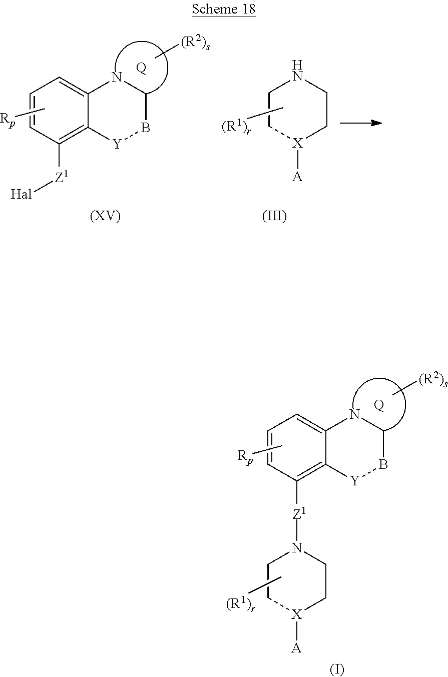

Compounds of formula (Ib), i.e. compounds of formula (I) where Y is O or —CH₂— and B is —CH₂—, may be prepared from compounds of formula (XVI) according to reaction scheme 19. Compounds of formula (XVI) may be reacted under similar conditions to those described for reaction schemes 3 and 4 to give compound of formula (Ib).

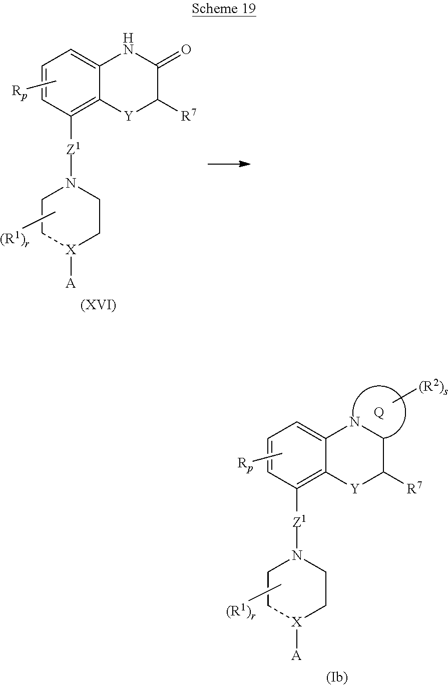

Compounds of formula (XVI) may be prepared by reacting compounds of formula (III) with compounds of formula (XVII) according to reaction scheme 20. Reaction conditions are as described for reaction scheme 18.

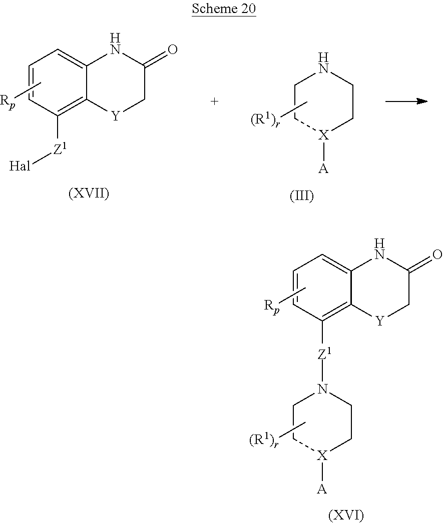

It will be appreciated that compounds of formula (I) or (IV) may be converted into other compounds of formula (I) or (IV) by synthetic methods known to the skilled person. Examples of such conversions are:

a) Compounds of formula (I) or (IV) where one or more R² is CO₂H may be prepared by hydrolysis of the corresponding compounds wherein R² is CO₂R⁷. Typically, hydrolysis is carried out in the presence of a base (e.g sodium hydroxide) in aqueous methanol at a high temperature and/or by application of microwaves.
b) Compounds of formula (I) or (IV), wherein R² is C(O)N(R³)(R⁴) may be prepared by reacting the corresponding compounds wherein R² is a suitable activated carboxyl group, with a compound of formula NH(R³)(R⁴). Suitable activated carboxyl groups include the acyl halide, the mixed anhydride, the activated ester (such as the thioester) or the derivative formed between the carboxylic acid group and a coupling agent (such as O-benzotriazol-1-yl-N,N,N′,N′-tetramethyluronium tetrafluoroborate). Typically the reaction is carried out at room temperature and in an aprotic solvent such as a hydrocarbon solvent, a halohydrocarbon solvent (such as dichloromethane) or an ether solvent (such as tetrahydrofuran) in the presence of a suitable base (such as diisopropylethylamine or dimethylamine).
c) Compounds of formula (I) or (IV), wherein R² is —C(O)N(C₁₋₆alkyl)OC₁₋₆alkyl may be prepared by reacting the corresponding compounds wherein R² is —CO₂R⁷ with trimethylaluminium and N,O-diC₁₋₆alkylhydroxylamine hydrochloride at room temperature.
d) Compounds of formula (I) or (IV), wherein R² is —C(O)R⁶ may be prepared by reacting the corresponding compounds wherein R² is —C(O)N(C₁₋₆alkyl)OC₁₋₆alkyl which the appropriate Grignard reagent in an organic solvent at low temperature.
e) Compounds of formula (I) or (IV), wherein R² is 3-methyl-5-isoxazolyl may be prepared in two steps by firstly reacting the corresponding compounds wherein R² is —C(O)R⁶, with N,N-dimethyl acetamide dimethyl acetal at elevated temperatures (such as 150 degC), followed by treatment with hydroxylamine hydrochloride in a suitable solvent (such as ethanol) at elevated temperatures (such as 150 degC).
f) Compounds of formula (I) or (IV), wherein R² is 3-methyl-1H-pyrazol-5-yl may be prepared in two steps by firstly reacting the corresponding compounds wherein R² is —C(O)R⁶, with N,N-dimethyl acetamide dimethyl acetal at elevated temperatures (such as 150 degC), followed by treatment with hydrazine monohydrate in a suitable solvent (such as ethanol) at elevated temperatures (such as 150 degC).
g) Compounds of formula (I) or (IV), wherein R² is cyano may be prepared by reacting the corresponding compounds wherein R² is —C(O)NH₂ with trifluoroacetic anhydride in the presence of an organic base (e.g pyridine). The reaction conveniently takes place in an aprotic solvent such as tetrahydrofuran at 0° C.
h) Compounds of formula (I) or (IV), wherein R² is hydrogen may be prepared by decarboxylation of a compound of formula (I) or (IV), wherein R² is CO₂H. The reaction may be carried out by heating in an organic solvent (such as a halogenated aromatic hydrocarbon).
i) Compounds of formula (I) or (IV) wherein R² is 3-methyl-1,2,4-oxadiazol-5-yl, may be prepared by reacting the corresponding compound of formula (I) or (IV), where R² is CO₂R⁷ with methyl carboxyamide oxime in the presence of a suitable base (such as sodium hydride).
j) Compounds of formula (I) or (IV), wherein R² is —C(═NOMe)R⁶ may be prepared in two steps by reacting the corresponding compounds of formula (I) or (IV), where R² is —C(O)R⁶ with methoxylamine hydrochloride in a suitable solvent (such as ethanol) at elevated temperature.
k) Compounds of formula (I) or (IV) wherein R² is —C(O)NH—NHC(O)Me, may be prepared by reacting the corresponding compounds of formula (I) or (IV) where R² is CO₂R⁷ with acetohydrazide in a suitable solvent (such as DCM) at low temperature (such as 0 degC).
l) Compounds of formula (I) or (IV) wherein R² is 5-methyl-1,3,4-oxadiazol-2-yl may be prepared by reacting the corresponding compounds of formula (I) or (IV) where R² is —C(O)NH—NHC(O)Me (see k) with triflic anhydride in a suitable solvent (such as pyridine) at low temperature (such as 0 degC).
m) Compounds of formula (I) or (IV) wherein R² is 1,3-oxazol-5-yl, may be prepared in two steps from the corresponding compounds of formula (I) or (IV) where R² is CO₂R⁷. In a first step the ester is reduced to the aldehyde using lithium aluminium hydride at low temperature (such as 0 degC) in a suitable solvent (such as THF). In a second step the aldehyde is treated with p-toluenesulfonylmethyl isocyanide.

It will be appreciated by those skilled in the art that it may be necessary to protect certain reactive substituents during some of the above procedures. Standard protection and deprotection techniques, such as those described in Greene T. W. Protective groups in organic synthesis, New York, Wiley (1981), can be used. For example, primary amines can be protected as phthalimide, benzyl, t-butyloxycarbonyl, benzyloxycarbonyl or trityl derivatives. Carboxylic acid groups can be protected as esters. Aldehyde or ketone groups can be protected as acetals, ketals, thioacetals or thioketals. Deprotection of such groups is achieved using conventional procedures well known in the art. For example, protecting groups such as t-butyloxycarbonyl may be removed using an acid such as hydrochloric or trifluororoacetic acid in a suitable solvent such as dichloromethane, diethylether, isopropanol or mixtures thereof.

Pharmaceutically acceptable salts may be prepared conventionally by reaction with the appropriate acid or acid derivative.

Further details for the preparation of compounds of formula (I) are found in the examples section hereinafter.

As mentioned above, the compounds of the invention possess high affinity for 5-HT₁ type receptors and/or are serotonin re-uptake inhibitors, and accordingly are useful in the treatment or prevention of diseases and conditions mediated by modulation of 5-HT₁ receptor and/or the serotonin re-uptake receptor.

Therefore according to a further aspect, the invention provides a compound of the invention for use as a medicament, suitably a human medicament.

According to a further aspect, the invention provides the use of the invention in the manufacture of a medicament for treating or preventing a disease or condition mediated by modulation of the 5-HT₁ receptor and/or the serotonin re-uptake receptor.

In an embodiment, diseases or conditions that may be mediated by modulation of the 5-HT₁ receptor and/or the serotonin re-uptake receptor are selected from the list consisting of: [the numbers in brackets after the listed diseases below refer to the classification code in Diagnostic and Statistical Manual of Mental Disorders, 4th Edition, published by the American Psychiatric Association (DSM-IV) and/or the International Classification of Diseases, 10th Edition (ICD-10)]:

i) Psychotic disorders for example Schizophrenia (including the subtypes Paranoid Type (295.30), Disorganised Type (295.10), Catatonic Type (295.20), Undifferentiated Type (295.90) and Residual Type (295.60)); Schizophreniform Disorder (295.40); Schizoaffective Disorder (295.70) (including the subtypes Bipolar Type and Depressive Type); Delusional Disorder (297.1) (including the subtypes Erotomanic Type, Grandiose Type, Jealous Type, Persecutory Type, Somatic Type, Mixed Type and Unspecified Type); Brief Psychotic Disorder (298.8); Shared Psychotic Disorder (297.3); Psychotic Disorder due to a General Medical Condition (including the subtypes with Delusions and with Hallucinations); Substance-Induced Psychotic Disorder (including the subtypes with Delusions (293.81) and with Hallucinations (293.82)); and Psychotic Disorder Not Otherwise Specified (298.9).
ii) Depression and mood disorders for example Depressive Episodes (including Major Depressive Episode, Manic Episode, Mixed Episode and Hypomanic Episode); Depressive Disorders (including Major Depressive Disorder, Dysthymic Disorder (300.4), Depressive Disorder Not Otherwise Specified (311)); Bipolar Disorders (including Bipolar I Disorder, Bipolar H Disorder (i.e. Recurrent Major Depressive Episodes with Hypomanic Episodes) (296.89), Cyclothymic Disorder (301.13) and Bipolar Disorder Not Otherwise Specified (296.80)); Other Mood Disorders (including Mood Disorder due to a General Medical Condition (293.83) which includes the subtypes With Depressive Features, With Major Depressive-like Episode, With Manic Features and With Mixed Features); Substance-Induced Mood Disorder (including the subtypes With Depressive Features, With Manic Features and With Mixed Features); and Mood Disorder Not Otherwise Specified (296.90).
iii) Anxiety disorders for example Social Anxiety Disorder, Panic Attack; Agoraphobia, Panic Disorder, Agoraphobia Without History of Panic Disorder (300.22); Specific Phobia (300.29) (including the subtypes Animal Type, Natural Environment Type, Blood-Injection-Injury Type, Situational Type and Other Type); Social Phobia (300.23); Obsessive-Compulsive Disorder (300.3); Posttraumatic Stress Disorder (309.81); Acute Stress Disorder (308.3); Generalized Anxiety Disorder (300.02); Anxiety Disorder Due to a General Medical Condition (293.84); Substance-Induced Anxiety Disorder, and Anxiety Disorder Not Otherwise Specified (300.00).
iv) Substance-related disorders for example Substance Use Disorders (including Substance Dependence, Substance Craving and Substance Abuse); Substance-Induced Disorders (including Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders (including Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9)); Amphetamine (or Amphetamine-Like)-Related Disorders (for example Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9)); Caffeine Related Disorders (including Caffeine Intoxication (305.90), Caffeine-Induced Anxiety Disorder, Caffeine-Induced Sleep Disorder and Caffeine-Related Disorder Not Otherwise Specified (292.9)); Cannabis-Related Disorders (including Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9)); Cocaine-Related Disorders (including Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9)); Hallucinogen-Related Disorders (including Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9)); Inhalant-Related Disorders (including Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9)); Nicotine-Related Disorders (including Nicotine Dependence (305.1), Nicotine Withdrawal (292.0) and Nicotine-Related Disorder Not Otherwise Specified (292.9)); Opioid-Related Disorders (including Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9)); Phencyclidine (or Phencyclidine-Like)-Related Disorders (including Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9)); Sedative-, Hypnotic-, or Anxiolytic-Related Disorders (including Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9)); Polysubstance-Related Disorder (including Polysubstance Dependence (304.80)); and Other (or Unknown) Substance-Related Disorders (including Anabolic Steroids, Nitrate Inhalants and Nitrous Oxide).
v) Sleep disorders for example primary sleep disorders such as Dyssomnias (including Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47)); primary sleep disorders such as Parasomnias (including Nightmare Disorder (307.47), Sleep Terror Disorder (307.46), Sleepwalking Disorder (307.46) and Parasomnia Not Otherwise Specified (307.47)); Sleep Disorders Related to Another Mental Disorder (including Insomnia Related to Another Mental Disorder (307.42) and Hypersomnia Related to Another Mental Disorder (307.44)); Sleep Disorder Due to a General Medical Condition; and Substance-Induced Sleep Disorder (including the subtypes Insomnia Type, Hypersomnia Type, Parasomnia Type and Mixed Type):
vi) Eating disorders such as Anorexia Nervosa (307.1) (including the subtypes Restricting Type and Binge-Eating/Purging Type); Bulimia Nervosa (307.51) (including the subtypes Purging Type and Nonpurging Type); Obesity; Compulsive Eating Disorder, Binge Eating Disorder; and Eating Disorder Not Otherwise Specified (307.50).
vii) Autism Spectrum Disorders including Autistic Disorder (299.00), Asperger's Disorder, Rett's Disorder, Childhood Disintegrative Disorder and Pervasive Developmental Disorder Not Otherwise Specified.
viii) Attention-Deficit/Hyperactivity Disorder (including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9)); Hyperkinetic Disorder, Disruptive Behaviour Disorders such as Conduct Disorder (including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23).
ix) Personality Disorders including the subtypes Paranoid Personality Disorder (301.0), Schizoid Personality Disorder (301.20), Schizotypal Personality Disorder (301.22), Antisocial Personality Disorder (301.7), Borderline Personality Disorder (301.83), Histrionic Personality Disorder (301.50), Narcissistic Personality Disorder (301.81), Avoidant Personality Disorder (301.82), Dependent Personality Disorder (301.6), Obsessive-Compulsive Personality Disorder (301.4) and Personality Disorder Not Otherwise Specified (301.9).
x) Enhancement of cognition including the treatment of cognition impairment in other diseases such as schizophrenia, bipolar disorder, depression, other psychiatric disorders and psychotic conditions associated with cognitive impairment, e.g. Alzheimer's disease.
xi) Sexual dysfunctions such as Sexual Desire Disorders (including Hypoactive Sexual Desire Disorder (302.71) and Sexual Aversion Disorder (302.79)); sexual arousal disorders (including Female Sexual Arousal Disorder (302.72) and Male Erectile Disorder (302.72)); orgasmic disorders (including Female Orgasmic Disorder (302.73), Male Orgasmic Disorder (302.74) and Premature Ejaculation (302.75)); sexual pain disorder (including Dyspareunia (302.76) and Vaginismus (306.51)); Sexual Dysfunction Not Otherwise Specified (302.70); paraphilias (including Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9)); gender identity disorders (including Gender Identity Disorder in Children (302.6) and Gender Identity Disorder in Adolescents or Adults (302.85)); and Sexual Disorder Not Otherwise Specified (302.9).

In a further embodiment, diseases or conditions that may be mediated by modulation of the 5-HT₁ receptor and/or the serotonin re-uptake receptor are selected from: group i), ii), iii) and xi) above.

In a further embodiment, the sexual dysfunction is premature ejaculation.

It will be appreciated that references herein to “treatment” extend to prophylaxis, prevention of recurrence and suppression or amelioration of symptoms (whether mild, moderate or severe) as well as the treatment of established conditions. The compound of the invention may be administered as the raw chemical but the active ingredient is suitably presented as a pharmaceutical formulation.

The compounds of the invention may be used in combination with the following agents to treat or prevent psychotic disorders: i) antipsychotics; ii) drugs for extrapyramidal side effects, for example anticholinergics (such as benztropine, biperiden, procyclidine and trihexyphenidyl), antihistamines (such as diphenhydramine) and dopaminergics (such as amantadine); iii) antidepressants; iv) anxiolytics; and v) cognitive enhancers for example cholinesterase inhibitors (such as tacrine, donepezil, rivastigmine and galantamine).

The compounds of the invention may be used in combination with antidepressants to treat or prevent depression and mood disorders.

The compounds of the invention may be used in combination with the following agents to treat or prevent bipolar disease: i) mood stabilisers; ii) antipsychotics; and iii) antidepressants.

The compounds of the invention may be used in combination with the following agents to treat or prevent anxiety disorders: i) anxiolytics; and ii) antidepressants.

The compounds of the invention may be used in combination with the following agents to treat or prevent male sexual dysfunction: i) phosphodiesterase V inhibitors, for example vardenafil and sildenafil; ii) dopamine agonists/dopamine transport inhibitors for example apomorphine and buproprion; iii) alpha adrenoceptor antagonists for example phentolamine; iv) prostaglandin agonists for example alprostadil; v) testosterone agonists such as testosterone; vi) serotonin transport inhibitors for example serotonin reuptake inhibitors; v) noradrenaline transport inhibitors for example reboxetine and vii) 5-HT1A agonists, for example flibanserine.

The compounds of the invention may be used in combination with the same agents specified for male sexual dysfunction to treat or prevent female sexual dysfunction, and in addition an estrogen agonist such as estradiol.

Antipsychotic drugs include Typical Antipsychotics (for example chlorpromazine, thioridazine, mesoridazine, fluphenazine, perphenazine, prochlorperazine, trifluoperazine, thiothixine, haloperidol, molindone and loxapine); and Atypical Antipsychotics (for example clozapine, olanzapine, risperidone, quetiapine, aripirazole, ziprasidone and amisulpride).

Antidepressant drugs include serotonin reuptake inhibitors (such as citalopram, escitalopram, fluoxetine, paroxetine, sertraline femoxetine, fluvoxamine, indalpine and zimeldine); dual serotonin/noradrenaline reuptake inhibitors (such as Venlafaxine, duloxetine and milnacipran); Noradrenaline reuptake inhibitors (such as reboxetine and venlafaxine); tricyclic antidepressants (such as amitriptyline, clomipramine, imipramine, maprotiline, nortriptyline and trimipramine); monoamine oxidase inhibitors (such as isocarboxazide, moclobemide, phenelzine and tranylcypromine); and others (such as bupropion, mianserin, mirtazapine, nefazodone and trazodone).

Mood stabiliser drugs include lithium, sodium valproate/valproic acid/divalproex, carbamazepine, lamotrigine, gabapentin, topiramate and tiagabine.

Anxiolytics include benzodiazepines such as alprazolam and lorazepam.

In addition the compounds of the invention may be administered in combination with 5-HT₃ antagonists (such as ondansetron, granisetron and metoclopramide); serotonin agonists (such as sumatriptan, rauwolscine, yohimbine and metocloprimide); and NK-1 antagonists.

It will be appreciated that the compounds of the combination or composition may be administered simultaneously (either in the same or different pharmaceutical formulations), separately or sequentially.

According to a further aspect, the invention provides a pharmaceutical composition comprising a compound of the invention, in association with one or more Pharmaceutically acceptable carrier(s), diluents(s) and/or excipient(s). The carrier, diluent and/or excipient must be “acceptable” in the sense of being compatible with the other ingredients of the composition and not deleterious to the recipient thereof.

The compounds of the invention may be administered in conventional dosage forms prepared by combining a compound of the invention with standard pharmaceutical carriers or diluents according to conventional procedures well known in the art. These procedures may involve mixing, granulating and compressing or dissolving the ingredients as appropriate to the desired preparation.

The pharmaceutical compositions of the invention may be formulated for administration by any route, and include those in a form adapted for oral, topical or parenteral administration to mammals including humans.

The compositions may be formulated for administration by any route. The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatine, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatine, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilising the compound and a sterile vehicle, water being typical. The compound, depending on the vehicle and concentration used, can be either suspended or dissolved in the vehicle. In preparing solutions the compound can be dissolved in water for injection and filter-sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilised powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilisation cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, suitably from 10-60% by weight, of the active material, depending on the method of administration. Where the compositions comprise dosage units, each unit will suitably contain from 50-500 mg of the active ingredient. The dosage as employed for adult human treatment will suitably range from 10 to 3000 mg per day, for instance 1500 mg per day depending on the route and frequency of administration. Such a dosage corresponds to 0.1 to 50 mg/kg per day.

It will be recognised by one of skill in the art that the optimal quantity and spacing of individual dosages of a compound of the invention will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of a compound of the invention given per day for a defined number of days, can be ascertained by those skilled in the art using conventional course of treatment determination tests.

All publications, including, but not limited to, patents and patent applications cited in this specification, are herein incorporated by reference as if each individual publication were specifically and individually indicated to be incorporated by reference herein as though fully set forth.

It will be appreciated that the invention includes the following further aspects. The embodiments described for the first aspect extend these further aspects. The diseases and conditions described above extend, where appropriate, to these further aspects.
i) A compound of the invention for use in treating or preventing a disease or condition mediated by modulation of the 5-HT₁ receptor and/or the serotonin re-uptake receptor.
ii) A method of treatment or prevention of a disease or condition mediated by modulation of the 5-HT₁ receptor and/or the serotonin re-uptake receptor in a mammal comprising administering an effective amount of a compound of the invention.

### EXAMPLES

The invention is illustrated by the Examples described below.

In the procedures that follow, after each starting material, reference to a description is typically provided. This is provided merely for assistance to the skilled chemist. The starting material may not necessarily have been prepared from the batch referred to.

Compounds are named using ACD/Name PRO 6.02 chemical naming software (Advanced Chemistry Development Inc., Toronto, Ontario, M5H2L3, Canada).

Proton Magnetic Resonance (NMR) spectra were recorded either on Varian instruments at 300, 400 or 500 MHz, or on a Bruker instrument at 300 MHz. Chemical shifts are reported in ppm (δ) using the residual solvent line as internal standard. Splitting patterns are designed as s, singlet; d, doublet; t, triplet; q, quartet; m, multiplet; b, broad. The NMR spectra were recorded at a temperature ranging from 25 to 90° C. When more than one conformer was detected the chemical shifts for the most abundant one is reported.

Mass spectra (MS) were taken on a 4 II triple quadrupole Mass Spectrometer (Micromass UK) or on a Agilent MSD 1100 Mass Spectrometer, operating in ES (+) and ES (−) ionization mode or on a Agilent LC/MSD 1100 Mass Spectrometer, operating in ES (+) and ES (−) ionization mode coupled with HPLC instrument Agilent 1100 Series [LC/MS—ES (+): analysis performed on a Supelcosil ABZ+Plus (33×4.6 mm, 3 μm) (mobile phase: 100% [water+0.1% HCO₂H] for 1 min, then from 100% [water+0.1% HCO₂H] to 5% [water+0.1% HCO₂H] and 95% [CH₃CN] in 5 min, finally under these conditions for 2 min; T=40° C.; flux=1 mL/min; LC/MS—ES (−): analysis performed on a Supelcosil ABZ+Plus (33×4.6 mm; 3 μm) (mobile phase: 100% [water+0.05% NH₃] for 1 min, then from 100% [water+0.05% NH₃ to 5% [water+0.05% NH₃] and 95% [CH₃CN] in 5 min, finally under these conditions for 2 min; T=40° C.; flux=1 mL/min]. In the mass spectra only one peak in the molecular ion cluster is reported.

Flash silica gel chromatography was carried out on silica gel 230-400 mesh (supplied by Merck AG Darmstadt, Germany) or over Varian Mega Be-Si pre-packed cartridges or over pre-packed Biotage silica cartridges.

SPE-SCX cartridges are ion exchange solid phase extraction columns by supplied by Varian. The eluent used with SPE-SCX cartridges is methanol followed by 2N ammonia solution in methanol.

In a number of preparation purification is performed using either Biotage manual flash chromatography (Flash+) or automatic flash chromatography (Horizon) systems. All these instruments work with Biotage Silica cartridge.

SPE-Si cartridges are silica solid phase extraction columns supplied by Varian.

| | |
|---|---|
| (+/−)-BINAP | racemic-2,2-(diphenylphosphino)-1,1-binaphthyl |
| 1,2-DCE and | 1,2-dichloroethane |
| DCE | |
| CH | cyclohexane |
| DAST | (diethylamino)sulfur trifluoride |
| DBU | 1.8-Diazabicyclo[5.4.0]undec-7-ene |
| DCM | dichloromethane |
| DEOXOFLUOR | bis(2-methoxymethyl)aminosulphur trifluoride |
| DIPEA | N,N-diisopropylethylamine |
| DMF | N,N′-dimethylformamide |
| EDC x HCl | 1-(3-dimethylaminopropyl)-3-ethylcarbodiimide |
| | hydrochloride |
| EDTA | Ethylenediaminetetraacetic acid |
| NMP | 1-methyl-2-pyrrolidone |
| rt | room temperature. |
| TBTU | O-(benzotriazol-1-yl)-N,N,N′N′-tetramethyluronium |
| | tetrafluoroborate |
| TEA | triethylamine |
| TFA | trifluoroacetic acid |
| THF | tetrahydrofuran |
| Triton B | benzyltrimethylammonium hydroxide |

### Description 1: 2-Nitrophenyl 2-propen-1-yl ether (D1)

To a solution of 2-nitrophenol (100 g, 719.4 mmol) in acetone (1.4 L) was added allyl bromide (68.5 ml, 791.4 mmol, 1.1 eq) and K₂CO₃ (110 g, 791.4 mmol, 1.1 eq) under N₂. The heterogeneous mixture was heated at reflux and stirred for 16 hours. The mixture was cooled down to room temperature, solids were filtered-off and washed with Et₂O. The combined organics were concentrated in vacuo to give the title compound (132 g, 737.4 mmol) as a pale orange viscous oil, that was used for the next step without further purification; MS (ES) m/z: 180.10 [MH⁺], C9H9NO3 requires 179.17; ¹H-NMR (300 MHz, CDCl₃) δ: 7.76 (m, 1H), 7.43 (m, 1H), 7.01-6.92 (m, 2H), 5.94 (m, 1H), 5.42 (m, 1H), 5.26 (m, 1H), 4.64-4.59 (m, 2 h).

### Description 2: 2-Nitro-6-(2-propen-1-yl)phenol (D2)

2-Nitrophenyl 2-propen-1-yl ether (D1) (132 g, 737.4 mmol) was stirred and heated in a sand bath with reflux apparatus, at 200° C. (internal temperature 180-160° C.) for 26 hours. After cooling, the crude product was purified by filtration through a silica pad (1 Kg) eluting with a gradient of cyclohexane/ethyl acetate (100:0 to 80:20) to afford the title compound as a pale yellow oil (89 g, 68%); MS (ES) m/z: 180.10 [MH⁺], C9H9NO3 requires 179.17; ¹H-NMR (300 MHz, CDCl₃) δ: 10.86 (s, 1H), 7.91 (m, 1H), 7.38 (m, 1H), 6.84 (m, 1H), 5.94 (m, 1H), 5.07-5.01 (m, 2H), 3.43-3.40 (m, 2H).

### Description 3: Methyl {[2-nitro-6-(2-propen-1-yl)phenyl]oxy}acetate (D3)

To a stirred solution of 2-nitro-6-(2-propen-1-yl)phenol (D2) (76 g, 424.6 mmol) in acetone (600 ml) at room temperature and under a nitrogen atmosphere, was added methylbromoacetate (422 ml, 445.8 mmol, 1.05 eq) and K₂CO₃ (61.6 g, 445.8 mmol, 1.05 eq). The mixture was heated at 60° C. and stirred for 3.5 hours. The mixture was cooled, the solid filtered-off and the solvent concentrated in vacuo. The residue was dissolved in ethyl acetate (1 L) and extracted with NaOH (1N sol, 3×200 ml), water (1×200 ml) and brine (1×200 ml). The combined organic layers were dried (MgSO₄) and concentrated in vacuo to give the title compound as a viscous pale yellow oil that was used for the next step without further purification; MS (ES) m/z: 252.10 [MH⁺], C12H13NO5 requires 251.24; ¹H-NMR (300 MHz, CDCl₃) δ: 7.66 (m, 1H), 7.39 (m, 1H), 7.14 (m, 1H), 5.88 (m, 1H), 5.10-4.95 (m, 2H), 4.56 (s, 2H), 3.75 (s, 3H), 3.49-3.46 (m, 2H).

### Description 4: 8-(2-Propen-1-yl)-2H-1,4-benzoxazin-3(4H)-one (D4)

To a stirred solution of methyl {[2-nitro-6-(2-propen-1-yl)phenyl]oxy}acetate (D3) (109 g, 434 mmol) in 1:1 MeOH/H₂O (1.8 L) were added iron powder (145 g, 2.6 mol, 6 eq) and NH₄Cl (232 g, 4.34 mol, 10 eq). The mixture was heated at 80° C. and stirred for 3.5 hours and then cooled to about 40 degC. The mixture was filtered through celite using 1:1 MeOH/DCM (1 L) as eluent. The solvent was evaporated to dryness and then DCM (2 litres) was added. The organic phase was washed with saturated aqueous ammonium chloride solution, water and brine. The mixture organic phase was dried (Na₂SO₄), and decolourising carbon powder added. The mixture was filtered, and the filtrate evaporated in vacuo to give a residue which was purified on a silica pad eluting using a gradient of cyclohexane/ethyl acetate (4:1 to 1:1) to afford the title compound (70 g, 86%) as a white solid; MS (ES) m/z: 190.10 [MH⁺], C11H11NO2 requires 189.21; ¹H-NMR (300 MHz, CDCl₃) δ: 8.10-7.85 (bs, 1H), 6.91-6.84 (m, 2H), 6.66 (m, 1H), 5.95 (m, 1H), 5.10-5.00 (m, 2H), 4.61 (s, 2H), 3.40-3.36 (m, 2H).

### Description 5: Ethyl 6(2-propen-1-yl)-4H-imidazo[5,1-c][1,4]-benzoxazine-3-carboxylate (D5)

Diethyl chlorophosphate (0.15 ml, 1.8 mmol) was added to a solution of 8-(2-propen-1-yl)-2H-1,4-benzoxazin-3(4H)-one (D4) (170 mg, 0.9 mmol) and potassium t-butoxide (110 mg, 0.9 mmol) in dry DMF (5 ml) at 0° C. After 10 minutes a solution of ethyl isocyanoacetate (0.15 ml, 1.35 mmol) and potassium t-butoxide (152 mg, 1.35 mmol) in dry DMF (2 ml) was added. The reaction mixture was stirred at 60° C. for 6 hours then cooled and quenched with water (5 ml). The DMF was evaporated in vacuo and the crude product purified by SPE-SI cartridge eluting with 30% ethyl acetate in cyclohexane to afford the title compound as a white solid (132 mg, 52%); MS (ES) m/z: 285.2 [MH⁺], C16H16N2O3 requires 284.3; ¹H-NMR (300 MHz, CDCl₃) δ: 8.2 (s, 1H), 7.55 (d, 1H), 7.3 (d, 1H), 7.2 (t, 1H), 6.25 (m, 1H), 5.7 (s, 2H), 5.2-5.3 (m, 2H), 4.65 (q, 2H), 3.65 (d, 2H), 1.6 (t, 3H).

### Description 6: Ethyl 6-(2-oxoethyl)-4H-imidazo[5,1-c][1,4]-benzoxazine-3-carboxylate

Osmium tetroxide (0.2 ml of a 4% by wt. solution in water, 0.125 eq) was added to a stirred solution of ethyl 6-(2-propen-1-yl)-4H-imidazo[5,1-c][1,4]benzoxazine-3-carboxylate (D5) (77 mg, 0.27 mmol) in THF/water (2:1; 1.5 ml). After 10 minutes sodium periodate (145 mg, 0.68 mmol) was added and the reaction mixture was stirred for 2 hours. After evaporation of THF the residue was partitioned between water (10 ml) and DCM (3×10 ml). The organic layers were combined, dried (Na₂SO₄) and concentrated in vacuo. The crude product was purified by SPE-Si cartridge, eluting with 4% methanol in DCM to afford the title compound as a white solid (52 mg, 67%); ¹H-NMR (300 MHz, CDCl₃) δ: 9.8 (s, 1H), 8 (s, 1H), 7.4 (t, 1H), 7-7.1 (m, 2H), 5.5 (s, 2H), 4.35 (q, 2H), 3.8 (s, 2H), 1.4 (t, 3H).

### Description 7: 6-(2-Propen-1-yl)-4H-imidazo[5,1-c][1,4]-benzoxazine-3-carboxylic acid (D7)

A solution of ethyl 6-(2-propen-1-yl)-4H-imidazo[5,1-c][1,4]benzoxazine-3-carboxylate (D5) (900 mg, 3.17 mmol) in a 1:1 mixture of methanol and 1M solution of sodium hydroxide (60 ml) was stirred at 60° C. for 30 minutes. The cooled reaction mixture was neutralised with acetic acid and then cooled to 0° C. The crude product was collected by filtration, washed with methanol and dried to afford the title compound as a white solid (570 mg, 70%); ¹H-NMR (300 MHz, CDCl₃) δ: 8.55 (s, 1H), 7.75 (d, 1H), 7.05-7.15 (m, 2H), 5.8 (m, 1H), 5.5 (s, 2H), 5.0-5.1 (m, 2H), 3.45 (d, 2H).

### Description 8: N,N-Dimethyl-6-(2-propen-1-yl)-4H-imidazo[5,1-c][1,4]-benzoxazine-3-carboxamide (D8)

1-Hydroxybenzotriazole (80 mg, 0.59 mmol) and 1-(3-dimethylaminopropyl)-3-ethylcarbodiimide hydrochloride (113 mg, 0.59 mmol) were added to a solution of 6-(2-propen-1-yl)-4H-imidazo[5,1-c][1,4]benzoxazine-3-carboxylic acid (D7) (150 mg, 0.59 mmol) and dimethylamine (0.35 ml of a 2M sol. in THF, 0.7 mmol) in a 2:1 solution of DMF/DCM (6 ml). The reaction mixture was stirred at room temperature for 4 hours, DMF was removed by SPE-SCX cartridge (eluting with methanol followed by 2N ammonia solution in methanol). The crude product was then purified by SPE-SI cartridge eluting with 5% methanol In DCM to afford the title compound as a white solid (68 mg, 41%); MS (ES) m/z: 284.2 [MH⁺], C16H16N2O3 requires 283.3; ¹H-NMR (300 MHz, CDCl₃) δ: 7.85 (s, 1H), 7.35 (d, 1H), 7.05 (d, 1H), 6.95 (t, 1H), 5.95 (M, 1H), 5.5 (s, 2H), 5.05 (m, 2H), 3.6 (s, 3H), 3.45 (d, 2H), 3.1 (s, 3H).

### Description 9: N,N-Dimethyl-6-(2-oxoethyl)-4H-imidazo[5,1-c][1,4]-benzoxazine-3-carboxamide (D9)

4-Methylmorpholine N-oxide (50 mg, 0.42 mmol) and osmium tetroxide (50 μl of a 4% by wt. solution in water, 0.035 eq) were added to a solution of N,N-dimethyl-6-(2-propen-1-yl)-4H-imidazo[5,1-c][1,4]-benzoxazine-3-carboxamide (D8) (60 mg, 0.21 mmol) in a 8:1 mixture of acetone/water (2.25 ml). The reaction mixture was stirred for 4 hours and then quenched with a saturated aqueous solution of sodium sulfite (10 ml). After 30 minutes stirring the mixture was extracted with ethyl acetate (3×10 ml). The combined organic layers were dried (Na₂SO₄) and concentrated in vacuo to give the intermediate 6-(2,3-dihydroxypropyl)-N,N-dimethyl-4H-imidazo[5,1-c][1,4]benzoxazine-3-carboxamide (40 mg, 0.14 mmol, 60%) which was taken-up in a 1:1 mixture of THF/water (2 ml) without further purification. Sodium periodate (43 mg, 0.21 mmol) was added and the mixture stirred for 1 hour. After evaporation of THF, the residue was partitioned between water (10 ml) and DCM (3×10 ml). The combined organic layers were dried (Na₂SO₄) and concentrated in vacuo. The crude material was purified by chromatography eluting with a 2% solution of methanol in DCM to give the corresponding 6-[2,2-bis(methyloxy)ethyl]-N,N-dimethyl-4H-imidazo[5,1-c][1,4]benzoxazine-3-carboxamide (25 mg). Hydrolysis of the acetal with HCl (0.15 ml, 1 M solution in diethyl ether) and further evaporation of the volatiles gave the title compound (20 mg) as a white solid; ¹H-NMR (300 MHz, CDCl₃) δ: 9.75 (s, 1H), 7.8 (s, 1H), 7.45 (t, 1H), 7.05 (d, 1H), 6.75 (d, 1H), 5.5 (s, 2H), 3.55 (s, 3H), 3.8 (s, 2H), 3.1 (s, 3H).

### Description 10: N-Cyclopentyl-6-(2-propen-1-yl)-4H-imidazo[5,1-c][1,4]-benzoxazine-3-carboxamide (D10)

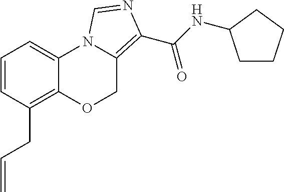

The title compound was prepared in 21% yield according to the general procedure of Description 8 starting from 6-(2-propen-1-yl)-4H-imidazo[5,1-c][1,4]benzoxazine-3-carboxylic acid (D7) (150 mg, 0.59 mmol) and cyclopentylamine (70 μL, 0.07 mmol); MS (ES) m/z: 324 [MH+], C19H21N3O2 requires 323.4; ¹H-NMR (300 MHz, CDCl₃) δ: 7.85 (s, 1H), 7.3 (d, 1H), 6.9-7.1 (m, 3H), 5.95 (m, 1H), 5.55 (s, 2H), 5.05 (m, 2H), 4.35 (q, 1H), 3.45 (d, 2H), 2-2.1 (m, 2H), 1.45-1.8 (m, 6H).

### Description 11: N-Cyclopentyl-6-(2-oxoethyl)-4H-imidazo[5,1-c][1,4]-benzoxazine-3-carboxamide (D11)

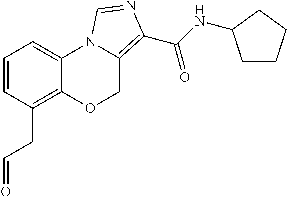

4-Methylmorpholine N-oxide (28 mg, 0.24 mmol) and osmium tetroxide (29 μl of a 4% by wt. solution in water, 0.035 eq) were added to a solution of N-cyclopentyl-6-(2-propen-1-yl)-4H-imidazo[5,1-c][1,4]benzoxazine-3-carboxamide (D10) (37 mg, 0.12 mmol) in a 8:1 mixture of acetone/water (2.25 ml). The reaction mixture was stirred for 4 hours and then quenched with a saturated aqueous solution of sodium sulfite (10 ml). After 30 minutes stirring the mixture was extracted with ethyl acetate (3×10 ml). The combined organic layers were dried (Na₂SO₄) and concentrated in vacuo to give the intermediate 6-(2,3-dihydroxypropyl)-N-cyclopentyl-4H-imidazo[5,1-c][1,4]benzoxazine-3-carboxamide (38 mg, 0.14 mmol, 90%) which was taken-up in a 1:1 mixture of THF/water (2 ml) without further purification. Sodium periodate (34 mg, 0.16 mmol) was added and the mixture stirred for 1 hour. After evaporation of the THF the residue was partitioned between water (10 ml) and DCM (3×10 ml). The combined organic layers were dried (Na₂SO₄) and concentrated in vacuo to give the title compound as a white solid (25 mg) which was used without further purification; ¹H-NMR (300 MHz, CDCl₃) δ: 9.8 (s, 1H), 7.8 (s, 1H), 7.45 (t, 1H), 6.7-7.1 (m, 3H), 5.5 (s, 2H), 4.4 (q, 1H), 3.8 (s, 2H), 1.4-1.9 (m, 8H).

### Description 12: 6-(2-Propen-1-yl)-4H-imidazo[5,1-c][1,4]-benzoxazine (D12)

A mixture of 6-(2-propen-1-yl)-4H-imidazo[5,1-c][1,4]benzoxazine-3-carboxylic acid (D7) (120 mg, 0.47 mmol) in 1,2-dichlorobenzene (1.5 ml) was irradiated in a microwave reactor (PersonalChemistry Emrys™ Optimiser, 300W, 250° C., 10 minutes). The solvent was removed by SPE-SCX cartridge (eluting with methanol followed by 2N ammonia solution in methanol) to afford the title compound as a white solid (61 mg, 100%); MS: (ES) m/z: 213.2 [MH⁺], C13H12N20 requires 212.3; ¹H-NMR (300 MHz, CDCl₃) δ: 8.6 (s, 1H), 7.6 (d, 1H), 7.2-7.35 (m, 3H), 6.25 (m, 1H), 5.5 (s, 2H), 5.35 (m, 2H), 3.65 (d, 2H).

### Description 13: 4H-imidazo[5,1-c][1,4]-benzoxazin-6-ylacetaldehyde (D13)

The title compound was prepared in 30% yield according to the procedure of Description 11 starting from 6-(2-propen-1-yl)-4H-imidazo[5,1-c][1,4]-benzoxazine (D12) (60 mg, 0.28 mmol). The reaction was left under stirring for 3 days and additional osmium tetroxide (50 μl, 0.026 eq) and 4-methylmorpholine N-oxide (66 mg, 0.56 mmol) were required for a good conversion to the 3-(4H-imidazo[5,1-c][1,4]benzoxazin-6-yl)-1,2-propanediol intermediate; ¹H-NMR (300 MHz, CDCl₃) δ: 9.8 (s, 1H), 8.15 (s, 1H), 7.7 (d, 1H), 7.3-7.45 (m, 3H), 5.5 (s, 2H), 3.75 (s, 2H).

### Description 14: 8-(3-Hydroxypropyl)-2H-1,4-benzoxazin-3(4H)-one (D14)

Disiamyl borane (18 mL of a 1.6 M solution in THF, 29 mmol) was added dropwise to a solution of 8-(2-propen-1-yl)-2H-1,4-benzoxazin-3(4H)-one (D4) (2.5 g, 13.2 mmol) in dry THF (45 ml) at 0° C. The solution was stirred for 4 hours at 0° C. and then overnight at room temperature. The reaction mixture was diluted with ethanol (8.7 ml) and then sodium hydroxide (3 ml of 6M aq. solution) and hydrogen peroxide (6 ml of a 35% by wt. aq. solution) were added dropwise. The mixture was stirred at 50° C. for 2 hours and then warmed to room temperature before diluting with water (300 ml) and extracting with ethyl acetate (3×300 ml). The combined organic layers were dried (Na₂SO₄) and concentrated in vacuo. The crude product was purified by SPE-SI cartridge, eluting with 40% cyclohexane in ethyl acetate, to afford the title compound as a white solid (1.9 g, 69%); ¹H-NMR (300 MHz, CDCl₃) δ: 8.1 (bs, 1H), 6.9 (m, 2H), 6.65 (d, 1H), 4.6 (s, 2H), 3.6 (t, 2H), 2.7 (t, 2H), 1.8 (dd, 2H).

### Description 15: 8-(3-{[tert-Butyl(dimethyl)silyl]oxy}propyl)-2H-1,4-benzoxazin-3(4H)-one (D15)

To a solution of 8-(3-hydroxypropyl)-2H-1,4-benzoxazin-3(4H)-one (D14) (0.5 g, 2.4 mmol) in dry DMF (35 ml) were added imidazole (1.6 g, 24 mmol) and tert-butyldimethylsilylchloride (3.5 g, 24 mmol). The reaction mixture was stirred at room temperature for 4 hours and then was quenched with a saturated aqueous solution of NaHCO₃ (200 ml) and extracted with ethyl acetate (3×200 ml). The combined organic layers were dried (Na₂SO₄) and concentrated in vacuo. The crude product was purified by SPE-SI cartridge, eluting with 30% cyclohexane in ethyl acetate, to afford the title compound (containing DMF ˜50%). The compound was used without further purification for the next step; MS: (ES) m/z: 322.2 [MH⁺], C17H27NO3Si requires 321.5; ¹H-NMR (300 MHz, CDCl₃) δ: 8.3 (s, 1H), 8 (s, 1H DMF), 6.9 (m, 2H), 6.7 (d, 1H), 4.6 (s, 2H), 3.7 (t, 2H), 2.95 (3H, DMF), 2.8 (3H, DMF) 2.65 (t, 2H), 1.9 (m, 2H), 0.9 (s, 9H), 0 (s, 6H).

### Description 16: Ethyl 6-(3-hydroxypropyl)-4H-imidazo[5,1-c][1,4]-benzoxazine-3-carboxylate (D16)

The title compound was prepared in 24% yield according to the procedure of Description 5 starting from 8-(3-{[tert-butyl(dimethyl)silyl]oxy}propyl)-2H-1,4-benzoxazin-3(4H)-one (D15) (2.4 mmol). The crude product was purified by SPE-SCX cartridge (eluting with methanol followed by 2N ammonia solution in methanol); MS: (ES) m/z: 303.2 [MH⁺], C16H18N2O4 requires 302.3; ¹H-NMR (500 MHz, DMSO-d⁶) δ: 8 (s, 1H), 7.3 (d, 1H), 7.1 (d, 1H), 6.9 (t, 1H), 5.5 (s, 2H), 4.3 (q, 2H), 3.7 (t, 2H), 2.8 (t, 2H), 1.8 (m, 2H), 1.4 (t, 3H).

### Description 17: Ethyl 6-(3-oxopropyl)-4H-imidazo[5,1-c][1,4]benzoxazine-3-carboxylate (D17)

To a solution of ethyl 6-(3-hydroxypropyl)-4H-imidazo[5,1-c][1,4]benzoxazine-3-carboxylate (D16) (170 mg, 0.56 mmol) in DCM (5 ml) was added Dess-Martin periodinane (263 mg, 0.56 mmol) portionwise. The reaction mixture was stirred at room temperature for 2 hours, filtered, quenched with water (10 ml) and extracted with DCM (3×10 ml). The combined organic layers were dried (Na₂SO₄) and concentrated in vacuo to afford the title compound (160 mg, 100%) which was used without further purification; MS: (ES) m/z: 301.2 [MH⁺], C16H16N2O4 requires 300.3; ¹H-NMR (300 MHz, CDCl₃) δ: 9.8 (s, 1H), 8.2 (m, 1H), 7.6-8 (m, 3H), 5.5 (s, 2H), 4.35 (q, 2H), 3 (m, 2H), 2.7 (m, 2H) 1.4 (t, 3H).

### Description 18: 8-(2-Propen-1-yl)-2H-1,4-benzoxazine-3(4H)-thione (D18)

A mixture of 8-(2-propen-1-yl)-2H-1,4-benzoxazin-3(4H)-one (D4) (2 g, 10.5 mmol) and Lawesson's reagent (2.2 g, 5.3 mmol) in dry toluene (35 ml) was heated at reflux for 1 hour. The mixture was cooled to room temperature and the solvent evaporated in vacuo. The crude material was purified by chromatography on silica gel eluting with 10% cyclohexane in ethyl acetate to afford the title compound as a white solid (1.9 g, 88%); ¹H-NMR (300 MHz, CDCl₃) δ: 9.6 (s, 1H), 6.85 (m, 2H), 6.75 (m, 1H), 5.8 (m, 1H), 5.05 (m, 2H), 4.75 (s, 2H), 3.35 (d, 2H).

### Description 19: 3-(Methylthio)-8(2-propen-1-yl)-2H-1,4-benzoxazine (D19)

To a mixture of 8-(2-propen-1-yl)-2H-1,4-benzoxazine-3(4H)-thione (D18) (100 mg, 0.49 mmol) and potassium hydroxide (69 mg, 1.23 mmol) in acetone (2 ml) was added methyl iodide (46 μL, 0.74 mmol) in two portions 15 minutes apart. The reaction mixture was heated under reflux for 2 hours, cooled, filtered to remove potassium iodide and evaporated in vacuo. The crude material was purified by chromatography on silica gel eluting with 10% cyclohexane in ethyl acetate to afford the title compound as a colourless oil (70 mg, 65%); MS: (ES) m/z: 220.2 [MH⁺], C12H13NOS requires 219.3; ¹H-NMR (300 MHz, CDCl₃) δ: 7.2 (m, 1H), 6.9 (bs, 2H), 5.9 (m, 1H), 5 (m, 2H), 4.5 (s, 2H), 3.3 (d, 2H), 2.5 (s, 3H).

### Description 20: N-[2,2-Bis(methyloxy)ethyl]-8-(2-propen-1-yl)-2H-1,4-benzoxazin-3-amine (D20)

A mixture of 3-(methylthio)-8-(2-propen-1-yl)-2H-1,4-benzoxazine (D19) (180 mg, 0.82 mmol) and aminoacetaldehyde dimethylacetal (134 μL, 1.23 mmol) in dry ethanol (5 ml) was heated at reflux for 9 hours. The volatiles were evaporated in vacuo and the crude product was purified by chromatography on silica gel eluting with 40% ethyl acetate in cyclohexane to afford the title compound as a colourless oil (93 mg, 41%); MS: (ES) m/z: 277.3 [MH⁺], C15H20N2O3 requires 276.3; ¹H-NMR (300 MHz, CDCl₃) δ: 7.2 (m, 1H), 6.9 (bs, 2H), 5.9 (m, 1H), 5 (m, 2H), 4.5 (s, 2H), 3.3 (d, 2H), 2.5 (s, 3H).

### Description 21: 6-(2-Propen-1-yl)-4H-imidazo[2,1-c][1,4]-benzoxazine (D21)

A mixture of N-[2,2-bis(methyloxy)ethyl]-8-(2-propen-1-yl)-2H-1,4-benzoxazin-3-amine (D20) (90 mg, 0.33 mmol) and conc. hydrochloric acid (0.8 ml) in methanol (1 ml) was heated at reflux for 3 hours. After concentration in vacuo the residue was dissolved in DCM (20 ml), washed with an aqueous saturated solution of NaHCO₃ (15 ml), dried (Na₂SO₄) and evaporated in vacuo. The crude material was purified by chromatography on silica gel eluting with 35% ethyl acetate in cyclohexane to afford the title compound as a yellow solid (65 mg, 92%); MS: (ES) m/z: 213.3 [MH⁺], C13H12N2O requires 212.3; ¹H-NMR (300 MHz, CDCl₃) δ: 7.4 (bs, 1H), 7.2 (bs, 2H), 7.05 (bs, 2H), 5.95 (m, 1H), 5.3 (s, 2H), 5.1 (m, 2H), 3.45 (d, 2H).

### Description 22 and 23

### 4H-imidazo[2,1-c][1,4]-benzoxazin-6-ylacetaldehyde (D22)

### 4H-imidazo[2,1-c][1,4]-benzoxazine-6-carbaldehyde (D23)

The title compounds were obtained using the procedure described in Description 11 starting from 6-(2-propen-1-yl)-4H-imidazo[2,1-c]-[1,4]-benzoxazine (D21) (200 mg, 0.94 mmol). The reaction was left under stirring for 2 days and additional osmium tetroxide (200 μl, 0.03 eq) and 4-Methylmorpholine N-oxide (250 mg, 2.1 mmol) were required. The reaction afforded a mixture of the two aldehydes D22 and D23 (˜7:3) which was used in the next step; MS (ES) m/z: for D22 215.3 [MH⁺] C12H10N2O2 requires 214.3; for D23 201.3 [MH⁺] C11H8N2O2 requires 200.2; ¹H-NMR (300 MHz, CDCl₃) δ: 10.5 (s, 1H D23), 9.8 (s, 1H D22), 7.7 (m, 1H D23), 7.5 (m, 1H D23), 7-7.4 (m, 5H D22+3H D23), 5.4 (s, 2H D23), 5.25 (s, 2H D22), 3.8 (s, 2H D22).

### Description 24: 1-Methyl-6-(2-propen-1-yl)-4H-[1,2,4]triazolo[3,4-c][1,4]-benzoxazine (D24)

A solution of 8-(2-propen-1-yl)-2H-1,4-benzoxazine-3(4H)-thione (D18) (1.38 g, 6.71 mmol) in absolute ethanol (50 ml) was added dropwise over 1 hour to a solution of hydrazine monohydrate (8 ml) in absolute ethanol (50 ml) at 80° C. The resulting reaction mixture was stirred at reflux (80° C.) for a further 40 minutes and concentrated in vacuo to afford the intermediate (3E)-8-(2-propen-1-yl)-2H-1,4-benzoxazin-3(4H)-one hydrazone which was immediately used in the following step without any further purification. The hydrazone was mixed with trimethyl orthoacetate (15 ml) and heated with stirring at 150° C. for 10 minutes under microwave irradiation. The resulting reaction mixture was evaporated in vacuo and purified by flash chromatography on silica gel, eluting with 5% methanol in DCM to give the title compound as a pale yellow solid (0.78 g, 51%); MS; (ES) m/z: 228.20 [MH⁺]. C₁₃H1₃ N₃O requires 227.27; ¹H-NMR (300 MHz, CDCl₃) δ: 7.36 (d, 1H), 7.11-7.03 (m, 2H), 6.01-5.84 (m, 1H), 5.25 (s, 2H), 5.08-5.01 (m, 2H), 3.43 (d, 2H), 2.76 (s, 3H).

### Description 25: (1-Methyl-4H-[1,2,4]triazolo[3,4-c][1,4]-benzoxazin-6-yl)acetaldehyde (D25)

4-Methylmorpholine N-oxide (145 mg, 1.24 mmol) and osmium tetroxide (0.20 ml of 4% by wt. solution in water) were added to a solution of 1-methyl-6-(2-propen-1-yl)-4H-[1,2,4]triazolo[3,4-c][1,4]-benzoxazine (D24) (142 mg, 0.62 mmol) in a 8:1 mixture of acetone/water (9 ml). The resulting reaction mixture was stirred at room temperature overnight, evaporated in vacuo and then purified by flash chromatography on silica gel, eluting with 5% methanol in DCM to give the intermediate 3-(1-methyl-4H-[1,2,4]triazolo[3,4-c][1,4]benzoxazin-6-yl)-1,2-propanediol (97 mg, 60%) as a white solid. This material was taken-up in a 1:1 mixture of THF/water (3 ml), sodium periodate (197 mg, 0.92 mmol) was added and the resulting mixture was stirred at room temperature for 1 hour. After evaporation of THF, the residue was partitioned between water (10 ml) and DCM (3×10 ml). The combined organic layers were dried (Na₂SO₄) and concentrated in vacuo to give the title compound as a white solid (50 mg, 35%) which may be used without further purification; MS (ES) m/z: 230.20 [MH⁺], C₁₂H₁₁N₃O₂ requires 229.24; ¹H-NMR (300 MHz, CDCl₃) δ: 9.78 (s, 1H), 7.49 (d, 1H), 7.15-7.12 (m, 2H), 5.28 (s, 2H), 3.82 (s, 2H), 2.79 (s, 3H).

### Description 26: 4-(2-Oxopropyl)-8-(2-propen-1-yl)-2H-1,4-benzoxazin-3(4H)-one (D26)

Sodium hydride (159 mg of a 60% w/w suspension in mineral oil, 3.98 mmol) was added in portions to a solution of 8-(2-propen-1-yl)-2H-1,4-benzoxazin-3(4H)-one (D4) (500 mg, 2.65 mmol) and 1-chloro-2-propanone (253 μl, 3.17 mmol) in dry DMF (6 ml) at 0° C. The reaction mixture was allowed to warm to room temperature and stirred for 7 hours then quenched with water (10 ml) and extracted with ethyl acetate (3×30 ml). The combined organic layers were dried (Na₂SO₄) and concentrated in vacuo to give the crude product which was purified by flash chromatography on silica gel, eluting with DCM/acetone (95/5), to afford the title compound as a white solid (314 mg, 49%); MS (ES) m/z: 246.20 [MH⁺]. C14H15NO3 requires 245.28; ¹H-NMR (300 MHz, CDCl₃) δ: 6.95-6.85 (m, 2H), 6.49 (m, 1H), 5.95 (m, 1H), 5.08-5.01 (m, 2H), 4.67 (s, 4H), 3.39 (m, 2H), 2.54 (s, 3H).

### Description 27: 2-Methyl-6-(2-propen-1-yl)-4H-imidazo[2,1-c][1,4]-benzoxazine (D27)

Ammonium acetate (396 mg, 5.14 mmol) was added to a solution of 4-(2-oxopropyl)-8-(2-propen-1-yl)-2H-1,4-benzoxazin-3(4H)-one (D26) (63 mg, 0.257 mmol) in acetic acid glacial (1 ml). The mixture was irradiated in a microwave reactor (PersonalChemistry Emrys™ Optimiser, 300W, 150° C., 10 min), then diluted with ethyl acetate (10 ml), poured into ice-water (10 ml) and basified with aqueous ammonium hydroxide solution (3 ml). The mixture was extracted with ethyl acetate (3×20 ml), the combined organics were dried (Na₂SO₄) and the solvent was removed under reduced pressure. The crude material was purified by flash chromatography on silica gel, eluting with DCM/acetone (95/5) to afford the title compound (47 mg, 81%); MS (ES) m/z: 227.10 [MH⁺], C14H14N2O requires 226.28; ¹H-NMR (300 MHz, CDCl₃) δ: 7.09-6.94 (m, 4H), 5.95 (m, 1H), 5.19 (s, 2H), 5.06-5.00 (m, 2H), 3.39 (m, 2H), 2.26 (s, 3H).

### Description 28: (2-Methyl-4H-imidazo[2,1-c][1,4]-benzoxazin-6-yl)acetaldehyde (D28)

The title compound was prepared according to the procedure of Description 6 starting from 2-methyl-6-(2-propen-1-yl)-4H-imidazo[2,1-c][1,4]benzoxazine (D27). The product was isolated in 36% yield by flash chromatography on silica gel using ethyl acetate/cyclohexane (1/1) as eluent; MS (ES) m/z: 229.20 [MH⁺]. C13H12N2O2 requires 228.25; ¹H-NMR (300 MHz, CDCl₃) δ: 9.77 (s, 1H), 7.22-6.91 (m, 4H), 5.24 (s, 2H), 3.77 (s, 2H), 2.31 (s, 3H).

### Description 29: 4-(1-Methyl-2-oxopropyl)-8-(2-propen-1-yl)-2H-1,4-benzoxazin-3(4H)-one (D29)

A mixture of 8-(2-propen-1-yl)-2H-1,4-benzoxazin-3(4H)-one (D4) (500 mg, 2.65 mmol), 3-chloro-2-butanone (294 μl, 2.91 mmol, 1.1 eq) and K₂CO₃ (402 mg, 2.91 mmol, 1.1 eq) in dry acetone (15 ml) was heated at reflux for 4 hours. Further 3-chloro-2-butanone (294 μl, 2.91 mmol, 1.1 eq) and K₂CO₃ (402 mg, 2.91 mmol, 1.1 eq) were added and the reaction mixture was heated at reflux for 18 hours. The mixture was cooled to room temperature, solids were filtered-off and washed with acetone (50 ml) and the combined filtrates concentrated in vacuo. The crude material was purified by flash chromatography on silica gel, eluting with DCM/acetone (95/5) to afford the title compound (626 mg, 91%) as a pale yellow viscous oil; MS (ES) m/z: 260.2 [MH⁺], C15H17NO3 requires 259.31; ¹H-NMR (300 MHz, CDCl₃) δ: 6.96-6.85 (m, 2H), 6.66-6.58 (m, 1H), 5.93 (m, 1H), 5.16-5.00 (m, 3H), 4.70-4.52 (m, 2H), 3.38 (m, 2H), 2.12 (s, 3H), 1.51 (m, 3H).

### Description 30: [4-(1-Methyl-2-oxopropyl)-3-oxo-3,4-dihydro-2H-1,4-benzoxazin-8-yl]acetaldehyde (D30)

The title compound was prepared in 64% yield according to the procedure of Description 6 starting from 4-(1-methyl-2-oxopropyl)-8-(2-propen-1-yl)-2H-1,4-benzoxazin-3(4H)-one (D29) (195 mg, 0.753 mmol). The product was purified by flash chromatography on silica gel using ethyl acetate/cyclohexane (1/1) as eluent; ¹H-NMR (300 MHz, CDCl₃) δ: 9.72 (s, 1H), 6.93 (m, 1H), 6.85 (m, 1H), 6.68 (m, 1H), 5.12 (m, 1H), 4.59 (m, 2H), 3.71 (s, 2H), 2.11 (s, 3H), 1.50 (m, 3H).

### Description 31: 4-(1-Methyl-2-oxopropyl)-8-{2-[4-(2-methyl-5-quinolinyl)-1-piperazinyl]-ethyl}-2H-1,4-benzoxazin-3(4H)-one (D31)

The title compound was isolated as a white solid in 92% yield according to the general procedure described in Example 1 starting from [4(1-methyl-2-oxopropyl)-3-oxo-3,4-dihydro-2H-1,4-benzoxazin-8-yl]acetaldehyde (D30) (127 mg, 0.487 mmol). The product was purified by flash chromatography on silica gel eluting with a gradient of DCM/methanol (99/1 to 98/2); MS (ES) m/z: 473.40 [MH⁺]. C28H32N4O3 requires 472.59; ¹H-NMR (500 MHz, DMSO-d6) δ: 8.36 (d, 1H), 7.60 (m, 2H), 7.40 (d, 1H), 7.12 (dd, 1H), 7.04 (m, 3H), 4.95 (quart., 1H), 4.7 (dd, 2H), 3.05 (bs, 4H), 2.85 (t, 2H), 2.75 (bs, 4H), 2.65 (m, 5H), 2.08 (s, 3H), 1.41 (d, 3H).

### Description 32: Methyl[3-oxo-8-(2-propen-1-yl)-2,3-dihydro-4H-1,4-benzoxazin-4-yl]acetate (D32)

The title compound was prepared in 95% yield according to the general procedure of Description 26. Thus, 8-(2-propen-1-yl)-2H-1,4-benzoxazin-3(4H)-one (D4) (1.0 g, 5.29 mmol) in DMF (15 ml) was treated with methyl bromoacetate (651 μl, 6.88 mmol, 1.3 eq) and sodium hydride (318 mg of 60% w/w suspension in mineral oil, 7.94 mmol, 1.5 eq). The mixture was stirred for 6 hours and the crude product was purified by flash chromatography on silica gel using ethyl acetate/cyclohexane (1/4) as eluent; MS (ES) m/z: 262.2 [MH⁺]. C14H15NO4 requires 261.28; ¹H-NMR (300 MHz, CDCl₃) δ: 6.96-6.90 (m, 2H), 6.65-6.60 (m, 1H), 5.95 (m, 1H), 5.10-5.01 (m, 2H), 4.67 (s, 2H), 4.66 (s, 2H), 3.78 (s, 3H), 3.39 (m, 2H).

### Description 33: Methyl[3-oxo-8-(2-oxoethyl)-2,3-dihydro-4H-1,4-benzoxazin-4-yl]acetate (D33)

The title compound was prepared in 66% yield according to the general procedure of Description 6 starting from methyl[3-oxo-8-(2-propen-1-yl)-2,3-dihydro-4H-1,4-benzoxazin-4-yl]acetate (D32) (1.0 g, 3.83 mmol). The product was purified by flash chromatography on silica gel using ethyl acetate/cyclohexane (3/7) as eluent; ¹H-NMR (300 MHz, CDCl₃) δ: 9.74 (m, 1H), 6.98 (m, 1H), 6.88 (m, 1H), 6.71 (m, 1H), 4.68 (s, 2H), 4.67 (s, 2H), 3.79 (s, 3H), 3.74 (m, 2H).

### Description 34: Methyl (8-{2-[4-(2-methyl-5-quinolinyl)-1-piperazinyl]ethyl}-3-oxo-2,3-dihydro-4H-1,4-benzoxazin-4-yl) acetate (D34)

The title compound was prepared in 90% yield according to the procedure of Example 1 starting from methyl[3-oxo-8-(2-oxoethyl)-2,3-dihydro-4H-1,4-benzoxazin-4-yl]acetate (D33) (300 mg, 1.14 mmol). The product was purified by flash chromatography on silica gel eluting with a gradient of DCM/methanol (99/1 to 98/2); MS (ES) m/z: 475.20 [MH⁺], C27H30N4O4 requires 474.56; ¹H-NMR (300 MHz, CDCl₃) δ: 8.37 (m, 1H), 7.71 (m, 1H), 7.58 (m, 1H), 7.24 (m, 1H), 7.07 (m, 1H), 6.95 (m, 2H), 6.64 (m, 1H), 4.70 (s, 2H), 4.66 (s, 2H), 3.79 (s, 3H), 3.16 (bs, 4H), 2.98-2.74 (bm, 8H), 2.73 (s, 3H).

### Description 35: (8-{2-[4-(2-Methyl-5-quinolinyl)-1-piperazinyl]ethyl}-3-oxo-2,3-dihydro-4H-1,4-benzoxazin-4-yl)acetic acid (D35)

To a suspension of methyl (8-{2-[4-(2-methyl-5-quinolinyl)-1-piperazinyl]ethyl}-3-oxo-2,3-dihydro-4H-1,4-benzoxazin-4-yl)acetate (D34) (300 mg, 0.633 mmol) in methanol (5 ml) was added 2N NaOH solution in methanol (5 ml). The mixture was allowed to stir at room temperature for 4 hours before addition of water (10 ml). The methanol was evaporated under reduced pressure and the residue was acidified with 6N aqueous HCl (5 ml). This mixture was applied to a SPE-SCX cartridge (eluting with 1:1 water/methanol then 2N ammonia solution in methanol) to afford the title compound as a white solid (290 mg, 99%); MS; (ES) m/z: 461.2 [MH⁺]. C26H28N4O4 requires 460.54; ¹H-NMR (300 MHz, DMSO-d6) δ: 8.30 (m, 1H), 7.58-7.52 (m, 2H), 7.34 (m, 1H), 7.07 (m, 1H), 6.90-6.80 (m, 2H), 6.75 (m, 1H), 4.60 (s, 2H), 4.21 (s, 2H), 3.01 (bs, 4H), 2.84-2.53 (bm, 11H).

### Description 36: 8-{2-[4-(2-Methyl-5-quinolinyl-1-piperazinyl]ethyl}-4-(3,3,3-trifluoro-2-oxopropyl)-2H-1,4-benzoxazin-3(4H)-one (D36)

To a suspension of (8-{2-[4-(2-methyl-5-quinolinyl)-1-piperazinyl]ethyl}-3-oxo-2,3-dihydro-4H-1,4-benzoxazin-4-yl)acetic acid (D35) (115 mg, 0.25 mmol) in toluene (10 ml) at room temperature was added oxalyl chloride (64 μl, 0.75 mmol, 3 eq) and a drop of DMF. The suspension was heated to 60° C. for 3 hours. Removal of the solvent under reduced pressure gave the crude acid chloride which was used directly in the subsequent reaction. To a suspension of this crude add chloride (120 mg, 0.25 mmol) in DCM (10 ml) at room temperature were added trifluoroacetic anhydride (315 μl, 1.5 mmol, 6 eq) and pyridine (162 μl, 2 mmol, 8 eq). After stirring for 2 hours, water (15 ml) was added before the reaction mixture was basified by the addition of NaHCO₃ (sat. aq. solution, 10 ml). The organic phase was separated and the remaining aqueous phase was extracted with DCM (3×50 ml). The combined organic phases were dried (MgSO₄) and evaporated under reduced pressure to give a crude product which was subjected to flash column chromatography on silica gel eluting with DCM/methanol (97/3) to afford the title compound D36 (hydrate of the ketone) as a yellow solid (79 mg, 60%); MS; (ES) m/z: 531.1 [MH⁺]. C27H29F3N4O4 requires 530.55; ¹H-NMR (300 MHz, DMSO-d6) δ: 8.37 (m, 1H), 7.70 (m, 1H), 7.58 (m, 1H), 7.23 (m, 1H), 7.08-6.81 (m, 4H), 5.15-4.20 (m, 4H), 3.14 (bs, 4H), 2.95-2.60 (bm, 11H).

### Description 37: 2-(2-Butyn-1-yloxy)-1-nitro-3-(2-propen-1-yl)benzene (D371

A mixture of 2-nitro-6-(2-propen-1-yl)phenol (D2) (500 mg, 2.79 mmol), 1-bromo-2-butyne (269 μl, 3.07 mmol, 1.1 eq) and K₂CO₃ (424 mg, 3.07 mmol, 1.1 eq) in acetone (20 ml) was heated at reflux for 4 hours. The mixture was cooled to room temperature before the solids were filtered-off and washed with acetone (30 ml). The combined organics were concentrated in vacuo and the crude product was purified by flash chromatography on silica gel eluting with ethyl acetate/cyclohexane (1/9) to afford the title compound (640 mg, 99%); ¹H-NMR (300 MHz, CDCl₃) δ: 7.65 (m, 1H), 7.39 (m, 1H), 7.13 (m, 1H), 5.90 (m, 1H), 5.11-5.02 (m, 2H), 4.60 (m, 2H), 3.49 (m, 2H), 1.77 (s, 3H).

### Description 38:[2-(2-Butyn-1-yloxy)-3-(2-propen-1-yl)phenyl]amine (D38)

To a solution of 2-(2-butyn-1-yloxy)-1-nitro-3-(2-propen-1-yl)benzene (D37) (640 mg, 2.77 mmol) in glacial acetic acid (10 ml) was added iron powder (619 mg, 11.1 mmol, 4 eq) at room temperature and the mixture was stirred for 16 hours. The solvent was evaporated in vacuo and ethyl acetate (50 ml) was added to the residue. The precipitate was removed by filtration and the filtrate was extracted successively with 1N NaOH (10 ml) and brine (10 ml) before drying (MgSO₄) and evaporation in vacuo to afford the title compound (540 mg, 97%); MS (ES) m/z: 202.10 [MH⁺]. C13H15NO requires 201.27; ¹H-NMR (300 MHz, CDCl₃) δ: 6.86 (m, 1H), 6.61-6.53 (m, 2H), 5.94 (m, 1H), 5.10-5.01 (m, 2H), 4.44 (m, 2H), 3.82 (bs, 2H), 3.40 (m, 2H), 1.87 (m, 3H).

### Description 39: 1-Azido-2-(2-butyn-1-yloxy)-3-(2-propen-1-yl/benzene (D39)

A solution of sodium nitrite (72 mg, 1.05 mmol, 1.05 eq) in water (1 ml) was added dropwise to a suspension of [2-(2-butyn-1-yloxy)-3-(2-propen-1-yl)phenyl]amine (D38) (201 mg, 1 mmol) in 4N HCl (3 ml) under vigorous stirring and ice/water cooling. The mixture was then neutralised by addition of aq. NaHCO₃ and a solution of sodium azide (65 mg, 1 mmol, 1 eq) in water (1 ml) was slowly added at 5° C. After 30 minutes the mixture was extracted with diethyl ether (3×20 ml) and the combined organics dried (MgSO₄) and evaporated under reduced pressure to give the title azide (222 mg, 98%) which was used for the next step without further purification; ¹H-NMR (300 MHz, CDCl₃) δ: 7.02 (m, 1H), 6.96-6.87 (m, 2H), 5.90 (m, 1H), 5.10-4.95 (m, 2H), 4.55 (s, 2H), 3.45 (m, 2H), 1.80 (s, 3H).

### Description 40: 3-Methyl-6-(2-propen-1-yl)-4H-[1,2,3]triazolo[5,1-c][1,4]-benzoxazine (D40)

A solution of 1-azido-2-(2-butyn-1-yloxy)-3-(2-propen-1-yl)benzene (D39) (222 mg, 0.98 mmol) in toluene (8 ml) was heated at reflux for 2.5 hours. The solvent was then evaporated under reduced pressure and the residue was purified by flash chromatography on silica gel eluting with ethyl acetate/cyclohexane (1/4) to afford the title compound (183 mg, 82%); MS (ES) m/z: 228.20 [MH⁺], C13H13N3O requires 227.27; ¹H-NMR (300 MHz, CDCl₃) δ: 7.86 (m, 1H), 7.10-7.00 (m, 2H), 5.90 (m, 1H), 5.25 (s, 2H), 5.05-4.98 (m, 2H), 3.39 (m, 2H), 2.33 (s, 3H).

### Description 41: (3-Methyl-4H-[1,2,3]triazolo[5,1-c][1,4]-benzoxazin-6-yl)acetaldehyde (D41)

The title compound was prepared in 66% yield according to the procedure of Description 6 starting from 3-methyl-6-(2-propen-1-yl)-4H-[1,2,3]triazolo[5,1-c][1,4]benzoxazine (D40) (183 mg, 0.806 mmol). The product was purified by flash chromatography on silica gel using ethyl acetate/cyclohexane (2/3) as eluent; MS (ES) m/z: 230.10 [MH⁺]. C12H11N3O2 requires 229.24; ¹H-NMR (300 MHz, CDCl₃) δ: 9.72 (s, 1H), 7.97 (m, 1H), 7.1 (m, 2H), 5.26 (s, 2H), 3.74 (s, 2H), 2.33 (s, 3H).

### Description 42: (3E)-8-(2-Propen-1-yl)-2H-1,4-benzoxazin-3(4H)-one oxime (D42)

A mixture of 8-(2-propen-1-yl)-2H-1,4-benzoxazine-3(4H)-thione (D18) (108 mg, 0.53 mmol), hydroxylamine hydrochloride (55 mg, 0.79 mmol, 1.5 eq) and sodium acetate (65 mg, 0.79 mmol, 1.5 eq) in dry ethanol (5 ml) was heated at reflux for 40 minutes. Water (15 ml) was added, ethanol was removed under reduced pressure and the aqueous layer was extracted with DCM (3×30 ml). The combined organics were dried (MgSO₄) and the solvent removed under reduced pressure. Flash column chromatography on silica gel (ethyl acetate/cyclohexane 1/4) gave the title compound as a white solid (105 mg, 97%); MS (ES) m/z: 205.10 [MH⁺]. C11H12N2O2 requires 204.23; ¹H-NMR (300 MHz, CDCl₃) δ: 6.82 (m, 1H), 6.70 (m, 1H), 6.63 (m, 1H), 6.16 (s, 1H), 5.90 (m, 1H), 5.02-4.95 (m; 2H), 4.51 (s, 2H), 3.32 (m, 2H).

### Description 43: 6-(2-Propen-1-yl)-4H-[1,2,4]oxadiazolo[3,4-c][1,4]-benzoxazin-1-one (D43)

(3E)-8-(2-Propen-1-yl)-2H-1,4-benzoxazin-3(41-1)-one oxime (D42) (108 mg, 0.529 mmol) in dry THF (4 ml) was treated with carbonyldiimidazole (94 mg, 0.582 mmol, 1.1 eq) at reflux for 1 hour before further carbonyldiimidazole (94 mg, 0.582 mmol) was added. After 2 hours at reflux the solvent was removed under reduced pressure and the residue was taken-up in DCM (20 ml), washed with water (10 ml), dried (Na₂SO₄) and evaporated under reduced pressure. The crude product was purified by flash chromatography on silica gel eluting with ethyl acetate/cyclohexane (1/4) to give the title compound (115 mg, 94%); ¹H-NMR (300 MHz, CDCl₃) δ: 7.98 (m, 1H), 7.32 (m, 1H), 7.12 (m, 1H), 5.97 (m, 1H), 5.20-5.00 (m, 4H), 3.43 (m, 2H).

### Description 44: (1-oxo-4H-[1,2,4]oxadiazolo[3,4-c][1,4]-benzoxazin-6-yl)acetaldehyde (D44)

The title compound was prepared in 71% yield according to the procedure of Description 6 starting from 6-(2-propen-1-yl)-4H-[1,2,4]oxadiazolo[3,4-c][1,4]benzoxazin-1-one (D43) (115 mg, 0.50 mmol). The product was purified by flash chromatography on silica gel using ethyl acetate/cyclohexane (1/4) as eluent; ¹H-NMR (300 MHz, CDCl₃) δ: 9.72 (s, 1H), 8.00 (m, 1H), 7.14-7.02 (m, 2H), 5.05 (s, 2H), 3.76 (s, 2H).

### Description 45: 2-(Trifluoromethyl)-5-quinolinyl trifluoromethanesulfonate (D45)

To an ice-cold solution of 2-(trifluoromethyl)-5-quinolinol (1.07 g, 5.03 mmol) and pyridine (2.05 ml, 25.1 mmol) in dry DCM (20 ml) was added trifluoromethane sulfonic anhydride (1.34 ml, 8.05 mmol). The mixture was warmed to room temperature and stirring was continued for 2 hours. Water (15 ml) was added and the mixture was extracted with DCM (3×20 ml). The combined organics were dried (MgSO₄) and evaporated in vacuo to give the title compound as a yellowish oil (1.61 g, 93%); MS (ES; m/z): 346 [MH⁺], C₁₁H₆F₆NO₃S requires 345.22; ¹H-NMR (300 MHz, DMSO-d₆) δ: 8.70 (d, 1H), 8.33 (d, 1H), 8.20 (d, 1H), 8.03 (2H, m).

### Description 46: 1,1-Dimethylethyl-4-[2-(trifluoromethyl)-5-quinolinyl]-1-piperazine-carboxylate (D46)

A mixture of 2-(trifluoromethyl)-5-quinolinyl trifluoromethanesulfonate (D45) (1.61 g, 4.68 mmol), Cs₂CO₃ (2.29 g, 7.02 mmol), Pd(OAc)₂ (147 mg, 0.655 mmol), (+/−)-BINAP (436 mg, 0.702 mmol) and N-Boc-piperazine (1.39 g, 7.48 mmol) in dry toluene (20 mL) was stirred at 90° C. overnight. After cooling to room temperature aq. saturated NH₄Cl solution (25 ml) was added and the mixture was extracted with ethyl acetate (3×30 ml). The combined organics were dried (MgSO₄), and evaporated in vacuo. The residue was purified by column chromatography on silica gel eluting with a gradient of cyclohexane/ethyl acetate (9:1 to 0:1) to afford the title compound as a yellow oil (1.27 g, 71%); MS (ES; m/z): 382 [MH⁺], C₁₉H₂₂F₃N₃O₂ requires 381.40; ¹H-NMR (300 MHz, CDCl₃) δ: 8.65 (d, 1H), 7.9 (d, 1H), 7.7 (m, 2H), 7.2 (d, 1H), 3.6-3.8 (m, 4H), 3.0-3.1 (m, 4H), 1.5 (s, 9H).

### Description 47: 5-(1-Piperazinyl)-2-(trifluoromethyl)quinoline (D47)

A solution of 1,1-dimethylethyl-4-[2-(trifluoromethyl)-5-quinolinyl]-1-piperazine-carboxylate (D46) (1.27 g, 3.33 mmol) in a 1:3 mixture of TFA/THF (20 ml) was stirred at room temperature overnight. After evaporation the crude material was purified by SPE-SCX cartridge (eluting with methanol followed by 2N ammonia solution in methanol) to afford the title compound (888 mg, 95%) as a yellow solid; MS (ES; m/z): 282 [MH⁺], C₁₄H₁₄F₃N₃ requires 281.28; ¹H-NMR (300 MHz, CDCl₃) δ: 8.7 (d, 1H), 7.9 (d, 1H), 7.7 (m, 2H), 7.2 (m, 1H), 3.2-3.0 (m, 9H).

### Description 48: (2-E,Z)-2-Buten-1-yl 2-nitrophenyl ether) (D48)

To a solution of 2-nitrophenol (5.1 g, 36.3 mmol) in acetone (45 ml) was added crotyl chloride (3.9 ml, 40.0 mmol, 1.1 eq) and K₂CO₃ (5.5 g, 40 mmol, 1.1 eq). The heterogeneous mixture was heated at reflux for 18 hours. The mixture was cooled and the solvent was concentrated in vacuo. A solution of NaOH 1M (200 ml) was added to the concentrated reaction mixture and the product was extracted with ethyl acetate (2×100 ml). The combined organic extracts were washed with NaOH 1M (2×100 ml), water (1×100 ml) and brine (1×100 ml), dried over anhydrous Na₂SO₄, filtered and concentrated to dryness in vacuo. A mixture of stereoisomers 8/2 of title compound (6.0 g, 85%) was obtained as an orange oil that may be used for the next step without further purification; ¹H-NMR (300 MHz, CDCl₃): δ: 7.81 (m, 1H), 7.45 (m, 1H), 7.06 (m, 1H), 6.98 (m, 1H), 5.92-5.58 (m, 2H), 4.74 (m, 2H, stereoisomer 1), 4.60 (m, 2H, stereoisomer 2), 1.74 (m, 3H).

### Description 49: 2-(1-Methyl-2-propen-1-yl)-6-nitrophenol (D49)

(2-E, Z)-2-Buten-1-yl 2-nitrophenyl ether (D48) (6.0 g, 31.1 mmol) was stirred and heated, in a sand bath with reflux apparatus, at 200° C. for 5 hours. After cooling, the crude product was purified by SPE-Si cartridge, eluting with cyclohexane/ethyl acetate (100:0 to 80:20) to afford the title compound (964 mg, 16%); ¹H-NMR (300 MHz, CDCl₃) δ: 11.04 (s, 1H), 7.98 (d, 1H), 7.47 (d, 1H), 6.90 (t, 1H), 6.02 (m, 1H), 5.11-5.05 (m, 2H), 4.00 (m, 1H), 1.33 (d, 3H).

### Description 50: Methyl {[2-(1-methyl-2-propen-1-yl)-6-nitrophenyl]oxy}acetate (D50)

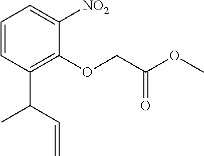

To a stirred solution of 2-(1-methyl-2-propen-1-yl)-6-nitrophenol (D49) (343 mg, 1.77 mmol) in acetone (10 ml) at room temperature was added methylbromoacetate (0.18 ml, 1.95 mmol) and K₂CO₃ (269 mg, 1.95 mmol). The mixture was heated to reflux for 3 hours and quenched with water (20 ml). The product was extracted with ethyl acetate (2×20 ml), dried over Na₂SO₄ and concentrated in vacuo. The crude product was purified by SPE-Si cartridge (cyclohexane/ethyl acetate 90:10) to afford the title compound (486 mg, quant.) as a yellow oil; ¹H-NMR (300 MHz, CDCl₃) δ: 7.71 (d, 1H), 7.48 (d, 1H), 7.23 (m, 1H+CDCl₃), 5.96 (m, 1H), 5.14-4.92 (m, 2H), 4.75-4.48 (d+d, 2H), 4.09 (m, 1H), 3.82 (s, 3H), 1.38 (d, 3H).

### Description 51: 8-(1-Methyl-2-propen-1-yl)-2H-1,4-benzoxazin-3(4H)-one (D51)

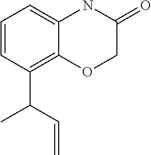

To a stirred solution of methyl {[2-(1-methyl-2-propen-1-yl)-6-nitrophenyl]oxy}acetate (D50) (486 mg, 1.83 mmol) in 1:1 MeOH/H₂O (8 ml) were added iron powder (614 mg, 11.0 mmol) and NH₄Cl (979 mg, 18.3 mmol). The mixture was heated at 80° C. and stirred for 4 hours before filtration through celite using 1:1 MeOH/DCM (1 L) as eluent. The volatile organics were removed in vacuo and the residual watery solution was extracted with DCM (2×50 ml). The combined organics were dried (Na₂SO₄) and concentrated in vacuo to afford the title compound (280 mg, 80%) as light brown solid that may be used for the next step without further purification; ¹H-NMR (300 MHz, CDCl₃) δ: 7.79 (bs, 1H), 7.00-6.82 (m, 2H), 6.61 (m, 1H), 6.00 (m, 1H), 5.11-4.96 (m, 2H), 4.58 (s, 2H), 3.82 (m, 1H), 1.31 (d, 3H).

### Description 52: Ethyl 6-(1-methyl-2-propen-1-yl)-4H-imidazo[5,1-c][1,4]-benzoxazine-3-carboxylate (D52)

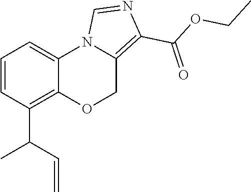

Diethyl chlorophosphate (0.80 ml, 5.36 mmol) was added to a solution of 8-(1-methyl-2-propen-1-yl)-2H-1,4-benzoxazin-3(4H)-one (D51) (545 mg, 2.68 mmol) and potassium t-butoxide (300 mg, 2.68 mmol) in dry DMF (8 ml) at 0° C. After 20 minutes a solution of ethyl isocyanoacetate (0.44 ml, 4.02 mmol) and potassium t-butoxide (451 mg, 4.02 mmol) in dry DMF (3.2 ml) was added. The reaction mixture was stirred at 60° C. for 8 hours then cooled and quenched with water (20 ml). The product was extracted with ethyl acetate (2×20 ml), dried over Na₂SO₄ and concentrated in vacuo. The crude product was purified by SPE-SI cartridge, eluting with 30% ethyl acetate in cyclohexane to afford the title compound as a white solid (200 mg, 25%); ¹H-NMR (300 MHz, CDCl₃) δ: 7.98 (s, 1H), 7.32 (d, 1H), 7.12 (d, 1H), 7.01 (t, 1H), 5.98 (m, 1H), 5.53 (s, 2H), 5.14-5.01 (m, 2H), 4.40 (q, 2H), 3.91 (m, 1H), 1.41 (t, 3H), 1.32 (d, 3H)

### Description 53: Ethyl 6-(1-methyl-2-oxoethyl)-4H-imidazo[5,1-c][1,4]-benzoxazine-3-carboxylate (D53)

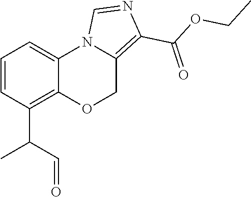

4-Methylmorpholine N-oxide (196 mg, 1.68 mmol) and osmium tetroxide (50 μl of a 4% by wt. solution in water) were added to a solution of ethyl 6-(1-methyl-2-propen-1-yl)-4H-imidazo[5,1-c][1,4]benzoxazine-3-carboxylate (D52) (200 mg, 0.67 mmol) in a 8:1 mixture of acetone/water (16.7 ml). The reaction mixture was stirred for 18 hours and then quenched with a saturated aqueous solution of sodium sulfite (20 ml). After 30 minutes stirring the mixture was extracted with ethyl acetate (3×20 ml). The combined organic layers were dried (Na₂SO₄) and concentrated in vacuo to give ethyl 6-(2,3-dihydroxy-1-methylpropyl)-4H-imidazo[5,1-c][1,4]benzoxazine-3-carboxylate (200 mg, 0.60 mmol, 90%) which was taken-up in a 1:1 mixture of THF/water (17 ml) without further purification. Sodium periodate (272 mg, 1.27 mmol) was added and the mixture stirred for 2 hours. After evaporation of THF the residue was partitioned between water (30 ml) and ethyl acetate (3×20 ml). The combined organic layers were dried (Na₂SO₄) and concentrated in vacuo to afford the title compound (151 mg, 84%) as white solid that was used for the next step without further purification; ¹H-NMR (300 MHz, CDCl₃) δ: 9.70 (s, 1H), 7.98 (s, 1H), 7.40 (d, 1H), 7.15-7.0 (d+t, 2H), 5.61-5.39 (d+d, 2H), 4.38 (q, 2H), 3.89 (m, 1H), 1.49-1.31 (t+d, 6H).

### Description 54: 2-Bromo-5-fluorophenyl 2-propen-1-yl ether (D54)

2-Bromo-5-fluorophenol (10 mmol), allyl bromide (1 ml, 1.1 equiv.), K₂CO₃ (1.5 g, 1.1 equiv.) in acetone (100 ml) were stirred at 60° C. overnight. Then, the reaction mixture was concentrated at reduced pressure, washed with aqueous NH₄Cl (100 ml) and extracted with Et₂O (3×75 ml). The collected organic phases were washed with 1M aqueous NaOH (100 ml), dried over Na₂SO₄ and concentrated to afford the title compound as yellow oil (1.50 g, 90%); ¹H-NMR (300 MHz, CDCl₃) δ: 7.35-7.30 (m, 1H), 6.50-6.30 (m, 2H), 5.95-5.75 (m, 1H), 5.25 (d, 1H), 5.15 (d, 1H), 4.40 ppm (d, 2H).

### Description 55: 1-Bromo-4-fluoro-2-(2-propen-1-yloxy)benzene (D55)

Diethylaluminium chloride (2 ml of a 1M solution in hexane, 2.0 equiv.) was added dropwise to a stirred solution of 2-bromo-5-fluorophenyl 2-propen-1-yl ether (D54) (213 mg, 1 mmol) in heptane (100 ml) cooled to 0° C. The resulting mixture was allowed to warm up to room temperature and stirred for 1 hour. Then, it was cooled to 0° C. and 1M aqueous HCl (50 ml) was added. The reaction mixture was extracted with Et₂O (3×50 ml) and the collected organic phases were washed with H₂O (75 ml), dried over Na₂SO₄, filtered and concentrated. The resulting crude material was purified by flash-chromatography on silica gel using cyclohexane/ethyl acetate (98:2) to afford the title compound (120 mg, 60%); ¹H-NMR (300 MHz, CDCl₃) δ: 7.25 (t, 1H), 6.55 (t, 1H), 6.00-5.70 (m, 1H), 5.60 (s, 1H), 5.10-4.95 (m, 2H), 3.40 ppm (d, 2H).

### Description 56: (3-Bromo-6-fluoro-2-hydroxyphenyl)acetaldehyde (D56)

Sodium periodate (7.4 g, 34.7 mmol) was added to 1-bromo-4-fluoro-2-(2-propen-1-yloxy)benzene (D55) (4.0 g, 17.3 mmol) stirred at room temperature in THF (70 ml) and H₂O (70 ml). After 5 min., 4% wt solution of OsO₄ in H₂O (6.3 ml, 5 mol %) was added. The resulting mixture was stirred at room temperature for 1 hour. The reaction mixture was washed sequentially with Et₂O (100 ml), H₂O (100 ml) and aqueous Na₂S₂O₃ (100 ml) then dried over Na₂SO₄. The resulting crude material was purified by chromatography on silica gel using cyclohexane/ethyl acetate (95:5) to afford the title compound as a colourless solid (2.0 g, 50%). On the basis of NMR spectra it was identified as a mixture ca. 30/70 of the title product and 7-bromo-4-fluoro-2,3-dihydro-1-benzofuran-2-ol; ¹H-NMR (300 MHz, CDCl₃) δ: 9.7 (s, 1H), 7.38 (dd, 1H), 6.65 (t, 1H), 5.78 (br s, 1H), 3.80 ppm (s, 2H). ¹H-NMR (300 MHz, CDCl₃) δ: 7.20-7.30 (m, 1H), 6.65 (t, 1H), 6.2 (br s, 1H), 3.55 (d, 1H), 3.45 (dd, 1H), 3.20 ppm (dd, 1H).

### Description 57: 6-Bromo-3-fluoro-2-{2-[4-(2-methyl-5-quinolinyl)-1-piperazinyl]ethyl}phenol (D57)

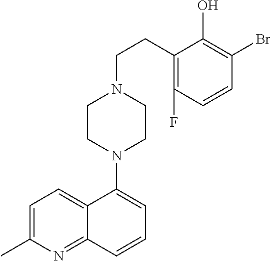

2-Methyl-5-(1-piperazinyl)quinoline (1.46 g, 6.4 mmol) and (3-bromo-6-fluoro-2-hydroxyphenyl)acetaldehyde (D56) (1.50 g, 6.4 mmol) were stirred at room temperature in DCM (10 ml) for 30 min., then Na₂SO₄ (0.91 g, 6.4 mmol) was added. The resulting mixture was stirred at room temperature for further 30 min. and then sodium triacetoxyborohydride (1.35 g, 6.4 mmol) was added and the mixture was stirred overnight. The reaction mixture was poured into aqueous NH₄Cl (50 ml) and extracted with DCM (3×50 ml). The collected organic phases were washed with aqueous NH₄Cl (75 ml) and H₂O (75 ml), then dried over Na₂SO₄ and concentrated. The resulting crude material was purified by chromatography on silica gel using DCM/MeOH (98:2) to afford the title compound as a yellow solid (2.0 g, 70%); MS: (ES/+) m/z: 444 [MH⁺]. C₂₂H₂₄FBrN₃O requires 443; ¹H-NMR (300 MHz, CDCl₃) δ(ppm): 8.35 (d, 1H), 7.75 (d, 1H), 7.65 (t, 1H), 7.40-7.25 (m, 2H), 7.15 (d, 1H), 6.50 (t, 1H), 3.30-2.25 (m, 12H), 2.75 ppm (s, 3H).

### Description 58: 2-[(2-Bromo-6-{2-[4-(2-methyl-5-quinolinyl)-1-piperazinyl]ethyl}phenyl)oxy]acetamide (D58)

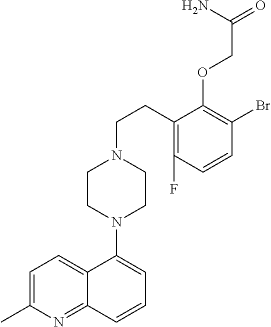

2-Bromo-6-{2-[4-(2-methyl-5-quinolinyl)-1-piperazinyl]ethyl}phenol (D57) (444 mg, 1 mmol), 2-bromoacetamide (151 mg, 1.1 mmol) and K₂CO₃ (145 mg, 1.1 mmol) were dissolved in acetone (10 ml) and stirred at 60° C. overnight. After evaporation under reduced pressure H₂O (50 ml) was added and the resulting mixture was extracted with DCM (3×50 ml). The combined organic phases were washed with H₂O (50 ml), dried over Na₂SO₄ and concentrated. The resulting crude material was purified by chromatography on silica gel using DCM/MeOH (96:4) to afford the title compound as a colourless solid (400 mg, 86%); MS (ES/+) m/z: 503 [MH⁺]. C₂₄H₂₆BrFN₄O₂ requires 502; ¹H-NMR (300 MHz, CDCl₃) δ(ppm): 8.35 (d, 1H), 7.70 (d, 1H), 7.55 (t, 1H), 7.40 (dd, 1H), 7.30-7.20 (m, 1H), 7.05 (d, 1H), 6.85 (t, 1H), 4.5 (s, 2H), 3.5 (br s, 2H), 3.10-2.6 (m, 15H).

### Description 59: 7-Fluoro-8-{2-[4-(2-methyl-5-quinolinyl)-1-piperazinyl]ethyl}-2H-1,4-benzoxazin-3(4H)-one (D59)

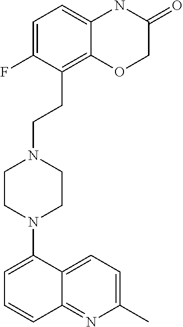

2-[(2-Bromo-6-{2-[4-(2-methyl-5-quinolinyl)-1-piperazinyl]ethyl}phenyl)oxy]-acetamide (D58) (400 mg, 0.80 mmol), CuI (152 mg, 0.80 mmol), N,N′-dimethylethylendiamine (71 mg, 0.80 mmol) and K₂CO₃ (220 mg, 1.6 mmol) were suspended in NMP (5 ml) and stirred at 150° C. for 1 h. The resulting reaction mixture was purified with SPE-(SCX) and then with chromatography on silica gel using DCM/MeOH (96:4) to afford the title compound as colourless solid (200 mg, 60%); MS: (ES/+) m/z: 421 [MH⁺]. C₂₄H₂₅FN₄O₂ requires 420; ¹H-NMR (300 MHz, CDCl₃) δ(ppm): 8.35 (d, 1H), 7.9 (s, 1H), 7.7 (d, 1H), 7.6 (t, 1H), 7.2 (m, 1H), 7.0 (d, 1H), 6.5-6.7 (m, 2H), 4.6 (s, 2H), 3.2 (br s, 4H), 3.0-2.6 ppm (m, 11H).

### Description 60: 2-[(2-Bromo-6-{2-[4-(2-methyl-5-quinolinyl)-1-piperazinyl]ethyl}phenyl)-oxy]propanamide (D60)

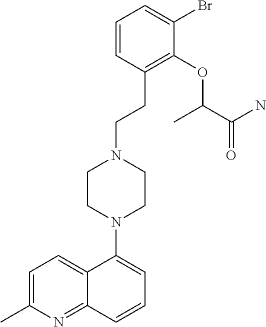

Sodium hydride (37 mg of a 60% w/w dispersion in mineral oil) was carefully added to 6-bromo-3-fluoro-2-{2-[4-(2-methyl-5-quinolinyl)-1-piperazinyl]ethyl}phenol (D57) (400 mg, 0.93 mmol) in dioxane (3 ml). The resulting yellow solution was stirred at room temperature for 10 min., then 2-bromopropionamide (140 mg, 1.05 mmol) was added. The reaction mixture was heated at 120° C. under microwave irradiation for 30 min., then diluted with MeOH (1 ml) and purified with SPE-(SCX) to afford the title compound (100%); MS (ES/+) m/z: 498 [MH⁺]. C₂₅H₂₉BrN₄O₂ requires 498; ¹H-NMR (300 MHz, CDCl₃) δ(ppm): 8.35 (d, 1H), 7.70 (d, 1H), 7.55 (t, 1H), 7.40 (d, 1H), 7.30-6.85 (m, 4H), 4.85 (q, 1H), 3.5 (br s, 2H), 3.10-2.6 (m, 15H), 1.45 (d, 3H).

### Description 61: 2-Methyl-8-{2-[4-(2-methyl-5-quinolinyl)-1-piperazinyl]ethyl}-2H-1,4-benzoxazin-3(4H)-one (D61)

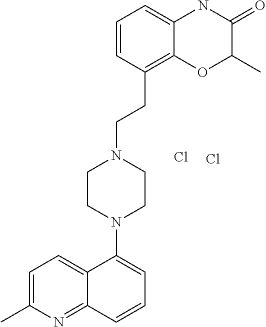

2-[(2-Bromo-6-{2-[4-(2-methyl-5-quinolinyl)-1-piperazinyl]ethyl}phenyl)oxy]-propanamide (D60) (370 mg, 0.74 mmol), CuI (141 mg, 0.74 mmol), N,N′-dimethylethylendiamine (65 mg, 0.74 mmol) and K₂CO₃(195 mg, 1.5 mmol) were suspended in NMP (5 ml) and stirred at 150° C. for 1 h. The resulting reaction mixture was purified with SPE-(SCX) and then with chromatography on silica gel using DCM/MeOH (97:3) to afford the title compound as colourless solid (150 mg, 50%); MS: (ES/+) m/z: 417 [MH⁺]. C₂₅H₂₈N₄O₂ requires 416; ¹H-NMR (300 MHz, CDCl₃) δ(ppm): 8.61 (br s, 1H), 8.41 (d, 1H), 7.35 (d, 1H), 7.60 (t, 1H), 7.35-7.25 (m, 1H), 7.10 (d, 1H), 7.96-6.86 (m, 2H), 6.76-6.66 (m, 1H), 4.7 (m, 1H), 3.1-2.6 (m, 15H), 1.63 (d, 3H).

### Description 62: 6-Bromo-3-fluoro-2-(2-{4-[2-(trifluoromethyl)-5-quinolinyl]-1-piperazinyl}ethyl)phenol (D62)

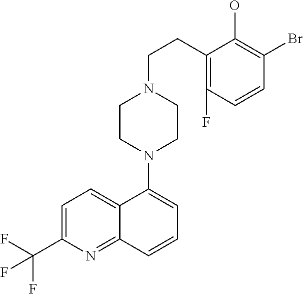

5-(1-piperazinyl)-2-(trifluoromethyl)quinoline (D47) (211 mg, 1 mmol) and (3-bromo-6-fluoro-2-hydroxyphenyl)acetaldehyde (D56) (231 mg, 1 mmol) were stirred at room temperature in DCM (3 ml) for 30 min., then Na₂SO₄ (142 mg, 1 mmol) was added. The resulting mixture was stirred at room temperature for a further 30 min. and then sodium triacetoxyborohydride (212 mg, 1 mmol) was added and the mixture was stirred overnight. The reaction mixture was poured into aqueous NH₄Cl (50 ml) and extracted with DCM (3×50 ml). The combined organic phases were washed with aqueous NH₄Cl (50 ml) and H₂O (50 ml), dried over Na₂SO₄ and concentrated. The resulting crude material was purified by chromatography on silica gel using DCM/MeOH (99:1) to afford the title compound as yellow solid (298 mg, 60%); MS: (ES/+) m/z: 499 [MH⁺]. C₂₂H₂₀BrF₄N₃O requires 498; ¹H-NMR (300 MHz, CDCl₃) δ(ppm): 8.65 (d, 1H), 7.96 (d, 1H), 7.8-7.7 (m, 2H), 7.4-7.3 (M, 2H), 6.48 (t, 1H), 3.1-2.9 (m, 12H).

### Description 63: 2-{[6-Bromo-3-fluoro-2-(2-{4-[2-(trifluoromethyl)-5-quinolinyl]-1-piperazinyl}ethyl)phenyl]oxy}acetamide (D63)

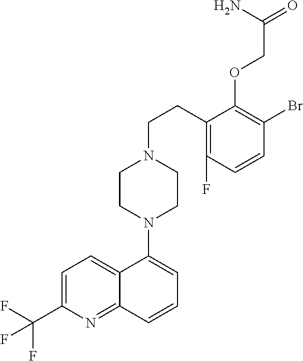

6-Bromo-3-fluoro-2-(2-{4-[2-(trifluoromethyl)-5-quinolinyl]-1-piperazinyl}-ethyl)phenol (D62) (285 mg, 0.57 mmol) and 2-bromoacetamide (86 mg, 0.63 mmol) and K₂CO₃ (226 mg, 1.71 mmol) in acetone (10 ml) were stirred at 40° C. for 48 hours. After removing the solvent under reduced pressure, H₂O (20 ml) was added and the mixture was extracted with DCM (3×25 ml). The combined organic phases were washed with H₂O (40 ml), dried over Na₂SO₄, and concentrated. The resulting crude material was purified by chromatography on silica gel using DCM/MeOH (96:4) to afford the title compound as colourless solid (200 mg, 63%); MS (ES/+) m/z: 556 [MH⁺], C₂₄H₂₃BrF₄N₄O₂ requires 555; ¹H-NMR (300 MHz, CDCl₃) δ(ppm): 8.65 (d, 1H), 7.90 (m, 1H), 7.7 (m, 2H), 7.4 (m, 1H), 7.20 (d, 1H), 7.0 (br s, 1H), 6.85 (t, 1H), 6.65 (br s, 1H), 4.5 (s, 2H), 3.1-2.6 (m, 12H).

### Description 64: 7-Fluoro-8-(2-{4-[2-(trifluoromethyl)-5-quinolinyl]-1-piperazinyl}ethyl)-2H-1,4-benzoxazin-3(4H)-one (D64)

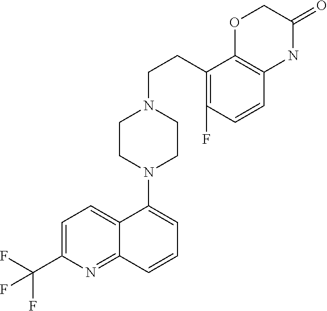

2-{[6-Bromo-3-fluoro-2-(2-{4-[2-(trifluoromethyl)-5-quinolinyl]-1-piperazinyl}-ethyl)phenyl]oxy}acetamide (D63) (200 mg, 0.36 mmol), CuI (68 mg, 0.36 mmol), N,N′-dimethylethylendiamine (32 mg, 0.36 mmol) and K₂CO₃ (100 mg, 0.72 mmol) were suspended in NMP (5 ml) and stirred at 150° C. for 1 hour. The resulting reaction mixture was purified with SPE-(SCX) and then with chromatography on silica gel using DCM/MeOH (96:4) to afford the title compound as a solid (165 mg, 100%); MS (ES/+) m/z: 475 [MH⁺], C₂₄H₂₂F₄N₄O₂ requires 474; ¹H-NMR (300 MHz, CDCl₃) δ(ppm): 8.65 (d, 1H), 7.70 (m, 1H), 7.8-7.6 (m, 3H), 7.2 (m, 1H), 6.7-6.6 (m, 2H), 4.6 (s, 2H), 3.1-2.6 (m, 12H).

### Description 65: 6-Bromo-3-fluoro-2-{2-[4(2-methyl-5-quinazolinyl)-1-piperazinyl]ethyl}phenol) (D65)

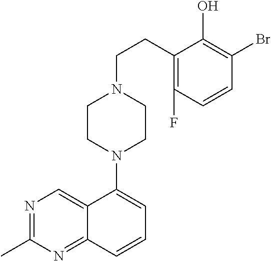

2-Methyl-5-(1-piperazinyl)quinazoline (180 mg, 0.79 mmol) and (3-bromo-6-fluoro-2-hydroxyphenyl)acetaldehyde (D56) (180 mg, 0.79 mmol) were stirred at room temperature in DCM (10 ml) for 30 min., then Na₂SO₄ (112 mg, 0.79 mmol) was added. The resulting mixture was stirred at room temperature for further 30 min. and then sodium triacetoxyborohydride (167 mg, 0.79 mmol) was added and the reaction mixture stirred overnight. The reaction mixture was poured into aqueous NH₄Cl (50 ml) and then extracted with DCM (3×40 ml). The combined organic phases were washed with aqueous NH₄Cl (30 ml) and H₂O (30 ml), dried over Na₂SO₄ and concentrated. The resulting crude product was purified by chromatography on silica gel using DCM/MeOH (98:2) to afford the title compound as a yellow solid (210 mg, 60%); MS (ES/+) m/z: 445 [MH⁺]. C₂₁H₂₂BrFN₄O requires 444; ¹H-NMR (300 MHz, CDCl₃) δ(ppm): 9.5 (s, 1H), 7.78 (t, 1H), 7.62 (d, 1H), 7.34-7.25 (m, 2H), 7.13 (d, 1H), 6.47 (t, 1H), 3.4-2.8 (m, 15H).

### Description 66: 2-[(6-Bromo-3-fluoro-2-{2-[4-(2-methyl-5-quinazolinyl)-1-piperazinyl]ethyl}phenyl)oxy]acetamide (D66)

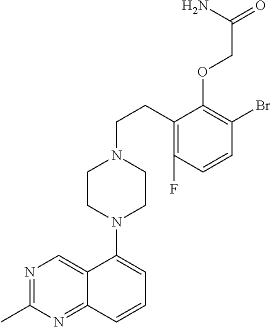

6-Bromo-3-fluoro-2-{2-[4-(2-methyl-5-quinazolinyl)-1-piperazinyl]ethyl}phenol (D65) (210 mg, 0.47 mmol), 2-bromoacetamide (74 mg, 0:52 mmol) and K₂CO₃ (186 mg, 1.41 mmol) in acetone (10 ml) were stirred at 40° C. for 48 hours. After removing the solvent under reduced pressure H₂O (30 ml) was added and the resulting mixture was extracted with DCM (3×25 ml). The combined organic phases were washed with H₂O (40 ml), dried over Na₂SO₄ and concentrated. The resulting crude product was purified by chromatography on silica gel using DCM/MeOH (95:5) to afford the title compound as a solid (200 mg, 85% yield); MS (ES/+) m/z: 502 [MH⁺]. C₂₃H₂₅BrFN₅O₂ required 501; ¹H-NMR (300 MHz, CDCl₃) δ(ppm): 9.5 (s, 1H), 7.75 (t, 1H), 7.55 (t, 1H), 7.4 (m, 1H), 7.05 (d, 1H), 6.9 (br s, 1H), 6.8 (t, 1H), 5.75 (br s, 1H), 4.5 (s, 2H), 3.1-2.6 (m, 15H).

### Description 67: 7-Fluoro-8-{2-[4-(2-methyl-5-quinazolinyl)-1-piperazinyl]ethyl}-2H-1,4-benzoxazin-3(4H)-one (D67)

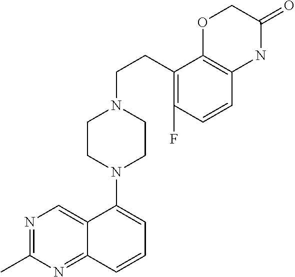

2-[(6-Bromo-3-fluoro-2-{2-[4-(2-methyl-5-quinazolinyl)-1-piperazinyl]-ethyl}phenyl)oxy]acetamide (D66) (200 mg, 0.40 mmol), CuI (76 mg, 0.40 mmol), N,N′-dimethylethylendiamine (35 mg, 0.40 mmol) and K₂CO₃ (105 mg, 0.80 mmol) were suspended in NMP (5 ml) and stirred at 150° C. for 1 hour. The resulting reaction mixture was purified with SPE-(SCX) and then with chromatography on silica gel using DCM/MeOH (96:4) to afford the title compound as a solid (90 mg, 53%); MS (ES/+) m/z: 422 [MH⁺], C₂₃H₂₄FN₅O₂ required 421; ¹H-NMR (300 MHz, CDCl₃) δ(ppm): 9.5 (s, 1H), 7.8-7.7 (m, 2H), 7.55 (d, 1H), 7.05 (d, 1H), 6.7-6.5 (m, 2H), 4.6 (s, 1H), 3.1-2.6 (m, 15H).

### Description 68: 1,1-Dimethylethyl 4-(2-formyl-5-quinolinyl)-1-piperazinecarboxylate (D68)

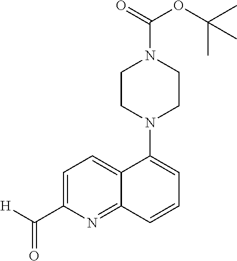

To a solution of 1,1-dimethylethyl 4-(2-methyl-5-quinolinyl)-1-piperazinecarboxylate (D165) (4.51 g, 13.8 mmol) in dioxane (20 ml) was added selenium dioxide (1.83 g, 16.5 mmol). The mixture was stirred at 90° C. for 1.5 hours, cooled to room temperature, and filtered and evaporated. The residue was purified by flash column chromatography eluting with cyclohexane/ethyl acetate (9:1 to 1:1) to afford the title compound as a yellow solid (3.35 g, 71%); MS (ES; m/z): 342[MH⁺], C₁₆H₂₃N₃O₃ requires 341.41; NMR (¹H, 300 MHz, CDCl₃) δ: 10.20 (s, 1H), 8.63 (d, 1H), 7.98 (q, 2H), 7.71 (t, 1H), 7.21 (d, 1H), 3.69 (bs, 4H), 3.03 (bs, 4H), 1.48 (s, 9H).

### Description 69: 1,1-Dimethylethyl 4-(2-cyano-5-quinolinyl)-1-piperazinecarboxylate (D69)

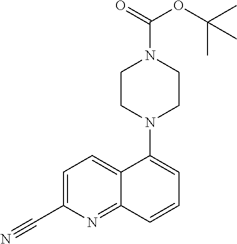

To a solution of 1,1-dimethylethyl 4-(2-formyl-5-quinolinyl)-1-piperazinecarboxylate (D68) (522 mg, 1.52 mmol) in 20% ammonia-water solution (15 ml) and THF (3 ml) was added iodine (427 mg, 1.68 mmol). The mixture was stirred at room temperature for 1 hour. Then aqueous Na₂S₂O₃ solution (5%, 10 ml) was added and the product was extracted with DCM (3×15 ml). The organic layer was dried (MgSO₄), filtered and evaporated in vacuo. The residue was purified by silica gel chromatography eluting with cyclohexane/ethyl acetate (8:2 to 0:1) affording as a yellow solid (265 mg, 51%); MS (ES; m/z): 339[MH⁺]. C₁₆H₂₂N₄O₂ requires 338.41; NMR (¹H, 300 MHz, DMSO-d₆) δ: 8.70 (d, 1H), 7.97 (d, 1H), 7.79 (m, 2H), 7.36 (m, 1H), 3.57 (bs, 4H), 2.95 (m, 4H), 1.40 (s, 9H).

### Description 70: 5-(1-piperazinyl)-2-quinolinecarbonitrile (D70)

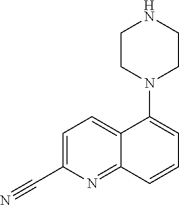

A solution of 1,1-dimethylethyl 4-(2-cyano-5-quinolinyl)-1-piperazinecarboxylate (D69) (1.04 g, 3.06 mmol) in a mixture of TFA/DCM (20 ml, 1:3) was stirred at room temperature overnight. After evaporation the crude material was purified by SPE-SCX purification to afford a yellow solid (633 mg, 87%). HPLC/MS analysis showed a second product (28%, m/z 312). This material may be used in the next step without further purification; MS (ES; m/z): 239 [MH⁺], C₁₄H₁₄N₄ requires 238.29.

### Description 71: 2-Oxo-1,2,3,4-tetrahydro-5-quinolinyl trifluoromethanesulfonate (D71)

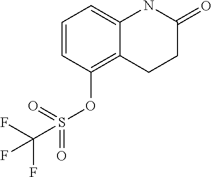

1,1,1-Trifluoro-N-phenyl-N-[(trifluoromethyl)sulfonyl]methanesulfonamide (1.21 g, 3.39 mmol) was added portion-wise to a stirred suspension of 5-hydroxy-3,4-dihydro-2(1H)-quinolinone (Davos, commercially available) (460 mg, 2.82 mmol) in CH₃CN (25 ml), and triethylamine (492 μl, 3.53 mmol) at 0° C. The reaction mixture was stirred for 15 hours at room temperature then quenched with water (20 ml) and extracted with DCM (3×50 ml). The combined organic layers were dried (Na₂SO₄) and concentrated in vacuo. The crude product was purified by SPE-SI cartridge, eluting with cyclohexane/ethyl acetate (7:3), to afford the title compound (812 mg, 97%); MS (ES) m/z: 296.00 [MH⁺], C10H8F3NO4S requires 295.24; ¹H-NMR (300 MHz, CDCl₃) δ: 7.92 (bs, 1H), 7.28 (m, 1H), 6.97 (m, 1H), 6.76 (m, 1H), 3.08 (t, 2H), 2.68 (t, 2H).

### Description 72: 5-(2-Propen-1-yl)-3,4-dihydro-2(1H)-quinolinone (D72)

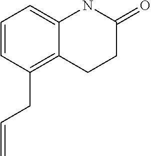

To a solution of 2-oxo-1,2,3,4-tetrahydro-5-quinolinyl trifluoromethanesulfonate (D71) (919 mg, 3.12 mmol) in dry DMF (9 ml) were added allyltributylstannane (1.16 ml, 3.74 mmol), LiCl (3 mg, 2%) and tetrakis(triphenylphosphine) palladium(0) (361 mg, 10%) at room temperature. The reaction mixture was stirred at 100° C. for 2 hours then cooled, quenched with water (10 ml) and extracted with DCM (3×50 ml). The combined organic layers were dried (Na₂SO₄) and concentrated in vacuo to give the crude product which was purified by flash chromatography on silica gel, eluting with cyclohexane/ethyl acetate (7:3), to afford the title compound (496 mg, 85%); MS (ES) m/z: 188.10 [MH⁺], C12H13NO requires 187.24; ¹H-NMR (300 MHz, CDCl₃) δ: 7.93 (bs, 1H), 7.13 (t, 1H), 6.88 (d, 1H), 6.65 (d, 1H), 6.00-5.87 (m, 1H), 5.11-4.95 (m, 2H), 3.42-3.38 (m, 2H), 2.94 (t, 2H), 2.62 (t, 2H).

### Description 73: (2-Oxo-[1,2,3,4]-tetrahydro-5-quinolinyl)acetaldehyde (D73)

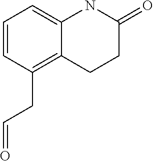

The title compound was prepared in 63% yield according to the procedure of Description 6 starting from 5-(2-propen-1-yl)-3,4-dihydro-2(1H)-quinolinone (D72) (380 mg, 2.03 mmol). The product was purified by flash chromatography on silica gel using ethyl acetate/cyclohexane (1:1) as eluent; MS (ES) m/z: 190.1 [MH⁺], C11H11NO2 requires 189.21; ¹H-NMR (300 MHz, CDCl₃) δ: 9.73 (t, 1H), 7.68 (bs, 1H), 7.19 (t, 1H), 6.90 (d, 1H), 6.71 (d, 1H), 3.77 (d, 2H), 2.88 (t, 2H), 2.63 (d, 2H).

### Description 74: 5-{2-[4-(2-Methyl-5-quinolinyl)-1-piperazinyl]ethyl}-3,4-dihydro-2(1H)-quinolinone (D74)

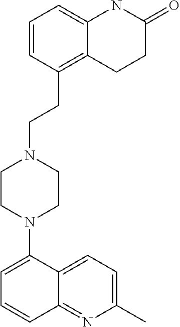

The title compound was prepared in 75% yield following the general reductive amination procedure of Example 1 starting from (2-oxo-1,2,3,4-tetrahydro-5-quinolinyl)acetaldehyde (D73) (242 mg, 1.28 mmol). The crude product was purified by flash chromatography on silica gel eluting with a gradient of methanol in DCM (1 to 3%) to afford the title compound (384 mg, 75%); MS (ES) m/z: 401.20 [MH⁺]. C25H28N4O requires 400.52; ¹H-NMR (500 MHz, DMSO-d6) δ: 10.01 (s, 1H), 8.34 (vbs, 1H), 7.58 (m, 2H), 7.37 (d, 1H), 7.09 (m, 1H), 7.07 (t, 1H), 6:84 (d, 1H), 6.72 (dd, 1H), 3.03 (bs, 4H), 2.89 (t, 2H), 2.80 (m, 2H), 2.74 (bs, 4H), 2.54 (m, 2H), 2.44 (t, 2H), 2.63 (bs, 3H).

### Description 75: 5-methyl-2-nitrophenyl 2-propen-1-yl ether (D75)

The title compound was prepared in quantitative yield (2.5 g) following the procedure of Description 1 starting from 5-methyl-2-nitrophenol (2.0 g, 13 mmol); ¹H-NMR (300 MHz, CDCl₃) δ: 7.71 (d, 1H), 6.77-6.72 (m, 2H), 5.95 (m, 1H), 5.46-5.23 (m, 2H), 4.61-4.58 (m, 2H), 2.33 (s, 3H).

### Description 76: 3-Methyl-6-nitro-2-(2-propen-1-yl)phenol (D76)

The title compound was prepared in 68% yield (1.7 g) following the procedure of Description 2 starting from 5-methyl-2-nitrophenyl 2-propen-1-yl ether (D75) (2.5 g, 13 mmol); ¹H-NMR (300 MHz, CDCl₃) δ: 11.06 (s, 1H), 7.89 (d, 1H), 6.78 (d, 1H), 5.95 (m, 1H), 5.05-4.93 (m, 2H), 3.52-3.49 (m, 2H), 2.37 (s, 3H).

### Description 77: Methyl {[3-methyl-6-nitro-2-(2-propen-1-yl)phenyl]oxy}acetate (D77)

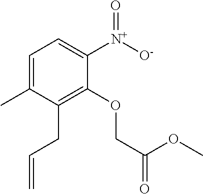

The title compound was prepared following the procedure of Description 3 starting from 3-methyl-6-nitro-2-(2-propen-1-yl)phenol (D76) (1.7 g, 8.81 mmol); MS (ES) m/z: 266.1 [MH⁺], C13H15NO5 requires 265.26; ¹H-NMR (300 MHz, CDCl₃) δ: 7.71 (d, 1H), 7.08 (d, 1H), 5.95 (m, 1H), 5.10-4.81 (m, 2H), 4.61 (s, 2H), 3.81 (s, 3H), 3.59-3.55 (m, 2H), 2.36 (s, 3H).

### Description 78: 7-Methyl-8-(2-propen-1-yl)-2H-1,4-benzoxazin-3(4H)-one (D78)

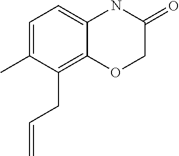

The title compound was prepared in 48% yield (860 mg) following the procedure of Description 4 starting from methyl {[3-methyl-6-nitro-2-(2-propen-1-yl)phenyl]oxy}acetate (D77) (2.33 g, 8.81 mmol); MS; (ES) m/z: 204.1 [MH⁺]. C12H13NO2 requires 203.24; ¹H-NMR (300 MHz, CDCl₃) δ: 7.79 (bs, 1H), 6.77 (d, 1H), 6.56 (d, 1H), 5.89 (m, 1H), 5.02-4.88 (m, 2H), 4.58 (s, 2H), 3.42-3.39 (m, 2H), 2.25 (s, 3H).

### Description 79: (7-Methyl-3-oxo-3,4-dihydro-2H-1,4-benzoxazin-8-yl)acetaldehyde (D79)

The title compound was prepared in 48% yield (860 mg) following the procedure of Description 6 starting from 7-methyl-8-(2-propen-1-yl)-2H-1,4-benzoxazin-3(4H)-one (D78) (2.33 g, 8.81 mmol); MS (ES) m/z: 206.1 [MH⁺]. C11H11NO3 requires 205.21; ¹H-NMR (300 MHz, CDCl₃) δ: 9.70 (s, 1H), 7.79 (bs, 1H), 6.81 (d, 1H), 6.66 (d, 1H), 4.56 (s, 2H), 3.75 (s, 2H), 2.38 (s, 3H).

### Description 80: 7-Methyl-8-{2-[4-(2-methyl-5-quinolinyl)-1-piperazinyl]ethyl}-2H-1,4-benzoxazin-3(4H)-one (D80)

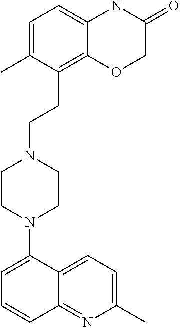

The title compound was prepared following the general reductive amination procedure of Example 1 starting from (7-methyl-3-oxo-3,4-dihydro-2H-1,4-benzoxazin-8-yl)acetaldehyde (D79) (242 mg, 1.28 mmol). The crude product was purified by flash chromatography on silica gel eluting with a gradient of methanol in DCM (1 to 3%) to afford the title compound (384 mg, 75%); MS (ES) m/z: 417.20 [MH⁺], C25H28N4O2 requires 416.52; ¹H-NMR (300 MHz, CDCl₃) δ: 10.52 (s, 1H), 8.33 (d, 1H), 7.57 (m, 2H), 7.35 (d, 1H), 7.09 (m, 1H), 6.74 (d, 1H), 6.63 (d, 1H), 4.53 (s, 2H), 3.04 (bm, 4H), 2.81-2.64 (m, 6H), 2.62 (s, 3H), 2.38-2.57 (m 2H), 2.24 (s, 3H).

### Description 81: 5-{[(1,1-Dimethylethyl)(dimethyl)silyl]oxy}-3,4-dihydro-2(1H)-quinolinone (D81)

To a stirred solution of 5-hydroxy-3,4-dihydro-2(1H)-quinolinone (640 mg, 3.93 mmol) in DMF (3.5 ml) were added t-butyldimethylsilyl chloride (651 mg, 4.32 mmol) and imidazole (321 mg, 4.71 mmol) at room temperature. After 4 hours water (10 ml) was added and the mixture was extracted with DCM (3×70 ml). The combined organic layers were dried (Na₂SO₄), concentrated in vacuo to give the title compound (997 mg, 91%) that was used in the next step without further purification; MS (ES) m/z: 278.2 [MH⁺], C15H23NO2Si requires 277.44; ¹H-NMR (300 MHz, CDCl₃) δ: 7.62 (bs, 1H), 7.03 (t, 1H), 6.52 (d, 1H), 6.36 (d, 1H), 2.95 (t, 2H), 2.61 (t, 2H), 1.03 (s, 9H), 0.25 (s, 6H).

### Description 82: Ethyl 6-{[(1,1-dimethylethyl)(dimethyl)silyl]oxy}-4,5-dihydroimidazo[1,5-a]-quinoline-3-carboxylate (D82)

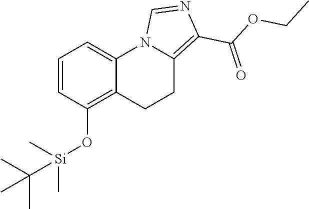

The title compound was prepared according to the procedure of Example 80 starting from 5-{[(1,1-dimethylethyl)(dimethyl)silyl]oxy}-3,4-dihydro-2(1H)-quinolinone (D81) (997 mg, 3.6 mmol). The crude product was purified by flash chromatography on silica gel eluting with ethyl acetate/cyclohexane (4:6) to afford the title compound (992 mg, 74%); MS (ES) m/z: 373.2 [MH⁺], C20H28N2O3Si requires 372.54; ¹H-NMR (300 MHz, CDCl₃) δ: 7.99 (s, 1H), 7.21 (t, 1H), 7.09 (d, 1H), 6.77 (d, 1H), 4.41 (q, 2H), 3.31 (t, 2H), 2.93 (t, 2H), 1.44 (t, 3H), 1.05 (s, 9H), 0.27 (s, 6H).

### Description 83: Ethyl 6-hydroxy-4,5-dihydroimidazo[1,5-a]quinoline-3-carboxylate (D83)

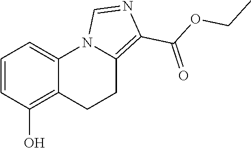

To a solution of ethyl 6-{[(1,1-dimethylethyl)(dimethyl)silyl]oxy}-4,5-dihydroimidazo[1,5-a]-quinoline-3-carboxylate (D82) (642 mg, 1.73 mmol) in THF (20 ml) was added tetrabutylammonium fluoride (3.45 ml of 1M sol. in THF, 3.45 mmol) and the resulting solution was stirred at room temperature. After 2 hours a saturated solution of ammonium chloride (10 ml) was added, THF was removed in vacuo and the resulting mixture was extracted with DCM (4×100 ml). The combined organic layers were dried (Na₂SO₄), concentrated in vacuo to give the title compound (446 mg, 100%) that may be used in the next step without further purification; MS (ES) m/z: 259.1 [MH⁺], C14H14N2O3 requires 258.28; ¹H-NMR (300 MHz, DMSO-d6) δ: 10.07 (bs, 1H), 8.72 (s, 1H), 7.27 (d, 1H), 7.17 (t, 1H), 6.84 (d, 1H), 4.26 (q, 2H), 3.18 (t, 2H), 2.82 (t, 2H), 1.30 (t, 3H).

### Description 84: Ethyl 6-{[(trifluoromethyl)sulfonyl]oxy}-4,5-dihydroimidazo[1,5-a]quinoline-3-carboxylate (D84)

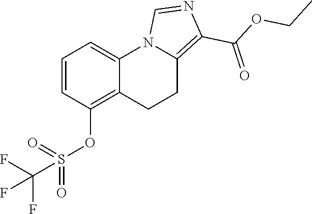

The title compound was prepared according to the procedure of Description 71 starting from ethyl 6-hydroxy-4,5-dihydroimidazo[1,5-a]quinoline-3-carboxylate (D83) (446 mg, 1.73 mmol). The crude product was purified by flash chromatography on silica gel eluting with dichloromethane/methanol (97:3) then with SCX cartridge, to afford the title compound (491 mg, 73%); MS (ES) m/z: 391.0 [MH⁺]. C15H13F3N2O5S requires 390.34; ¹H-NMR (300 MHz, CDCl₃) δ: 8.03 (s, 1H), 7.55-7.40 (m, 2H), 7.26 (m, 1H), 4.41 (q, 2H), 3.39 (t, 2H), 3.06 (t, 2H), 1.43 (t, 3H).

### Description 85: Ethyl 6-(2-propen-1-yl)-4,5-dihydroimidazo[1,5-a]quinoline-3-carboxylate (D85)

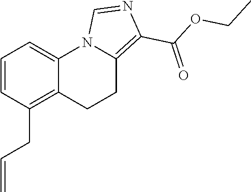

The title compound was prepared in quantitative yield according to the procedure of Description 72 starting from ethyl 6-{[(trifluoromethyl)-sulfonyl]oxy}-4,5-dihydroimidazo[1,5-a]quinoline-3-carboxylate (D84) (491 mg, 1.26 mmol). The crude product was purified by flash chromatography on silica gel eluting with ethyl acetate/cyclohexane (7:3) to afford the title compound (354 mg, quant.); MS (ES) m/z: 283.2 [MH⁺], C17H18N2O2 requires 282.34; ¹H-NMR (300 MHz, CDCl₃) δ: 7.99 (s, 1H), 7.40-7.28 (m, 2H), 7.13 (d, 1H), 5.95 (m, 1H), 5.13-4.95 (m, 2H), 4.40 (q, 2H), 3.47 (m, 2H), 3.30 (t, 2H), 2.89 (t, 2H), 1.43 (t, 3H).

### Description 86: Ethyl 6-(2-oxoethyl)-4,5-dihydroimidazo[1,5-a]quinoline-3-carboxylate (D86)

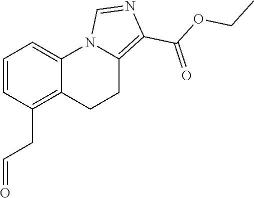

The title compound was prepared in 85% yield (305 mg) following the procedure of Description 6 starting from ethyl 6-(2-propen-1-yl)-4,5-dihydroimidazo[1,5-a]quinoline-3-carboxylate (D85) (354 g, 1.25 mmol); ¹H-NMR (300 MHz, CDCl₃) δ: 9.77 (t, 1H), 8.01 (s, 1H), 7.46 (d, 1H), 7.35 (t, 1H), 7.14 (d, 1H), 4.39 (q, 2H), 3.86 (d, 2H), 3.32 (t, 2H), 2.83 (t, 2H), 1.43 (t, 3H).

### Description 87: 5-(2-Propen-1-yl)quinoline (D87)

Allyltributylstannane (2.4 ml, 8 mmol) was added to a mixture of 5-bromo quinoline (1.5 g, 7.24 mmol), tris(dibenzylideneacetone)dipalladium(0) (331 mg, 0.36 mmol) and triphenylphosphine (760 mg, 2.9 mmol) in toluene (50 ml). The reaction mixture was stirred overnight at 120° C. and then was quenched with water (30 ml) and 1N hydrochloric acid (10 ml). The organic layer was extracted with 1M aqueous HCl (3×20 ml) and then the combined aqueous phases were basified with solid NaHCO₃, then extracted with DCM (3×50 ml). The combined organic extracts were dried over anhydrous Na₂SO₄ and evaporated in vacuo. The crude material was purified by SPE-SI cartridge eluting with 5% ethyl acetate in cyclohexane to afford the title compound as a pale yellow solid (1 g, 81%); MS (ES) m/z: 170.1 [MH⁺], C12H11N requires 169.23; ¹H-NMR (300 MHz, CDCl₃) δ: 8.75 (t, 1H), 8.20 (d, 1H), 7.85 (d, 1H), 7.45 (t, 1H), 5.95-5.8 (m, 1H), 5.0-4.8 (m, 2H), 3.65 (d, 2H).

### Description 88: 5-(2-Propen-1-yl)quinoline-N-oxide (D88)

3-Chloroperbenzoic acid (55%, 13.4 g, 42.6 mmol) was added portion-wise to a solution of 5-(2-propen-1-yl)quinoline (D87) (6 g, 35.5 mmol) in DCM (100 ml) at 0° C. The reaction mixture was stirred at room temperature for 2 hours and then quenched with saturated aqueous NaHCO₃ (100 ml). The mixture was extracted with DCM (3×50 ml), and the combined organic extracts were dried over anhydrous Na₂SO₄ and evaporated in vacuo. The crude material was purified by SPE-SI cartridge eluting with 40% ethyl acetate in cyclohexane to afford the title compound as a pale brown solid (5.4 g, 82%); MS (ES) m/z: 186.2 [MH⁺], C12H11NO requires 185.24; ¹H-NMR (300 MHz, CDCl₃) δ: 8.7 (bs, 1H), 8.50 (bs, 1H), 7.9 (bs, 1H), 7.7 (bs, 1H), 7.5 (bs, 1H), 7.3 (bs, 1H), 6 (bs, 1H), 5.1 (d, 1H), 5.0 (d, 1H), 3.8 (bs, 2H).

### Description 89: Ethyl nitro[5-(2-propen-1-yl)-2-quinolinyl]acetate (D89)

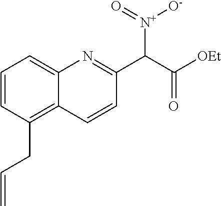

Ethyl nitroacetate (1.3 ml, 11.9 mmol) was added dropwise to a mixture of 5-(2-propen-1-yl)quinoline-N-oxide (D88) (2 g, 10.8 mmol) and acetic anhydride (5 ml). After 30 min. stirring the formation of a precipitate was observed, the reaction was quenched with water (10 ml) and filtered. The solid was washed with methanol (10 ml) and dried under vacuum to afford the title compound as a yellow solid (1.4 g, 43%); MS (ES) m/z: 301.1 [MH⁺]. C16H16N2O4 requires 300.31; ¹H-NMR (300 MHz, CDCl₃) δ: 8.2 (bd, 1H), 7.7-7.4 (m, 3H), 7.3 (bd, 1H), 6.1-5.9 (bm, 1H), 5.2-4.9 (bm, 2H), 4.5-4.3 (bm, 2H), 3.7 (bs, 2H), 1.4-1.3 (bm, 3H).

### Description 90: Ethyl 6-(2-propen-1-yl)imidazo[1,5-a]quinoline-3-carboxylate (D90)

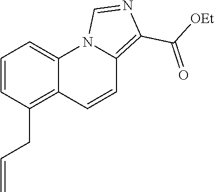

Zinc powder (720 mg, 11 mmol) was added to a mixture of ethyl nitro[5-(2-propen-1-yl)-2-quinolinyl]acetate (D89) (550 mg, 1.83 mmol) in glacial acetic acid (15 ml). The reaction mixture was stirred at room temperature for 4 hours and then quenched with water (50 ml) and diluted with DCM (50 ml), cooled to 0° C. and neutralized with solid NaHCO₃. The aqueous layer was separated and extracted with DCM (2×50 ml) and then the combined organic phases were washed with saturated aqueous NaHCO₃ (3×50 ml), dried over anhydrous Na₂SO₄ and evaporated in vacuo. The intermediate ethyl amino[5-(2-propen-1-yl)-2-quinolinyl]acetate was quickly treated with triethyl orthoformate (5 ml) and heated with stirring at 150° C. for 7 min. under microwave irradiation. The resulting reaction mixture was passed through SCX cartridge to remove triethyl orthoformate and then was purified by SPE-SI cartridge eluting with 50% ethyl acetate in cyclohexane to afford the title compound as a pale yellow solid (245 g, 48%); MS (ES) m/z: 281.20 [MH⁺], C17H16N2O2 requires 280.33; ¹H-NMR (300 MHz, CDCl₃) δ: 8.6 (s, 1H), 8.1 (d, 1H), 7.9 (d, 1H), 7.5-7.6 (m, 2H), 7.3 (d, 1H), 6.0-5.9 (m, 1H), 5.1 (d, 1H), 5.0 (d, 1H), 4.45 (q, 2H), 3.8 (d, 2H), 1.4 (t, 3H).

### Description 91: Ethyl 6-(2-oxoethyl)imidazo[1,5-a]quinoline-3-carboxylate (D91)

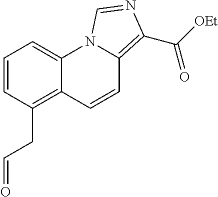

Osmium tetroxide (0.63 ml of a 4% by wt. solution in water) was added to a stirred solution of ethyl 6-(2-propen-1-yl)imidazo[1,5-a]quinoline-3-carboxylate (D90) (245 mg, 0.88 mmol) in THF/water (2:1; 9 ml). After 10 min. sodium periodate (470 mg, 2.2 mmol) was added and the reaction mixture was stirred for an additional 20 min. After evaporation of the THF the residue was partitioned between water (10 ml) and ethyl acetate (3×10 ml). The organic layers were combined, dried (Na₂SO₄) and concentrated in vacuo affording the title compound (220 mg). The crude product may be used without further purification for the next step; ¹H-NMR (300 MHz, CDCl₃) δ: 9.8 (s, 1H), 8.7 (s, 1H), 8.15 (d, 1H), 8.05 (d, 1H), 7.65 (t, 1H), 7.3 (m, 2H), 4.45 (q, 2H), 4.15 (s, 2H), 1.4 (t, 3H).

### Description 92: 6-(2-Propen-1-yl)tetrazolo[1,5-a]quinoline (D92)

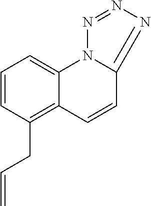

A mixture of 5-(2-propen-1-yl)quinoline-N-oxide (D88) (500 mg, 2.7 mmol) and methanesulfonyl chloride (0.22 ml, 2.7 mmol) in dry acetonitrile (5 ml) was stirred for 3 hours at room temperature. Trimethylsilyl azide (0.36 ml, 2.7 mmol) was added dropwise to the suspension and then the mixture was heated to reflux for 24 hours. Evaporation in vacuo of the volatiles and purification by SPE-Si cartridge eluting with DCM afforded the title compound (318 mg, 56%); MS (ES) m/z: 211.1 [MH⁺], C12H10N4 requires 210.2; ¹H-NMR (300 MHz, CDCl₃) δ: 8.6 (d, 1H), 8.1 (d, 1H), 7.9-7.7 (m, 2H), 7.6 (d, 1H), 6.1-5.9 (m, 1H), 5.15 (d, 1H), 5.0 (d, 1H), 3.8 (d, 2H).

### Description 93: Tetrazolo[1,5-a]quinolin-6-ylacetaldehyde (D93)

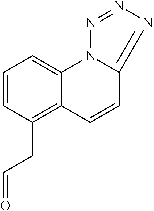

Osmium tetroxide (0.34 ml of a 4% by wt. solution in water) was added to a stirred solution of 6-(2-propen-1-yl)tetrazolo[1,5-a]quinoline (D92) (100 mg, 0.48 mmol) in THF/water (2:1; 4.5 ml). After 10 min. sodium periodate (256 mg, 1.2 mmol) was added and the reaction mixture was stirred for an additional 15 min. After evaporation of the THF, the residue was partitioned between water (10 ml) and ethyl acetate (3×10 ml). The organic layers were combined, dried (Na₂SO₄) and concentrated in vacuo. Trituration of the crude material with DCM/ethyl acetate (1:1, 2 ml) afforded the title compound (62 mg, 61%); MS (ES) m/z: 213.1 [MH⁺], C11H8N4O requires 212.2; ¹H-NMR (300 MHz, DMSO-d⁶) δ: 9.8 (s, 1H), 8.6 (d, 1H), 8.3 (d, 1H), 8.1 (d, 1H), 7.95 (t, 1H), 7.7 (d, 1H), 4.5 (s, 2H).

### Description 94: 5-(2-Propen-1-yl)-2(1H)-quinolinone (D94)

Trifluoroacetic anhydride (5.1 ml, 36.2 mmol) was added to a stirred solution of 5-(2-propen-1-yl)quinoline-N-oxide (D88) (670 mg, 3.62 mmol) in DMF (12 ml). The mixture was stirred at room temperature overnight and then quenched with saturated aqueous NaHCO₃ (20 ml) and extracted with ethyl acetate (3×20 ml). The combined organic phases were dried over Na₂SO₄ and then evaporated in vacuo. The crude material was purified by SPE-Si eluting with 30% cyclohexane in ethyl acetate to afford the title compound as a white solid (360 mg, 54%); ¹H-NMR (300 MHz, CDCl₃) δ: 13.4 (s, 1H), 8.2 (d, 1H), 7.5 (t, 1H), 7.4 (d, 1H), 7.3 (d, 1H), 6.8 (d, 1H), 6.1-5.9 (m, 1H), 5.2 (d, 1H), 5.0 (d, 1H), 3.7 (d, 2H).

### Description 95: 5-(2-Propen-1-yl)-2(1H)-quinolinethione (D95)

A mixture of 5-(2-propen-1-yl)-2(1H)-quinolinone (D94) (1.1 g, 6 mmol) and Lawesson reagent (1.2 g, 3 mmol) in dry toluene (30 ml) was heated at reflux for 30 min. The mixture was cooled to room temperature and the solvent evaporated in vacuo. The crude material was purified by SPE-SI cartridge eluting with 30% ethyl acetate in cyclohexane to afford the title compound as a white solid (0.96 g, 80%); MS (ES) m/z: 202.1 [MH⁺], C12H11NS requires 201.3; ¹H-NMR (300 MHz, CDCl₃) δ: 12.5 (s, 1H), 7.8 (d, 1H), 7.6-7.3 (m, 3H), 7.1 (d, 1H), 6.0-5.8 (m, 1H), 5.05 (d, 1H), 4.95 (d, 1H), 3.7 (d, 2H).

### Description 96: 1-Methyl-6-(2-propen-1-yl)[1,2,4]triazolo[4,3-a]quinoline (D96)

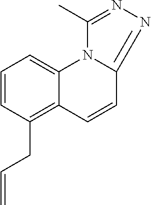

A solution of 5-(2-propen-1-yl)-2(1H)-quinolinethione (D95) (150 mg, 0.75 mmol) in absolute ethanol (1 ml) was added dropwise to a solution of hydrazine monohydrate (1 ml) in absolute ethanol (1 ml) at 100° C. The resulting reaction mixture was stirred at 100° C. for 6 hours and concentrated in vacuo to afford the intermediate 5-(2-propen-1-yl)-2(1H)-quinolinone hydrazone which was immediately used in the following step without any further purification. The hydrazone was mixed with trimethyl orthoacetate (2 ml) and heated with stiffing at 150° C. for 10 min. under microwave irradiation. The resulting reaction mixture was purified by SCX cartridge and then by chromatography (SPE-SI cartridge) eluting with 10% methanol in DCM. The title compound was obtained after trituration with diethyl ether (110 mg, 65%); MS (ES) m/z: 224.1 [MH⁺], C14H13N3 requires 223.3; ¹H-NMR (300 MHz, CDCl₃) δ: 8.2 (d, 1H), 7.8-7.5 (m, 3H), 7.4 (d, 1H), 6.2-6.0 (m, 1H), 5.2 (d, 1H), 5.0 (d, 1H), 3.8 (d, 2H), 3.2 (s, 3H), 1.6 (d, 3H).

### Description 97: (1-Methyl[1,2,4]triazolo[4,3-a]quinolin-6-yl)acetaldehyde (D97)

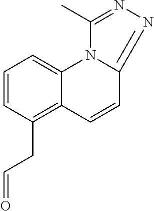

### 4-Methylmorpholine N-oxide (98 mg, 0.84 mmol) and osmium tetroxide (106 μl of a 4% by wt. solution in water, 0.035 eq) were added to a solution of 1-methyl-6-(2-propen-1-yl)[1,2,4]triazolo[4,3-a]quinoline (D96) (94 mg, 0.42 mmol) in a 8:1 mixture of acetone/water (4.5 ml). The reaction mixture was stirred for 4 hours and then quenched with a saturated aqueous solution of sodium sulfite (15 ml). After 30 minutes stirring the mixture was extracted with DCM (3×20 ml). The combined organic layers were dried (Na₂SO₄) and concentrated in vacuo to give the intermediate 3-(1-methyl[1,2,4]triazolo[4,3-a]quinolin-6-yl)-1,2-propanediol (80 mg) which was taken-up in a 1:1 mixture of THF/water (2 ml) without further purification. Sodium periodate (133 mg, 0.62 mmol) and acetone (1 ml) were added and the mixture was stirred for 10 min. After evaporation of the THF, the residue was partitioned between water (10 ml) and DCM (3×20 ml). The combined organic layers were dried (Na₂SO₄) and concentrated in vacuo (methanol was used to favour the solubilization in DCM) to give the title compound as a white solid (¹H-NMR analysis showed the following mixture of compounds 40% of (1-methyl[1,2,4]triazolo[4,3-a]quinolin-6-yl)acetaldehyde plus 60% of 1-(methyloxy)-2-(1-methyl[1,2,4]triazolo[4,3-a]quinolin-6-yl)ethanol; 35 mg). The mixture was used without further purification; ¹H-NMR (300 MHz, CDCl₃) (1-methyl[1,2,4]triazolo[4,3-a]quinolin-6-yl)acetaldehyde δ: 9.9 (s, 1H), 8.3 (d, 1H), 7.8-7.4 (m, 4H), 4.0 (d, 2H), 3.2 (s, 3H) and 1-(methyloxy)-2-(1-methyl[1,2,4]triazolo[4,3-a]quinolin-6-yl)ethanol δ: 8.2 (d, 1H), 7.8 (d, 1H), 7.8-7.4 (m, 3H), 5.9 (bs, 1H), 3.4 (s, 3H), 3.3 (bs, 2H), 3.1 (s, 3H).

### Description 98: Ethyl 6-(2-propen-1-yl)[1,2,3]triazolo[1,5-a]quinoline-3-carboxylate (D98)

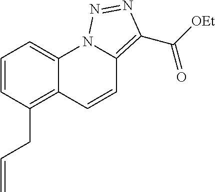

Following the procedure of Description 90, ethyl nitro[5-(2-propen-1-yl)-2-quinolinyl]acetate (D89) (1.2 g, 4 mmol) in glacial acetic add (15 ml) was reacted with zinc powder (1.57 g, 24 mmol) for 2 hours. A portion of the crude intermediate ethyl amino[5-(2-propen-1-yl)-2-quinolinyl]acetate (200 mg) was quickly dissolved in aqueous HCl (2.5 ml, 37%) and after 5 min. stifling a solution in water of sodium nitrite (62 mg in 1.5 ml of water) was added dropwise. After 2 hours reaction the precipitate was filtered and purified by SPE-SI cartridge eluting with 50% ethyl acetate in cyclohexane to afford the title compound (68 mg); MS (ES) m/z 282.20 [MH]⁺. C16H15N3O2 requires 281.3; ¹H-NMR (400 MHz, CDCl₃) δ: 8.8 (d, 1H), 8.15 (d, 1H), 8.0 (d, 1H), 7.6 (d, 1H), 6.2-6.0 (m, 1H), 5.2 (d, 1H), 5.1 (d, 1H), 4.6 (q, 2H), 3.9 (d, 2H), 1.5 (t, 3H).

### Description 99: Ethyl 6-(2-oxoethyl)[1,2,3]triazolo[1,5-a]quinoline-3-carboxylate (D99)

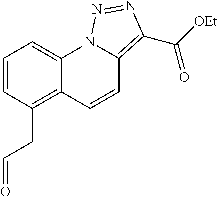

Osmium tetroxide (0.16 ml of a 4% by wt. solution in water) was added to a stirred solution of ethyl 6-(2-propen-1-yl)[1,2,3]triazolo[1,5-a]quinoline-3-carboxylate (D98) (64 mg, 0.23 mmol) in THF/water (2:1; 3 ml). After 15 min. sodium periodate (122 mg, 0.57 mmol) was added and the reaction mixture was stirred for 1 hour. The reaction was diluted with water (5 ml) and extracted with DCM (3×10 ml). The organic layers were combined, dried (Na₂SO₄) and concentrated in vacuo. The crude material was purified by SPE-Si cartridge eluting with 40% cyclohexane in ethyl acetate to afford the title compound (37 mg, 57%); ¹H-NMR (300 MHz, CDCl₃) δ: 9.85 (s, 1H), 8.9 (d, 1H), 8.15 (d, 1H), 7.9-7.7 (m, 2H), 4.5 (q, 2H), 4.2 (s, 2H), 1.5 (t, 3H).

### Description 100: 1,1-Dimethylethyl 4-[2-(difluoromethyl)-5-quinolinyl]-1-piperazinecarboxylate (D100)

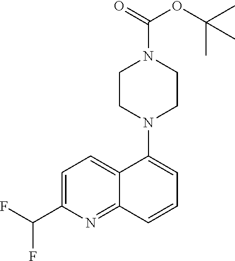

(Diethylamino)sulphur trifluoride (DAST) (428 μl, 3.27 mmol) was added to a solution of 1,1-dimethylethyl 4-(2-formyl-5-quinolinyl)-1-piperazinecarboxylate (D68) (1.015 g, 2.97 mmol) in DCM (10 ml) at room temperature. After 4 hours additional DAST (194 μl, 1.49 mmol) was added and stirring was continued for an additional 1 hour. The reaction was quenched with water (30 ml) and extracted with DCM (3×30 ml). The organic layer was dried (MgSO₄) and evaporated in vacuo to a residue which was purified by flash column chromatography eluting with cyclohexane/ethyl acetate (9:1 to 8:2) to afford the title compound (886 mg, 82%) as a yellow solid; MS (ES; m/z): 364 [MH⁺] C₁₉H₂₃F₂N₃O₂ requires 363.41; ¹H-NMR (300 MHz, CDCl₃) δ: 8.65 (d, 1H), 7.84 (d, 1H), 7.67 (m, 2H), 7.17 (d, 1H), 6.75 (t, 1H), 3.68 (bm, 4H), 3.02 (bm, 4H), 1.49 (s, 9H).

### Description 101: Methyl 3-oxo-2-[5-(2-propen-1-yl)-2-quinolinyl]butanoate (D101)

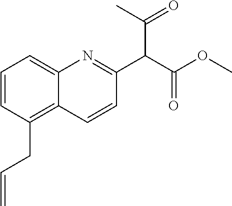

Methyl acetoacetate (1.3 ml) was added dropwise to a mixture of 5-(2-propen-1-yl)quinoline-N-oxide (D88) (2.0 g, 10.81 mol) in acetic anhydride (5 ml), maintaining the internal temperature below 30° C. The reaction mixture was stirred at 30° C. over night and then was poured into ice/water. The precipitate was filtered washed with water and then dried under vacuo at 40° C. to afford the title compound as a yellow solid (2.94 g, 96%); MS (ES) m/z: 284 [MH⁺], C17H17NO3 requires 283.3.

### Description 102: Methyl[5-(2-propen-1-yl)-2-quinolinyl]acetate (D102)

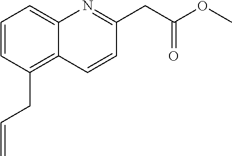

Methyl 3-oxo-2-[5-(2-propen-1-yl)-2-quinolinyl]butanoate (D101) (2.83 g, 10 mmol) was added portion-wise with stirring to 10% solution of HCl (10 ml) over 10 minutes. After stirring for an additional 20 minutes at room temperature, the reaction mixture was made basic with a 10% solution in water of potassium carbonate (35 ml) and then was extracted with DCM (3×50 ml). The organic layer was dried over Na₂SO₄ and then evaporated in vacuo. The residue was purified by flash chromatography eluting with 10% cyclohexane in ethyl acetate to 25% cyclohexane in ethyl acetate to afford the title compound as an orange liquid (1.62 g, 67%); MS (ES) m/z: 242 [MH⁺]. C15H15NO2 requires 241.3.

### Description 103: Methyl 6-(2-propen-1-yl)[1,2,3]triazolo[1,5-a]quinoline-3-carboxylate (D103)

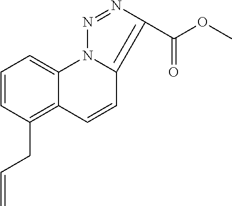

To a mixture of methyl[5-(2-propen-1-yl)-2-quinolinyl]acetate (D102) (620 mg) and ethyl [5-(2-propen-1-yl)-2-quinolinyl]acetate (100 mg obtained according to the procedure reported above for D102 but starting from ethyl acetoacetate in Description 101) (2.96 mmol tot) in dry acetonitrile (15 ml) were added DBU (0.464 ml, 3.1 mmol) and the p-acetamidobenzensulfonyl azide (768 mg, 3.1 mmol) at 0° C. The reaction mixture was stirred for 20 minutes and then quenched at 0° C. with ammonium chloride (20 ml). The mixture was extracted with DCM (3×20 ml), and the organic layer was dried over Na₂SO₄ and then evaporated in vacuo. The residue was purified by flash chromatography eluting with 10% cyclohexane in ethyl acetate to 20% cyclohexane in ethyl acetate to afford the title compound as a white solid (500 mg, 63%); HPLC MS; m/z: 268 [MH⁺]. C15H13N3O2 requires 267.3; presence of =10% of ethyl 6-(2-propen-1-yl)[1,2,3]triazolo[1,5-a]quinoline-3-carboxylate detected.

### Description 104: Methyl 6-(2-oxoethyl)[1,2,3]triazolo[1,5-a]quinoline-3-carboxylate (D104)

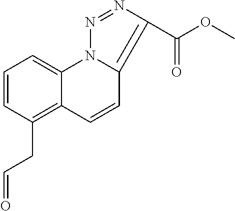

Osmium tetroxide (1.9 ml of a 4% by wt. solution in water) was added to a stirred solution of methyl 6-(2-propen-1-yl)[1,2,3]triazolo[1,5-a]quinoline-3-carboxylate (D103) (450 mg, 1.68 mmol) in THF/water (2:1; 21 ml). After 30 min. sodium periodate (900 mg, 4.21 mmol) was added and the reaction mixture was stirred for 30 min. The reaction was diluted with water (30 ml) and extracted with ethyl acetate (3×30 ml). The organic layers were combined, dried (Na₂SO₄) and concentrated in vacuo. The crude material was purified by SPE-Si cartridge eluting with 50% cyclohexane in ethyl acetate to afford the title compound (360 mg, 80%); ¹H-NMR (300 MHz, CDCl₃) δ: 9.9 (s, 1H), 8.9 (d, 1H), 8.2 (d, 1H), 7.8 (m, 2H), 7.6 (bs, 1H), 4.2 (s, 2H), 4.1 (s, 3H).

### Description 105: Ethyl 1-methyl-6-(2-propen-1-yl)imidazo[1,5-a]quinoline-3-carboxylate (D105)

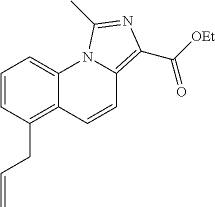

Zinc powder (1.4 g) was added to a mixture of ethyl nitro[5-(2-propen-1-yl)-2-quinolinyl]acetate (D89) (1 g) in glacial acetic acid (25 ml). The reaction mixture was stirred at room temperature for 2 hours and then filtered. To 14 ml of the filtered solution triethyl orthoacetate (10 ml) was added and heated with stirring at 150° C. for 7 min. under microwave irradiation (Personal Chemistry Emrys™ Optimiser, 300W). The resulting reaction mixture was evaporated in vacuo and then purified by SPE-Si cartridge eluting with 50% ethyl acetate in cyclohexane to afford the title compound as a pale yellow solid (210 mg); MS; (ES) m/z: 295.1 [MH⁺]. C18H18N2O2 requires 294.4.

### Description 106: Ethyl 1-methyl-6-(2-oxoethyl)imidazo[1,5-a]quinoline-3-carboxylate (D106)

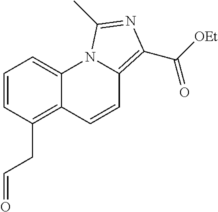

Osmium tetroxide (0.5 ml of a 4% by wt. solution in water) was added to a stirred solution of ethyl 1-methyl-6-(2-propen-1-yl)imidazo[1,5-a]quinoline-3-carboxylate (D105) (205 mg, 0.7 mmol) in THF/water (2:1; 6 ml). After 10 minutes sodium periodate (372 mg, 1.74 mmol) was added and the reaction mixture was stirred for additional 30 minutes. The reaction was diluted with water (15 ml) and extracted with ethyl acetate (3×15 ml). The organic layers were combined, dried (Na₂SO₄) and concentrated in vacuo. The crude material was purified by SPE-Si cartridge eluting with 30% cyclohexane in ethyl acetate to afford the title compound as a white solid (90 mg, 43%); MS (ES) m/z: 297.2 [MH]⁺. C17H16N2O3 requires 296.3.

### Description 107: 5-Bromo-6-methylquinoline (D107)

The title compound was prepared in 86% yield following the procedure described in Chem. Heterocyl Compd (Engl Transl) 1988, vol 24(8), 892; MS: (ES) m/z: 222.0 [MH⁺]. C10H8BrN requires 221.08.

### Description 108: 6-Methyl-5-(2-propen-1-yl)quinoline (D108)

To a solution of 5-bromo-6-methylquinoline (D107) (5.6 g, 25.2 mmol) in dry toluene (40 ml) were added allyltributylstannane (8.5 ml, 27.7 mmol), and tetrakis(triphenylphosphine)palladium(0) (1.4 g, 5%) at room temperature. The reaction mixture was stirred at 125° C. for 5 hours then cooled, quenched with water (40 ml) and extracted with DCM (3×100 ml). The combined organic layers were dried (Na₂SO₄) and concentrated in vacuo to give the crude product which was purified by SPE Si, eluting with cyclohexane/ethyl acetate (15:5), to afford the title compound (3.74 g, 81%); MS (ES) m/z: 184.10 [MH⁺], C13H13N requires 183.25.

### Description 109: 6-Methyl-5-(2-propen-1-yl)quinoline 1-oxide (D109)

The title compound was prepared in 82% yield according to the procedure used for the preparation of Description 88 starting from 6-methyl-5-(2-propen-1-yl)quinoline (D108); MS: (ES/+) m/z 200.1 [MH⁺]. C₁₃H₁₃NO requires 199.25.

### Description 110: Ethyl [6-methyl-5-(2-propen-1-yl)-2-quinolinyl](nitro)acetate (D110)

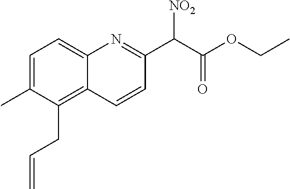

The title compound was prepared in 36% yield according to the synthetic procedure used for the preparation of Description 89 starting from 6-methyl-5-(2-propen-1-yl)quinoline 1-oxide (D109). The crude material was purified by SPE-SI cartridge eluting with 0.5% MeOH in DCM; MS: (ES/+) m/z: 315.0 [MH⁺]. C₁₇H₁₈N₂O₄ requires 314.34.

### Description 111: Ethyl 7-methyl-6-(2-propen-1-yl)imidazo[1,5-a]quinoline-3-carboxylate (D111)

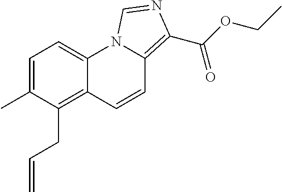

To an ice cooled suspension of ethyl [6-methyl-5-(2-propen-1-yl)-2-quinolinyl](nitro)acetate (D110) (300 mg, 0.95 mmol) in glacial acetic acid (4 ml) was added zinc (374 mg, 5.7 mmol) portion wise. After addition of all the zinc, the reaction mixture was allowed to warm to room temperature and stirred at room temperature for 1 hour. The zinc was filtered off and washed with water (30 ml). The solution was poured into 1M of EDTA (50 ml), basified to pH 8 with 1M NaOH. The whole solution was then taken to pH 6, extracted with EtOAc (2×30 ml). The combined organic phases were washed with 1M NaOH (40 ml), dried over anhydrous Na₂SO₄ and evaporated in vacuo. The intermediate ethyl amino[6-methyl-5-(2-propen-1-yl)-2-quinolinyl]acetate was quickly treated with triethyl orthoformate (4 ml) and heated with stirring at 150° C. for 10 min. under microwave irradiation. A light yellow solid precipitated from the reaction mixture, it was collected by filtration and washed with ether to afford 80 mg of the title compound as a pale yellow solid. The filtrate was passed through SCX cartridge to remove triethyl orthoformate and then purified by SPE-SI cartridge eluting with 20% ethyl acetate in cyclohexane to afford the title compound (100 mg, 35.8%); ¹H-NMR (300 MHz, CDCl₃) δ: 8.5 (s, 1. H), 8.1 (d, 1H), 7.9 (d, 1H), 7.5 (d, 1H), 7.6 (d, 1H), 7.3 (d, 1H), 6.1-5.9 (m, 1H), 5.1 (d, 1H), 4.8 (d, 1H), 4.45 (q, 2H), 3.8 (d, 2H), 2.45 (s, 3H), 1.4 (t, 3H).

### Description 112: Ethyl 7-methyl-6-(2-oxoethyl)imidazo[1,5-a]quinoline-3-carboxylate (D112)

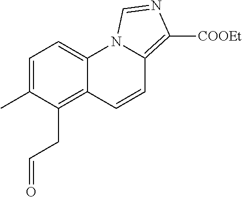

The title compound was prepared according to the synthetic procedure used for the preparation of Description 91 starting from ethyl 7-methyl-6-(2-propen-1-yl)imidazo[1,5-a]quinoline-3-carboxylate (D111). The crude material was purified by SPE-SI cartridge eluting with 50% to 100% ethyl acetate in cyclohexane to afford the title compound in 75% yield; ¹H-NMR (300 MHz, CDCl₃) δ: 9.85 (s, 1H), 8.65 (s, 1H), 8.1 (d, 1H), 8.0 (d, 1H), 7.5 (d, 1H), 7.4 (d, 1H), 4.5 (q, 2H), 4.2 (s, 2H), 2.5 (s, 3H), 1.4 (t, 3H).

### Description 113: (2Z)-3-(Dimethylamino)-1-(6-{2-[4-(2-methyl-5-quinolinyl)-1-piperidinyl]ethyl}-4H-imidazo[5,1-c][1,4]-benzoxazin-3-yl)-2-buten-1-one (D113)

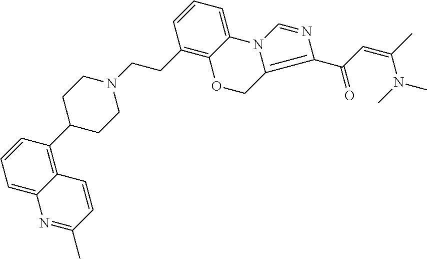

1-(6-{2-[4-(2-Methyl-5-quinolinyl)-1-piperidinyl]ethyl}-4H-imidazo[5,1-c][1,4]benzoxazin-3-yl)ethanone (free base of E114) (75.01 mg, 0.16 mmol) was suspended in N,N-dimethyl acetamide dimethylacetale (1.50 ml) and heated with stirring at 150° C. under microwave irradiation for 5 minutes. Then for a further 5 minutes. The precipitate was filtered off from the reaction mixture and washed with diethyl ether to afford the title compound (7.30 mg, 0.10 mmol, 63% yield) as a yellow solid; MS (ES) m/z: 536.3 [MH⁺]. C₃₃H₃₇N₃O₂ requires 535.69.

### Description 114: 2-(5-methyl-1,3,4-oxadiazol-2-yl)-1-benzofuran-4-yl trifluoromethanesulfonate (D114)

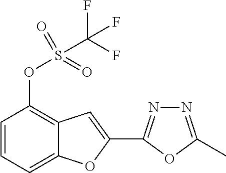

1,1,1-Trifluoro-N-phenyl-N-[(trifluoromethyl)sulfonyl]methanesulfonamide (2.54 g, 7.11 mmol) was added portionwise to an ice-cooled stirred suspension of 2-(5-methyl-1,3,4-oxadiazol-2-yl)-1-benzofuran-4-ol (WO 2000071517) (1.28 g, 5.92 mmol) and triethylamine (1.10 ml, 7.89 mmol) in dry CH₃CN (40 ml). The reaction mixture was stirred overnight at room temperature, then quenched with water (30 ml) and extracted with EtOAc (3×30 ml). The combined organic layers were dried (Na₂SO₄) and concentrated in vacuo. The crude reaction mixture was purified by Horizon-Si eluting with 30% EtOAc in c-hexanes to afford the title compound (1.60 g, 4.60 mmol, 78% yield) as white solid; MS; (ES) m/z: 349.1 [MH⁺]. C₁₂H₇F₃N₂O₅S requires 348.26.

### Description 115: 1,1-Dimethylethyl 4-[2-(5-methyl-1,3,4-oxadiazol-2-yl)-1-benzofuran-4-yl]-1-piperazinecarboxylate (D115)

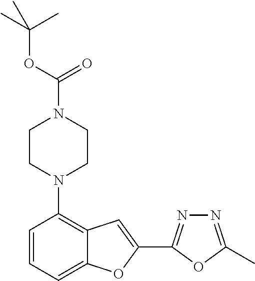

A mixture of 2-(5-methyl-1,3,4-oxadiazol-2-yl)-1-benzofuran-4-yl trifluoromethanesulfonate (D114) (1.31 g, 3.76 mmol), Cs₂CO₃ (1.84 g, 5.64 mmol), Pd(OAc)₂ (84.41 mg, 0.38 mmol), (+/−)-BINAP (0.35 g, 0.56 mmol) and N-Boc-piperazine (0.91 g, 4.88 mmol) in dry toluene (40 ml) was stirred at 100° C. for 4 hours. After cooling to room temperature, aqueous saturated NH₄Cl solution (30 ml) was added and the mixture was extracted with ethyl acetate (3×30 ml). The combined organics were dried (Na₂SO₄) and evaporated in vacuo. The residue was purified by Biotage-Si with cyclohexanes/ethyl acetate 70/30 elution to afford the title compound (0.94 g, 2.44 mmol, 65% yield) as a yellow oil; ¹H-NMR (300 MHz, DMSO) δ: 7.8 (s, 1H), 7.4 (m, 2H), 7.8 (d, 1H), 3.5-3.4 (bm, 4H), 3.3 (s, 3H), 3.2-3.1 (bm, 4H), 1.4 (s, 9H).

### Description 116: 1-[2-(5-Methyl-1,3,4-oxadiazol-2-yl)-1-benzofuran-4-yl]piperazine (D116)

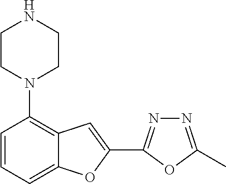

A solution of 1,1-dimethylethyl 4-[2-(5-methyl-1,3,4-oxadiazol-2-yl)-1-benzofuran-4-yl]-1-piperazinecarboxylate (D115) (0.83 g, 2.16 mmol) in a 1:3 mixture of TFA/THF (12 ml) was stirred at room temperature overnight. After evaporation the crude material was purified by SCX cartridge (eluting with methanol followed by 2N ammonia solution in methanol) to afford the title compound (0.77 g, 92% yield) as a yellow solid; MS (ES; m/z): 285.1 [MH⁺]. C₁₅H₁₆N₄O₂ requires 284.32.

### Description 117: Ethyl 2-[8-(2-propen-1-yl)-2H-1,4-benzoxazin-3-yl]hydrazinecarboxylate (D117)

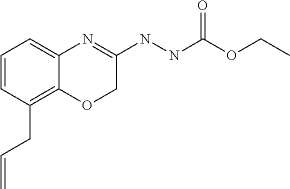

A mixture of 8-(2-propen-1-yl)-2H-1,4-benzoxazine-3(4H)-thione (D18) (1 mmol, 203 mg) and ethyl carbazate (3 mmol, 312 mg) in anhydrous ethanol (4 ml) was refluxed for 4.5 hours. The reaction mixture was cooled and the resulting solid was filtered and triturated with ethyl ether. The title compound was obtained in 75% yield (210 mg); MS: (ES) m/z: 276.2 [MH⁺]. C14H17N3O3 requires 275.3.

### Description 118: 6-(2-Propen-1-yl)-2,4-dihydro-1H-[1,2,4]triazolo[3,4-c][1,4]-benzoxazin-1-one (D118)

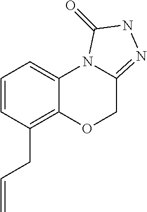

A solution of ethyl 2-[8-(2-propen-1-yl)-2H-1,4-benzoxazin-3-yl]hydrazinecarboxylate (D117) (80 mg, 0.291 mmol) in DMF (1 ml) was heated with stirring at 200° C. for 10 minutes under microwave irradiation. The solution was concentrated and the resulting solid was triturated with diethyl ether to give the title compound (50 mg, 74%); MS (ES) m/z: 230.0 [MH⁺]. C12H11N3O2 requires 229.2.

### Description 119: 2-Methyl-6-(2-propen-1-yl)-2,4-dihydro-1H-[1,2,4]triazolo[3,4-c][1,4]-benzoxazin-1-one (D119)

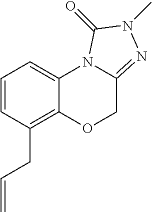

Sodium hydride (15 mg of a 60% w/w suspension in mineral oil, 0.37 mmol) was added to a solution of 6-(2-propen-1-yl)-2,4-dihydro-1H-[1,2,4]triazolo[3,4-c][1,4]benzoxazin-1-one (D118) (60 mg, 0.26 mmol) and iodomethane (20 μl, 0.31 mmol) in dry DMF (3 ml) at 0° C. The reaction mixture was allowed to warm to room temperature and stirred for 1 hour then quenched with water (5 ml) and extracted with DCM (3×15 ml). The combined organic layers were dried (Na₂SO₄) and concentrated in vacuo to give the title compound (66 mg) which may be used for the next step without any further purification; MS (ES) m/z: 244.0 [MH⁺]. C13H13N3O2 requires 243.2.

### Description 120: (2-methyl-1-oxo-2,4-dihydro-1H-[1,2,4]triazolo[3,4-c][1,4]-benzoxazin-6-yl)acetaldehyde (D120)

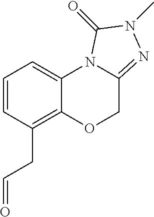

The title compound was prepared according to the procedure of Description 6 starting from 2-methyl-6-(2-propen-1-yl)-2,4-dihydro-1H-[1,2,4]triazolo[3,4-c][1,4]benzoxazin-1-one (D119) (64 mg, 0.26 mmol). The crude product was used in the next step without purification; ¹H-NMR (400 MHz, CDCl₃) δ: 9.77 (s, 1H), 8.24 (m, 1H), 7.14 (m, 1H), 7.05 (m, 1H), 5.03 (s, 2H), 3.78 (s, 2H), 3.54 (s, 3H).

### Description 121: 7-Methyl-8-{2-[4-(2-methyl-5-quinolinyl)-1-piperidinyl]ethyl}-2H-1,4-benzoxazin-3(4H)-one (D121)

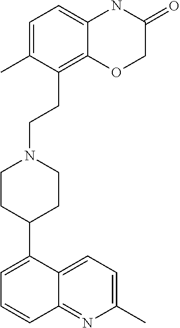

The title compound was prepared in 92% yield following the general reductive amination procedure of Example 1 starting from (7-methyl-3-oxo-3,4-dihydro-2H-1,4-benzoxazin-8-yl)acetaldehyde (D79) (100 mg, 0.488 mmol) and 2-methyl-5-(4-piperidinyl)quinoline (165 mg, 0.732 mmol). The crude product was purified by flash chromatography on silica gel eluting with a gradient of methanol in DCM (2 to 3%) to afford the title compound (186 mg, 92%); MS (ES) m/z: 416.10 [MH⁺]. C26H29N3O2 requires 415.53.

### Description 122: 6-Methyl-2-oxo-1,2,3,4-tetrahydro-5-quinolinol trifluoromethanesulfonate (D122)

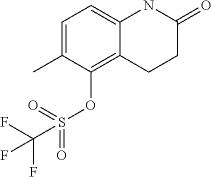

The title compound was prepared following the procedure of Description 71 starting from 5-hydroxy-6-methyl-3,4-dihydro-2(1H)-quinolinone (see Organic Preparations and Procedures International, 25 (2), 223-8, 1993] (2.0 g, 11.3 mmol). The crude product was purified by flash chromatography on silica gel eluting with ethyl acetate/cyclohexane (7/3) to afford the title compound (3.9 g); MS (ES) m/z: 310.00 [MH⁺]. C11H10F3NO4S requires 309.26.

### Description 123: 6-Methyl-5-(2-Propen-1-yl)-3,4-dihydro-2(1H)-quinolinone (D123)

The title compound was prepared following the procedure of description 72 from 6-methyl-2-oxo-1,2,3,4-tetrahydro-5-quinolinyl trifluoromethanesulfonate (D122) (3.5 g, 11.3 mmol). The crude product was purified by flash chromatography on silica gel eluting with ethyl acetate/cyclohexane (7/3) and trituration with diethyl ether, to afford the title compound (1.97 g, 85%); MS (ES) m/z: 202.10 [MH⁺]. C13H15NO requires 201.27.

### Description 124: Ethyl 7-methyl-6-(2-propen-1-yl)-4,5-dihydroimidazo[1,5-a]quinoline-3-carboxylate (D124)

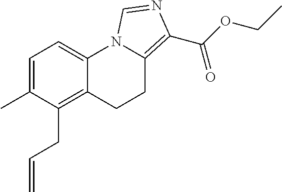

The title compound was prepared following the procedure of Example 80 using 6-methyl-5-(2-propen-1-yl)-3,4-dihydro-2(1H)-quinolinone (D123) (700 mg, 3.48 mmol). The crude product was purified by flash chromatography on silica gel eluting with ethyl acetate/cyclohexane (1/1) to afford the title compound (863 mg, 84%); MS (ES) m/z: 297.10 [MH⁺]. C18H20N₂O₂ requires 296.37.

### Description 125: Ethyl 7-methyl-6-(2-oxoethyl)-4,5-dihydroimidazo[1,5-a]quinoline-3-carboxylate (D125)

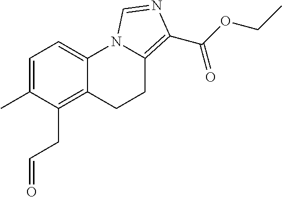

The title compound was prepared following the procedure of Description D6 using ethyl 7-methyl-6-(2-propen-1-yl)-4,5-dihydroimidazo[1,5-a]quinoline-3-carboxylate (D124) (863 mg, 2.92 mmol). The crude product was purified by flash chromatography on silica gel eluting with ethyl acetate to afford the title compound (602 mg, 69%); ¹H NMR (300 MHz, CDCl₃) δ ppm 1.43 (t, 3H) 2.35 (s, 3H) 2.85 (t, 2H), 3.32 (t, 2H) 3.91 (s, 2H) 4.40 (q, 2H) 7.22 (d, 1H) 7.36 (d, 1H) 8.02 (s, 1H), 9.78 (s, 1H).

### Description 126: 6-(2-Propen-1-yl)-4H-tetrazolo[5,1-c][1,4]-benzoxazine (D126)

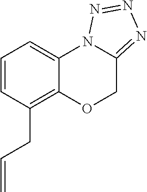

A solution of hydrazine hydrate (380 μl, 12.2 mmol) in dry THF (2 ml) was treated dropwise with a solution of 8-(2-propen-1-yl)-2H-1,4-benzoxazine-3(4H)-thione (D18) (500 mg, 2.44 mmol) in THF (25 ml) at 20° C. After stirring for 1.5 hours the solvent was removed under reduced pressure to afford the intermediate hydrazone which was used in the following step without any further purification. To a suspension of hydrazone (495 mg, 2.44 mmol) in 0.5N HCl (15 ml) a solution of sodium nitrite (252 mg, 3.66 mmol) in water (2 ml) was added dropwise at 5° C. After stirring for 4 hours the mixture was neutralized with a saturated solution of NaHCO₃ and extracted with DCM (3×30 ml). The combined organic layers were dried (Na₂SO₄) and concentrated in vacuo. The crude product was purified by flash chromatography on silica gel eluting with ethyl acetate/cyclohexane (2/8) to afford the title compound (235 mg, 45%); MS (ES) m/z: 215.10 [MH⁺], C11H10N4O requires 214.23.

### Description 127: 5-(2-Propen-1-yl)-3,4-dihydro-2(1H)-quinolinethione (D127)

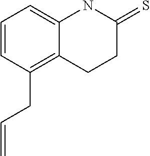

The title compound was prepared following the procedure of Description 18 using 5-(2-propen-1-yl)-3,4-dihydro-2(1H)-quinolinone (D72) (404 mg, 2.16 mmol). The crude product was purified by flash chromatography on silica gel eluting with ethyl acetate/cyclohexane (4/6) to afford the title compound (290 mg, 66%); MS (ES) m/z: 204.10 [MH⁺]. C12H13NS requires 203.31.

### Description 128: 6-(2-Proper-1-yl)-4,5-dihydrotetrazolo[1,5-a]quinoline (D128)

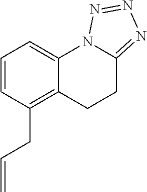

The title compound was prepared following the procedure of Description 126 starting from 5-(2-propen-1-yl)-3,4-dihydro-2(1H)-quinolinethione (D127) (290 mg, 1.43 mmol). The crude product was purified by flash chromatography on silica gel eluting with ethyl acetate/cyclohexane (3/7) to afford the title compound D128 (225 mg, 74%); MS (ES) m/z: 213.10 [MH⁺]. C12H12N4 requires 212.25.

### Description 129: 4H-Tetrazolo[5,1-c][1,4]-benzoxazin-6-ylacetaldehyde (D129)

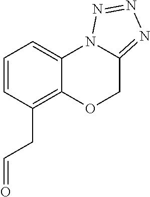

The title compound was prepared following the procedure of description D6 using 6-(2-propen-1-yl)-4H-tetrazolo[5,1-c][1,4]benzoxazine (D126) (235 mg, 1.09 mmol). The crude product was purified by flash chromatography on silica gel eluting with ethyl acetate/cyclohexane (4/6) to afford the title compound (144 mg, 61%); ¹H NMR (300 MHz, CDCl₃) δ ppm 3.77 (s, 2H) 5.56 (s, 2H) 7.14 (d, 2H) 7.90 (t, 1H) 9.72 (s, 1H).

### Description 130: 4,5-Dihydrotetrazolo[1,5-a]quinolin-6-ylacetaldehyde (D130)

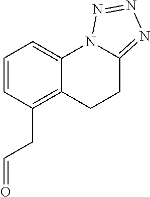

The title compound was prepared following the procedure of Description 6 using 6-(2-propen-1-yl)-4,5-dihydrotetrazolo[1,5-a]quinoline (D128) (225 mg, 1.06 mmol). The crude product was purified by flash chromatography on silica gel eluting with ethyl acetate/cyclohexane (4/6 to 10/0) to afford the title compound (132 mg, 58%); ¹H NMR (300 MHz, CDCl₃) δ ppm 2.95 (t, 2H) 3.27 (t, 2H) 3.82 (s, 2H) 7.19 (m, 1H) 7.38 (t, 1H) 7.97 (d, 1H) 9.72 (s, 1H).

### Description 131: 2-(Methylthio)-5-(2-propen-1-yl)-3,4-dihydroquinoline (D131)

The title compound was prepared following the procedure of Description 19 using 5-(2-propen-1-yl)-3,4-dihydro-2(1H)-quinolinethione (D127) (150 mg, 0.74 mmol). The crude product was used in the next step without any further purification; MS (ES) m/z: 218.10 [MH⁺]. C13H15NS requires 217.33.

### Description 132: Ethyl (2E/Z)-nitro-[8-(2-propen-1-yl)-2H-1,4-benzoxazin-3(4H)-ylidene]-ethanoate (D132)

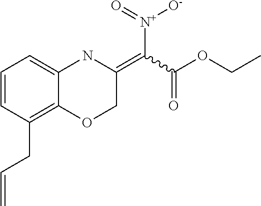

To a mixture of 3-(methylthio)-8-(2-propen-1-yl)-2H-1,4-benzoxazine (D19) (441 mg, 2.01 mmol) and ethyl nitroacetate (2.23 ml, 20.1 mmol), at room temperature and under nitrogen atmosphere, was added Triton-B (40% in methanol, 841 μl, 2.01 mmol). The reaction mixture was heated at 50° C. for 2 hours then quenched with water (10 ml) and extracted with DCM (3×30 ml): The combined organic layers were dried (Na₂SO₄) and concentrated in vacuo to give the crude product which was purified by flash chromatography on silica gel, eluting with ethyl acetate/cyclohexane (1/9), to afford the title compound (411 mg, 67%) as a mixture of E/Z isomers; MS (ES) m/z: 305.10 [MH⁺]. C15H16N2O5 requires 304.30.

### Description 133: Ethyl (2E/Z)-nitro[5-(2-propen-1-yl)-3,4-dihydro-2(1H)-quinolinylidene]-ethanoate (D133)

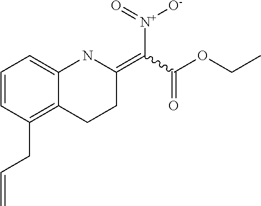

The title compound was prepared following the procedure of Description 132 using 2-(methylthio)-5-(2-propen-1-yl)-3,4-dihydroquinoline (D131) (261 mg, 1.20 mmol). The crude product was purified by flash chromatography on silica gel, eluting with ethyl acetate/cyclohexane (1/9), to afford the title compound (264 mg, 73%) as a mixture of E/Z isomers; MS (ES) m/z: 303.20 [MH⁺]. C16H18N2O4 requires 302.33.

### Description 134: Ethyl 6-(2-propen-1-yl)-4H-[1,2,3]triazolo[5,1-c][1,4]-benzoxazine-3-carboxylate (D134)

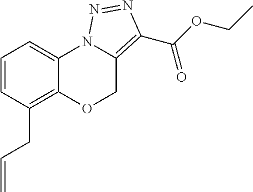

To a solution of ethyl (2E/Z)-nitro[8-(2-propen-1-yl)-2H-1,4-benzoxazin-3(4H)-ylidene]ethanoate (D132) (280 mg, 0.921 mmol) in glacial acetic acid (3 ml) was added zinc (356 mg, 5.53 mmol) at 0° C. The reaction mixture was allowed to warm to room temperature and stirred for 45 minutes, then was cooled to 5° C. before the addition of trichloroacetic acid (30 mg, 0.184 mmol) and amyl nitrite (151 mg, 1.28 mmol). The reaction mixture was allowed to warm to room temperature and stirred for additional 1.5 hours then quenched with water (10 ml) and extracted with DCM (3×30 ml). The combined organic layers were dried (Na₂SO₄) and concentrated in vacuo to give the crude product which was purified first by SCX cartridge then by flash chromatography on silica gel, eluting with ethyl acetate/cyclohexane (2/8), to afford the title compound (154 mg, 58%); MS (ES) m/z: 286.00 [MH⁺]. C15H15N3O3 requires 285.30.

### Description 135: Ethyl 6-(2-propen-1-yl)-4,5-dihydro[1,2,3]triazolo[1,5-a]quinoline-3-carboxylate (D135)

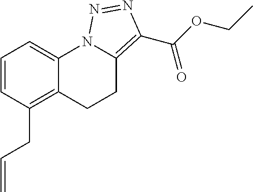

The title compound was prepared following the procedure of Description 134 using ethyl (2E/Z)-nitro[5-(2-propen-1-yl)-3,4-dihydro-2(1H)-quinolinylidene]-ethanoate (D133) (200 mg, 0.662 mmol). The crude product was purified by flash chromatography on silica gel, eluting with ethyl acetate/cyclohexane (2/8), to afford the title compound (97 mg, 52%); MS (ES) m/z: 284.00 [MH⁺], C16H17N3O2 requires 283.33.

### Description 136: Ethyl 6-(2-oxoethyl)-4H-[1,2,3]triazolo[5,1-c][1,4]-benzoxazine-3-carboxylate (D136)

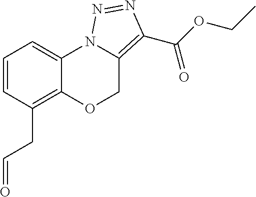

The title compound was prepared following the procedure of Description 6 using ethyl 6-(2-propen-1-yl)-4H-[1,2,3]triazolo[5,1-c][1,4]benzoxazine-3-carboxylate (D134) (154 mg, 0.540 mmol). The crude product was purified by flash chromatography on silica gel eluting with ethyl acetate/cyclohexane (2/8) to afford the title compound (96 mg, 62%); ¹H NMR (300 MHz, CDCl₃) δ ppm 1.37 (t, 3H) 3.76 (s, 2H) 4.40 (q, 2H) 5.57 (s, 2H) 7.12 (m, 2H) 8.01 (m, 1H) 9.72 (s, 1H).

### Description 137: Ethyl 6-(2-oxoethyl)-4,5-dihydro[1,2,3]triazolo[1,5-a]quinoline-3-carboxylate (D137)

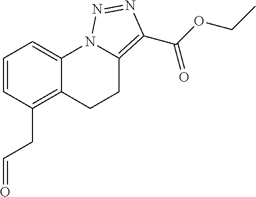

The title compound was prepared following the procedure of Description D6 using ethyl 6-(2-propen-1-yl)-4,5-dihydro[1,2,3]triazolo[1,5-a]quinoline-3-carboxylate (D135) (97 mg, 0.343 mmol). The crude product was purified by flash chromatography on silica gel eluting with ethyl acetate/cyclohexane (2/8 to 4/6) to afford the title compound (71 mg, 73%); ¹H NMR (300 MHz, CDCl₃) δ ppm 1.40 (t, 3H) 2.87 (t, 2H) 3.33 (t, 2H) 3.83 (s, 2H) 4.41 (q, 2H) 7.18 (m, 1H) 7.38 (t, 1H) 8.12 (d, 1H) 9.73 (s, 1H).

### Description 138: 6-{2-[4-(2-Methyl-5-quinolinyl)-1-piperazinyl]ethyl}-4H-imidazo[5,1-c][1,4]-benzoxazine-3-carbaldehyde (D138)

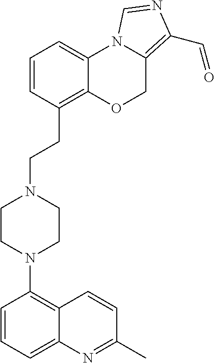

To a stirred solution of N-methyl-N-(methyloxy)-6-{2-[4-(2-methyl-5-quinolinyl)-1-piperazinyl]ethyl}-4H-imidazo[5,1-c][1,4]benzoxazine-3-carboxamide (E25) (120 mg, 0.234 mmol) and dry THF (1 ml) cooled at 0° C. was added LiAl₄H (0.117 ml of 1M sol. in THF, 0.117 mmol) and the resulting mixture was stirred for 1 hour at 0° C. The reaction mixture was quenched with water and then extracted with ethyl acetate (3×20 ml). The combined organic layers were dried (Na₂SO₄) and evaporated in vacuo and the resulting crude oil was purified by SPE-Si cartridge (2 g) eluting with 2% methanol in DCM to afford the title compound as a white solid (77 mg, 75%); MS (ES) m/z: 454.4 [MH⁺]. C₂₇H₂₇N₅O₃ requires 453.54.

### Description 139: (2Z)-3-(Dimethylamino)-1-(6-{2-[4-(2-methyl-5-quinolinyl)-1-piperazinyl]ethyl}-4H-imidazo[5,1-c][1,4]-benzoxazin-3-yl)-2-buten-1-one (D139)

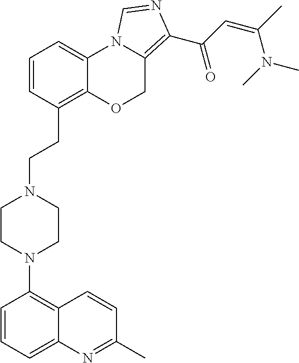

The freebase of 1-(6-{2-[4-(2-methyl-5-quinolinyl)-1-piperazinyl]ethyl}-4H-imidazo[5,1-c][1,4]benzoxazin-3-yl)ethanone hydrochloride (E35) (85 mg, 0.182 mmol), was suspended in N,N dimethylacetamide dimethylacetale and the mixture was irradiated in a microwave reactor (PersonalChemistry Emrys™ Optimiser, 300W, 150° C., 5 min+5 min). A cream solid precipitated from the reaction mixture, it was collected by filtration and triturated with ethyl ether to afford the title compound (67 mg, 69%); MS (ES) m/z: 537.3 [MH⁺]. C₃₂H₃₆N₆O₂ requires 536.68.

### Description 140:1,1-Dimethylethyl 4-hydroxy-4-(5-quinolinol)-1-piperidinecarboxylate (D140)

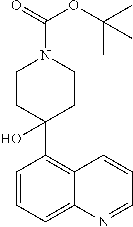

5-Bromoquinoline (2 g, 0.0096 mol) was added under N₂, at −70° C., to a stirred solution of n-BuLi (12 ml of 1.6M sol. in hexane, 0.0192 mmol) in 20 ml of a 1:1 mixture of dry THF:diethyl ether. The reaction mixture was stirred 30 minutes then a solution of 1,1-dimethylethyl 4-oxo-1-piperidinecarboxylate (1.9 g, 0.0095) in THF (20 ml) was added and the reaction mixture further stirred for 30 minutes. The reaction mixture was allowed to warm at 0° C. then quenched with water and extracted with ethyl acetate (3×20 ml). The combined organic layers were dried (Na₂SO₄) and evaporated in vacuo to afford 2 g of crude material. The obtained mixture was purified by Horizon column (40M) eluting with 10% ethyl acetate in cyclohexane to afford the title compound as a cream solid (1.9 g, 60%); MS (ES) m/z: 329.2 [MH⁺]. C₁₉H₂₄N₂O₃ requires 328.41.

### Description 141: 1,1-Dimethylethyl 4-fluoro-4-(5-quinolinol)-1-piperidinecarboxylate (D141)

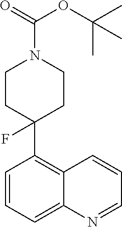

1,1-Dimethylethyl 4-hydroxy-4-(5-quinolinyl)-1-piperidinecarboxylate (D140) (100 mg, 0.3 mmol) dissolved in dry DCM (3 ml) was added at −78° C. to a stirred solution of DEOXOFLUOR (0.144 ml of a 50% sol in THF, 0.335 mmol) in dry DCM (3 ml). The reaction mixture was stirred for 2 hours at −78° C. then for 1 night at room temperature. The reaction mixture was quenched with NH₄Cl saturated solution and extracted with DCM (3×10 ml). The combined organic layers were dried (Na₂SO₄) and evaporated in vacuo to afford a crude material. The obtained mixture was purified by SPE-Si cartridge (2 g) eluting with 2% methanol in DCM to afford the title compound as a white foam (85 mg, 83%); MS (ES) m/z: 331.2 [MH⁺]. C₁₉H₂₃FN₂O₂ requires 330.4.

### Description 142: 5-(4-Fluoro-4-piperidinyl)quinoline (D142)

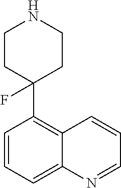

1,1-Dimethylethyl 4-fluoro-4-(5-quinolinyl)-1-piperidinecarboxylate (D141) (170 mg, 0.51 mmol) was dissolved in a 3:1 mixture TFA:DCM (6:2 ml) and stirred overnight at room temperature. The solvent was evaporated in vacuo and the crude mixture was purified by SPE-SCX cartridge (2 g) (eluting with methanol followed by 2N ammonia solution in methanol) to afford the title compound as a white solid (90 mg, 76%); MS (ES) m/z: 231.1 [MH⁺]. C₁₄H₁₅FN₂ requires 230.28.

### Description 143: 2-(Difluoromethyl)-5-(1-piperazinyl)quinoline (D143)

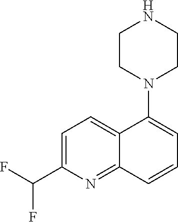

A solution of 1,1-dimethylethyl 4-[2-(difluoromethyl)-5-quinolinyl]-1-piperazinecarboxylate (D100) (886 mg, 2.438 mmol) in a mixture of TFA/DCM (10 ml, 1:1) was stirred at room temperature overnight. After evaporation, the crude material was purified by SCX extraction to afford the title compound (616 mg, 96%) as a yellow solid; MS (ES; m/z): 264 [MH⁺]. C₁₄H₁₅F₂N₃ requires 263.29; ¹H NMR (300 MHz, CDCl₃) δ: 8.65 (d, 1H), 7.80 (d, 1H), 7.66 (m, 2H), 7.17 (d, 1H), 6.74 (t, 1H), 3.1 (m, 9H).

### Description 144: 1,1-Dimethylethyl 4-[2-(hydroxymethyl)-5-quinolinyl]-1-piperazine-carboxylate (D144)

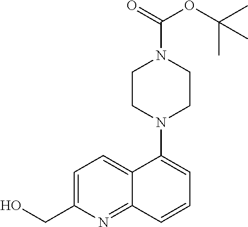

A solution of 1,1-dimethylethyl 4-(2-formyl-5-quinolinyl)-1-piperazine-carboxylate (D68) (1.505 g, 4.41 mmol) and sodium borohydride (834 mg, 22.05 mmol) in ethanol (20 ml) was stirred for 5 hours at room temperature. The mixture was evaporated in vacuo and the residue dissolved in water (100 ml). The suspension was extracted with DCM (3×100 ml) and washed with brine (100 ml). The organic layer was dried (MgSO₄) and evaporated to afford the title compound (1.515 g, quant.); MS (ES; m/z): 344 [MH⁺]. C₁₉H₂₅N₃O₃ requires 343.42; ¹H NMR (300 MHz, CDCl₃) δ: 8.4 (m, 1H), 7.7 (m, 1H), 7.6 (m, 1H), 7.2 (m, 1H), 7.1 (m, 1H), 4.9 (m, 2H), 4.4 (s, 1H), 3.7 (bm, 4H), 3.0 (bm, 4H), 1.4 (s, 9H).

### Description 145: 1,1-Dimethylethyl 4-[2-(fluoromethyl)-5-quinolinyl]-1-piperazinecarboxylate (D145)

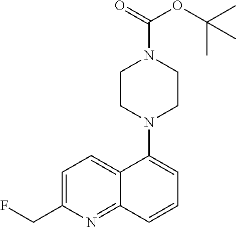

DAST (636 μl, 782 mg, 4.852 mmol) was added to a cooled solution (−78° C.) of 1,1-dimethylethyl 4-[2-(hydroxymethyl)-5-quinolinyl]-1-piperazinecarboxylate (D144) (1.515 g, 4.41 mmol) in DCM (20 ml). After 1.5 hours additional DAST (289 μl, 355 mg, 2.20 mmol) was added and stirring was continued for another 3 hours while the reaction was allowed to warm to room temperature. The reaction was quenched with water (100 ml) and extracted with DCM (100 ml). The organic layer was dried (MgSO₄) and evaporated in vacuo. The residue was purified by flash column chromatography (cyclohexanes:EtOAc 9:1 to 8:2) to afford (1.087 mg, 71%) as a yellow solid; MS (ES; m/z): 346 [MH⁺]. C₁₉H₂₄FN₃O₂ requires 345.42; NMR (¹H, 300 MHz, CDCl₃) δ: 8.56 (d, 1H), 7.74 (d, 1H), 7.6 (m, 2H), 7.09 (d, 1H), 5.62 (d, 2H), 3.68 (bm, 4H), 3.0 (bm, 4H), 1.48 (s, 9H).

### Description 146: 2-(Fluoromethyl)-5-(1-piperazinyl)quinoline (D146)

A solution of 1,1-dimethylethyl 4-[2-(fluoromethyl)-5-quinolinyl]-1-piperazinecarboxylate (D145) (1.087 g, 3.147 mmol) in a mixture of TFA/DCM (15 ml, 1:2) was stirred at room temperature overnight. After evaporation, the crude material was purified by SCX extraction to afford the title compound (725 mg, 94%) as a yellow solid; MS (ES; m/z): 246 [MH⁺], C₁₄H₁₆FN₃ requires 245.30; ¹H NMR (300 MHz, CDCl₃) δ: 8.56 (d, 1H), 7.72 (d, 1H), 7.6 (m, 2H), 7.10 (d, 1H), 5.62 (d, 2H), 3.1 (bm, 9H).

### Description 147: 5-(4-{[(1,1-Dimethylethyl)oxy]carbonyl}-1-piperazinyl)-2-quinolinecarboxylic acid (D147)

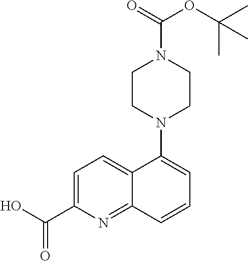

1,1-Dimethylethyl 4-(2-formyl-5-quinolinyl)-1-piperazine-carboxylate (D68) (1.0 g, 2.93 mmol, 1 eq.) was dissolved in DMSO (12 ml) and the mixture cooled to 5° C. Buffer solution ((3 ml; pH=3, citric acid/sodium hydroxide/hydrochloric acid) was added. To the resulting yellow suspension, a 1M aqueous solution of NaClO₂ (4 ml, 1.36 eq.) was added dropwise over 1 hour at 5° C. The mixture was then stirred at 25° C. for 1 hour. TLC analysis showed incomplete reaction. Further 1M NaClO₂ was added dropwise (12 ml) and DMSO (2 ml). After 1 hour stirring at 25° C., the mixture was diluted with water (10 ml) and extracted with AcOEt (2×10 ml). The organic layer was dried (Na₂SO₄) evaporated and the residue chromatographed over silica gel (230-400 Mesh) eluting with DCM/methanol 9/1 to afford the title compound (0.876 g, 83%) as a yellow oil; C₁₉H₂₃N₃O₄ requires 357.41; NMR (¹H, 300 MHz, CDCl₃) δ: 8.62 (d, 1H), 8.06 (d, 1H), 7.85 (d, 1H), 7.69 (t, 1H), 7.25 (d, 1H), 3.59 (bm, 4H), 2.98 (bm, 4H), 1.43 (s, 9H).

### Description 148: 1,1-Dimethylethyl 4-{2-[(dimethylamino)carbonyl]-5-quinolinyl}-1-piperazinecarboxylate (D148)

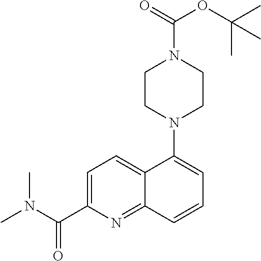

A solution of 5-(4-{[(1,1-dimethylethyl)oxy]carbonyl}-1-piperazinyl)-2-quinolinecarboxylic acid (D147) (219 mg, 0.753 mmol), EDC×HCl (216 mg, 1.129 mmol), HOBt (173 mg, 1.129 mmol) and dimethyl amine (2.0M in THF, 1.88 ml, 3.765 mmol) in dry DMF (5 ml) was stirred at room temperature for 20 hours. The reaction was diluted with DCM (20 ml) and washed with sat. NaHCO₃ solution (20 ml). The organic layer was dried (MgSO₄) and evaporated in vacuo to give (171 mg, 75%). The product may be used crude in the next step.

### Description 149: 1,1-Dimethylethyl 4-{2-[(methylamino)carbonyl]-5-quinolinyl}-1-piperazinecarboxylate (D149)

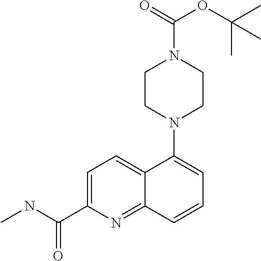

The title compound (296 mg, 87%) was obtained by the procedure of description 148 using 5-(4-{[(1,1-dimethylethyl)oxy]carbonyl}1-piperazinyl)-2-quinolinecarboxylic acid (D147) (376 mg, 1.052 mmol) and methyl amine (2.0M/THF). The title compound may be used in the next step without further purification.

### Description 150: N,N-Dimethyl-5(1-piperazinyl)-2-quinolinecarboxamide (D150)

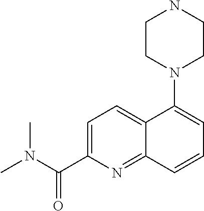

A solution of 1,1-dimethylethyl 4-{2-[dimethylamino)carbonyl]-5-quinolinyl}-1-piperazinecarboxylate (D148) (171 mg, 0.444 mmol) in a mixture DCM containing 10% TFA (5 ml) was stirred at room temperature overnight. After evaporation the crude material was purified by SCX extraction to afford the title compound (102 mg, 81%); MS (ES; m/z): 285 [MH⁺]. C₁₆H₂₀N₄O requires 284.36.

### Description 151: N-Methyl-5-(1-piperazinyl)-2-quinolinecarboxamide (D151)

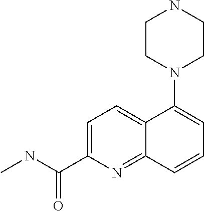

A solution of 1,1-dimethylethyl 4-{2-[(methylamino)carbonyl]-5-quinolinyl}-1-piperazinecarboxylate (D149) (296 mg, 0.770 mmol) in a mixture DCM containing 10% TFA (5 ml) was stirred at room temperature overnight. After evaporation the crude material was purified by SCX extraction to afford the title product (96 mg, 46%); MS (ES; m/z): 271 [MH⁺]. C₁₅H₁₈N₄O requires 270.33.

### Descriptions 152 and 153: 2-Methyl-1-[(1S)-1-phenylethyl]-4-piperidinone (D152 and D153)

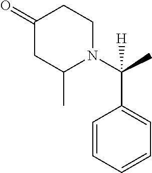

The title compounds D152 and D153 were prepared according to the experimental procedure reported in WO2004/094380. The crude mixture was purified by Biotage column (65M) eluting with 15% ethyl acetate in cyclohexane to afford the diastereoisomers D152 and D153 as pale yellow oils; Diastereoisomer 1 (D152): ¹H NMR (500 MHz, CHLOROFORM-d) δ ppm 1.15 (d, J=5.86 Hz, 3H) 1.34 (d, J=6.83 Hz, 3H) 2.16-2.39 (m, 3H) 2.60-2.80 (m, 3H) 3.35-3.42 (m, 1H) 4.02 (q, 1H) 7.24-7.28 (m, 1H) 7.34 (t, 2H) 7.45 (d, J=6.83 Hz, 2H); Diastereoisomer 2 (D153): ¹H NMR (500 MHz, CHLOROFORM-d) δ ppm 1.05 (d, J=6.83 Hz, 3H) 1.43 (d, J=6.83 Hz, 3H) 2.10-2.12 (m, 1H) 2.29-2.36 (m, J=12.69 Hz, 1H) 2.51-2.60 (m, 2H) 2.92-3.00 (m, 2H) 3.14-3.20 (m, 1H) 3.92 (q, 1H) 7.24-7.29 (m, 1H) 7.31-7.38 (m, 4H).

### Description 154: 2-Methyl-4-piperidinone acetate (D154)

2-Methyl-1-[(18)-1-phenylethyl]-4-piperidinone (Diastereomer 1) (D152) (18 g, 0.083 mol) was dissolved in acetic acid (90 ml) under nitrogen in a hydrogenation flask. Pd/C 10% (900 mg, 5% w/w) was added and the reaction mixture was vigorously stirred in a Parr apparatus under H₂ at 4 atm for 2 days. Catalyst was filtered off and the solution evaporated in vacuo to yield the title acetate salt as pale yellow oil (14 g, 97%); MS (ES) m/z: 114.1 [MH⁺]. C₆H₁₁NO requires 113.16.

### Description 155: 2-Methyl-4-piperidinone acetate (D155)

The title compound was prepared with 100% yield according to the experimental procedure of preparation of Description 154 starting from 2-Methyl-1-[(1S)-1-phenylethyl]-4-piperidinone (Diastereomer 2) (D153); MS (ES) m/z: 114.1 [MH⁺]. C₆H₁₁NO requires 113.16.

### Description 156: 1,1-Dimethylethyl 2-methyl-4-oxo-1-piperidinecarboxylate (D156)

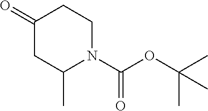

To a solution of 2-methyl-4-piperidinone acetate (D154) (14 g, 0.081 mol) in NaOH 1M (30 ml) was added di-tert butyl carbonate (18 g, 0.081 mol) and the reaction mixture was stirred overnight at 20° C. The reaction mixture was diluted with ethyl acetate (50 ml) and extracted several time (50 ml). The combined organic layers were dried (Na₂SO₄) and evaporated in vacuo to give a crude oil that was purified on a silica pad by eluting with 11% ethyl acetate in cyclohexane to afford the title compound (6.95 g, 40%) as a pale yellow oil; MS (ES) m/z: 214.1 [MH⁺]. C₁₁H₁₉NO₃ requires 213.28.

### Description 157: 1,1-Dimethylethyl 2-methyl-4-oxo-1-piperidinecarboxylate (D157)

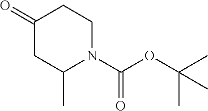

The title compound was prepared according to the experimental procedure reported for the preparation of Description 56 starting from 2-methyl-4-piperidinone acetate (D155); MS (ES) m/z: 214.1 [MH⁺]. C₁₁H₁₉NO₃ requires 213.28.

### Description 158: 1,1-Dimethylethyl 2-methyl-4-{[(trifluoromethyl)sulfonyl]oxy}-3,6-dihydro-1(2H)-pyridinecarboxylate (D158)

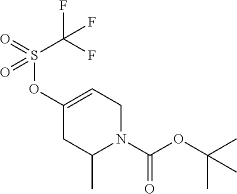

To a solution of 1,1-dimethylethyl 2-methyl-4-oxo-1-piperidinecarboxylate (D156) (6.95 g, 0.0325 mol) in dry THF (60 ml) cooled at −78° C. was added LiHDMS (35.8 ml of a 1M sol. in hexane, 0.0358 mol) followed by dropwise addition of a solution of 1,1,1-trifluoro-N-phenyl-N-[(trifluoromethyl)sulfonyl]methanesulfonamide (11.5 g, 0.0325 mol) in THF (60 ml). After 30 minutes at −78° C., the reaction mixture was allowed to warm at room temperature and further stirred 4 hours. NH₄Cl saturated solution (200 ml) was added and the mixture extracted with ethyl acetate (3×100 ml). The combined organic layers were dried (Na₂SO₄) and evaporated in vacuo to afford 10 g of crude material. The obtained mixture was purified by Horizon column (65M) eluting with 3% ethyl acetate in cyclohexane to afford a 1:1 mixture of the double bond regioisomers as cream solid; MS (ES) m/z: 246.1, 290.0, 346.0 [MH⁺]. C₁₂H₁₈F₃NO₅S requires 345.3

### Description 159: 1,1-Dimethylethyl 6-methyl-4-{[(trifluoromethyl)sulfonyl]oxy}-3,6-dihydro-1(2H)-pyridinecarboxylate) (D159)

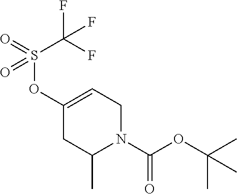

The title compound was prepared according to the experimental procedure of Description 158 starting from 1,1-Dimethylethyl 2-methyl-4-oxo-1-piperidinecarboxylate (D157); MS (ES) m/z: 246.1, 290.0, 346.0 [MH⁺]. C₁₂H₁₈F₃NO₅S requires 345.3

### Description 160: 2-Methyl-5-(4,4,5,5-tetramethyl-1,3,2-dioxaborolan-2-yl)quinoline (D160)

A mixture of 2-methyl-5-quinolinyl trifluoromethanesulfonate (preparation reported in WO 2004046124 A1) (13.5 g 0.046 mol), bis(pinacolato)diboron (13 g, 0.051 mol), [1,1′-bis(diphenylphosphino)ferrocene] dichloro palladium complex (1.88 g, 0.0023 mol), 1,1′-bis(diphenylphosphino)ferrocene (1.279, 0.0023 mol) and potassium acetate (13.5 g, 0.138 mol) in dry 1,4 dioxane (120 ml) was heated overnight at 80° C. Solvent was evaporated to dryness and the crude material purified on a Horizon column (65M) eluting with 10% ethyl acetate in cyclohexane to afford the title compound as a cream solid (10 g, 81%); MS (ES) m/z: 270.2 [MH⁺]. C₁₆H₂₀BNO₂ requires 269.15.

### Description 161 (racemic mixture): 1,1-Dimethylethyl 6-methyl-4-(2-methyl-5-quinolinyl)-3,6-dihydro-1(2H)-pyridinecarboxylate (D161)

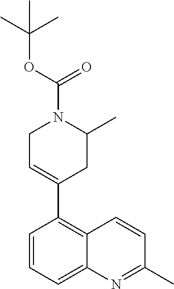

A mixture of the double bond regioisomers of 1,1-dimethylethyl 2-methyl-4-{[(trifluoromethyl)sulfonyl]oxy}-3,6-dihydro-1(2H)-pyridinecarboxylate (D158) (1.65 g, 4.778 mmol), 2-Methyl-5-(4,4,5,5-tetramethyl-1,3,2-dioxaborolan-2-yl)quinoline (D160) (1.35 g, 5.017 mmol), [1,1′-bis(diphenylphosphino)ferrocene] dichloro palladium complex (0.39 mg, 0.4778 mol) and potassium acetate (2 g, 14.33 mmol) in dry DMF (100 ml) was heated at reflux for 4 hours. Solvent was evaporated to dryness and the crude material purified by Horizon column (40M) eluting with a linear gradient starting from 10% ethyl acetate in cyclohexane to 30% ethyl acetate in cyclohexane to afford the title compound as cream solid (736 mg, 45%); MS (ES) m/z: 339.2 [MH⁺]. C₂₁H₂₆N₂O₂ requires 338.45.

### Description 162 (racemic mixture of enantiomers): 1,1-Dimethylethyl 2-methyl-4-(2-methyl-5-quinolinyl)-1-piperidine-carboxylate (D162)

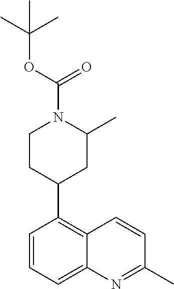

1,1-Dimethylethyl 6-methyl-4-(2-methyl-5-quinolinyl)-3,6-dihydro-1(2H)-pyridinecarboxylate (D161) (736 mg, 2.17 mmol) was dissolved in ethanol 95% (8.5 ml) in a hydrogenation flask. Pd/C 10% (84 mg) was added and the reaction mixture was vigorously stirred under hydrogen at atmospheric pressure for 9 days. Catalyst was filtered off and the solution evaporated in vacuo. The crude material was purified on a Horizon column (40M) eluting with a linear gradient starting from 100% cyclohexane to 20% ethyl acetate in cyclohexane to afford the title compound as an off white solid (350 mg, 47%); MS (ES) m/z: 341.3 [MH⁺]. C₂₁H₂₈N₂O₂ requires 340.46

### Description 163 (racemic mixture of enantiomers): 2-Methyl-5-(2-methyl-4-piperidinyl)quinoline (D163)

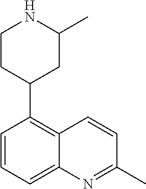

1,1-Dimethylethyl 2-methyl-4-(2-methyl-5-quinolinyl)-1-piperidinecarboxylate (D162) (350 mg, 1.03 mmol) was dissolved in a 3:1 mixture TFA:DCM (3:1 ml) and stirred overnight at room temperature. The solvent was evaporated in vacuo and the crude mixture was purified by SPE-SCX cartridge (2 g) (eluting with methanol followed by 2N ammonia solution in methanol) to afford the title compound as a white solid (212 mg, 86%); MS (ES) m/z: 241.2 [MH⁺]. C₁₆H₂₀N₂ requires 240.36

### Description 164: 1,1-Dimethylethyl 4-(2-methyl-5-quinolinyl)-1-piperazinecarboxylate (D164)

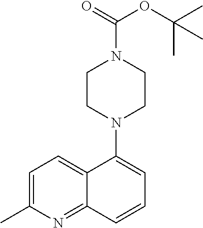

To a solution of 2-methyl-5-(1-piperazinyl)quinoline (3.00 g, 13.19 mmol) in dioxane (10 ml) and saturated aqueous NaHCO₃ solution (10 ml) was added BOC₂O (3.17 g, 14.52 mmol) and stirred at room temperature overnight. Water was added and the product was extracted with ethyl acetate (2×). The organic layer was dried (MgSO₄), filtered and evaporated in vacuo to afford the title compound (4.51 g, quant.); MS (ES; m/z): 328 [MH⁺]; C₁₉H₂₅N₃O₂ (327.43); NMR (¹H, 300 MHz, CDCl₃) δ: 8.36 (d, 1H), 7.72 (d, 1H), 7.56 (t, 1H), 7.26 (d, 1H), 7.02 (d, 1H), 3.68 (m, 4H), 3.00 (m, 4H), 2.72 (s, 3H), 1.48 (s, 9H).

### Description 165: 1,1-dimethylethyl 4(2-formyl-5-quinolinyl)-1-piperazinecarboxylate (D165)

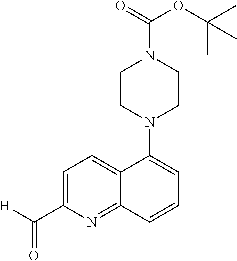

To a solution of intermediate 164 (4.51 g, 13.77 mmol) in dioxane (20 ml) was added SeO₂ (1.83 g, 16.52 mmol). The mixture was stirred at 90° C. for 1.5 h and then cooled to room temperature and subsequently filtered and evaporated. The residue was purified by flash column chromatography (cyclohexanes:EtOAc 9:1 to 1:1) to afford the title compound (3.35 g, 71%) as a yellow solid; MS (ES; m/z): 342 [MH⁺], C₁₉H₂₃N₃O₃ (341.41); NMR (¹H, 300 MHz, CDCl₃) δ: 10.20 (s, 1H), 8.63 (d, 1H), 7.98 (q, 2H), 7.71 (t, 1H), 7.21 (d, 1H), 3.69 (bs, 4H), 3.03 (bs, 4H), 1.48 (s, 9H).

### Description 166: 2-Bromophenyl-2-propenyl ether (D166)

A round flask equipped with a refrigerant was charged with o-bromophenol (500 g), the acetone (5000 ml) and K₂CO₃ (440 g). Then the allyl bromide (385 g) was added dropwise (over 30 min). The mixture was heated at reflux for 2 hours. The reaction was cooled down and the solid was filtered. The solution was concentrated to 1500 ml under vacuum and then 5000 ml of cyclohexane were added. The solution was concentrated again to 1500 ml and then 5000 ml of cyclohexane was added. Finally the mixture was concentrated to 1500 ml and 1500 ml of cyclohexane were added.

### Description 167: 2-Bromo-6-(2-propen-1-yl)phenol

The Et₂AlCl in hexanes (2885 ml) was added to the product of the reaction mixture of Description 166 dropwise controlling the temperature below 30° C. (internal temperature). The reaction was monitored by HPLC. When complete, the reaction mixture was cooled down to 5° C. (internal temperature) and HCl 1N (3000 ml) was added dropwise controlling the gas evolution at the beginning (pH control). Water (50 ml) was then added dropwise. The phases were separated and the organic phase was extracted with cyclohexane (2500 ml). The solution was concentrated to 1500 ml under vacuum and 5000 ml of THF were added. The solution was concentrated again to 1500 ml, and then 5000 ml of THF were added. Finally the mixture was concentrated to 3075 ml.

### Description 168: (3-bromo-2-hydroxyphenyl)acetaldehyde

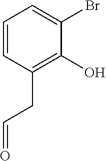

A solution of sodium periodate (1537.5 g) and water (11375 ml) was prepared and left to stir overnight. The solution was filtered. To the THF solution of 2-bromo-6-(2-propen-1-yl)phenol (D167) was added water (920 ml) and then K₂OsO₄.2H₂O (1.18 g). The mixture was stirred at room temperature for 30 min. Then the aqueous sodium periodate solution was added dropwise over 3 hours, controlling that the internal temperature below 20-25° C. The reaction was monitored by HPLC. When complete, water (5000 ml) and DCM (4000 ml) was added to the reaction mixture. The mixture was stirred for 15 min. The phases were separated and the organic phase was washed with water (5000 ml). To the organic phase was added Si-tiol resin (28.5 g, a scavenger for heavy metal) and the solution was heated at reflux for 2 hr. On cooling to room temperature the solution was passed through a CUNO filter to remove metal contaminants. The solution was then concentrated to 2500 ml vol under vacuum.

### Description 169: 2-Bromo-6-{2-[4-(2-methyl-5-quinolinyl)-1-piperazinyl]ethyl}phenol

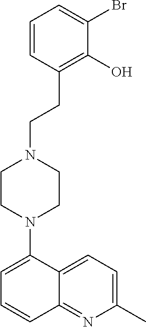

A reactor was charged with the piperazine (314 g), DCM (2590 ml) and sodium triacetoxyborohydride (399.6 g). The mixture was stirred for 15 min at room temperature (a thick solution was formed). (3-Bromo-2-hydroxyphenyl)acetaldehyde (D168) in DCM (370 g in 2500 ml DCM) was added dropwise whilst controlling the internal temperature. The reaction was monitored by HPLC. To the reaction mixture was add NaOH 10% until the pH turned basic (ca. 1110 ml) (the pH was checked using paper strips or a pH meter). Then the phases were separated and the organic phase was washed with water (3700 ml). To the organic phase was added 3700 ml of THF, then the mixture was concentrated to 3700 ml at atmospheric pressure (T=45-55° C.). Then 3700 ml of THF were added and the mixture was concentrated again to 3330 ml at atmospheric pressure (T=55-57° C.). The reaction mixture was cooled to room temperature slowly (ca. 1 h). The mixture was seeded and heptane (5550 ml) was added dropwise. The mixture was stirred overnight at r.t. The solid was filtered and the cake washed with 925 ml of THF/heptane 1:2. The solid was dried in the vacuum oven at 40° C.

### Description 170: 1-[(2-bromo-6-{2-[4-(2-methyl-5-quinolinyl)-1-piperazinyl]ethyl}phenyl)oxy]-2-propanone

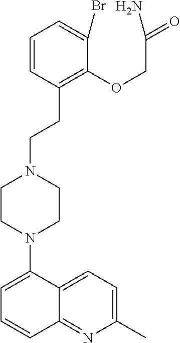

To the reactor was added 2-bromo-6-{2-[4-(2-methyl-5-quinolinyl)-1-piperazinyl]ethyl}phenol (D169), phenol (550 g), methyl ethyl ketone (5500 ml), chloroacetamide (302.5 g), potassium carbonate (325 mesh) (550 g) and 18,6-crown ether (17.05 g). The mixture was heated to reflux for 90 min (heterogeneous solution). The reaction was monitored by HPLC. On completion, the reaction mixture was cooled to room temperature. DCM (8250 ml) was added and then water (8250 ml). The organic phase was separated. The aqueous phase was monitored by HPLC, if there was still some product then the aqueous phase was re-extracted with 5500 ml of DCM. To the organic phase was added 5500 ml of THF and the mixture was concentrated to 5500 ml at atmospheric pressure (T=45-55° C.). Then 5500 ml of THF were added, the mixture was concentrated again to 4950 ml at atmospheric pressure (T=55-57° C.). The reaction mixture was cooled to room temperature slowly (ca. 1 h). The mixture was seeded and heptane (8250 ml) was added dropwise. The mixture was stirred overnight at room temperature. The solid was filtered and the cake washed with 1375 ml of THF/heptane 1:2. The solid was dried in a vacuum oven at 40° C.

### Description 171: 8-{2-[4-(2-Methyl-5-quinolinyl-1-piperazinyl]ethyl}-2H-1,4-benzoxazin-3(4H)-one

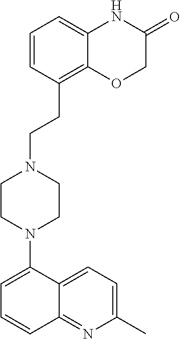

A reactor was charged under nitrogen with DMF (4750 ml), CuI (75.05 g) and N,N′-dimethyl ethylenediamine (97.85 g). The reaction mixture was stirred under N₂ until the mixture turned dark blue (ca. 2 hr). Potassium carbonate (325 mesh) (306 g) and 1-[(2-bromo-6-{2-[4-(2-methyl-5-quinolinyl)-1-piperazinyl]ethyl}phenyl)oxy]-2-propanone (D170) (475 g) were added to the reaction mixture. The heterogeneous mixture was heated to 110° C. (internal temperature) for 16 hours. The reaction was monitored by HPLC. The reaction mixture was cooled to room temperature and water (9500 ml) and DCM (9500 ml) were added. The reaction mixture was stirred at 30 degC (internal temperature) for 1 hr. Then the mixture was cooled down to room temperature and the phases were separated. The aqueous phase was monitored by HPLC, if there was still some product present then it was re-extracted with 4750 ml of DCM. The organic phase was washed first with NH₄OH 3.5% (4750 ml) and then with water (4750 ml) four times (until no more DMF was observed by HPLC). The organic phase was passed through a CUNO filter to remove metal contaminants. To the organic phase was added 4750 ml of THF, then the mixture was concentrated to 4750 ml at atmospheric pressure (T=45-55° C.). Then 4750 ml THF were added, the mixture was concentrated again to 4750 ml at atmospheric pressure (T=55-57° C.). The mixture was cooled down to room temperature slowly (ca. 1 h). The mixture was seeded and heptane (7125 ml) was added dropwise. The mixture was stirred overnight at room temperature. The solid was filtered and the cake washed with 1187 ml of THF/heptane 1:2. The solid was dried in a vacuum oven at 40° C.

### EXAMPLES

### Example 1

### Ethyl 6-{2-[4-(2-methyl-5-quinolinyl)-1-piperazinyl]ethyl}-4H-imidazo[5,1-c][1,4]-benzoxazine-3-carboxylate dihydrochloride (E1)

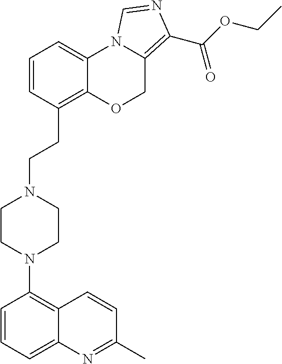

A mixture of ethyl 6-(2-oxoethyl)-4H-imidazo[5,1-c][1,4]benzoxazine-3-carboxylate (D6) (50 mg, 0.17 mmol) and 2-methyl-5-piperazin-1-yl-quinoline (58 mg, 0.21 mmol) in dry 1,2-dichloroethane (4 ml) was stirred at room temperature under nitrogen for 15 minutes. Sodium triacetoxyborohydride (44 mg, 0.21 mmol) was then added and the resulting reaction mixture was stirred for 3 hours, quenched with a saturated aqueous solution of ammonium chloride and extracted with DCM (×3). The combined organics were dried (Na₂SO₄) and concentrated in vacuo. The crude product was purified by SPE cartridge (silica gel) eluting with 4% methanol in DCM to afford the free base of the title compound as a white solid (61 mg, 70%); MS (ES) m/z: 498.5 [MH⁺]; C₁₆H₁₆N₂O₃ requires 497.6; ¹H-NMR (300 MHz, CDCl₃) δ: 8.4 (d, 1H), 8 (s, 1H), 7.7 (d, 1H), 7.55 (t, 1H), 7.3 (d, 1H), 7.2 (m, 1H), 7.1 (d, 1H), 6.95-7.05 (m, 2H), 5.55 (s, 2H), 4.4 (q, 2H), 2.7-3.15 (m, 12H), 3.7 (s, 3H), 1.4 (t, 3H). The free base (20 mg, 0.04 mmol) was dissolved in dry methanol (1 ml) and treated with HCl (0.068 ml of a 1.25 M solution in methanol, 0.084 mmol) at 0° C. The resulting suspension was stirred at 0° C. for 4 h. Evaporation of the volatiles and trituration with diethyl ether gave the title compound (20 mg, 87%) as a yellow solid; ¹H-NMR (500 MHz, DMSO-d⁶) δ: 10.67 (bs, 1H), 8.8-8.6 (bs, 1H), 7.89 (dd, 1H), 7.82 (s, 2H), 7.64 (bs, 1H), 7.35 (s, 1H), 7.29 (dd, 1H), 7.2 (d, 1H), 4.3 (q, 2H), 3.76 (dd, 2H), 3.6-3.3 (m, 8H), 2.8 (s, 3H), 1.33 (t, 3H).

Example 1 was also prepared as follows.

Into a reactor was added 8-{2-[4-(2-methyl-5-quinolinyl)-1-piperazinyl]ethyl}-2H-1,4-benzoxazin-3(4H)-one (D171) (274 g) and THF (2740 ml). The mixture was cooled to 0° C. and a solution of ^{t}BuOK in THF 1M (748 ml) was added dropwise controlling the temperature at 0° C. Diethylchlorophosphate (126 ml) was added at 0° C. and the reaction mixture stirred at 0° C. for 30 min. The reaction mixture was cooled to −5° C. and ethylisocyanoacetate (87.7 ml) and ^{t}BuOK in THF 1M (811 ml) were added maintaining the temperature below 0° C. The reaction mixture was allowed to stir at −5° C. for 1.5 hours. The reaction was monitored by HPLC. The reaction mixture was quenched by adding ammonium chloride solution (20%; 4250 ml) and then the THF was evaporated to 4658 ml. DCM (5480 ml) was added and the mixture was heated to 30 degC. After stirring for 30 mins., the phases were separated and the aqueous phase was re-extracted with DCM (2740 ml) if product was still present (after monitoring by HPLC). The organic phases were combined and washed with water (2740 ml). The organic phase was passed through a CUNO filter to revove metal contaminants. The organic phase was concentrated to 822 ml (P=1000 mbar, T=66° C.) and to it was added 2740 ml of acetone and mixture concentrated to 2192 ml (P=950 mbar T=45-55° C.). Then a further 274 ml of acetone was added and the mixture was concentrated again to 1370 ml at (P=950 mbar T=55-57° C.). On cooling a sample was taken to monitor by NMR the amount of DCM. If DCM remained, more solvent exchange has to be done, until the amount of DCM was 52.25%. Then the mixture was cooled to r.t. and was stirred overnight. The solid was filtered and the cake washed with 685 ml of acetone. The solid was dried in a vacuum oven at 40° C.

### Example 2

### 6-{2-[4-(2-Methyl-5-quinolinyl)-1-piperazinyl]ethyl}-4H-imidazo[5,1-c][1,4]-benzoxazine-3-carboxylic acid (E2)

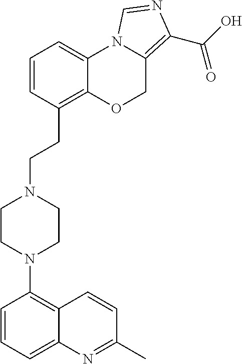

To a solution of ethyl 6-{2-[4-(2-methyl-5-quinolinyl)-1-piperazinyl]ethyl}-4H-imidazo[5,1-c][1,4]benzoxazine-3-carboxylate (E1) (220 mg, 0.44 mmol) in methanol (3 ml) was added NaOH (3 ml of a 10% aq. solution) and the resulting white suspension was heated for 5 minutes under microwave Irradiation at 120° C. The resulting pale yellow solid was filtered off, suspended in water (15 ml) and the solution was neutralised (pH=7) with an aqueous solution of acetic acid. The resulting off-white solid precipitate was filtered and washed with diethyl ether (3×20 ml) affording the title compound (0.168 g, 81%); MS (ES) m/z: 470.3 [MH⁺]. C₂₇H₂₇N₅O₃ requires 469.54; ¹H-NMR (300 MHz, DMSO-d⁶) δ: 8.49 (bs, 1H), 8.3 (d, 1H), 7.7 (d, 1H), 7.55 (bs, 1H), 7.35 (d, 1H), 7.2 (d, 1H), 7.07 (bm, 2H), 5.5 (s, 2H), 3.05-2.75 (vbm, 15H).

The potassium salt of Example 2 was prepared as follows.

Ethyl 6-{2-[4-(2-methyl-5-quinolinyl)-1-piperazinyl]ethyl}-4H-imidazo[5,1-c][1,4]benzoxazine-3-carboxylate dihydrochloride (E1) (115 g) was suspended in a solution of 1.5M of KOH in methanol (805 ml). The suspension was heated under reflux for 1.5 h. The reaction mixture was cooled to room temperature and filtered. The solid was washed with methanol (287.5 ml). The white solid was dried in a vacuum oven at 40° C.

### Example 3

### N-Cyclopentyl-6-{2-[4-(2-methyl-5-quinolinyl)-1-piperazinyl]ethyl}-4H-imidazo[5,1-c][1,4]-benzoxazine-3-carboxamide dihydrochloride (E3)

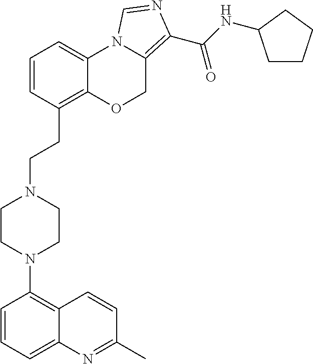

The title compound was prepared in 40% yield following the general procedure of Example 1 starting from N-cyclopentyl-6-(2-oxoethyl)-4H-imidazo[5,1-c][1,4]benzoxazine-3-carboxamide (D11) (25 mg, 0.08 mmol) and 2-methyl-5-piperazin-1-yl-quinoline (20 mg, 0.09 mmol) (WO2004/046124); MS (ES) m/z: 537.3 [MH⁺], C₃₂H₃₆N₆O₂ requires 536.6; ¹H-NMR (500 MHz, DMSO-d⁶) δ: 10.79 (vbs, 1H), 8.67 (vbs, 1H), 8.58 (s, 1H), 7.9 (d, 1H), 7.8 (d+vbs, 3H), 7.6 (vbs, 1H), 7.32 (bs, 1H), 7.24 (d, 1H), 7.15 (t, 1H), 5.59 (s, 2H), 4.18 (m, 1H), 3.7-3 (vm, 12H), 2.78 (bs, 3H), 1.83 (m, 2H), 1.67 (m, 2H), 1.52 (m, 4H).

### Example 4

### 6-{2-[4-(2-Methyl-5-quinolinyl)-1-piperazinyl]ethyl}-4H-imidazo[5,1-c][1,4]-benzoxazine dihydrochloride (E4)

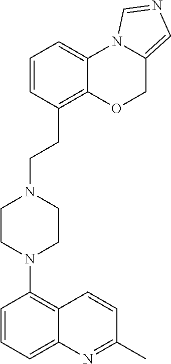

The title compound was prepared in 40% yield following the general reductive amination procedure of Example 1 starting from 4H-imidazo[5,1-c][1,4]benzoxazin-6-ylacetaldehyde (D13) (20 mg, 0.08 mmol) and 2-methyl-5-piperazin-1-yl-quinoline (20 mg, 0.1 mmol); MS (ES) m/z: 426.3 [MH⁺], C₂₆H₂₇N₅O requires 425.5; ¹H-NMR (500 MHz, DMSO-d⁶) δ: 10.64 (bs, 1H), 8.64 (s, 1H), 8.47 (d, 1H), 7.81 (d, 1H), 7.7 (s, 2H), 7.48 (d, 1H), 7.3-7.1 (m, 3H), 7.07 (s, 1H), 5.35 (s, 2H), 3.8-3.1 (vbm, 12H), 2.68 (s, 3H).

### Example 5

### Ethyl 6-{3-[4-(2-methylquinolin-5-yl]piperazin-1-yl]propyl}-4H-imidazo[5,1-c][1,4]-benzoxazine-3-carboxylate dihydrochloride (E5)

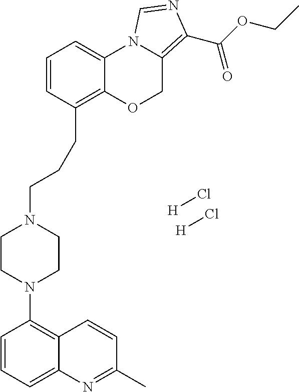

The title compound was prepared in 40% yield following the general reductive amination procedure of Example 1 starting from ethyl 6-(3-oxopropyl)-4H-imidazo[5,1-c][1,4]benzoxazine-3-carboxylate (D17) (20 mg, 0.08 mmol) and 2-methyl-5-piperazin-1-yl-quinoline (20 mg, 0.1 mmol); MS (ES) m/z: 512.4 [MH⁺]. C30H33N5O3 requires 511.6; ¹H-NMR (500 MHz, DMSO-d⁶) δ: 10.92 (bs, 1H), 8.71 (s, 1H), 8.64 (s, 1H), 7.83 (m, 3H), 7.67 (bs, 2H), 7.33 (bs, 1H), 7.26 (d, 1H), 7.15 (t, 1H), 5.6 (s, 2H), 4.3 (q, 2H), 3.64 (bd, 2H), 3.0-3.3 (vbm, 8H), 2.81 (bs, 3H), 2.77 (dd, 2H), 2.12 (bs, 2H), 1.33 (s, 3H).

### Example 6

### 6-{3-[4-(2-Methyl-5-quinolinyl)-1-piperazinyl]propyl}-4H-imidazo[5,1-c][1,4]-benzoxazine-3-carboxylic acid (E6)

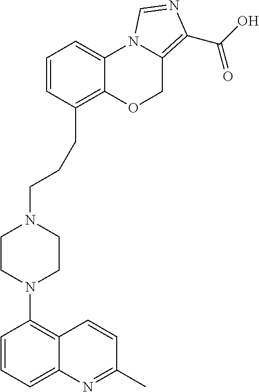

Ethyl 6-{3-[4-(2-methylquinolin-5-yl)piperazin-1-yl]propyl}-4H-imidazo[5,1-c][1,4]benzoxazine-3-carboxylate dihydrochloride (E5) (100 mg, 0.2 mmol) was dissolved in methanol (1 ml) and sodium hydroxide (1 ml of 1M aq. solution) was added. The reaction mixture was irradiated in a microwave reactor (PersonalChemistry Emrys™ Optimiser, 300W, 120° C., 5 minutes). The crude material was purified by SPE-SCX cartridge and then treated with trifluoroacetic acid (0.2 mmol) to afford the title compound; MS (ES) m/z: 484.3 [MH⁺], C28H29N5O3 requires 483.6; ¹H-NMR (300 MHz, DMSO-d⁶) δ: 9.6 (bs, 1H), 8.6 (s, 1H), 8.4 (d, 1H), 7.8 (d, 1H), 7.65 (m, 1H), 7.4 (d, 1H), 7-7.2 (m, 4H), 5.55 (s, 2H), 3.0-3.7 (vbm, 10H), 2.81 (m, 2H), 2.77 (s, 3H), 2.12 (bs, 2H).

### Example 7

### 6-{3-[4-(2-Methyl-5-quinolinyl)-1-piperazinyl]propyl}-4H-imidazo[5,1-c][1,4]-benzoxazine dihydrochloride (E7)

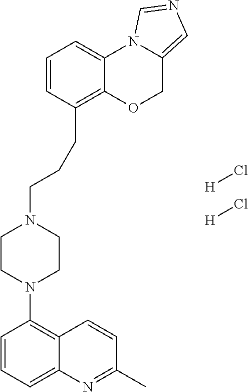

6-{3-[4-(2-Methyl-5-quinolinyl)-1-piperazinyl]propyl}-4H-imidazo[5,1-c][1,4]-benzoxazine-3-carboxylic acid (E6) (30 mg, 0.06 mmol) in 1,2-dichlorobenzene (1.5 ml) was irradiated in a microwave reactor (PersonalChemistry Emrys™ Optimiser, 300W, 250° C., 10 min). The solvent was removed by SPE-SCX cartridge (eluting with methanol followed by 2N ammonia solution in methanol) to afford the free base of the title compound as a white solid (20 mg, 72%). The free base was dissolved in dry methanol (1 ml) and HCl (80 μl of a 1.25M solution in methanol, 0.1 mmol) was slowly added at 0° C. The resulting suspension was stirred at 0° C. for 4 h. Evaporation of the volatiles and trituration with diethyl ether gave the title compound as a yellow solid (20 mg); MS (ES) m/z: 440.4 [MH⁺], C27H29N5O requires 439.6; ¹H-NMR (500 MHz, DMSO-d⁶) δ: 10.4 (bs, 1H), 8.4 (s, 1H), 8.3 (d, 1H), 7.6-7.75 (m, 3H), 7.4 (d, 1H), 7.1-7.2 (m, 3H), 7 (bs, 1H), 5.3 (s, 2H), 3.1-3.6 (bm, 10H), 2.7 (m, 2H), 2.6 (bs, 3H), 2.1 (m, 2H).

### Examples 8 and 9

### 6-{2-[4-(2-Methyl-5-quinolinyl)-1-piperazinyl]ethyl}-4H-imidazo[2,1-c][1,4]-benzoxazine dihydrochloride (E8) and 6-{[4-(2-Methyl-5-quinolinyl)-1-piperazinyl]methyl}-4H-imidazo[2,1-c][1,4]benzoxazine dihydrochloride (E9)

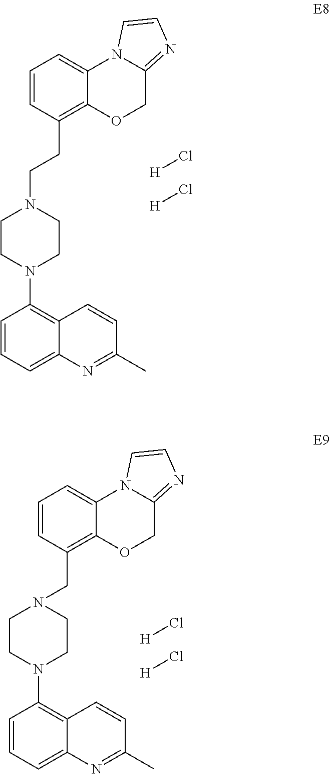

The title compounds were prepared as a mixture following the general reductive amination procedure of Example 1 starting from the mixture of aldehydes 4H-imidazo[2,1-c][1,4]benzoxazin-6-ylacetaldehyde and 4H-imidazo-[2,1-c][1,4]benzoxazine-6-carbaldehyde (D22 and D23) (50 mg, 0.2 mmol). The free base of the title compounds E8 and E9 (13.8 mg and 14 mg, 20%) were separated by chromatography on silica gel eluting with 2% methanol in DCM. The free bases were dissolved in dry methanol (0.5 ml) and HCl (2.2 eq of a 1.25M solution in methanol) was slowly added at 0° C. The resulting suspension was stirred at 0° C. for 4 h. Evaporation of the volatiles and trituration with diethyl ether gave the title compounds as yellow solids.

Example 8: MS (ES) m/z: 426.3 [MH⁺], C26H27N5O requires 425.5; ¹H-NMR (500 MHz, DMSO-d⁶) δ: 11.07 (bs, 1H), 8.78 (bm, 1H), 8.05 (s, 1H), 7.88 (bm, 2H), 7.7 (bm, 2H), 7.38 (bm, 1H), 7.25 (bs, 1H), 7.24 (d, 1H), 7.18 (t, 1H), 5.43 (s, 2H), 3.8-3.2 (vbm, 12H), 2.84 (s, 3H).

Example 9: MS (ES) m/z: 412.4 [MH⁺], C25H25N5O requires 411.5; ¹H-NMR (500 MHz, DMSO-d⁶) δ: 10.64 (bs, 1H), 8.8 (bs, 1H), 8.0 (bm, 1H), 7.9 (bs, 3H), 7.8 (bs, 1H), 7.7 (d, 1H), 7.1-7.4 (bm, 3H), 5.5 (s, 2H), 4.5 (s, 2H), 3.8-3.1 (vbm, 8H), 2.7 (s, 3H).

### Example 10

### 1-Methyl-6-{2-[4-(2-methyl-5-quinolinyl)-1-piperazinyl]ethyl}-4H-[1,2,4]triazolo[3,4-c][1,4]-benzoxazine dihydrochloride (E10)

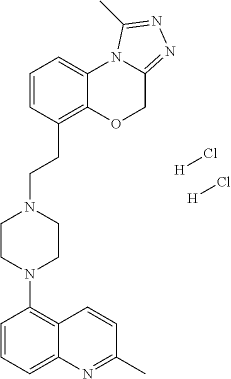

The title compound was prepared in 71% yield following the general reductive amination procedure of Example 1 starting from (1-methyl-4H-[1,2,4]triazolo[3,4-c][1,4]benzoxazin-6-yl)acetaldehyde (D25) (45 mg, 0.2 mmol) and 2-methyl-5-piperazin-1-yl-quinoline (54 mg, 0.24 mmol); MS (ES) m/z: 441.3 [MH⁺], C26H28N6O requires 440.6; ¹H-NMR (500 MHz, DMSO-d⁶) δ: 11.21 (bs, 1H), 8.92 (bs, 1H), 7.97 (bs, 2H), 7.81 (bd, 1H), 7.72 (d, 1H), 7.45 (bd, 1H), 7.34 (d, 1H), 7.25 (t, 1H), 5.46 (s, 2H), 3.75 (d, 2H), 3.3-3.6 (bm, 8H), 3.24 (m, 2H), 2.91 (s, 3H), 2.74 (s, 3H).

### Example 11

### 1-Methyl-6-{2-[4-(2-methyl-5-quinolinyl)-1-piperidinyl]ethyl}-4H-[1,2,4]triazolo[3,4-c][1,4]-benzoxazine dihydrochloride (E11)

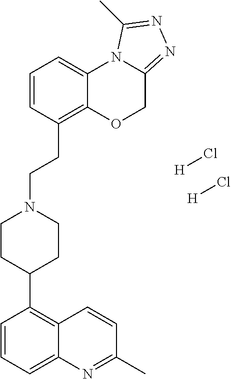

The title compound was prepared in 35% yield following the general reductive amination procedure of Example 1 starting from (1-methyl-4H-[1,2,4]triazolo[3,4-c][1,4]benzoxazin-6-yl)acetaldehyde (D25) (50 mg, 0.22 mmol) and 2-methyl-5-(4-piperidinyl)quinoline (54.77 mg, 0.24 mmol); MS (ES) m/z: 440.10 [MH⁺], C₂₇H₂₉N₅O requires 439.57; ¹H-NMR (500 MHz, DMSO-d⁶) δ: 10.3 (bs, 1H), 8.02 (bs, 1H), 8.06 (bs, 1H), 7.95 (bs, 1H), 7.82 (bs, 1H), 7.74 (d, 1H), 7.64 (bs, 1H), 7.34 (d, 1H), 7.25 (t, 1H), 5.46 (s, 2H), 3.76 (d, 2H), 3.80-3.10 (vbm, 6H), 2.86 (bs, 3H), 2.74 (s, 3H), 2.3-2.1 (m, 5H).

### Example 12

### 1-Methyl-6-{2-[(2R)-2-methyl-4-(2-methyl-5-quinolinyl)-1-piperazinyl]ethyl}-4H-[1,2,4]triazolo[3,4-c][1,4]-benzoxazine dihydrochloride (E12)

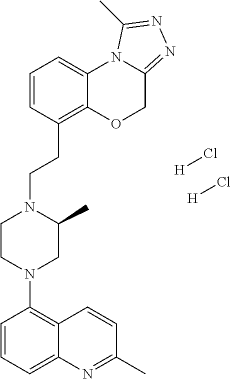

The title compound was prepared in 47% yield following the general reductive amination procedure of Example 1 starting from (1-methyl-4H-[1,2,4]triazolo[3,4-c][1,4]benzoxazin-6-yl)acetaldehyde (D25) (26 mg, 0.11 mmol) and 2-methyl-5-[(3R)-3-methyl-1-piperazinyl]quinoline (40 mg, 0.16 mmol) (WO2004/046124); MS (ES) m/z: 455.70 [MH⁺], C₂₇H₃₀N₆O requires 454.58; ¹H-NMR (500 MHz, DMSO-d⁶) δ: 10.85 (bs, 1H), 8.85 (bs, 1H), 7.88 (bs, 2H), 7.72 (bs, 1H), 7.7-7.6 (bs, 1H), 7.4-7.3 (bs, 1H), 7.38 (d, 1H), 7.25 (t, 1H), 5.48 (s, 2H), 4-3.2 (vbm, 11H), 2.85 (bs, 3H), 2.74 (s, 3H), 1.46-1.6 (2d, 3H).

### Example 13

### 6-{2-[4-(7-Fluoro-2-methyl-5-quinolinyl)-1-piperazinyl]ethyl}-1-methyl-4H-[1,2,4]triazolo[3,4-c][1,4]-benzoxazine dihydrochloride (E13)

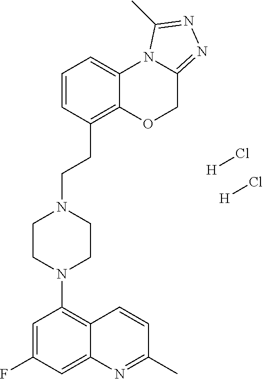

The title compound was prepared in 53% yield following the general reductive amination procedure of Example 1 starting from (1-methyl-4H-[1,2,4]triazolo[3,4-c][1,4]benzoxazin-6-yl)acetaldehyde (D25) (25 mg, 0.11 mmol) and 7-fluoro-2-methyl-5-(1-piperazinyl)quinoline (38 mg, 0.15 mmol) (WO2004/046124); MS (ES) m/z: 459.30 [MH⁺], C26H₂₇N₆OF requires 458.54; ¹H-NMR (500 MHz, DMSO-d⁶) δ: 10.44 (bs, 1H), 8.44 (bs, 1H), 7.73 (d, 1H), 7.47 (d, 1H), 7.43 (m, 1H), 7.34 (d, 1H), 7.32 (t, 1H), 7.22 (m, 1H), 5.46 (s, 2H), 3.74 (d, 2H), 3.6-3.1 (m, 10H), 2.69 (bs, 3H), 2.74 (s, 3H).

### Examples 14 to 24

### General Procedure for Amide Formation

To a stirred solution of 6-{2-[4-(2-methyl-5-quinolinyl)-1-piperazinyl]ethyl}-4H-imidazo[5,1-c][1,4]benzoxazine-3-carboxylic acid (E2) (1 eq) and DIPEA (1.1 eq) in DMF was added TBTU (1.1 eq) and the resulting solution was stirred for 1 hour at room temperature. The desired amine (1.1 eq) was added and the solution stirred for 1 hour. The crude solution was applied to a SPE-SCX cartridge (eluting with methanol followed by 2N ammonia solution in methanol). After evaporation of solvent from the ammonia fractions and trituration with diethyl ether, the free base of the desired compound was isolated in pure form. The free base was dissolved in dry methanol and treated with HCl (2.1 eq. of 1 M solution in diethyl ether) at 0° C. Evaporation of solvent and trituration with diethyl ether gave the final compounds in pure form.

### Example 14

### 6-{2-[4-(2-methyl-5-quinolinyl)-1-piperazinyl]ethyl}-4H-imidazo[5,1-c][1,4]benzoxazine-3-carboxamide dihydrochloride (E14)

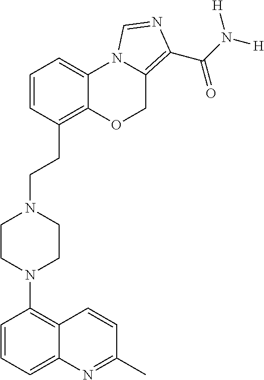

To a stirred solution of 6-{2-[4-(2-Methyl-5-quinolinyl)-1-piperazinyl]ethyl}-4H-imidazo[5,1-c][1,4]benzoxazine-3-carboxylic acid (E2) (30 mg, 0.06 mmol) and DIPEA (0.012 ml) in DMF (1 ml) was added TBTU (22.6 mg, 0.07 mmol) and the resulting solution was stirred for 1 hour at room temperature. Hexamethyldisilazane (0.013 ml, 0.07 mmol) was added and the solution stirred for 1 h. The excess of amine was scavenged by using a PS-isocyanate resin and after filtration of the resin and evaporation of the solvent, the crude product was purified by SPE cartridge (silica gel) eluting with 3% methanol in DCM to afford the free base of the title compound as a white solid. The free base was dissolved in dry methanol and treated with HCl (2.1 eq. of 1 M solution in diethyl ether) at 0° C. Evaporation of solvent and trituration with diethyl ether gave the final compound in pure form (22 mg, 65%); MS (ES) m/z: 469.4 [MH⁺], C₂₇H₂₈N₆O₂ requires 468.56; ¹H-NMR (300 MHz, DMSO-d⁶) δ: 11.4 (bs, 1H), 9.05 (d, 1H), 8.59 (s, 1H), 8.08 (d, 1H), 8.01 (t, 1H), 7.91 (d, 1H), 7.84 (dd, 1H), 7.51-7.49 (bd, 2H), 7.33 (bs, 1H), 7.26 (d, 1H), 7.15 (t, 1H), 5.59 (s, 2H), 3.81-3.72 (bd, 2H), 3.53-3.49 (bd, 4H), 3.43-3.39 (bd, 4H), 3.22-3.17 (m, 2H), 2.97 (s, 3H).

The free base of Example 14 was also prepared as follows.

Potassium 6-{2-[4-(2-methyl-5-quinolinyl)-1-piperazinyl]ethyl}-4H-imidazo[5,1-c][1,4]benzoxazine-3-carboxylate (E2) (63.5 g) was suspended into DMF (952.5 ml) under N₂. The suspension was stirred for 15 min at room temperature and then DIPEA (23.5 ml) and TBTU (45.72 g) were added. The suspension turned orange-brown and became a clear orange-brown solution after 45 min at room temperature. To the clear reaction mixture, HMDS (28.5 ml) was added at room temperature. During the addition a white precipitate formed and the suspension was stirred for a further 2 h at room temperature. Water (127 ml) was added to the reaction mixture, the suspension stirred for 30 min and then filtered (the filtration was very slow). The cake was washed with a mixture of water/THF (2/1, 315 ml) and then with MTBE (63 ml). The resulting solid was dried in a vacuum oven at 40° C. for 24 h.

Example 14 (i.e. the dihydrochloride salt), was also prepared from the free base as follows.

6-{2-[4-(2-Methyl-5-quinolinyl)-1-piperazinyl]ethyl}-4H-imidazo[5,1-c][1,4]benzoxazine-3-carboxamide (250.13 mg) was slurried in 90:10 v/v THF/H₂O mix (15 ml). The mixture was heated to just below reflux and allowed to cool to around room temperature. Concentrated hydrochloric acid (89 μl) was then added to the suspension, which immediately turned the slurry yellow and encouraged significant dissolution of solid. A few large agglomerates of a deep yellow/orange solid remained out of solution, but these were broken up with stirring. The mixture was heated again to near reflux, and practically all of the solid entered solution leaving being a homogeneous yellow solution. On cooling significant quantities of yellow solid began precipitating from solution. The mixture was then transferred into temperature cycling apparatus, and cycled between 0-40 degC over the weekend to maximise the sample crystallinity. The solid was isolated by filtration and dried for 48 hours in vacuo at 40 degC.

The free base of Example 14 was also prepared as follows.

6-{2-[4-(2-Methyl-5-quinolinyl)-1-piperazinyl]ethyl}-4H-imidazo[5,1-c][1,4]benzoxazine-3-carboxamide (250.52 mg) was slurried in 90:10 v/v MeOH/H₂O mix (15 ml). The mixture was heated to just below reflux, and allowed to cool to around room temperature. The mixture was reheated to just below reflux and again allowed to cool. The mixture was then transferred into temperature cycling apparatus, and cycled between 0-40 degC over the weekend to maximise the sample crystallinity. The solid was isolated by filtration and dried for 48 hours in vacuo at 40 degC.

The free base of Example 14 was also prepared as follows.

6-{2-[4-(2-Methyl-5-quinolinyl)-1-piperazinyl]ethyl}-4H-imidazo[5,1-c][1,4]benzoxazine-3-carboxamide dihydrochloride (E14) (6 g) were suspended in DCM (25 ml) and sodium bicarbonate (30 ml) were added. The suspension change colour from yellow to white and the mixture is left stirred for 30 mins. The solid was filtered and washed with water (15-20 ml). The filtercake was dried in an oven at 40 deg for 12 hrs, to give the title product (4 g).

The mesylate salt of 6-{2-[4-(2-Methyl-5-quinolinyl)-1-piperazinyl]ethyl}-4H-imidazo[5,1-c][1,4]benzoxazine-3-carboxamide was prepared as follows.

6-{2-[4-(2-Methyl-5-quinolinyl)-1-piperazinyl]ethyl}-4H-imidazo[5,1-c][1,4]benzoxazine-3-carboxamide (250.77 mg) was slurried in 90:10 v/v MeOH/H₂O mix (15 ml). The mixture was heated to just below reflux, and allowed to cool to around room temperature. Methanesulfonic acid (70 μl) was then added to the suspension, immediately turning it yellow. The mixture was again heated to just below reflux, at which point all of the solid entered solution leaving being a homogeneous yellow solution. On cooling significant quantities of yellow solid began precipitating from solution. The mixture was then transferred into temperature cycling apparatus, and cycled between 0-40 degC overnight to maximise the sample crystallinity. The solid was isolated by filtration and dried for 120 hours in vacuo at 40 degC.

### Example 15

### N-Methyl-6-{2-[4-(2-methyl-5-quinolinyl-1-piperazinyl]ethyl}-4H-imidazo[5,1-c][1,4]-benzoxazine-3-carboxamide dihydrochloride (E15)

The title compound was prepared in 44% yield from 6-{2-[4-(2-methyl-5-quinolinyl)-1-piperazinyl]ethyl}-4H-imidazo[5,1-c][1,4]benzoxazine-3-carboxylic acid (E2) following the general procedure for amide formation described above using methylamine (2M solution in THF); MS (ES) m/z: 483.4 [MH⁺], C₂₈H₃₀N₆O₂ requires 482.58; ¹H-NMR (300 MHz, DMSO-d⁶) δ: 11.29 (bs, 1H), 9.045 (d, 1H), 8.60 (s, 1H), 8.07-7.97 (m, 2H), 7.89 (d, 1H), 7.83 (dd, 1H), 7.49 (d, 1H), 7:37 (bs, 1H), 7.24 (t, 1H), 7.15 (t, 1H), 5.59 (s, 2H), 3.70-3.38 (bm, 10H), 3.22-3.15 (m, 2H), 2.96 (s, 3H), 2.74 (d, 3H).

### Example 16

### N-(1-Methylethyl)-6-{2-[4-(2-methyl-5-quinolinyl)-1-piperazinyl]ethyl}-4H-imidazo[5,1-c][1,4]-benzoxazine-3-carboxamide dihydrochloride (E16)

The title compound was prepared in 83% yield from 6-{2-[4-(2-methyl-5-quinolinyl)-1-piperazinyl]ethyl}-4H-imidazo[5,1-c][1,4]benzoxazine-3-carboxylic acid (E2) following the general procedure for amide formation described above using isopropylamine; MS (ES) m/z: 511.4 [MH⁺], C₃₀H₃₄N₆O₂ requires 510.64; ¹H-NMR (300 MHz, DMSO-d⁶) δ: 11.41 (bs, 1H), 9.07 (d, 1H), 8.61 (s, 1H), 8.09 (d, 1H), 8.02 (t, 1H), 7.92 (d, 1H), 7.87 (s, 1H), 7.85 (dd, 1H), 7.51 (d, 1H), 7.25 (d, 1H), 7.14 (t, 1H), 5.59 (s, 2H), 4.11-4.01 (m, 1H), 3.75-3.71 (bd, 2H), 3.53-3.49 (bd, 4H), 3.43-3.39 (bd, 4H), 3.22-3.15 (m, 2H), 2.98 (s, 3H), 1.15 (d, 6H).

### Example 17

### N-Cyclopropyl-6-{2-[4-(2-methyl-5-quinolinyl)-1-piperazinyl]ethyl}-4H-imidazo[5,1-c][1,4]-benzoxazine-3-carboxamide dihydrochloride (E17)

The title compound was prepared in 60% yield from 6-{2-[4-(2-methyl-5-quinolinyl)-1-piperazinyl]ethyl}-4H-imidazo[5,1-c][1,4]benzoxazine-3-carboxylic acid (E2) following the general procedure for amide formation described above using cyclopropylamine; MS (ES) m/z: 509.4 [MH⁺], C₃₀H₃₂N₆O₂ requires 508.62; ¹H-NMR (300 MHz, DMSO-d⁶) δ: 11.24 (bs, 1H), 9.06 (d, 1H), 8.58 (s, 1H), 8.18 (d, 1H), 8.05 (s, 1H), 8.01 (t, 1H), 7.91 (d, 1H), 7.85 (dd, 1H), 7.50 (d, 1H), 7.25 (d, 1H), 7.14 (t, 1H), 5.59 (s, 2H), 4.11-3.71 (bm, 2H), 3.50-3.45 (bd, 4H), 3.42-3.38 (bd, 4H), 3.22-3.15 (m, 2H), 2.98 (s, 3H), 2.82 (m, 1H), 0.65-0.62 (bd, 6H).

### Example 18

### N-Cyclobutyl-6-{2-[4-(2-methyl-5-quinolinyl)-1-piperazinyl]ethyl}-4H-imidazo[5,1-c][1,4]benzoxazine-3-carboxamide dihydrochloride (E18)

The title compound was prepared in 83% yield from 6-{2-[4-(2-methyl-5-quinolinyl)-1-piperazinyl]ethyl}-4H-imidazo[5,1-c][1,4]benzoxazine-3-carboxylic acid (E2) following the general procedure for amide formation described above using cyclobutylamine; MS (ES) m/z: 523.4 [MH⁺], C₃₁H₃₄N₆O₂ requires 522.65; ¹H-NMR (300 MHz, DMSO-d⁶) δ: 11.39 (bs, 1H), 9.07 (d, 1H), 8.62 (s, 1H), 8.37 (d, 1H), 8.11 (d, 1H), 8.01 (t, 1H), 7.91 (d, 1H), 7.85 (dd, 1H), 7.50 (d, 1H), 7.25 (d, 1H), 7.14 (t, 1H), 5.59 (s, 2H), 4.46-4.35 (m, 1H), 3.78-3.71 (bm, 2H), 3.56-3.49 (bd, 4H), 3.43-3.39 (bd, 4H), 3.22-3.15 (m, 2H), 2.98 (s, 3H), 2.17-2.10 (m, 4H), 1.65-1.58 (m, 2H).

### Example 19

### -(Cyclopropylmethyl)-6-{2-[4-(2-methyl-5-quinolinyl-1-piperazinyl]ethyl}-4H-imidazo[5,1-c][1,4]-benzoxazine-3-carboxamide dihydrochloride (E19)

The title compound was prepared in 60% yield from 6-{2-[4-(2-methyl-5-quinolinyl)-1-piperazinyl]ethyl}-4H-imidazo[5,1-c][1,4]benzoxazine-3-carboxylic acid (E2) following the general procedure for amide formation described above using cyclopropanemethylamine; MS (ES) m/z: 523.4 [MH⁺], C₃₁H₃₄N₆O₂ requires 522.65; ¹H-NMR (300 MHz, DMSO-d⁶) δ: 11.50 (bs, 1H), 9.05 (d, 1H), 8.60 (s, 1H), 8.21 (bs, 1H), 8.01 (t, 1H), 7.91 (d, 1H), 7.85 (dd, 1H), 7.50 (d, 1H), 7.25 (d, 1H), 7.14 (t, 1H), 5.59 (s, 2H), 3.74 (bd, 2H), 3.53-3.49 (bd, 4H), 3.42-3.38 (bd, 4H), 3.22-3.15 (m, 2H), 3.10 (t, 2H), 2.97 (s, 3H), 1.04-1.00 (m, 1H), 0.40 (d, 2H), 0.22 (d, 2H).

### Example 20

### N-Methyl-N-(1-methylethyl)-6-{2-[4-(2-methyl-5-quinolinyl)-1-piperazinyl]-ethyl}-4H-imidazo[5,1-c][1,4]-benzoxazine-3-carboxamide dihydrochloride (E20)

The title compound was prepared in 57% yield from 6-{2-[4-(2-methyl-5-quinolinyl)-1-piperazinyl]ethyl}-4H-imidazo[5,1-c][1,4]benzoxazine-3-carboxylic acid (E2) following the general procedure for amide formation described above using N-methylisopropylamine; MS (ES) m/z: 525.4 [MH⁺], C₃₁H₃₆N₆O₂ requires 524.67; ¹H-NMR (300 MHz, DMSO-d⁶) δ: 11.37 (bs, 1H), 9.05 (d, 1H), 8.61 (s, 1H), 8.08 (d, 1H), 8.01 (t, 1H), 7.90 (d, 1H), 7.85 (dd, 1H), 7.50 (d, 1H), 7.24 (dd, 1H), 7.15 (t, 1H), 5.52 (s, 2H), 4.11-4.01 (m, 1H), 3.71 (bd, 2H), 3.52-3.49 (bd, 4H), 3.42-3.38 (bd, 4H), 3.22-3.15 (m, 2H), 2.97 (s, 3H), 2.50-2.47 (bs, 3H), 1.22-1.16 (bm, 6H).

### Example 21

### 3-(1-Azetidinylcarbonyl)-6-{2-[4-(2-methyl-5-quinolinyl)-1-piperazinyl]ethyl}-4H-imidazo[5,1-c][1,4]-benzoxazine dihydrochloride (E21)

The title compound was prepared in 48% yield from 6-{2-[4-(2-methyl-5-quinolinyl)-1-piperazinyl]ethyl}-4H-imidazo[5,1-c][1,4]benzoxazine-3-carboxylic acid (E2) following the general procedure for amide formation described above using cyclobutylamine; MS (ES) m/z 509.4 [MH⁺], C₃₀H₃₂N₆O₂ requires 508.62; ¹H-NMR (300 MHz, DMSO-d⁶) δ: 10.98 (bs, 1H), 8.98 (bd, 1H), 8.57 (s, 1H), 7.98-7.96 (bd, 2H), 7.87-7.81 (bm, 2H), 7.47 (bt, 1H), 7.24 (dd, 1H), 7.15 (t, 1H), 5.59 (s, 2H), 4.54 (t, 2H), 4.00 (t, 2H), 3.75-3.34 (bm, 10H), 3.20-3.15 (bm, 2H), 2.92 (s, 3H), 1.96 (m, 2H).

### Example 22

### 6-{2-[4-(2-Methyl-5-quinolinyl)-1-piperazinyl]ethyl}-3-(1-pyrrolidinyl-carbonyl)-4H-imidazo[5,1-c][1,4]benzoxazine dihydrochloride (E22)

The title compound was prepared in 81% yield from 6-{2-[4-(2-methyl-5-quinolinyl)-1-piperazinyl]ethyl}-4H-imidazo[5,1-c][1,4]benzoxazine-3-carboxylic acid (E2) following the general procedure for amide formation described above using pyrrolidine; MS (ES) m/z: 523.4 [MH⁺], C₃₁H₃₄N₆O₂ requires 522.65; ¹H-NMR (300 MHz, DMSO-d⁶) δ: 11.56 (bs, 1H), 9.06 (d, 1H), 8.57 (s, 1H), 8.10 (d, 1H), 8.02 (t, 1H), 7.91 (d, 1H), 7.82 (dd, 1H), 7.51 (d, 1H), 7.24 ((dd, 1H), 7.15 (t, 1H), 5.59 (s, 2H), 4.01 (t, 2H), 3.72 (bd, 2H), 3.53-3.35 (vbm, 10H), 3.21-3.17 (bm, 2H), 2.98 (s, 3H), 1.94-1.75 (m, 4H).

### Example 23

### 6-{2-[4-(2-Methyl-5-quinolinyl)-1-piperazinyl]ethyl}-3-(1-Piperidinyl-carbonyl)-4H-imidazo[5,1-c][1,4]-benzoxazine dihydrochloride (E23)

The title compound was prepared in 94% yield from 6-{2-[4-(2-methyl-5-quinolinyl)-1-piperazinyl]ethyl}-4H-imidazo[5,1-c][1,4]benzoxazine-3-carboxylic acid (E2) following the general procedure for amide formation described above using piperidine; MS (ES) m/z: 537.4 [MH⁺], C₃₂H₃₆N₆O₂ requires 536.68; ¹H-NMR (300 MHz, DMSO-d⁶) δ: 11.55 (bs, 1H), 9.06 (d, 1H), 8.57 (s, 1H), 8.10 (d, 1H), 8.02 (t, 1H), 7.91 (d, 1H), 7.82 (dd, 1H), 7.51 (d, 1H), 7.24 ((dd, 1H), 7.15 (t, 1H), 5.53 (s, 2H), 4.46-4.05 (vbm, 2H), 3.72 (bd, 2H), 3.53-3.49 (bd, 4H), 3.44-3.35 (bd, 6H), 3.21-3.17 (bm, 2H), 2.98 (s, 3H), 1.63-1.62 (bd, 2H), 1.54-1.53 (bd, 4H).

### Example 24

### 6-{2-[4-(2-Methyl-5-quinolinyl)-1-piperazinyl]ethyl}-3-(4-morpholinyl carbonyl)-4H-imidazo[5,1-c][1,4]-benzoxazine dihydrochloride (E24)

The title compound was prepared in 33% yield from 6-{2-[4-(2-methyl-5-quinolinyl)-1-piperazinyl]ethyl}-4H-imidazo[5,1-c][1,4]benzoxazine-3-carboxylic acid (E2) following the general procedure for amide formation described above using morpholine; MS (ES) m/z: 537.4 [MH⁺], C₃₁H₃₄N₆O₃ requires 538.65; ¹H-NMR (300 MHz, DMSO-d⁶) δ: 11.46 (bs, 1H), 9.04 (d, 1H), 8.59 (s, 1H), 8.06 (d, 1H), 8.00 (t, 1H), 7.90-7.83 (m, 2H), 7.48 (d, 1H), 7.26 (d, 1H), 7.15 (t, 1H), 5.56 (s, 2H), 4.31 (bm, 4H), 3.93-3.56 (vbm, 6H), 3.53-3.49 (bd, 4H), 3.42-3.38 (bd, 4H), 3.22-3.17 (bm, 2H), 2.95 (s, 3H).

### Example 25

### N-Methyl-N-(methyloxy)-6-{2-[4-(2-methyl-5-quinolinyl)-1-piperazinyl]ethyl}-4H-imidazo[5,1-c][1,4]-benzoxazine-3-carboxamide (E25)

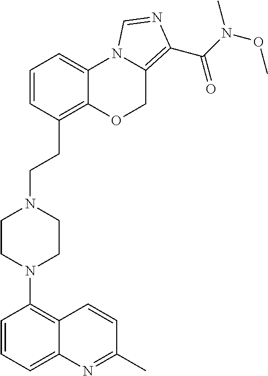

To a stirred solution of 6-{2-[4-(2-methyl-5-quinolinyl)-1-piperazinyl]ethyl}-4H-imidazo[5,1-c][1,4]benzoxazine-3-carboxylic acid (E2) (54 mg, 0115 mmol) and DIPEA (0.24 ml, 0.138 mmol) in DMF (1 ml) at room temperature was added TBTU (41 mg, 0.12 mmol). After stirring for 1 hour N,O-dimethylhydroxylamine hydrochloride amine (13.5 mg, 0.138 mmol) was added and the reaction mixture was stirred for a further 1 hour. The reaction mixture was applied to a SPE-SCX cartridge (eluting with methanol followed by 2N ammonia solution in methanol) to afford, after triturating with diethyl ether, the title compound (40 mg, 68%); MS (ES) m/z: 513.4 [MH⁺], C₂₉H₃₂N₆O₃ requires 512.61; ¹H-NMR (300 MHz, DMSO-d⁶) δ: 8.38 (d, 1H), 7.93 (s, 1H), 7.72 (d, 1H), 7.57 (t, 1H), 7.32 (d, 1H), 7.26 (bd, 1H), 7.15 (d, 1H), 7.09-6.99 (m, 2H), 5.53 (s, 2H), 3.84 (bs, 3H), 3.55 (bs, 3H), 3.15 (bs, 4H), 2.99-2.94 (bm, 2H), 2.85 (bm, 4H), 2.75-2.72 (bm, 5H).

### Example 26

### 6-{2-[4-(2-Methyl-5-quinolinyl)-1-piperazinyl]ethyl}-4H-imidazo[5,1-c][1,4]-benzoxazine-3-carbonitrile dihydrochloride (E26)

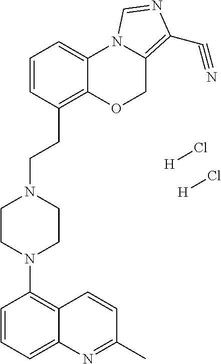

To an ice cooled stirred solution of 6-{2-[4-(2-methyl-5-quinolinyl)-1-piperazinyl]ethyl}-4H-imidazo[5,1-c][1,4]benzoxazine-3-carboxamide (free base of E14) (23 mg, 0.049 mmol) in THF (0.5 ml) and pyridine (0.008 ml, 0.098 mmol), trifluoroacetic anhydride (0.008 ml, 0.054 mmol) was added and the resulting solution was stirred at 0° C. for 1 hour. The crude reaction mixture was applied to a SPE-SCX cartridge (eluting with methanol followed by 2N ammonia solution in methanol). After evaporation of solvent from the ammonia fractions, the resulting mixture was further purified by SPE cartridge (silica gel, 2 g) eluting with DCM/methanol (98:2) to afford the free base of the title compound (7 mg, 32%) as an off-white solid. The free base was treated with HCl (2.1 eq. of 1M solution in diethyl ether) in dry methanol at 0° C. Evaporation of solvent and trituration with diethyl ether gave the title compound as a yellow solid; MS (ES) m/z 451.3 [MH⁺], C₂₇H₂₆N₆O requires 450.54; ¹H-NMR (500 MHz, DMSO-d⁶) δ: 10.93 (vbs, 1H), 8.92 (vbs, 1H), 8.79 (s, 1H), 7.95 (bs, 2H), 7.91 (d, 1H) 7.83 (bs, 1H), 7.46 (bs, 1H), 7.34 (d, 1H), 7.24 (t, 1H), 5.56 (s, 2H), 4-3 (vbm, 12H), 2.91 (bs, 3H).

### Example 27

### 2-Methyl-6-{2-[4-(2-methyl-5-quinolinyl)-1-piperazinyl]ethyl}-4H-imidazol-[2,1-c][1,4]-benzoxazine dihydrochloride (E27)

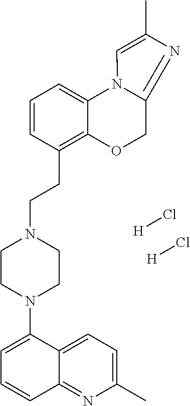

A mixture of (2-methyl-4H-imidazo[2,1-c][1,4]benzoxazin-6-yl)acetaldehyde (D28) (42 mg, 0.18 mmol) and 2-methyl-5-piperazin-1-yl-quinoline (63 mg, 0.28 mmol, 1.5 eq) in dry 1,2-dichloroethane (4 ml) was stirred at room temperature for 30 minutes before sodium triacetoxyborohydride (58 mg, 0.28 mmol, 1.5 eq) was added. The resulting mixture was stirred for 3 hours, quenched with NaHCO₃ (10 ml of a saturated aq. solution) and extracted with DCM (3×10 ml). The combined organic layers were dried (Na₂SO₄) and concentrated in vacuo. The crude product was purified by SPE-SI cartridge eluting with a gradient of DCM/methanol (99/1 to 97/3) to afford the corresponding free base of the title compound E27 as a white solid (21 mg, 26%). To a solution of the free base (21 mg, 0.05 mmol) in 3:1 MeOH/DCM (4 ml), HCl (84 μl of a 1.25 M solution in methanol, 0.105 mmol, 2.2 eq) was slowly added at 0° C. After 2 hours stirring at room temperature evaporation of the volatiles gave the title compound E27 as a yellow solid (23 mg); MS (ES) m/z: 440.30 [MH⁺], C27H29N5O requires 439.57; ¹H-NMR (500 MHz, DMSO-d6) δ: 10.47 (bs, 1H), 8.55 (bs, 1H), 7.75 (bs, 2H), 7.68 (s, 1H), 7.60 (m, 2H), 7.29 (bs, 1H), 7.2 (m, 2H), 5.36 (s, 2H), 3.75-3.00 (vbm, 12H), 2.74 (s, 3H), 2.21 (s, 3H).

### Example 28

### 1,2-Dimethyl-6-{2-[4-(2-methyl-5-quinolinyl)-1-piperazinyl]ethyl}-4H-imidazo[2,1-c][1,4]-benzoxazine dihydrochloride (E28)

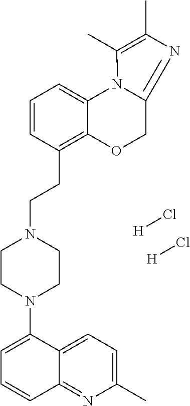

Ammonium acetate (163 mg, 2.12 mmol, 20 eq) was added to a solution of 4-(1-methyl-2-oxopropyl)-8-{2-[4-(2-methyl-5-quinolinyl)-1-piperazinyl]ethyl}-2H-1,4-benzoxazin-3(4H)-one (D31) (50 mg, 0.106 mmol) in glacial acetic acid (1 ml). The mixture was irradiated in a microwave reactor (PersonalChemistry Emrys™ Optimiser, 300W, 150° C., 10 min), then diluted with ethyl acetate (10 ml), poured into ice-water (10 ml) and basified with an aqueous solution of ammonium hydroxide (3 ml). The mixture was extracted with ethyl acetate (3×30 ml). The combined organics were dried (Na₂SO₄) and the solvent was removed under reduced pressure. The crude material was purified by flash chromatography on silica gel eluting with a gradient of methanol in DCM (1 to 3%) to afford the free base of the title compound (44 mg, 92%). The free base (44 mg) was dissolved in 3:1 MeOH/DCM (4 ml) and treated with HCl (2.2 eq. of 1.25 M solution in methanol) at 0° C. The resulting mixture was stirred at room temperature for 2 h. Evaporation of the volatiles gave the title compound (44 mg) as a yellow solid; MS (ES) m/z: 454.40 [MH⁺], C28H31N5O requires 453.59; ¹H-NMR (500 MHz, DMSO-d6) δ: 10.65 (vbs, 1H), 8.64 (vbs, 1H), 7.79 (bs, 2H), 7.68 (d, 1H), 7.61 (bs, 1H), 7.32 (bs, 1H), 7.27 (d, 1H), 7.20 (t, 1H), 5.27 (s, 2H), 3.75 (d, 2H), 3.70-3.00 (vbm, 10H), 2.77 (s, 3H), 2.48 (s, 3H), 2.18 (s, 3H).

### Example 29

### 6-{2-[4-(2-Methyl-5-quinolinyl)-1-piperazinyl]ethyl}-2-(trifluoromethyl)-4H-imidazo[2,1-c][1,4]-benzoxazine dihydrochloride (E29)

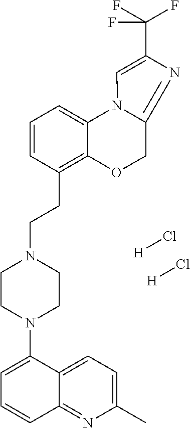

The title compound was prepared in 58% yield as a yellow solid in a similar fashion to Example 28 from 8-{2-[4-(2-methyl-5-quinolinyl)-1-piperazinyl]ethyl}-4-(3,3,3-trifluoro-2-oxopropyl)-2H-1,4-benzoxazin-3(4H)-one (D36) (79 mg, 0.149 mmol) and ammonium acetate (230 mg, 2.98 mmol, 20 eq.) in acetic acid (2 ml). The crude product was purified by flash chromatography on silica gel eluting with a gradient of methanol in DCM (1 to 2%) to afford the free base of the title compound (42 mg, 58%). This was treated with HCl (2.2 eq of 1.25M solution in MeOH) to afford the title compound. MS (ES) m/z: 494.30 [MH⁺], C27H26F3N5O requires 493.54; ¹H-NMR (500 MHz, DMSO-d6) δ: 10.52 (vbs, 1H), 8.74 (s, 1H), 8.63 (vbs, 1H), 7.79 (m, 3H), 7.61 (bs, 1H), 7.30 (m, 2H), 7.22 (t, 1H), 5.47 (s, 2H), 3.75 (d, 2H), 3.70-3.10 (vbm, 10H), 2.77 (s, 3H).

### Example 30

### 3-Methyl-6-{2-[4-(2-methyl-5-quinolinyl)-1-piperazinyl]ethyl}-4H-[1,2,3]triazolo[5,1-c][1,4]-benzoxazine dihydrochloride (E30)

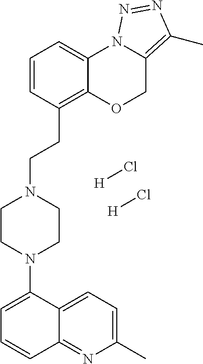

The title compound was prepared in 81% yield following the general reductive amination procedure of Example 1 starting from (3-methyl-4H-[1,2,3]triazolo[5,1-c][1,4]benzoxazin-6-yl)acetaldehyde (D41) (60 mg, 0.262 mmol). The crude product was purified by flash chromatography on silica gel eluting with a gradient of methanol in DCM (1 to 3%) to afford the free base of the title compound (93 mg, 81%). Treatment with HCl (2.2 eq of 1.25M solution in MeOH) in 4:1 methanol/DCM (5 ml) at 0° C. gave the title compound as a solid; MS (ES) m/z: 441.20 [MH⁺], C26H28N6O requires 440.55; ¹H-NMR (500 MHz, DMSO-d6) δ: 10.51 (vbs, 1H), 8.62 (vbs, 1H), 7.93 (d, 1H), 7.78 (bs, 2H), 7.60 (vbs, 1H), 7.37 (d, 1H), 7.32 (bs, 1H), 7.24 (t, 1H), 5.58 (s, 2H), 3.75-3.10 (vbm, 12H), 2.76 (s, 3H), 2.34 (s, 3H).

### Example 31

### 6-{2-[4-(2-Methyl-5-quinolinyl)-1-piperazinyl]ethyl}-4H-[1,2,4]-oxadiazolo[3,4-c][1,4]-benzoxazin-1-one dihydrochloride (E31)

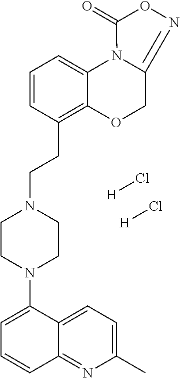

The title compound was prepared in 81% yield (46 mg) following the general reductive amination procedure of Example 1 starting from (1-oxo-4H-[1,2,4]oxadiazolo[3,4-c][1,4]benzoxazin-6-yl)acetaldehyde (D44) (30 mg, 0.129 mmol). The crude product was purified by flash chromatography on silica gel eluting with a gradient of methanol in DCM (1% to 2%) to afford the free base of the title compound (46 mg, 81%). Treatment with HCl (2.2 eq of 1.25M solution in MeOH) in 5:1 methanol/DCM (5 ml) at 0° C. gave the title compound as a solid; MS (ES) m/z: 444.20 [MH⁺], C25H25N5O3 requires 443.51; ¹H-NMR (500 MHz, DMSO-d6) δ: 10.50 (vbs, 1H), 8.51 (vbs, 1H), 7.81 (d, 1H), 7.68 (bs, 2H), 7.50 (vbs, 1H), 7.21-7.10 (m, 3H), 5.32 (s, 2H), 3.66-3.05 (bm, 12H), 2.67 (s, 3H).

### Example 32

### 3-methyl-6-{2-[4-(2-methyl-5-quinolinyl)-1-piperidinyl]ethyl}-4H-[1,2,3]triazolo[5,1-c][1,4]-benzoxazine dihydrochloride (32)

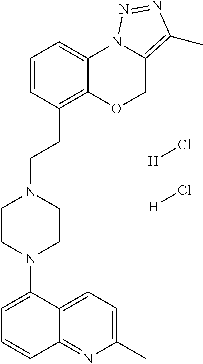

The title compound was prepared in 45% yield (25 mg) following the general reductive amination procedure of Example 1 starting from (3-methyl-4H-[1,2,3]triazolo[5,1-c][1,4]benzoxazin-6-yl)acetaldehyde (D41) (25 mg, 0.109 mmol) and 2-methyl-5-(4-piperidinyl)quinoline (30 mg, 0.130 mmol). The crude product was purified by flash chromatography on silica gel eluting with a gradient of methanol in DCM (1% to 3%) to afford the free base of the title compound (22 mg, 45%). Treatment with HCl (2.2 eq of 1.25M solution in MeOH) in 4:1 methanol/DCM (5 ml) at 0° C. gave, the title compound as a solid; ¹H-NMR (500 MHz, DMSO-d6) δ: 10.33 (vbs, 1H), 9.01 (vbs, 1H), 8.06 (bs, 1H), 7.93 (m, 2H), 7.82 (bs, 1H), 7.62 (bs, 1H), 7.23 (t, 1H), 5.58 (s, 2H), 3.39-3.10 (vbm, 9H), 2.86 (bs, 3H), 2.34 (s, 3H), 2.20 (m, 4H).

### Example 33

### 1-Methyl-6-(2-{4-[2-(trifluoromethyl)-5-quinolinyl]-1-piperazinyl}ethyl)-4H-[1,2,4]triazolo[3,4-c][1,4]-benzoxazine hydrochloride (E33)

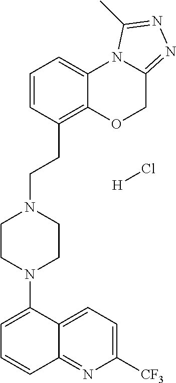

To a mixture of 5-(1-piperazinyl)-2-(trifluoromethyl)quinoline (D47) (39 mg, 0.137 mmol) and (1-methyl-4H-[1,2,4]triazolo[3,4-c][1,4]benzoxazin-6-yl)acetaldehyde (D25) (21 mg, 0.092 mmol) in DCM (5 ml) was added sodium triacetoxyborohydride (42 mg, 0.84 mmol). After stirring the mixture at room temperature overnight, water (10 ml) was added and the mixture extracted with DCM (3×15 ml). The combined organics were dried (MgSO₄) and evaporated in vacuo. The residue was purified with a reversed phase preparative mass directed HPLC system to give the free base of the title compound as a colourless oil (14 mg, 30%). The free base was dissolved in dry DCM/diethyl ether and treated with HCl (1.1 eq. of 1M solution in diethyl ether).

Evaporation of solvent and trituration in ethyl ether gave the title compound. MS (ES; m/z): 495 [MH⁺], C26H26F3N6O requires 494.52; ¹H-NMR (400 MHz, DMSO-d₆) δ: 10.85 (bs, 1H), 8.86 (d, 1H), 7.99 (d, 1H), 7.94 (d, 1H), 7.9 (d, 1H), 7.73 (dd, 1H), 7.48 (d, 1H), 7.35 (d, 1H), 7.25 (t, 1H), 5.47 (s, 2H), 3.77 (bd, 2H), 3.6-3.45 (m, 4H), 3.32 (t, 2H), 3.47 (m, 2H), 3.32 (m, 2H); ¹⁹F-NMR (400 MHz, DMSO-d₆) δ: −66.04.

### Example 34

### N,N-Dimethyl-6-{2-[4-(2-methyl-5-quinolinyl-1-piperazinyl]ethyl}-4H-imidazo[5,1-c][1,4]-benzoxazine-3-carboxamide dihydrochloride (E34)

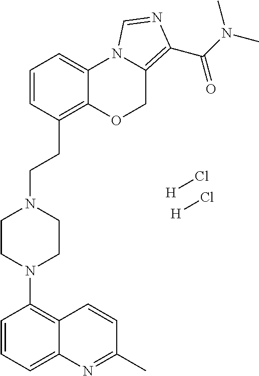

The title compound was prepared in 60% yield following the general procedure described for Example 1 starting from N,N-dimethyl-6-(2-oxoethyl)-4H-imidazo[5,1-c][1,4]benzoxazine-3-carboxamide (D9) (20 mg, 0.07 mmol) and 2-methyl-5-piperazin-1-yl-quinoline (19 mg, 0.084 mmol); MS: (ES) m/z: 497.3 [MH⁺], C29H32N6O2 requires 496.6; ¹H-NMR (500 MHz, DMSO-d⁶) δ: 11 (vbs, 1H), 8.77 (vbs, 1H), 8.57 (s, 1H), 7.9 (bs+d, 3H), 7.69 (bs, 1H), 7.36 (bs, 1H), 7.25 (d, 1H), 7.15 (t, 1H), 5.54 (s, 2H), 3.7-3.2 (vbm, 12H), 2.96-2.82 (2s, 6H), 2.5 (m, 3H).

### Example 35

### 1-(6-{2-[4-(2-Methyl-5-quinolinyl)-1-piperazinyl]ethyl}-4H-imidazo[5,1-c][1,4]-benzoxazin-3-yl)ethanone hydrochloride (E35)

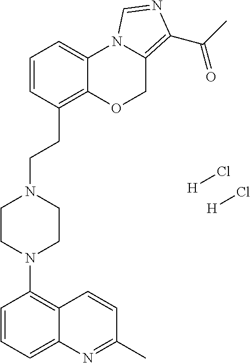

To an ice cooled stirred solution of N-methyl-N-(methyloxy)-6-{2-[4-(2-methyl-5-quinolinyl)-1-piperazinyl]ethyl}-4H-imidazo[5,1-c][1,4]benzoxazine-3-carboxamide (E25) (40 mg, 0.078 mmol) in THF (1 ml), methyl magnesium bromide (0.03 ml of a 3M solution in diethyl ether, 0.09 mmol) was added and the resulting solution was stirred for 1 hour at 0° C. The reaction mixture was poured into cold aqueous hydrochloric acid (2 ml of 2.5 M solution), then treated with NaHCO₃ (15 ml) and extracted with DCM (3×15 ml). The combined organic layers were dried (Na₂SO₄) and evaporated in vacuo to give a brown oil that was purified by SPE cartridge (silica gel, 2 g) eluting with DCM/methanol (98:2) to afford the free base of the title compound (22 mg, 60%) as a white solid. The free base was treated with HCl (2.1 eq. of 1 M solution in diethyl ether) in dry methanol at 0° C. Evaporation of solvent and trituration with diethyl ether gave the title compound as a yellow solid; MS; (ES) m/z: 468.4 [MH⁺], C₂₈H₂₉N₅O₃ requires 467.57; ¹H-NMR (500 MHz, DMSO-d⁶) δ: 11.41 (bs, 1H), 9.08 (d, 1H), 8.66 (d, 1H), 8.2-7.08 (m, 4H) 7.52 (dd, 1H), 7.3 (dd, 1H), 7.19 (t, 1H), 5.62 (s, 2H), 4.2-3.2 (vbm, 12H), 2.99 (s, 3H), 2.5 (s, 3H).

### Example 36

### N,N-Dimethyl-6-{3-[4-(2-methyl-5-quinolinyl)-1-piperazinyl]propyl}-4H-imidazo[5,1-c][1,4]-benzoxazine-3-carboxamide dihydrochloride (E36)

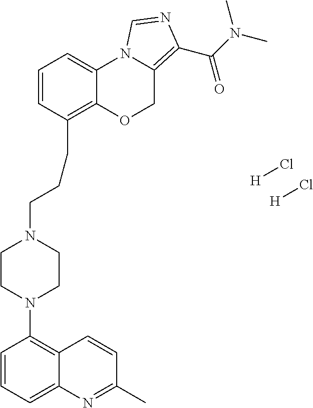

To a stirred solution of 6-{3-[4-(2-methyl-5-quinolinyl)-1-piperazinyl]propyl}-4H-imidazo[5,1-c][1,4]benzoxazine-3-carboxylic acid (E6) (32 mg, 0.07 mmol) and DIPEA (23 μL, 0.13 mmol) in DMF (1 ml) was added TBTU (24 mg, 0.075 mmol). The reaction mixture was stirred at room temperature for 1 hour and then dimethylamine in THF (37 μL of a 2M solution, 0.075 mmol) was added and the solution stirred for 1 hour. The crude solution was purified by SPE-SCX cartridge (eluting with methanol followed by 2N ammonia solution in methanol) and then triturated with diethyl ether to afford the corresponding free base of the title compound as a solid (20 mg, 59%). The free base was dissolved in dry methanol (1 ml) and HCL (68 μl of a 1.25M solution in methanol, 0.09 mmol) was slowly added at 0° C. The resulting suspension was stirred at 0° C. for 4 h. Evaporation of the volatiles gave the title compound as a yellow solid (22 mg); MS (ES) m/z: 511.4 [MH⁺], C30H34N6O2 requires 510.6; ¹H-NMR (500 MHz, DMSO-d⁶) δ: 10.4 (bs, 1H), 8.6 (s, 1H), 8.4 (d, 1H), 7.8 (d, 1H), 7.6-7.7 (m, 2H), 7.4 (bs, 1H), 7.0-7.2 (m, 3H), 5.5 (s, 2H), 3.1-3.6 (bm, 10H), 2.9 (s, 3H), 2.5-2.8 (bm, 8H), 2.1 (bs, 2H).

### Example 37

### Ethyl 6-{2-[4-(2-methyl-5-quinolinyl)-1-piperidinyl]ethyl}-4H-imidazo[5,1-c][1,4]-benzoxazine-3-carboxylate (E37)

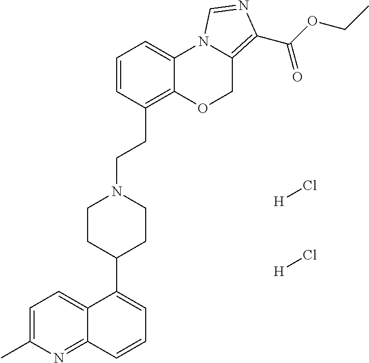

A mixture of ethyl 6-(2-oxoethyl)-4H-imidazo[5,1-c][1,4]benzoxazine-3-carboxylate (D6) (62 mg, 0.27 mmol) and 2-methyl-5-piperidyne-1-yl-quinoline (70 mg, 0.31 mmol) in dry 1,2-dichloroethane (10 ml) was stirred at room temperature under nitrogen for 40 min. Sodium triacetoxyborohydride (65 mg, 0.31 mmol) was then added and the resulting reaction mixture was stirred for 3 hours, quenched with a saturated aqueous solution of NaHCO₃ (10 ml) and extracted with DCM (3×10 ml). The organic layers were combined, dried (Na₂SO₄) and concentrated in vacuo. The crude product was purified by column chromatography on silica, eluting with 1% methanol in DCM to afford the title compound as a pale yellow solid (68 mg, 50%); MS (ES) m/z: 497.4 [MH⁺], C30H32N4O3 requires 496.5; ¹H-NMR (500 MHz, CDCl₃) δ: 8.30 (d, 1H), 7.98 (s, 1H), 7.63 (d, 1H), 7.40 (d, 1H), 7.34 (dd, 1H), 7.28 (d, 1H), 7.15 (dd, 1H), 7.03 (t, 1H), 5.53 (s, 2H), 4.39 (q, 2H), 3.23 (m, 3H), 2.92 (dd, 2H), 2.73 (s, 3H), 2.65 (dd, 2H), 2.29 (m, 2H), 1.95 (m, 4H), 1.41 (t, 3H).

### Example 38

### N-Methyl-6-{2-[4-(2-methyl-5-quinolinyl)-1-piperidinyl]ethyl}-4H-imidazo[5,1-c][1,4]-benzoxazine-3-carboxamide dihydrochloride (E38)

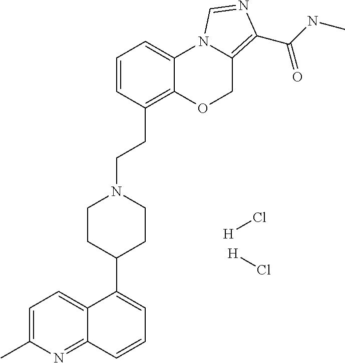

A solution of trimethylaluminium (2.0M in hexanes, 150 μl, 0.3 mmol) and methylamine (2.0M in THF, 150 μl, 0.3 mmol) in DCM (1 ml) was stirred at room temperature for 15 min. Ethyl 6-{2-[4-(2-methyl-5-quinolinyl)-1-piperidinyl]ethyl}-4H-imidazo[5,1-c][1,4]benzoxazine-3-carboxylate (E37) (30 mg, 0.061 mmol) was added and stirring was continued for another 3 hours at 40° C. After reaction was completed, water was added dropwise until no more gas evolved and the final volume added reached ca. 3 ml. Aqueous 1M NaOH (5 ml) was added and the aqueous solution was extracted with DCM (3×20 ml). The combined organic phases were dried (Na₂SO₄) and evaporated and the residue purified by Si-gel chromatography eluting with DCM/MeOH (98:2 to 96:4) affording the title compound as a pale yellow solid (22 mg, 76%). The free base was dissolved in dry methanol (3 ml), 2.1 eq. of hydrochloric acid (1.25 M solution in MeOH) was slowly added and the resulting suspension was stirred for 2 hours. Evaporation of solvent and trituration in diethyl ether gave the desired dihydrochloride salt (26 mg) as yellow solid; MS (ES) m/z: 481.2 [MH⁺], C29H31N5O2 requires 480.5; ¹H-NMR (500 MHz, DMSO) δ: 10.67 (bs, 1H), 9.24 (bs, 1H), 8.60 (s, 1H), 8.21 (dd, 1H), 8.17 (q, 1H), 8.06 (t, 1H), 7.96 (d, 1H), 7.85 (dd, 1H), 7.71 (m, 1H), 7.28 (d, 1H), 7.17 (t, 1H), 5.62 (s, 2H), 3.84 (m, 1H), 3.7-3.2 (m, 4H), 3.2 (d, 2H), 2.95 (s, 3H), 2.77 (d, 3H), 2.25 (q, 2H), 2.11 (d, 2H).

### Example 39

### 6-{2-[4-(2-Methyl-5-quinolinyl)-1-piperidinyl]ethyl}-3-(4-morpholinylcarbonyl)-4H-imidazo[5,1-c][1,4]-benzoxazine dihydrochloride (E39)

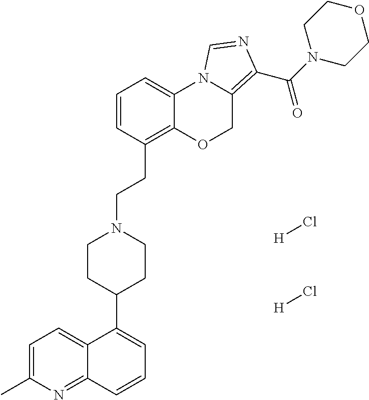

A solution of trimethylaluminium (2.0M in hexanes, 150 μl, 0.3 mmol) and morpholine (30 μl, 0.3 mmol) in DCM (1 ml) was stirred at room temperature for 15 min. Ethyl 6-{2-[4-(2-methyl-5-quinolinyl)-1-piperidinyl]ethyl}-4H-imidazo[5,1-c][1,4]benzoxazine-3-carboxylate (E37) (30 mg, 0.061 mmol) was added and stirring was continued for another 3 hours at 40° C. After reaction was completed, water was added dropwise until no more gas evolved and the final volume reached ca. 3 ml. Aqueous 1M NaOH (5 ml) was added and the aqueous solution was extracted with DCM (3×15 ml). The combined organic phases were dried (Na₂SO₄) and evaporated and the residue was purified by Si-gel chromatography eluting with DCM/MeOH (98:2 to 96:4) to afford the title compound as a pale yellow solid (24 mg, 75%). The free base was dissolved in dry MeOH (3 ml), 2.1 eq. of hydrochloric acid (1.25 M solution in methanol) was slowly added and the resulting suspension was stirred 2 hours. Evaporation of solvent and trituration in diethyl ether gave the desired dihydrochloride salt (28 mg) as yellow solid; MS (ES) m/z: 538 [MH⁺], C32H37N5O3 requires 537.66; ¹H-NMR (500 MHz, DMSO) δ: 10.7 (bs, 1H), 9.2 (bs, 1H), 8.60 (s, 1H), 8.2 (dd, 1H), 8.0 (t, 1H), 7.96 (d, 1H), 7.85 (dd, 1H), 7.71 (m, 1H), 7.28 (d, 1H), 7.17 (t, 1H), 5.52 (s, 2H), 4.3 (m, 2H), 3.84 (m, 1H), 3.7-3.2 (m, 4H), 3.2 (d, 2H), 2.95 (s, 3H), 2.25 (q, 2H), 2.11 (d, 2H).

### Example 40

### 6-(2-{4-[2-(Trifluoromethyl)-5-quinolinyl]-1-piperazinyl}ethyl)-4H-imidazo[5,1-c][1,4]-benzoxazine-3-carboxamide (E40)

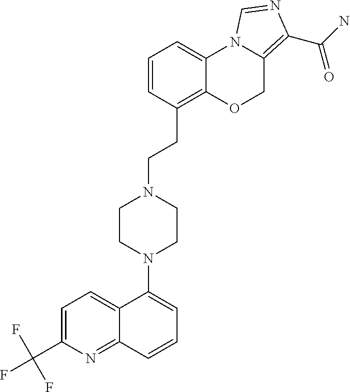

A solution of 6-(2-{4-[2-(trifluoromethyl)-5-quinolinyl]-1-piperazinyl}ethyl)-4H-imidazo[5,1-c][1,4]benzoxazine-3-carboxylic acid ethyl ester (E56) (32 mg, 0.058 mmol) and a catalytic amount of potassium cyanide in a 7M solution of NH3 in MeOH (20 ml) was stirred at room temperature for 3 days. The reaction was filtered and the filtrate was dried under high vacuum to afford the title compound (18 mg, 59%) as a solid; MS (ES; m/z): 523[MH+], C27H25F3N6O2 requires 522.53; ¹H NMR (400 MHz, DMSO-d6) δ: 8.79 (d, 1H), 8.54 (s, 1H), 7.95 (d, 1H), 7.84 (m, 2H), 7.76 (d, 1H), 7.51 (dd, 1H), 7.37 (t, 1H), 7.33 (bs, 1H), 7.24 (dd, 1H), 7.1 (t, 1H), 5.54 (s, 2H), 3.31 (bs, 4H), 2.89 (t, 2H), 2.79 (bs, 4H), 2.7 (m, 2H); ¹⁹F NMR (400 MHz, DMSO-d6) δ: −66.05.

### Example 41

### N-(Methyloxy)-6-{2-[4-(2-methyl-5-quinolinyl)-1-piperazinyl]ethyl}-4H-imidazo[5,1-c][1,4]-benzoxazine-3-carboxamide dihydrochloride (E41)

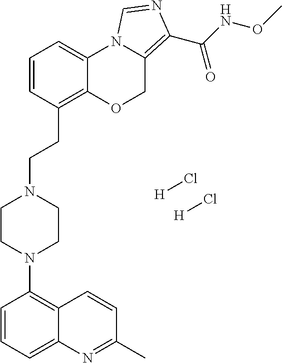

A solution of trimethylaluminium (2.0M in hexanes, 2.4 ml, 4.82 mmol) and methoxylamine hydrochloride (402 mg, 4.82 mmol) in DCM (8 ml) was stirred at room temperature for 30 minutes. Ethyl 6-{2-[4-(2-methyl-5-quinolinyl)-1-piperazinyl]ethyl}-4H-imidazo[5,1-c][1,4]benzoxazine-3-carboxylate (E1) (400 mg, 0.803 mmol) was added and stirring was continued for another 4 hours at 54° C. After reaction was completed, water was added dropwise until no more gas evolved and the final volume added reached ca. 20 ml. The aqueous solution was extracted with DCM (3×30 ml). In case the organic and aqueous phase did not separate well, aqueous 1M NaOH was added. The combined organic phases were dried (Na2SO4) and evaporated. The residue was triturated with Et2O to afford the free base of the desired material (284 mg, 71%). The free base (25 mg) was dissolved in dry MeOH (1 ml) and 2.1 eq. of hydrochloric acid (1 M solution in ethyl ether) was slowly added at 0° C. The resulting suspension was stirred 2 hours at 0° C. Evaporation of solvent and trituration in ethyl ether gave the title compound as a yellow solid (31 mg); MS (ES) m/z: 499.6 [MH⁺]. C₂₈H₃₀N₆O₃ requires 498.6; ¹H-NMR (500 MHz, DMSO-d⁶) δ: 11.62 (bs, 1H), 11.37 (bs, 1H), 9.06 (d, 1H), 8.62 (s, 1H), 8.09 (d, 1H), 8.03 (t, 1H), 7.92 (d, 1H), 7.86 (dd, 1H), 7.52 (d, 1H), 7.28 (dd, 1H), 7.18 (t, 1H), 5.62 (s, 2H), 4-3.3 (vbm, 10H), 3.69 (s, 3H), 3.22 (dd, 2H), 2.98 (s, 3H).

### Example 42

### Ethyl 6-{2-[(2R)-2-methyl-4-(2-methyl-5-quinolinyl-1-piperazinyl]ethyl}-4H-imidazo[5,1-c][1,4]-benzoxazine-3-carboxylate (E42)

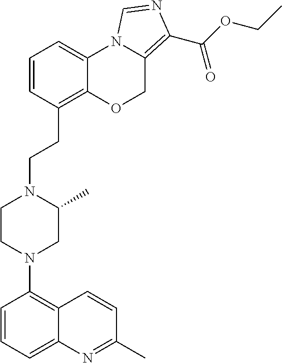

To a solution of 2-methyl-5-[(3R)-3-methyl-1-piperazinyl]quinoline (WO2004/046124) (200 mg, 0.84 mmol) and ethyl 6-(2-oxoethyl)-4H-imidazo[5,1-c][1,4]benzoxazine-3-carboxylate (D6) (200 mg, 0.69 mmol) in DCM (10 ml) was added sodium triacetoxyborohydride (178 mg, 0.84 mmol). After stirring the mixture at room temperature overnight, water (15 ml) was added and the product extracted with DCM (3×20 ml). The organic layer was dried (MgSO₄), filtered and evaporated in vacuo. The residue was purified by column chromatography on silica eluting with a mixture DCM/MeOH (98:2) to give the title compound as white foam (212 mg, 60%); MS (ES) m/z: 512.2 [MH⁺], C₃₀H₃₃N5O₃ requires 511.6; ¹H-NMR (500 MHz, DMSO-d⁶) δ: 8.44 (d, 1H), 8.03 (s, 1H), 7.76 (d, 1H), 7.62 (t, 1H), 7.4 (d, 1H), 7.3 (m, 1H), 7.2 (d, 1H), 7.1 (m, 2H), 5.59 (s, 2H), 4.45 (quart., 2H), 3.-2.7 (vbm, 11H), 2.77 (s, 3H), 1.46 (t, 3H), 1.2 (m, 3H).

### Example 43

### Ethyl 6-{2-[4-(7-fluoro-2-methyl-5-quinolinyl)-1-piperazinyl]ethyl}-4H-imidazo[5,1-c][1,4]-benzoxazine-3-carboxylate (E43)

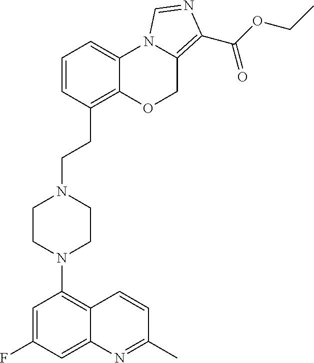

The title compound was prepared in 62% yield following the procedure of Example 42 using 7-fluoro-2-methyl-5-(1-piperazinyl)quinoline (see WO2004/046124) (205 mg, 0.84 mmol); MS (ES) m/z: 516.6 [MH⁺]. C₂₉H₃₀FN5O₃ requires 515.59; ¹H-NMR (500 MHz, DMSO-d⁶) δ: 8.33 (d, 1H), 8.03 (s, 1H), 7.39 (dd, 1H), 7.36 (dd, 1H), 7.23 (d, 1H), 7.2 (dd, 1H), 7.08 (t, 1H), 6.87 (dd, 1H), 5.58 (s, 2H), 4.44 (quart., 2H), 3.18 (bs, 4H), 2.99 (dd, 2H), 2.86 (bs, 4H), 2.76 (m, 5H), 1.46 (t, 3H).

### Example 44

### Ethyl 6-{2-[4-(2-methyl-5-quinazolinyl)-1-piperazinyl]ethyl}-4H-imidazo[5,1-c][1,4]-benzoxazine-3-carboxylate (E44)

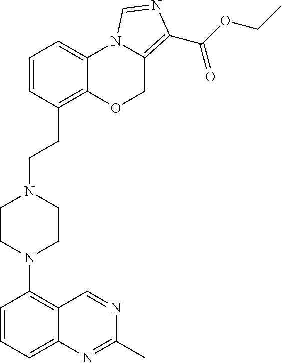

The title compound was prepared with 74% yield following the procedure of Example 42 using 2-methyl-5-(1-piperazinyl)quinazoline (see WO2004/046124) (228 mg, 0.84 mmol); MS (ES) m/z: 499.1 [MH⁺], C₂₉H₃₀FN5O₃ requires 498.58; ¹H-NMR (500 MHz, DMSO-d⁶) δ: 9.61 (s, 1H), 8.03 (s, 1H), 7.79 (t, 1H), 7.62 (d, 1H), 7.4 (dd, 1H), 7.2 (dd, 1H), 7.1 (m, 2H), 5.58 (s, 2H), 4.44 (quart., 2H), 3.25 (bs, 4H), 3 (dd, 2H), 2.91 (s, 3H); 2.87 (bs, 4H), 2.76 (dd, 2H), 1.46 (t, 3H).

### Example 45

### 6-{2-[(2R)-2-Methyl-4-(2-methyl-5-quinolinyl)-1-piperazinyl]ethyl}-4H-imidazo[5,1-c][1,4]-benzoxazine-3-carboxylic acid (E45)

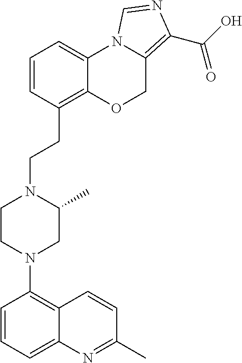

To a solution of ethyl 6-{2-[(2R)-2-methyl-4-(2-methyl-5-quinolinyl)-1-piperazinyl]ethyl}-4H-imidazo[5,1-c][1,4]benzoxazine-3-carboxylate (E42) (212 mg, 0.45 mmol) in MeOH (4 ml) was added NaOH (4 ml of a 10% aqueous solution) and the resulting white suspension was heated for 5 min. under microwave irradiation at 120° C. The resulting pale yellow solid was filtered off, suspended in water and the solution was neutralised (pH=7) with acetic acid. The precipitate was filtered-off and washed with diethyl ether (3×20 ml) to give the title compound (164 mg, 81%) as an off-white solid; MS; (ES) m/z: 484.6 [MH⁺]; C₂₈H₂₉N₅O₃ requires 482.57; ¹H-NMR (300 MHz, DMSO-d⁶) δ: 8.53 (s, 1H), 8.37 (d, 1H), 7.74 (dd, 1H), 7.58 (m, 2H), 7.4 (d, 1H), 7.21 (dd, 1H), 7.08 (m, 2H), 5.52 (s, 2H), 3.2-2.5 (m, 11H), 2.63 (s, 3H), 1.10 (d, 3H).

### Example 46

### Ethyl 6-{2-[4-(7-fluoro-2-methyl-5-quinolinyl)-1-piperazinyl]ethyl}-4H-imidazo[5,1-c][1,4]-benzoxazine-3-carboxylate (E46)

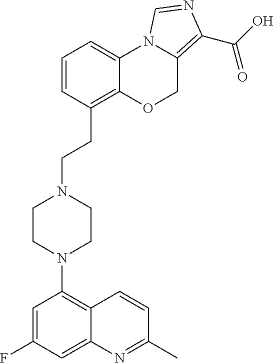

The title compound was prepared following the procedure of Example 45 starting from ethyl 6-{2-[4-(7-fluoro-2-methyl-5-quinolinyl)-1-piperazinyl]ethyl}-4H-imidazo[5,1-c][1,4]benzoxazine-3-carboxylate (E43) (223 mg, 0.43 mmol); MS (ES) m/z: 488.6 [MH⁺], C₂₇H₂₆FN₅O₃ requires 487.53; ¹H-NMR (300 MHz, DMSO-d⁶) δ: 7.63 (d, 1H), 7.42 (s, 1H), 6.78 (d, 1H), 6.57 (d, 1H), 6.5-6.0 (m, 4H), 4.74 (s, 2H), 2.49 (s, 3H), 2.37 (m, 4H), 2.2 (dd, 2H), 2.12 (m, 4H), 2.0 (dd, 2H).

### Example 47

### 6-{2-[4-(2-Methyl-5-quinazolinyl)-1-piperazinyl]ethyl}-4H-imidazo[5,1-c][1,4]-benzoxazine-3-carboxylic acid (E47)

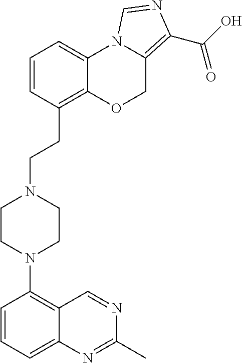

The title compound was prepared following the procedure of Example 45 starting from ethyl 6-{2-[4-(2-methyl-5-quinazolinyl)-1-piperazinyl]ethyl}-4H-imidazo[5,1-c][1,4]benzoxazine-3-carboxylate (E44) (255 mg, 0.512 mmol); MS (ES) m/z: 488.6 [MH⁺], C₂₇H₂₆FN₅O₃ requires 487.53; ¹H-NMR (300 MHz, DMSO-d⁶) δ: 8.8 (s, 1H), 7.47 (s, 1H), 7.06 (t, 1H), 6.77 (d, 2H), 6.46 (d, 1H), 6.41 (d, 1H), 6.25 (t, 1H), 4.7 (s, 2H), 2.55 (m, 4H), 2.49 (s, 3H), 249 (m, 4H), 2.36 (dd, 2H), 2.30 (dd, 2H).

### Examples 48-53

### General Procedure for Amide Formation

To a suspension of the carboxylic add (0.1 mmol) in DMF (1.5 ml) and DIPEA (0.11 mmol) was added TBTU (0.11 mmol) and the mixture stirred at room temperature for 1.5 hours. The appropriate amine was added (0.11 mmol) and the reaction stirred at room temperature overnight. The crude reaction mixture was loaded on SCX cartridge (5 g) and the ammonia fractions evaporated in vacuo to afford the desired amide in pure form. The free base was dissolved in dry MeOH, 2.1 eq. of hydrochloric acid (1 M solution in diethyl ether) was slowly added at 0° C. The resulting suspension was stirred 2 hours at 0° C. Filtration or evaporation of solvent and trituration with diethyl ether gave the desired dihydrochloride salt as a yellow solid.

### Example 48

### N-Methyl-6-{2-[(2R)-2-methyl-4-(2-methyl-5-quinolinyl)-1-piperazinyl]ethyl}-4H-imidazo[5,1-c][1,4]-benzoxazine-3-carboxamide dihydrochloride (E48)

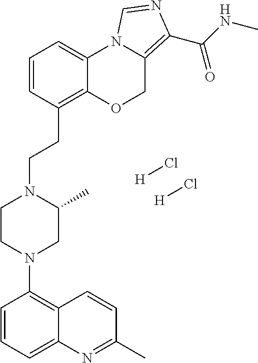

The title compound was prepared in 52% yield according to the general amide formation procedure starting from 6-{2-[(2R)-2-methyl-4-(2-methyl-5-quinolinyl)-1-piperazinyl]ethyl}-4H-imidazo[5,1-c][1,4]benzoxazine-3-carboxylic acid (E45) (48.3 mg, 0.1 mmole) and methylamine (0.055 ml of 2M sol. in THF, 0.11 mmol); MS (ES) m/z: 497.7 [MH⁺], C₂₉H₃₂N₆O₂ requires 496.61; ¹H-NMR (500 MHz, DMSO-d⁶) δ: 11.51 (bs, 1H), 9.14 (d, 1H), 8.60 (s, 1H), 8.16 (m, 1H),), 8.11 (d, 1H), 8.03 (t, 1H), 7.92 (d, 1H), 78.4 (d, 1H), 7.5 (d, 1H), 7.3 (d, 1H), 7.15 (t, 1H), 5.62 (s, 2H), 4.0-2.9 (m., 11H), 2.99 (s, 3H), 2.74 (d, 3H), 1.48 (d, 3H).

### Example 49

### 6-{2-[4-(7-Fluoro-2-methyl-5-quinolinyl)-1-piperazinyl]ethyl}-N-methyl-4H-imidazo[5,1-c][1,4]-benzoxazine-3-carboxamide dihydrochloride (E49)

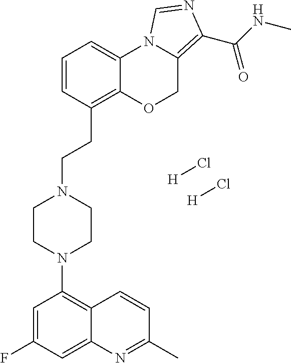

The title compound was prepared in 61% yield according to the general amide formation procedure starting from ethyl 6-{2-[4-(7-fluoro-2-methyl-5-quinolinyl)-1-piperazinyl]ethyl}-4H-imidazo[5,1-c][1,4]benzoxazine-3-carboxylate (E46) (48.7 mg, 0.1 mmole) and methylamine (0.055 ml of 2M sol. in THF, 0.11 mmol); MS (ES) m/z: 501.6 [MH⁺], C₂₈H₂₉FN₆O₂ requires 500.58; ¹H-NMR (500 MHz, DMSO-d⁶) δ: 11.39 (bs, 1H), 8.94 (d, 1H), 8.60 (s, 1H), 8.16 (bm, 1H), 7.82 (m, 3H), 7.43 (d, 1H), 7.25 (d, 1H), 7.15 (t, 1H), 5.59 (s, 2H), 3.75-3.40 (m, 10H), 3.2 (t, 2H), 2.93 (s, 3H), 2.74 (d, 3H).

### Example 50

### N-Methyl-6-{2-[4-(2-methyl-5-quinazolinyl)-1-piperazinyl]ethyl}-4H-imidazo[5,1-c][1,4]-benzoxazine-3-carboxamide dihydrochloride (E50)

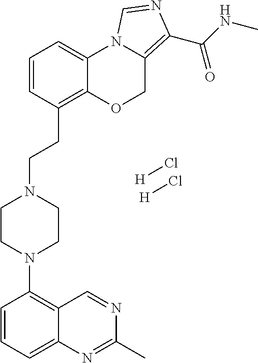

The title compound was prepared in 72% yield according to the general amide formation procedure starting from ethyl 6-{2-[4-(2-methyl-5-quinazolinyl)-1-piperazinyl]ethyl}-4H-imidazo[5,1-c][1,4]benzoxazine-3-carboxylic acid (E47) (47 mg, 0.1 mmol) and methylamine (0.055 ml of 2M sol. in THF, 0.11 mmol); MS (ES) m/z: 484.6 [MH⁺], C₂₇H₂₉N₇O₂ requires 483.57; ¹H-NMR (500 MHz, DMSO-d₆₆) δ: 11.95, 11.14 (s, 1H), 9.69 (d, 1H), 8.62 (s, 1H), 8.17 (d, 1H), 7.97 (t, 1H), 7.84 (d, 1H), 7.67 (d, 1H), 7.33 (d, 1H), 7.25 (dd, 1H), 7.16 (t, 1H), 5.60 (s, 2H), 3.71 (d., 2H), 3.6 (d, 2H), 3.52, 3.4, 3.19 (vm, 8H), 3.15 (s, 3H), 2.75 (d, 3H).

### Example 51

### 6-{2-[(2R)-2-Methyl-4-(2-methyl-5-quinolinyl)-1-piperazinyl]ethyl}-3-(4-morpholinylcarbonyl)-4H-imidazo[5,1-c][1,4]-benzoxazine dihydrochloride (E51)

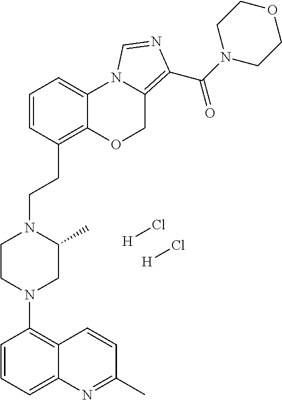

The title compound was prepared in 52% yield according to the general amide formation procedure starting from 6-{2-[(2R)-2-methyl-4-(2-methyl-5-quinolinyl)-1-piperazinyl]ethyl}-4H-imidazo[5,1-c][1,4]benzoxazine-3-carboxylic acid (E45) (48.3 mg, 0.1 mmol) and morpholine (0.01 ml, 0.11 mmol); MS (ES) m/z: 553.6 [MH⁺], C₃₂H₃₆N₆O3₂ requires 552.68; ¹H-NMR (500 MHz, DMSO-d⁶) δ: 11.7 (bs, 1H), 9.15 (d, 1H), 8.61 (s, 1H), 8.16 (d, 1H),), 8.03 (t, 1H), 7.92 (d, 1H), 7.86 (d, 1H), 7.51 (d, 1H), 7.31 (d, 1H), 7.15 (t, 1H), 5.57 (s, 2H), 4.0-2.9 (m., 19H), 2.99 (s, 3H), 1.48 (d, 3H).

### Example 52

### 6-{2-[4-(7-Fluoro-2-methyl-5-quinolinyl)-1-piperazinyl]ethyl}-3-(4-morpholinylcarbonyl)-4H-imidazo[5,1-c][1,4]-benzoxazine dihydrochloride (E52)

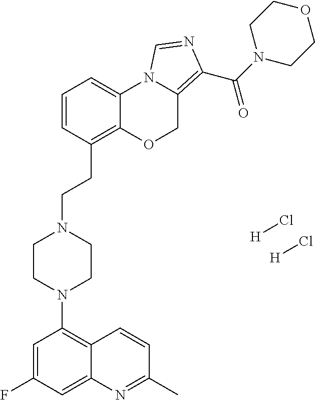

The title compound was prepared in 61% yield according to the general amide formation procedure starting from 6-{2-[4-(7-fluoro-2-methyl-5-quinolinyl)-1-piperazinyl]ethyl}-4H-imidazo[5,1-c][1,4]benzoxazine-3-carboxylate (E46) (49 mg, 0.1 mmol) and morpholine (0.01 ml, 0.11 mmol); MS (ES) m/z: 557.6 [MH⁺], C₃₁H₃₃FN₆O₂ requires 556.64.

### Example 53

### 6-{2-[4-(2-Methyl-5-quinazolinyl)-1-piperazinyl]ethyl}-3-(4-morpholinylcarbonyl)-4H-imidazo[5,1-c][1,4]-benzoxazine dihydrochloride (E53)

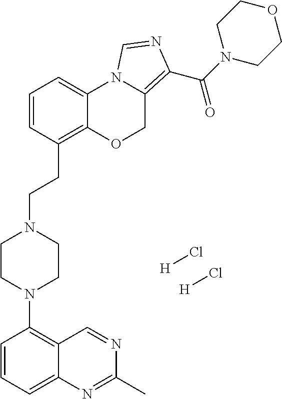

The title compound was prepared in 72% yield according to the general amide formation procedure starting from 6-{2-[4-(2-methyl-5-quinazolinyl)-1-piperazinyl]ethyl}-4H-imidazo[5,1-c][1,4]benzoxazine-3-carboxylic acid (E47) (47 mg, 0.1 mmol) and morpholine (0.055 ml, 0.11 mmol); MS (ES) m/z: 540.6 [MH⁺], C₃₀H₃₃N₇O₃ requires 539.64.

### Example 54

### 3-(3-Methyl-1,2,4-oxadiazol-5-yl)-6-{2-[4-(2-methyl-5-quinolinyl)-1-piperazinyl]ethyl}-4H-imidazo[5,1-c][1,4]-benzoxazine dihydrochloride (E54)

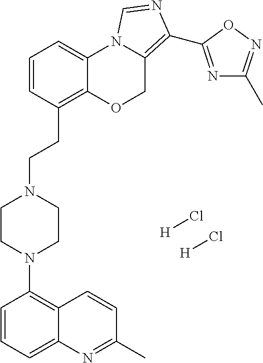

Methyl carboxyamide oxime (17 mg, 0.22 mmol) was added to a suspension of sodium hydride (85 mg of 60% suspension in oil, 0.22 mmol) in dry THF (5 ml) followed after 10 minutes by the addition of ethyl 6-{2-[4-(2-methyl-5-quinolinyl)-1-piperazinyl]ethyl}-4H-imidazo[5,1-c][1,4]benzoxazine-3-carboxylate (E1) (100 mg, 0.2 mmol). After 10 min. DMF (1 ml) was added and the reaction was stirred at room temperature overnight. The reaction mixture was quenched with water (1 ml) and extracted with ethyl acetate (3×15 ml). After drying and evaporation of the organic solvents, the crude material was triturated with diethyl ether to afford the free base of the title compound as yellow solid (77 mg, 0.15 mmol). This material was dissolved in dry methanol, 2.1 eq. of hydrochloric acid (1 M solution in diethyl ether) was slowly added at 0° C. The resulting suspension was stirred for 2 hours at 0° C. Evaporation of solvent and trituration with diethyl ether gave the title compound as a yellow solid in pure form (87 mg); MS (ES) m/z: 508.6 [MH⁺], C₂₉H₂₉N₇O₂ requires 507.6; ¹H-NMR (500 MHz, DMSO-d⁶) δ: 11.22 (s, 8.92 (d, 1H), 8.81 (s, 1H), 7.96 (m, 3H), 7.8 (bd, 1H), 7.45 (d, 1H), 7.31 (d, 1H), 7.20 (t, 1H), 5.7 (s, 2H), 3.74 (d, 2H), 3.6-3.4 (vm., 8H), 3.21 (m, 2H), 2.90 (s, 3H), 2.40 (s, 3H).

### Example 55

### Ethyl 6-{2-[(2R)-2-methyl-4-(2-methyl-5-quinolinyl)-1-piperazinyl]-ethyl}-imidazo[1,5-a]quinoline-3-carboxylate dihydrochloride (E55)

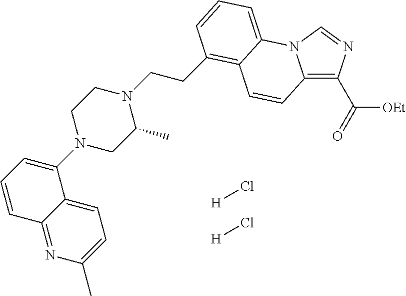

A mixture of ethyl 6-(2-oxoethyl)imidazo[1,5-a]quinoline-3-carboxylate (D91) (73 mg, 0.26 mmol), 2-methyl-5-[(3R)-3-methyl-1-piperazinyl]quinoline (WO2004046124) (75 mg, 0.31 mmol) in 1,2-dichloroethane (4 ml) was stirred at room temperature for 30 min. Sodium triacetoxyborohydride (66 mg, 0.31 mmol) was then added and the resulting reaction mixture was stirred overnight. The crude reaction was evaporated in vacuo and then purified by SPE-Si cartridge eluting with 4% MeOH in DCM to afford the free base of the title compound as a white solid (95 mg, 72%). The free base (15 mg, 0.03 mmol) was dissolved in dry MeOH (0.5 ml) and treated with HCl (0.053 ml of a 1.25 M solution in MeOH) at 0° C. The resulting suspension was stirred at room temperature for 1 hour. Evaporation of the volatiles and trituration with diethyl ether gave the title compound (11 mg, 63%) as a yellow solid; MS (ES) m/z: 508.3 [MH⁺], C31H34N5O2 requires 507.6; ¹H-NMR (500 MHz, DMSO-d⁶) δ: 11.7 (bs, 1H), 9.33 (s, 1H), 9.05 (bs, 1H), 8.5 (d, 2H), 8.03 (m, 4H), 7.9 (d, 1H), 7.79 (t, 1H), 7.64 (dd, 1H), 7.52 (m, 1H), 4.37 (q, 2H), 3.64 (bm+water, 11H), 2.95 (s, 3H), 1.50 (d, 2H), 1.37 (t, 3H).

### Example 56

### 6-(2-{4-[2-(Trifluoromethyl)-5-quinolinyl]-1-piperazinyl}ethyl)-4H-imidazo[5,1-c][1,4]benzoxazine-3-carboxylic acid ethyl ester (E56)

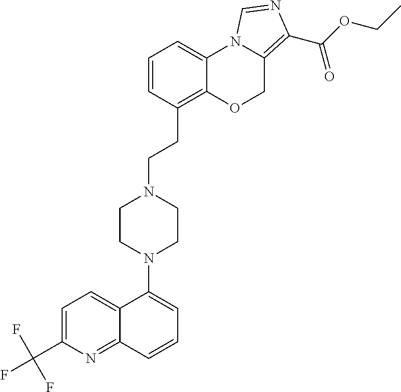

To a solution of 5-(1-piperazinyl)-2-(trifluoromethyl)quinoline (D47) (235 mg, 0.84 mmol) and ethyl 6-(2-oxoethyl)-4H-imidazo[5,1-c][1,4]benzoxazine-3-carboxylate (D6) (200 mg, 0.69 mmol) in dichloroethane (10 ml) was added sodium triacetoxyborohydride (178 mg, 0.84 mmol). After stirring the mixture at room temperature overnight, water (15 ml) was added and the product was extracted with DCM (3×20 ml). The combined organic phases were dried (MgSO₄), filtered and evaporated in vacuo. The residue was purified by chromatography on silica gel eluting with DCM/MeOH (98:2) to afford the title compound as a yellow solid (204 mg, 54%); MS (ES; m/z): 552[MH⁺], C₂₉H₂₈F₃N₅O₃ requires 551.57; ¹H-NMR (300 MHz, CDCl₃) δ: 9.69 (d, 1H), 7.98 (s, 1H), 7.90 (d, 1H), 7.73-7.67 (m, 2H), 7.36 (d, 1H), 7.33 (m, 1H), 7.14 (d, 1H), 7.02 (t, 1H), 5.55 (s, 2H), 4.40 (q, 2H), 3.15 (m, 4H), 2.98-2.68 (m, 8H), 1.39 (t, 3H).

### Example 57

### N-Methyl-6-(2-{4-[2-(trifluoromethyl)-5-quinolinyl]-1-piperazinyl}ethyl)-4H-imidazo[5,1-c][1,4]-benzoxazine-3-carboxamide (E57)

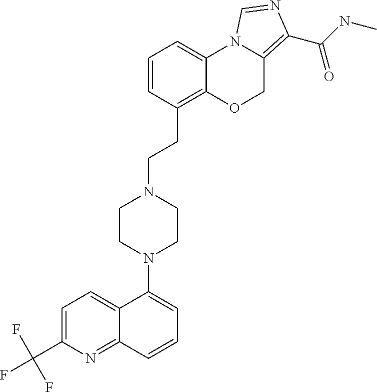

A solution of trimethylaluminium (2.0M in hexanes, 226 μl, 0.45 mmol) and methylamine (2.0M in THF, 226 μl, 0.45 mmol) in DCM (1 ml) was stirred at room temperature for 15 min. 6-(2-{4-[2-(Trifluoromethyl)-5-quinolinyl]-1-piperazinyl}ethyl)-4H-imidazo[5,1-c][1,4]benzoxazine-3-carboxylic acid ethyl ester (E56) (45 mg, 0.081 mmol) was added and stirring was continued for another 3 hours at 40° C. After the reaction was complete, water was added drop-wise until no more gas evolved and the final volume added reached ca. 3 ml. The aqueous solution was extracted with DCM (3×10 ml). In case the organic and aqueous phase did not separate well, aqueous 1M NaOH was added. The combined organic phases were dried (Na₂SO₄) and evaporated. The residue was purified by chromatography on silica gel eluting with DCM/MeOH (98:2 to 96:4) to afford the title compound as a colourless solid (36 mg, 83%); MS (ES; m/z): 537[MH⁺]. C₂₈H₂₇F₃N₆O₂ requires 536.56; ¹H-NMR (400 MHz; DMSO-d₆) δ: 8.79 (d, 1H), 8.56 (s, 1H), 8.14 (q, 1H), 7.95 (d, 1H), 7.84 (m, 2H), 7.75 (dd, 1H), 7.37 (quint., 1H), 7.24 (dd, 1H), 7.1 (t, 1H), 5.55 (s, 2H), 3.1 (bs, 4H), 2.89 (t, 2H), 2.8 (m, 4H), 2.76 (d, 3H), 2.67 (t, 2H); ¹⁹F-NMR (400 MHz, DMSO-d₆) δ: −66.05.

### Example 58

### N,N-Dimethyl-6-(2-{4-[2-(trifluoromethyl)-5-quinolinyl]-1-piperazinyl}ethyl)-4H-imidazo[5,1-c][1,4]-benzoxazine-3-carboxamide (E58)

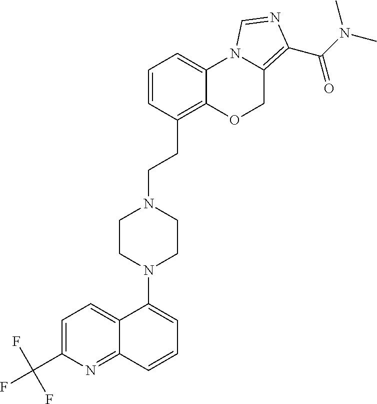

The title compound (12 mg, 40%) was obtained as colourless solid using the procedure of Example 57 using 6-(2-{4-[2-(trifluoromethyl)-5-quinolinyl]-1-piperazinyl}ethyl)-4H-imidazo[5,1-c][1,4]benzoxazine-3-carboxylic acid ethyl ester (E56) (30 mg, 0.054 mmol) and dimethylamine; MS (ES; m/z): 551 [MH⁺], C₂₉H₂₉F₃N₆O₂ requires 550.59; ¹H-NMR (400 MHz, DMSO-d₆) δ: 8.79 (d, 1H), 8.55 (s, 1H), 7.65 (d, 1H), 7.84 (d, 2H), 7.76 (dd, 1H), 7.37 (quint, 1H), 7.24 (dd, 1H), 7.1 (t, 1H), 5.48 (s, 2H), 3.49 (bs, 3H), 3.1 (bs, 4H), 2.98 (bs, 3H), 2.89 (t, 2H), 2.78 (bs, 4H), 2.66 (t, 2H); ¹⁹F-NMR (400 MHz, DMSO-d₆) δ: −66.05.

### Example 59

### 3-(4-Morpholinylcarbonyl)-6-(2-{4-[2-(trifluoromethyl)-5-quinolinyl]-1-piperazinyl}ethyl)-4H-imidazo[5,1-c][1,4]-benzoxazine (E59)

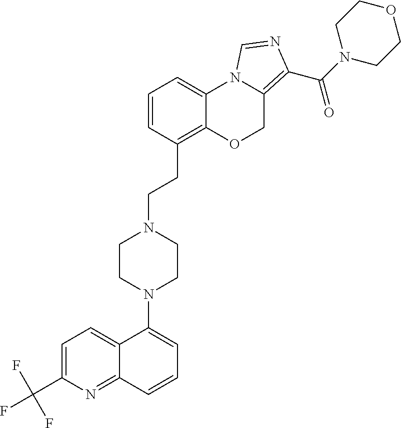

The title compound (29 mg, 78%) was obtained as a colourless solid using the procedure of Example 57 using 6-(2-{4-[2-(trifluoromethyl)-5-quinolinyl]-1-piperazinyl}ethyl)-4H-imidazo[5,1-c][1,4]benzoxazine-3-carboxylic acid ethyl ester (E56) (35 mg, 0.063 mmol) and morpholine; MS (ES; m/z): 593 [MH⁺], C₃₁H₃₁F₃N₆O₃ requires 592.62; ¹H-NMR (400 MHz, DMSO-d₆) δ: 8.79 (d, 1H), 8.56 (s, 1H), 7.95 (d, 1H), 7.84 (m, 2H), 7.77 (dd, 1H), 7.37 (quint., 1H), 7.24 (dd, 1H), 7.11 (t, 1H), 5.51 (s, 2H), 4.3 (vbs, 2H), 3.66 (m, 4H), 3.6 (vbs, 2H), 3.1 (bs, 4H), 2.89 (t, 2H), 2.78 (bs, 4H), 2.68 (t, 2H); ¹⁹F-NMR (400 MHz, DMSO-d₆) δ: −66.05.

### Example 60

### 6-(2-{4-[2-Cyano-5-quinolinyl]-1-piperazinyl}ethyl)-4H-imidazo[5,1-c][1,4]-benzoxazine-3-carboxylic acid ethyl ester (E60)

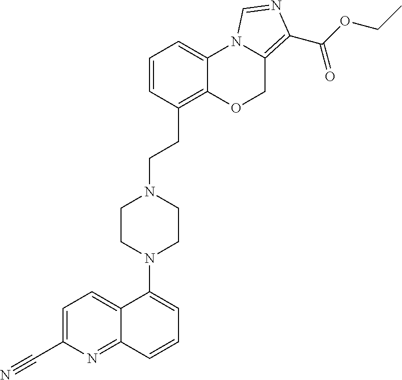

To a solution of 5-(1-piperazinyl)-2-quinolinecarbonitrile (D70) (200 mg, 0.839 mmol) and ethyl 6-(2-oxoethyl)-4H-imidazo[5,1-c][1,4]-benzoxazine-3-carboxylate (D6) (250 mg, 0.873 mmol) in DCM (10 ml) was added sodium triacetoxyborohydride (266 mg, 1.26 mmol). After stirring the mixture at room temperature overnight, water (15 ml) was added and the mixture extracted with DCM (3×20 ml). The combined organics were dried (MgSO₄), filtered and evaporated in vacuo. The residue was purified by chromatography on silica gel eluting with ethyl acetate/MeOH (1:0 to 9:1) to afford a yellow solid which contained the title compound (373 mg, HPLC/MS: 75% pure). A portion of this material (20 mg) was purified by mass-directed preparative HPLC; MS (ES; m/z): 509 [MH⁺], C₂₉H₂₈N₆O₃ requires 508.58; ¹H-NMR (400 MHz, DMSO-d₆) δ: 8.71 (d, 1H), 8.61 (s, 1H), 8.02 (d, 1H), 7.9-7.7 (m, 3H), 7.39 (dd, 1H), 7.26 (dd, 1H), 7.12 (t, 1H), 5.56 (s, 2H), 4.28 (quart., 2H), 3.09 (bs, 4H), 2.89 (t, 2H), 2.78 (bs, 4H), 2.66 (m, 2H), 1.33 (t, 3H); IR (cm⁻¹): 2230.65 (CN), 1728.58 (CO).

### Example 61

### Ethyl 7-fluoro-6-{2-[4-(2-methyl-5-quinolinyl)-1-piperazinyl}ethyl]-4H-imidazo[5,1-c][1,4]-benzoxazine-3-carboxylate (E61)

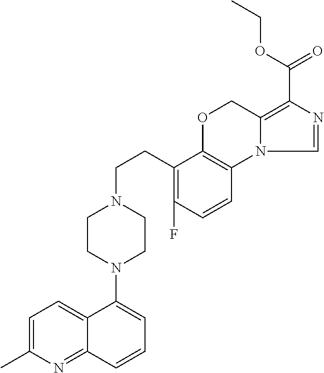

Potassium tert-butoxide (57 mg, 0.50 mmol) was added to a solution of 7-fluoro-8-{2-[4-(2-methyl-5-quinolinyl)-1-piperazinyl]ethyl}-2H-1,4-benzoxazin-3(4H)-one (D59) (200 mg, 0.48 mmol) in THF (5 ml) at 0° C. After stirring at this temperature for 30 min., the reaction was cooled to −20° C. and diethyl chlorophosphate (83 μL, 0.58 mmol) was added. After stirring at 0° C. for 1 hour, the reaction was cooled to −78° C. and ethyl isocyanoacetate (57 ul, 0.50 mmol) was added followed by potassium tort-butoxide (57 mg, 0.50 mmol). The reaction mixture was allowed to warm-up to room temperature and then stirred overnight. It was then poured into brine (15 ml) and extracted with DCM (3×20 ml). The combined organic phases were dried over Na₂SO₄ and concentrated. The resulting crude material was purified with chromatography on silica gel eluting with DCM/MeOH (96:4) to afford the title compound (120 mg, 50%); MS (ES/+) m/z 516 [MH⁺], C₂₉H₃₀FN₅O₃ requires 515; ¹H-NMR (300 MHz, CDCl₃) δ(ppm): 8.4 (d, 1H), 7.9 (s, 1H), 7.55 (d, 1H), 7.6 (t, 1H), 7.35 (dd, 1H), 7.3 (d, 1H), 7.08 (d, 1H), 6.85 (t, 1H), 5.6 (s, 2H), 4.4 (q, 2H) 3.2-2.6 (m, 15H), 1.4 (t, 3H).

### Example 62

### 7-Fluoro-6-{2-[4-(2-methyl-5-quinolinyl)-1-piperazinyl]ethyl}-3-(4-morpholinylcarbonyl)-4H-imidazo[5,1-c][1,4]-benzoxazine dihydrochloride (E62)

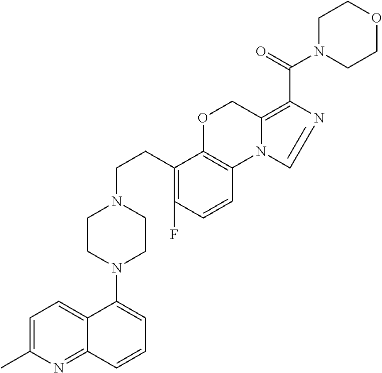

The title compound was prepared according to the procedure of Example 57 starting from ethyl 7-fluoro-6-{2-[4-(2-methyl-5-quinolinyl)-1-piperazinyl]ethyl}-4H-imidazo[5,1-c][1,4]benzoxazine-3-carboxylate (free base of E61) and morpholine; MS (ES/+) m/z: 557 [MH⁺], C₃₁H₃₃FN₆O₃ requires 556; ¹H-NMR (400 MHz, DMSO) δ(ppm): 10.43 (br s, 1H), 8.61 (s, 1H), 8.60 (br s, 1H), 7.95 (dd, 1H); 7.78 (br s, 2H), 7.60 (br s, 1H), 7.32 (br s, 1H), 7.15 (t, 1H), 5.63 (s, 2H), 4.34 (br s, 2H), 3.79 (d, 2H), 3.7-3.0 (m, 16H), 2.77 (br s, 3H).

### Example 63

### 7-Fluoro-N-methyl-6-{2-[4-(2-methyl-5-quinolinyl)-1-piperazinyl]ethyl}-4H-imidazo[5,1-c][1,4]-benzoxazine-3-carboxamide dihydrochloride (E63)

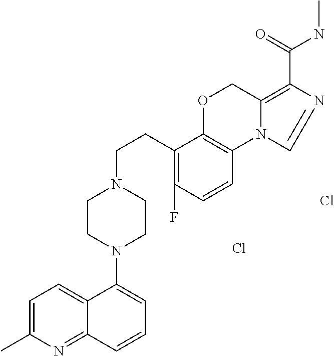

The title compound was prepared according to the procedure of Example 57 starting from ethyl 7-fluoro-6-{2-[4-(2-methyl-5-quinolinyl)-1-piperazinyl]ethyl}-4H-imidazo[5,1-c][1,4]benzoxazine-3-carboxylate (E61) and N-methylamine; MS: (ES/+) m/z: 501[MH⁺]. C₂₈H₂₉FN₆O₂ requires 500; ¹H-NMR (400 MHz, DMSO) δ(ppm): 10.64 (br s, 1H), 8.93 (br s, 1H), 8.60 (s, 1H), 8.2 (q, 1H), 7.9 (m, 3H), 7.82 (br s, 1H), 7.46 (br d, 1H), 7.15 (t, 1H), 7.15 (t, 1H), 5.67 (s, 2H), 3.8 (d, 2H), 3.7-3.0 (m, 8H), 3.22 (m, 2H), 2.9 (br s, 3H), 2.78 (d, 3H).

### Examples 64 and 65

### 7-Fluoro-6-{2-[4-(2-methyl-5-quinolinyl)-1-piperazinyl]ethyl}-4H-imidazo[5,1-c][1,4]-benzoxazine-3-carboxamide dihydrochloride (E64) and 7-Fluoro-6-{2-[4-(2-methyl-5-quinolinyl)-1-piperazinyl]ethyl}-4H-imidazo[5,1-c][1,4]-benzoxazine-3-carbonitrile dihydrochloride (E65)

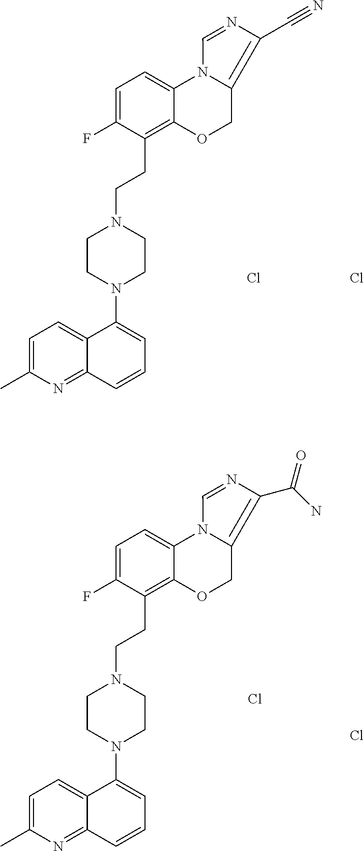

A mixture of ethyl 7-fluoro-6-{2-[4-(2-methyl-5-quinolinyl)-1-piperazinyl]ethyl}-4H-imidazo[5,1-c][1,4]benzoxazine-3-carboxylate (E61) (47 mg, 0.09 mmol), trimethyaluminium (230 μL of a 2M sol. in hexanes, 0.45 mmol) and ammonia (0.91 ml of a 0.5M sol. in 1,4-dioxane, 0.45 mmol) in DCM (3 ml) were microwave-irradiated at 100° C. for 1 hour. The reaction mixture was taken-up in MeOH (1 ml) and purified with SPE-(SCX) and then using column chromatography on silica gel eluting with DMC/MeOH (97:3) to afford the title compounds E64 (9 mg) and E65 (17 mg).

Example 64: MS (ES/+) m/z: 487 [MH⁺], C₂₇H₂₇FN₆O₂ requires 486; ¹H-NMR (400 MHz, DMSO) δ(ppm): 10.4 (br s, 1H), 8.8 (br s, 1H), 8.59 (s, 1H), 7.94 (dd, 1H), 7.85 (br s, 2H), 7.72 (br s, 1H), 7.57 (br s, 1H), 7.39 (br, s, 1H), 7.36 (br, s, 1H), 7.15 (t, 1H), 5.66 (s, 2H), 3.8 (m, 2H), 3.7-3 (m, 10H), 2.83 (br s, 3H).

Example 65: MS (ES/+) m/z: 469 [MH⁺], C₂₇H₂₅FN₆O requires 468; ¹H-NMR (400 MHz, DMSO) δ(ppm): 10.51 (br, 1H), 8.79 (s, 1H), 8.8 (br s, 1H), 7.99 (dd, 1H), 7.85 (br s, 2H), 7.71 (br s, 1H), 7.38 (br s, 1H), 7.21 (t, 1H), 5.61 (s, 2H), 3.79 (m, 2H), 3.6-3.1 (m, 10H), 2.83 (br s, 3H).

### Example 66

### Ethyl 4-methyl-6-{2-[4-(2-methyl-5-quinolinyl)-1-piperazinyl]ethyl}-4H-imidazo[5,1-c][1,4]-benzoxazine-3-carboxylate (E66)

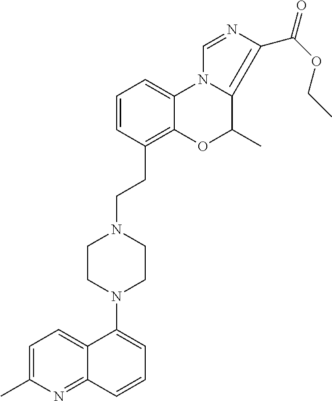

Potassium tert-butoxide (42 mg, 0.37 mmol) was added to a solution of 2-methyl-8-{2-[4-(2-methyl-5-quinolinyl)-1-piperazinyl]ethyl}-2H-1,4-benzoxazin-3(4H)-one (D61) (150 mg, 0.36 mmol) in THF (5 ml) at 0° C. After stirring at this temperature for 30 min., the mixture was cooled to −20° C. and diethylchlorophosphate (62 μL, 0.43 mmol) was added. After stirring at 0° C. for 1 hour, the reaction was cooled to −78° C. and ethyl isocyanoacetate (42 μL, 0.37 mmol) was added followed by potassium tert-butoxide (42 mg, 0.37 mmol). The reaction mixture was allowed to warm-up to room temperature and then stirred overnight. The mixture was then poured into brine (15 ml) and extracted with DCM (3×20 ml). The combined organic phases were dried over Na₂SO₄ and concentrated to give crude product which was purified using chromatography on silica gel eluting with DCM/MeOH (97:3) to afford the title compound (60 mg, 30%); MS (ES/+) m/z: 512 [MH⁺]. C₃₀H₃₃N₅O₃ requires 511; ¹H-NMR (300 MHz, CDCl₃) δ(ppm): 8.35 (d, 1H), 7.70 (d, 1H), 7.55 (t, 1H), 7.3-7-0 (m, 6H), 6.15 (q, 1H), 3.2-2.6 (m, 15H), 1.5 ppm (d, 3H).

### Example 67

### N,4-Dimethyl-6-{2-[4-(2-methyl-5-quinolinyl)-1-piperazinyl]ethyl}-4H-imidazo[5,1-c][1,4]-benzoxazine-3-carboxamide dihydrochloride (E67)

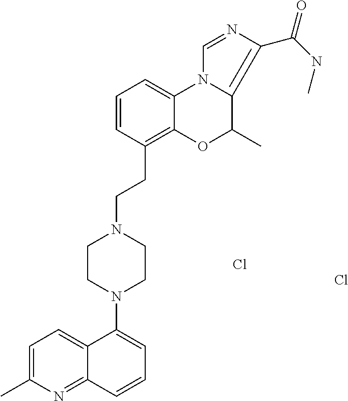

The title compound was prepared in 42% yield according to the procedure of Example 57 starting from ethyl 4-methyl-6-{2-[4-(2-methyl-5-quinolinyl)-1-piperazinyl]ethyl}-4H-imidazo[5,1-c][1,4]benzoxazine-3-carboxylate (E66) and methylamine; MS (ES/+) m/z: 497 [MH⁺], C₂₉H₃₂N₆O₂ requires 496; ¹H-NMR (400 MHz, DSMO) δ(ppm): 10.72 (br s, 1H), 8.88 (br s, 1H), 8.6 (s, 1H), 8.17 (q, 1H), 7.91 (br s, 2H), 7.87 (d, 1H), 7.8 (m, 1H), 7.44 (m, 1H), 7.28 (d, 1H), 7.17 (t, 1H), 6.18 (q. 1H), 3.78 (m, 2H), 3.6-3.1 (m, 8H), 3.19 (m, 2H), 2.88 (br s, 3H), 2.77 (d, 3H), 1.51 (d, 3H).

### Example 68

### Ethyl 7-fluoro-6-(2-{4-[2-(trifluoromethyl)-5-quinolinyl]-1-piperazinyl}-ethyl)-4H-imidazo[5,1-c][1,4]-benzoxazine-3-carboxylate (E68)

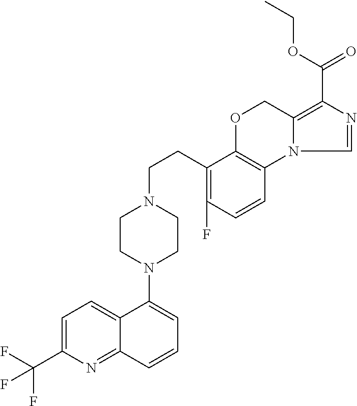

Potassium tert-butoxide (350 μl of 1M sol. in THF, 0.35 mmol) was added to a solution of 7-fluoro-8-(2-{4-[2-(trifluoromethyl)-5-quinolinyl]-1-piperazinyl}-ethyl)-2H-1,4-benzoxazin-3(4H)-one (D64) (165 mg, 0.348 mmol) in THF (5 ml) at 0° C. After stirring at this temperature for 30 min., the reaction was cooled to −20° C. and diethylchlorophosphate (60 μL, 0.42 mmol) was added. After stirring at 0° C. for 1 hour, the reaction was cooled to −78° C. and ethyl isocyanoacetate (40 μL, 0.35 mmol) was added followed by potassium tert-butoxide (350 μL of 1M sol. in THF, 0.35 mmol). The reaction mixture was allowed to warm-up to room temperature and then stirred overnight. It was then poured into brine (15 ml) and extracted with DCM (3×20 ml). The combined organic phases were dried over Na₂SO₄ and concentrated to a residue which was purified using column chromatography on silica gel eluting with DCM/MeOH (97:3) to afford the title compound (50 mg, 25%); MS (ES/+) m/z: 570 [MH⁺], C₂₉H₂₇F₄N₅O₃ requires 569; ¹H-NMR (300 MHz, CDCl₃) δ(ppm): 8.65 (d, 1H), 7.70 (m, 1H), 7.8-7.6 (m, 3H), 7.2 (m, 1H), 6.7-6.6 (m, 2H), 5.6 (s, 2H), 4.4 (q, 2H), 3.1-2.6 (m, 12H), 1.4 (t, 3H).

### Example 69

### 7-Fluoro-N-methyl-6-(2-{4-[2-(trifluoromethyl)-5-quinolinyl]-1-piperazinyl}ethyl)-4H-imidazo[5,1-c][1,4]-benzoxazine-3-carboxamide hydrochloride (E69)

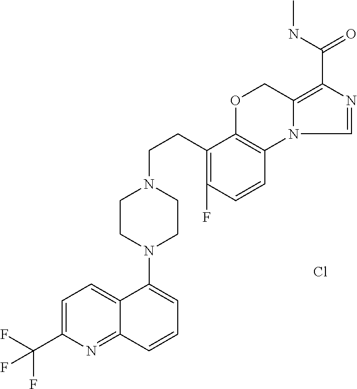

The title compound was prepared in 50% yield according to the procedure of Example 57 starting from ethyl 7-fluoro-6-(2-{4-[2-(trifluoromethyl)-5-quinolinyl]-1-piperazinyl}ethyl)-4H-imidazo[5,1-c][1,4]-benzoxazine-3-carboxylate (E68) and methylamine; MS (ES/+) m/z: 555 [MH⁺], C₂₈H₂₆F₄N₆O₂ requires 554; ¹H-NMR (400 MHz, CDCl₃) δ(ppm): 8.65 (d, 1H), 7.95 (d, 1H), 7.9 (s, 1H), 7.8-7.7 (m, 3H), 7.35-7-25 (m, 2H), 6.85 (t, 1H), 5.6 (s, 2H), 3.1-2.6 (m, 15H).

### Example 70

### 7-Fluoro-6-(2-{4-[2-(trifluoromethyl)-5-quinolinyl]-1-piperazinyl}ethyl)-4H-imidazo[5,1-c][1,4]-benzoxazine-3-carbonitrile hydrochloride (E70)

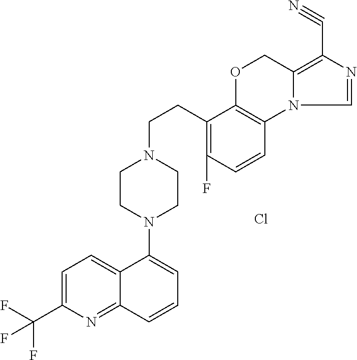

A mixture of ethyl 7-fluoro-6-(2-{4-[2-(trifluoromethyl)-5-quinolinyl]-1-piperazinyl}ethyl)-4H-imidazo[5,1-c][1,4]benzoxazine-3-Carboxylate (E68) (25 mg, 0.043 mmol), trimethylaluminium (110 μL of a 2M sol. in hexanes, 0.213 mmol) and ammonia (0.43 ml of a 0.5 M sol. in 1,4-dioxane, 0.213 mmol) in DCM (3 ml) was irradiated at 100° C. for 1 hour. The reaction mixture was taken-up in MeOH (1 ml) and purified with SPE-(SCX) and then by mass-directed preparative HPLC to afford the title compound (4 mg, 18%); MS: (ES/+) m/z: 523 [MH⁺], C₂₇H₂₂F₄N₆O requires 522; ¹H-NMR (400 MHz, CDCl₃) δ(ppm): 9.7 (d, 1H), 8 (s, 1H), 7.95 (d, 1H), 7.8-7.7 (m, 2H), 7.4-7.25 (m, 2H), 6.9 (t, 1H), 5.6 (s, 2H), 3.1-2.6 (m 12H).

### Example 71

### Ethyl 7-fluoro-6-{2-[4-(2-methyl-5-quinazolinyl)-1-piperazinyl]ethyl}-4H-imidazo[5,1-c][1,4]-benzoxazine-3-carboxylate (E71)

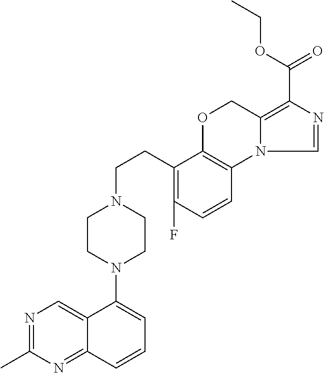

Potassium tert-butoxide (215 ul of 1M sol. in THF, 0.22 mmol) was added to a solution of 7-fluoro-8-{2-[4-(2-methyl-5-quinazolinyl)-1-piperazinyl]ethyl}-2H-1,4-benzoxazin-3(4H)-one (D67) (90 mg, 0.21 mmol) in THF (5 ml) at 0° C. After stirring at this temperature for 30 min., the mixture was cooled to −20° C. and diethylchlorophosphate (45 μL, 0.25 mmol) was added. After stirring at 0° C. for 1 hour, the reaction mixture was cooled to −78° C. and ethyl isocyanoacetate (29 μL, 0.21 mmol) was added followed by potassium tert-butoxide (215 ul of 1M sol. in THF, 0.22 mmol). The reaction mixture was allowed to warm-up to room temperature and then stirred overnight. It was then poured into brine (15 ml) and extracted with DCM (3×20 ml). The combined organic phases were dried over Na₂SO₄ and concentrated to a residue which was purified using chromatography on silica gel eluting with DCM/MeOH (96:4) to afford the title compound (20 mg, 20%); MS (ES/+) m/z 517 [MH⁺], C₂₈H₂₉FN₆O₃ requires 516; ¹H-NMR (300 MHz, CDCl₃) δ(ppm): 9.5 (s, 1H), 8.0 (S, 1H), 7.75 (t, 1H), 7.6 (d, 1H), 7.4-7.3 (m, 1H), 7.05 (d, 1H), 6.85 (t, 1H), 5.6 (s, 2H), 4.4 (q, 2H), 3.4-2.6 (m, 15H), 1.45 (t, 3H).

### Example 72

### 7-Fluoro-N-methyl-6-{2-[4-(2-methyl-5-quinazolinyl)-1-piperazinyl]ethyl}-4H-imidazo[5,1-c][1,4]-benzoxazine-3-carboxamide dihydrochloride (E72)

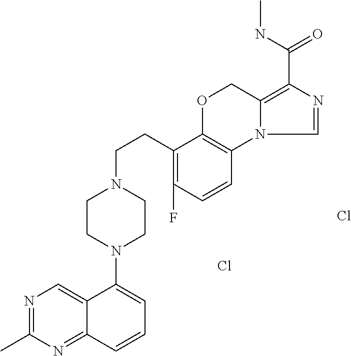

The title compound was prepared in 50% yield according to the procedure of Example 57 starting from ethyl 7-fluoro-6-{2-[4-(2-methyl-5-quinazolinyl)-1-piperazinyl]ethyl}-4H-imidazo[5,1-c][1,4]benzoxazine-3-carboxylate (E71) and methylamine; MS (ES/+) m/z: 502 [MH⁺], C₂₇H₂₈FN₇O₂ required 501; ¹H-NMR (400 MHz, DMSO) δ(ppm): 10.5 (br s, 1H), 9.63 (s, 1H), 8.60 (s, 1H), 8.18 (q, 1H), 7.9 (m, 2H), 7.62 (d, 1H), 7.30 (d, 1H), 7.14 (t, 1H), 5.66 (s, 2H), 3.78 (d, 2H), 3.61 (t, 2H), 3.5-3.3 (m, 4H), 3.30 (t, 2H), 3.19 (dd, 2H), 2.79 (s, 3H), 2.77 (d, 3H).

### Example 73

### Ethyl 6-{2-[4-(2-methyl-5-quinolinyl)-1-piperazinyl]ethyl}-4H-imidazo[5,1-c][1,4]-benzoxazine-3-carboxylate dihydrochloride (E73)

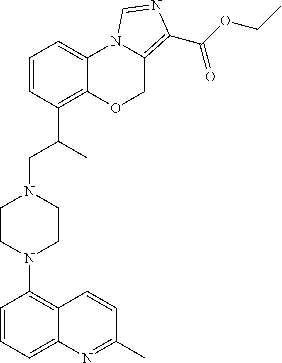

A mixture of ethyl 6-(1-methyl-2-oxoethyl)-4H-imidazo[5,1-c][1,4]benzoxazine-3-carboxylate (D53) (151 mg, 0.50 mmol) and 2-methyl-5-piperazin-1-yl-quinoline (WO2004046124) (108 mg, 0.0.48 mmol) in dry 1,2-dichloroethane (12.5 ml) was stirred at room temperature for 1 hour. Sodium triacetoxyborohydride (127 mg, 0.60 mmol) was added and the resulting mixture stirred for 18 hours, then quenched with water (30 ml) and extracted with ethyl acetate (3×25 ml). The combined organics were dried (Na₂SO₄) and concentrated in vacuo. The crude product was purified by SPE cartridge (silica gel) eluting with 2% MeOH in DCM to afford the title compound as a colourless oil (200 mg, 78%); MS (ES) m/z 512.4 [MH⁺]; ¹H-NMR (300 MHz, CDCl₃) δ: 8.36 (d, 1H), 7.98 (s, 1H), 7.69 (d, 1H), 7.54 (t, 1H), 7.33 (d, 1H), 7.25-7.22 (m, 1H+CDCl₃), 7.17 (d, 1H), 7.12-6.96 (m, 2H), 5.52 (s, 2H), 4.39 (q, 2H), 3.51 (m, 1H), 3.10-3.0 (m, 4H), 2.76-2.5 (m, 9H), 1.41 (t, 3H), 1.30 (d, 3H).

### Examples 74 and 75

### N-Methyl-6-{1-methyl-2-[4-(2-methyl-5-quinolinyl)-1-piperazinyl]ethyl}-4H-imidazo[5,1-c][1,4]-benzoxazine-3-carboxamide dihydrochloride

### Enantiomer 1 (E74)

### Enantiomer 2 (E75)

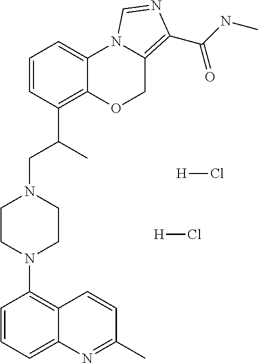

A mixture of trimethylaluminium (214 μl of a 2.0M sol. in hexanes, 0.43 mmol) and methylamine (214 μl of a 2.0M In THF, 0.43 mmol) in DCM (1.7 ml) was stirred at room temperature for 15 min. Ethyl 6-{2-[4-(2-methyl-5-quinolinyl)-1-piperazinyl]ethyl}-4H-imidazo[5,1-c][1,4]benzoxazine-3-carboxylate (free base of E73) (44 mg, 0.086 mmol) was added and stirring was continued for another 2 hours at 54° C. After reaction was completed, water was added dropwise until no more gas evolved and the final volume added reached ca. 4 ml. The aqueous solution was extracted with DCM (3×10 ml). In case the organic and aqueous phase did not separate well, 1M aqueous NaOH was added. The combined organic phases were dried (Na₂SO₄) and evaporated to afford the free base of the title compound (30 mg). The racemic mixture was then separated by semi-preparative SFC (Gilson) chromatography [CHIRALCEL AD-H, 25×2.1 cm; modifier 30% (Ethanol+0.1% isopropylamine), flow rate=22 ml/min; pressure 192 bar T=36° C.; UV wavelength: 220 nm; loop=1 ml to obtain enantiomer 1 (5 mg) and enantiomer 2 (8 mg). The enantiomeric excess of both enantiomers were verified by analytical SFC (Berger) conditions: Chiral column: CHIRALPAK AD-H, 25×0.46 cm; modifier. 30% (Ethanol+0.1% isopropylamine), flow rate=2.5 ml/min; pressure 180 bar T=35° C.; UV wavelength: 220 nm; loop=10 microl

Enantiomer 1 (E74)—(100% a/a by UV, retention time 17.5 min, e.e=100%)
Enantiomer 2 (E75)—(100% a/a by UV, retention time 25.6 min, e.e=100%)

The free base of E74 was dissolved in dry methanol (1 ml) and HCl (8 μl of a 1.25M solution in MeOH, 0.1 mmol) was slowly added at 0° C. The resulting suspension was stirred at 0° C. for 4 hours. Evaporation of the volatiles and trituration with diethyl ether gave the title compound as a yellow solid (4 mg).

The free base of E75 was dissolved in dry methanol (1 ml) and HCl (13 μl of a 1.25M solution in MeOH, 0.1 mmol) was slowly added at 0° C. The resulting suspension was stirred at 0° C. for 4 hours. Evaporation of the volatiles and trituration with diethyl ether gave the title compound as a yellow solid (5.3 mg).

Enantiomer 1 (E74): ¹H-NMR (500 MHz, DMSO-d₆) δ: 9.70 (bs, 1H), 8.58 (s, 1H), 8.42 (bs, 1H), 8.15 (bs, 1H), 7.84 (d, 2H), 7.67 (bs, 2H), 7.46 (bs., 1H), 7.34 (d, 1H), 7.19 (m, 1H), 5.62 (s, 2H), 3.8-3.1 (bm, 11H+water), 2.74 (d, 3H), 2.67 (s, 3H), 1.37 (d, 3H).

Enantiomer 2 (E75): ¹H-NMR (500 MHz, DMSO-d₆) δ: 9.48 (bs, 1H), 8.58 (s, 1H), 8.36 (m, 1H), 8.14 (d, 1H), 7.84 (d, 2H), 7.64 (m, 2H), 7.42 (m., 1H), 7.34 (d, 1H), 7.17 (m, 1H), 5.62 (s, 2H), 3.8-3.1 (bm, 11H+water), 2.98 (s, 3H), 2.74 (d, 3H), 1.37 (d, 3H).

### Example 76

### 6-{2-[4-(7-Fluoro-2-methyl-5-quinolinyl)-1-piperazinyl]ethyl}-3-methyl-4H-[1,2,3]triazolo[5,1-c][1,4]-benzoxazine dihydrochloride (E76)

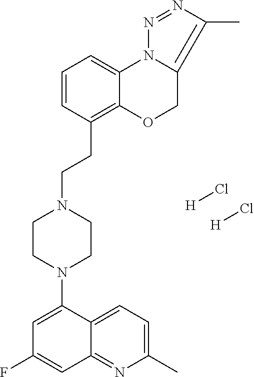

The title compound was prepared following the general reductive amination procedure of Example 1 starting from (3-methyl-4H-[1,2,3]triazolo[5,1-c][1,4]benzoxazin-6-yl)acetaldehyde (D41) (20 mg, 0.087 mmol) and 7-fluoro-2-methyl-5-(1-piperazinyl)quinoline (WO2004/046124) (32 mg, 0.131 mmol). The crude product was purified by flash chromatography on silica gel eluting with a gradient of MeOH in DCM (1 to 3%) to afford the free base of the title compound (27 mg, 68%). Treatment with HCl (2.2 eq. of 1.25M solution in MeOH) in 4:1 methanol/DCM (5 ml) at 0° C. gave the title compound as a solid; MS (ES) m/z: 459.20 [MH⁺], C26H27FN6O requires 458.54; ¹H-NMR (500 MHz, DMSO-d6) δ: 10.57 (bs, 1H), 8.52 (bd, 1H), 7.93 (dd, 1H), 7.53 (d, 1H), 7.47 (d, 1H), 7.36 (dd, 1H), 7.24 (m, 2H), 5.57 (s, 2H), 3.75 (d, 2H), 3.60-3.10 (vbm, 10H), 2.73 (s, 3H), 2.34 (s, 3H).

### Example 77

### Ethyl 6-{2-[4-(2-methyl-5-quinolinyl)-1-piperazinyl]ethyl}-4,5-dihydroimidazo[1,5-a]quinoline-3-carboxylate dihydrochloride (E77)

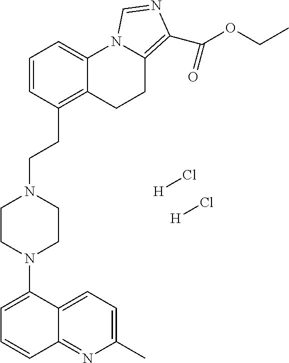

Diethyl chlorophosphate (0.24 ml, 1.67 mmol) was added to a solution of 5-{2-[4-(2-methyl-5-quinolinyl)-1-piperazinyl]ethyl}-3,4-dihydro-2(1H)-quinolinone (D74) (334 mg, 0.835 mmol) and potassium t-butoxide (140 mg, 1.25 mmol) in dry DMF (15 ml) at −5° C. After 20 min. a solution of ethyl isocyanoacetate (0.14 ml, 1.25 mmol) and potassium t-butoxide (140 mg, 1.25 mmol) in dry DMF (2 ml) was added. The reaction mixture was stirred at room temperature for 24 hours then quenched with water (5 ml) and extracted with DCM (3×50 ml). The combined organic layers were dried (Na₂SO₄), concentrated in vacuo and the crude product was purified by flash chromatography on silica gel eluting with a gradient of methanol in DCM (1 to 3%), to afford the free base of the title compound (116 mg, 28%). Treatment with HCl (2.2 eq. of 1.25M solution in MeOH) in 1:1 methanol/DCM (4 ml) at 0° C. gave the title compound as a solid; MS (ES) m/z: 496.2 [MH⁺], C30H33N5O2 requires 495.62; ¹H-NMR (400 MHz, DMSO-d6) δ: 10.87 (bs, 1H), 8.66 (bs, 1H), 8.57 (s, 1H), 7.81 (m, 3H), 7.63 (bs, 1H), 7.43 (t, 1H), 7.35 (bs, 1H), 7.29 (d, 1H), 4.29 (q, 2H), 3.78 (d, 2H), 3.52-3.35 (m, 6H), 3.40-3.20 (m, 6H), 3.05 (m, 2H), 2.79 (s, 3H), 1.33 (t, 3H).

### Example 78

### N-Methyl-6-{2-[4-(2-methyl-5-quinolinyl)-1-piperazinyl]ethyl}-4,5-dihydroimidazo[1,5-a]quinoline-3-carboxamide dihydrochloride (E78)

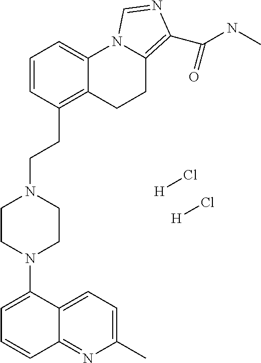

The title compound was prepared according to the procedure of Example 57 starting from ethyl 6-{2-[4-(2-methyl-5-quinolinyl)-1-piperazinyl]ethyl}-4,5-dihydroimidazo[1,5-a]quinoline-3-carboxylate (free base of E77) (53 mg, 0.107 mmol) and methylamine. The crude product was purified by flash chromatography on silica gel eluting with a gradient of MeOH in DCM (1 to 3%) to afford the free base of the title compound (33 mg, 65%). Treatment with HCl (2.2 eq. of 1.25M solution in MeOH) in 3:1 methanol/DCM (4 ml) at 0° C. gave the title compound as a solid; MS (ES) m/z: 481.20 [MH⁺], C29H32N6O requires 480.61; ¹H-NMR (400 MHz, DMSO-d6) δ: 10.52 (bs, 1H), 8.55 (bs, 1H), 8.49 (s, 1H), 8.00 (q, 1H), 7.79 (d, 1H), 7.76 (bs, 2H), 7.58 (bs, 1H), 7.41 (t, 1H), 7.32 (bs, 1H), 7.27 (d, 1H), 3.79 (d, 2H), 3.52-3.30 (m, 6H), 3.40-3.20 (m, 6H), 3.00 (m, 2H), 2.77 (2s, 6H).

### Example 79

### 6-{2-[4-(2-Methyl-5-quinolinyl)-1-piperazinyl]ethyl}-3-(4-morpholinylcarbonyl)-4,5-dihydroimidazo[1,5-a]quinoline dihydrochloride (E79)

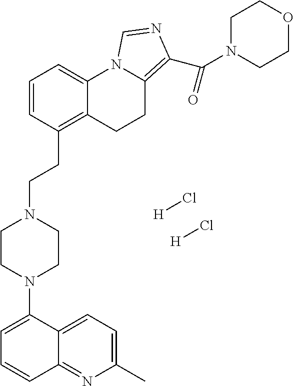

The title compound was prepared in 82% yield according to the procedure of Example 57 starting from ethyl 6-{2-[4-(2-methyl-5-quinolinyl)-1-piperazinyl]ethyl}-4,5-dihydroimidazo[1,5-a]quinoline-3-carboxylate (free base of E77) (53 mg, 0.107 mmol) and morpholine. The crude product was purified by flash chromatography on silica gel eluting with a gradient of MeOH in DCM (1 to 5%) to afford the free base of the title compound (47 mg, 82%). Treatment with HCl (2.2 eq. of 1.25M solution in MeOH) in 4:1 methanol/DCM (5 ml) at 0° C. gave the title compound as a solid; MS (ES) m/z: 537.40 [MH⁺], C32H36N6O2 requires 536.68; ¹H-NMR (400 MHz, DMSO-d6) δ: 10.65 (bs, 1H), 8.57 (bs, 1H), 8.50 (s, 1H), 7.79 (d, 1H), 7.77 (bs, 2H), 7.58 (bs, 1H), 7.42 (t, 1H), 7.31 (bs, 1H), 7.28 (d, 1H), 4.18 (bs, 2H), 3.78 (d, 2H), 3.66 (m, 6H), 3.52-3.30 (m, 6H), 3.40-3.20 (m, 6H), 3.00 (m, 2H), 2.77 (s, 3H).

### Example 80

### Ethyl 7-methyl-6-{2-[4-(2-methyl-5-quinolinyl)-1-piperazinyl]ethyl}-4H-imidazo[5,1-c][1,4]-benzoxazine-3-carboxylate (E80)

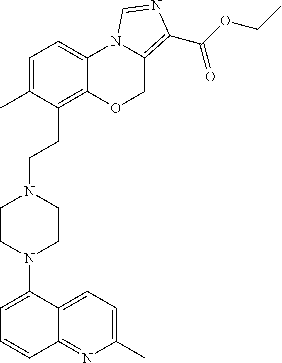

Potassium t-butoxide (132 μl of 1M sol. in THF, 0.132 mmol) was added to a solution of 7-methyl-8-{2-[4-(2-methyl-5-quinolinyl)-1-piperazinyl]ethyl}-2H-1,4-benzoxazin-3(4H)-one (D80) (50 mg, 0.120 mmol) in THF (3 ml) at 0° C. After stirring at CC for 20 min., the reaction was cooled to −20° C. and diethylchlorophosphate (22 μl, 0.156 mmol) was added slowly. After stirring at 0° C. for 30 min., the reaction was cooled to −78° C. and ethyl isocyanoacetate (15 μl, 0.132 mmol) was added followed by potassium t-butoxide (132 μl of 1M sol. in THF, 0.132 mmol). After stirring at ambient temperature for 4 hours, the reaction was quenched with a saturated aqueous solution of NH₄Cl (4 ml) and extracted with DCM (3×50 ml). The combined organic layers were dried (Na₂SO₄), concentrated in vacuo and the crude product was purified by flash chromatography on silica gel eluting with a gradient of MeOH in DCM (1 to 3%), to afford the title compound (29 mg, 48%); MS; (ES) m/z: 512.40 [MH⁺]. C30H33N5O3 requires 511.62.

### Example 81

### N,7-Dimethyl-6-{2-[4-(2-methyl-5-quinolinyl)-1-piperazinyl]ethyl}-4H-imidazo[5,1-c][1,4]-benzoxazine-3-carboxamide dihydrochloride (E81)

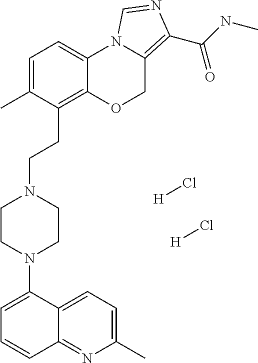

The title compound was prepared according to the procedure of Example 57 starting from ethyl 7-methyl-6-{2-[4-(2-methyl-5-quinolinyl)-1-piperazinyl]-ethyl}-4H-imidazo[5,1-c][1,4]benzoxazine-3-carboxylate (E80) (29 mg, 0.06 mmol). The crude product was purified by flash chromatography on silica gel eluting with a gradient of MeOH in DCM (1 to 3%) to afford the free base of the title compound (24 mg, 86%). Treatment with HCl (2.2 eq. of 1.25M solution in MeOH) in 4:1 methanol/DCM (5 ml) at 0° C. gave the title compound as a solid; MS (ES) m/z: 497.20 [MH⁺], C29H32N6O2 requires 496.61; ¹H-NMR (400 MHz, DMSO-d6) δ: 10.50 (vbs, 1H), 8.70 (vbs, 1H), 8.53 (s, 1H), 8.14 (q, 1H), 7.80 (bs, 2H), 7.72 (d, 1H), 7.64 (vbs, 1H), 7.35 (bs, 1H), 7.03 (d, 1H), 5.58 (s, 2H), 3.79 (d, 2H), 3.60-3.10 (m, 8H), 3.15 (m, 2H), 2.78 (s, 3H), 2.74 (d, 3H), 2.39 (s, 3H).

### Example 82

### Ethyl 6-{2-[4-(2-methyl-5-quinolinyl)-1-piperidinyl]ethyl}-4,5-dihydroimidazo[1,5-a]quinoline-3-carboxylate dihydrochloride (E82)

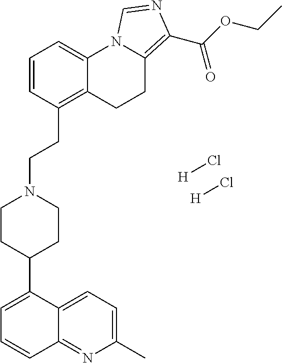

The title compound was prepared following the general reductive amination procedure of Example 1 starting from ethyl 6-(2-oxoethyl)-4,5-dihydroimidazo[1,5-a]quinoline-3-carboxylate (D86) (50 mg, 0.18 mmol) and 2-methyl-5-(4-piperidinyl)quinoline (WO2004/046124) (60 mg, 0.26 mmol). The crude product was purified by flash chromatography on silica gel eluting with a gradient of MeOH in DCM (1 to 3%) to afford the free base of the title compound (75 mg, 84%). Treatment with HCl (2.2 eq. of 1.25M solution in MeOH) in 4:1 MeOH/DCM (5 ml) at 0° C. gave the title compound as a solid; MS (ES) m/z: 495.4 [MH⁺], C31H34N4O2 requires 494.64; ¹H-NMR (500 MHz, DMSO-d6) δ: 11.07 (bs, 1H), 9.29 (bd, 1H), 8.66 (s, 1H), 8.23 (d, 1H), 8.09 (t, 1H), 7.98 (d, 1H), 7.82 (d, 1H), 7.75 (d, 1H), 7.43 (t, 1H), 7.32 (d, 1H), 4.29 (q, 2H), 3.85 (m, 1H), 3.80 (bd, 2H), 3.7-3.2 (bm, 8H), 3.06 (t, 2H), 2.97 (s, 3H), 2.30 (m, 2H), 2.12 (bd, 2H), 1.33 (t, 3H).

### Example 83

### N-Methyl-6-{2-[4-(2-methyl-5-quinolinyl)-1-piperidinyl]ethyl}-4,5-dihydroimidazo[1,5-a]quinoline-3-carboxamide dihydrochloride (E83)

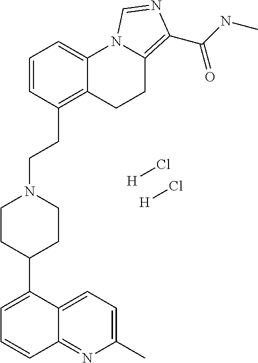

The title compound was prepared according to the procedure of Example 57 starting from ethyl 6-{2-[4-(2-methyl-5-quinolinyl)-1-piperidinyl]ethyl}-4,5-dihydroimidazo[1,5-a]quinoline-3-carboxylate (free base of E82) (33 mg, 0.07 mmol). The crude product was purified by flash chromatography on silica gel eluting with a gradient of MeOH in DCM (1 to 5%) to afford the free base of the title compound (25 mg, 78%). Treatment with HCl (2.2 eq. of 1.25M solution in MeOH) in 1:1 MeOH/DCM (2 ml) at 0° C. gave the title compound as a solid; MS (ES) m/z: 480.3 [MH⁺], C30H33N5O requires 479.62; ¹H-NMR (500 MHz, DMSO-d6) δ: 10.25 (bs, 1H), 8.72 (bs, 1H), 8.47 (s, 1H), 7.97 (d, 1H), 7.92 (bs, 1H), 7.80 (bs, 1H), 7.77 (d, 1H), 7.61 (bs, 1H), 7.50 (bs, 1H), 7.38 (t, 1H), 7.26 (d, 1H), 3.78 (d, 2H), 3.72 (m, 1H), 3.6-3.1 (bm, 8H), 2.98 (t, 2H), 2:74 (m, 6H), 2.12 (m, 4H).

### Example 84

### 6-{2-[4-(2-Methyl-5-quinolinyl)-1-piperazinyl]ethyl}-4,5-dihydroimidazo[1,5-a]quinoline-3-carboxamide dihydrochloride (E84)

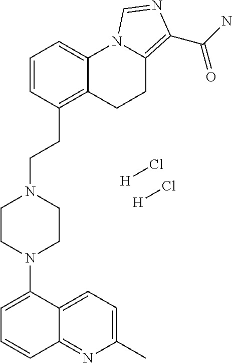

The title compound was prepared in 70% yield from 6-{2-[4-(2-methyl-5-quinolinyl)-1-piperazinyl]ethyl}-4,5-dihydroimidazo[1,5-a]quinoline-3-carboxylate (E180) (122 mg, 0.261 mmol) following the general procedure for amide formation (see Examples 48-53) using hexamethyldisilazane (1.1 eq). Treatment with HCl (2.2 eq. of 1.25M solution in MeOH) in 1:1 MeOH/DCM (6 ml) at 0° C. gave the title compound as a solid; MS (ES) m/z: 467.30 [MH⁺], C28H30N6O requires 466.59; ¹H-NMR (500 MHz, DMSO-d6) δ: 10.50 (bs, 1H), 8.50 (bs, 1H), 8.46 (s, 1H), 7.77 (d, 1H), 7.72 (s, 2H), 7.52 (bs, 1H), 7.40 (t, 1H), 7.35 (s, 1H), 7.26 (bs, 1H), 7.25 (d, 1H), 7.14 (s, 1H), 3.76 (d, 2H), 3.50 (d, 4H), 3.4-3.1 (bm, 8H), 2.98 (t, 2H), 2.71 (s, 3H).

### Example 85

### Ethyl 6-{2-[4-(2-methyl-5-quinolinyl)-1-piperazinyl]ethyl}imidazo[1,5-a]-quinoline-3-carboxylate dihydrochloride (E85)

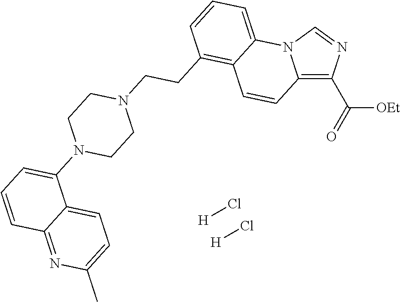

A mixture of ethyl 6-(2-oxoethyl)imidazo[1,5-a]quinoline-3-carboxylate (D91) (30 mg mg, 0.11 mmol) and 2-methyl-5-piperazin-1-yl-quinoline (WO2004/046124) (30 mg, 0.13 mmol) in 1,2-dichloroethane was stirred at room temperature for 30 min. Sodium triacetoxyborohydride (27.5 mg, 0.13 mmol) was then added and the resulting reaction mixture was stirred for 8 hours, quenched with a saturated aqueous solution of NaHCO₃ (10 ml) and extracted with ethyl acetate (3×10 ml). The combined organics were dried (Na₂SO₄) and concentrated in vacuo. The crude product was purified by SPE-Si cartridge eluting with 2% MeOH in DCM to afford the free base of the title compound as a white solid (17 mg, 31%). The free base (16 mg, 0.032 mmol) was dissolved in dry MeOH (0.5 ml) and treated with HCl (0.057 ml of a 1.25 M solution in methanol) at 0° C. The resulting suspension was stirred at room temperature for 1 hour. Evaporation of the volatiles and trituration with diethyl ether gave the title compound (17 mg, 94%) as a yellow solid; MS (ES) m/z: 494.20 [MH]⁺, C30H31N5O2 requires 493.61; ¹H-NMR (300 MHz, DMSO-d⁶) δ: 10.8 (bs, 1H), 9.3 (s, 1H), 8.6-8.4 (m, 2H), 7.9-7.8 (m, 2H), 7.9-7.6 (m, 3H), 7.6-7.4 (m, 2H), 7.25 (bs, 1H), 4.3 (q, 2H), 3.9-3.2 (bm+water, 12H), 2.8 (s, 3H), 1.35 (t, 3H).

### Example 86

### Ethyl 6-{2-[4-(2-methyl-5-quinolinyl)-1-piperidinyl]ethyl}imidazo[1,5-a]-quinoline-3-carboxylate dihydrochloride (E86)

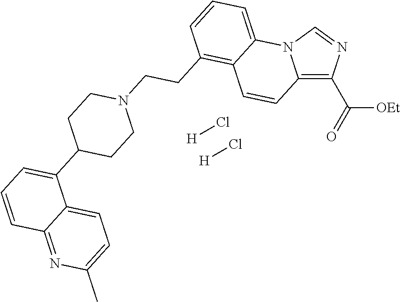

A mixture of ethyl 6-(2-oxoethyl)imidazo[1,5-a]quinoline-3-carboxylate (D91) (115 mg, 0.41 mmol), 2-methyl-5-(4-piperidinyl)quinoline (WO2004/046124) (111 mg, 0.49 mmol) in 1,2-dichloroethane (5 ml) was stirred at room temperature for 30 min. Sodium triacetoxyborohydride 104 mg, 0.49 mmol) was then added and the resulting reaction mixture was stirred for 6 hours and then concentrated in vacuo. The crude product was purified by SPE-Si cartridge eluting with 5% methanol in DCM to afford the free base of the title compound as a white solid (105 mg, 52%). The free base (15 mg, 0.03 mmol) was dissolved in dry MeOH (0.5 ml) and treated with HCl (0.054 ml of a 1.25 M solution in MeOH) at 0° C. The resulting suspension was stirred at room temperature for 1 hour. Evaporation of the volatiles and trituration with diethyl ether gave the title compound (16 mg, 94%) as a pale yellow solid; ¹H-NMR (500 MHz, DMSO-d⁶) δ: 11.12 (bs, 1H), 9.32 (s, 1H), 8.86 (bs, 1H), 8.52 (d, 1H), 8.1-8.0 (dd bm, 3H), 7.88 (bm, 1H), 7.79 (t, 1H), 7.68 (bm, 1H), 7.61 (d, 1H), 7.60 (bm, 1H), 4.38 (q, 2H), 3.81 (m, 3H), 3.63 (m, 3H), 3.3 (m+water, 4H), 2.8 (s, 3H), 2 28 (q, 2H), 2.14 (d, 2H), 1.39 (t, 3H).

### Example 87

### N-Methyl-6-{2-[4-(2-methyl-5-quinolinyl)-1-piperazinyl]ethyl}imidazo[1,5-a]quinoline-3-carboxamide dihydrochloride (E87)

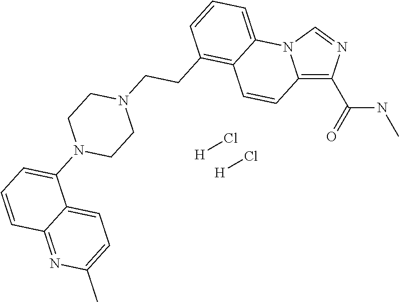

A solution of trimethylaluminium (0.19 ml of 2.0M sol. in hexanes, 0.37 mmol) and methylamine (0.19 ml of 2.0M sol. in THF, 0.37 mmol) in dry DCM (0.5 ml) was stirred at room temperature for 15 min. Ethyl 6-{2-[4-(2-methyl-5-quinolinyl)-1-piperazinyl]ethyl}imidazo[1,5-a]quinoline-3-carboxylate (free base of E85) (35 mg, 0.074 mmol) in dry DCM (1 ml) was added and stirring was continued for another 6 hours at 56° C. After reaction was completed, water was added dropwise at 0° C. followed by 1M NaOH until the organic and aqueous phase separated. The mixture was extracted with DCM (3×10 ml) and the combined organic phases were dried (Na₂SO₄) and then evaporated in vacuo. The residue was triturated with diethyl ether affording the free base of the title compound as a white solid (29 mg, 76%). The free base (22.5 mg, 0.047 mmol) was dissolved in dry MeOH (1 ml) and treated with HCl (0.083 ml of a 1.25 M solution in MeOH) at 0° C. The resulting suspension was stirred at room temperature for 1 hour. Evaporation of the volatiles and trituration with diethyl ether gave the title compound (22 mg, 85%) as a yellow solid; ¹H-NMR (500 MHz, DMSO-d⁶) δ: 11.19 (bs, 1H), 9.26 (s, 1H), 8.7 (bs, 1H), 8.47 (d, 1H), 8.23 (q, 1H), 8.13 (d, 1H), 7.87 (d, 1H), 7.9-7.6 (bm, 3H), 7.75 (t, 1H), 7.55 (d, 1H), 7.38 (bs, 1H), 3.84 (bd, 2H), 3.7-3.2 (bm+water, 10H), 2.83 (s, 3H), 2.80 (s, 3H).

### Example 88

### 6-{2-[4-(2-Methyl-5-quinolinyl)-1-piperazinyl]ethyl}imidazo[1,5-a]quinoline-3-carboxamide dihydrochloride (E88)

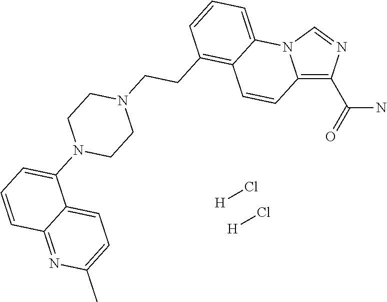

A mixture of ethyl 6-{2-[4-(2-methyl-5-quinolinyl)-1-piperazinyl]ethyl}-imidazo[1,5-a]quinoline-3-carboxylate (free base of E85) (31 mg, 0.063 mmol) and potassium hydroxide (0.4 ml, 1M solution in MeOH) was stirred at 80° C. for 2 hours. After SPE-SCX purification the ammonium 6-{2-[4-(2-methyl-5-quinolinyl)-1-piperazinyl]ethyl}imidazo[1,5-a]quinoline-3-carboxylate was isolated (0.063 mmol) and used, without further purification, to prepare the free base of the title compound following the general procedure for amide formation using hexamethyldisilazane (0.016 ml, 1.1 eq). The mixture of reaction was then evaporated in vacuo and purified by SCX. The free base (15 mg, 0.032 mmol) was dissolved in dry MeOH (0.5 ml) and treated with HCl (0.071 ml of a 1.25 M solution in ethanol) at 0° C. The resulting suspension was stirred at room temperature for 1 hour. Evaporation of the volatiles and trituration with diethyl ether gave the title compound as a yellow solid (16 mg, overall yield 47%); MS (ES) m/z: 465.2 [MH⁺], C28H28N6O requires 464.6; ¹H-NMR (500 MHz, DMSO-d⁶) δ: 11.42 (bs, 1H), 9.23 (s, 1H), 8.88 (bs, 1H), 8.46 (d, 1H), 8.1 (bd, 1H), 7.93 (bs, 2H), 7.87 (d, 1H), 7.8 (bs, 1H), 7.73 (t, 1H), 7.58 (bs, 1H), 7.54 (d, 1H), 7.45 (bs, 1H), 7.25 (bs, 1H), 3.82 (d, 2H), 3.6 (dd, 2H), 3.54 (d, 2H), 3.5-3.2 (m+water, 6H), 2.88 (s, 3H).

### Example 89

### N-Methyl-6-{2-[4-(2-methyl-5-quinolinyl)-1-piperidinyl]ethyl}imidazo[1,5-a]quinoline-3-carboxamide dihydrochloride (E89)

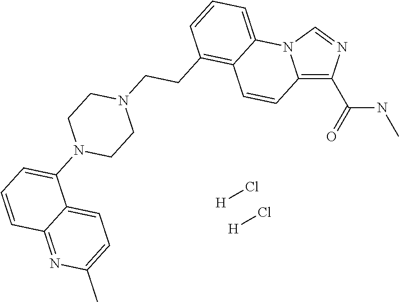

A solution of trimethylaluminium (0.1 ml of a 2.0M sol. in hexanes, 0.2 mmol) and methylamine (0.1 ml of a 2.0M sol. in THF, 0.2 mmol) in dry DCM (0.5 ml) was stirred at room temperature for 15 min. Ethyl 6-{2-[4-(2-methyl-5-quinolinyl)-1-piperidinyl]ethyl}imidazo[1,5-a]quinoline-3-carboxylate (free base of E86) (20 mg, 0.04 mmol) in dry DCM (1 ml) was added and stirring was continued for another 8 hours at 60° C. After reaction was completed, water was added dropwise at 0° C. followed by 1M NaOH until the organic and aqueous phase separated. The mixture was extracted with DCM (3×10 ml) and the combined organic phases dried (Na₂SO₄) and then evaporated in vacuo. The residue was triturated with diethyl ether affording the free base of the title compound as a white solid (19 mg, 98%). The free base (18 mg, 0.038 mmol) was dissolved in dry MeOH (0.5 ml) and treated with HCl (0.067 ml of a 1.25 M solution in MeOH) at 0° C. The resulting suspension was stirred at room temperature for 1 hour. Evaporation of the volatiles and trituration with diethyl ether gave the title compound (18 mg, 86%) as a pale yellow solid; ¹H-NMR (500 MHz, DMSO-d⁶) δ: 10.93 (bs, 1H), 9.23 (s, 1H), 8.69 (bs, 1H), 8.44 (d, 1H), 8.21 (d, 1H), 8.1 (d, 1H), 7.86 (d, 1H), 7.72 (t, 1H), 7.54 (d, 1H), 8.0-7.4 (bm, 4H), 3.82 (d, 2H), 3.74 (t, 1H), 3.58 (t, 2H), 3.5-3.2 (m+water, 4H), 2.81 (d, 3H), 2.71 (s, 3H), 2.23 (q, 2H), 2.11 (d, 2H).

### Example 90

### N-(Cyclopropylmethyl)-6-{2-[4-(2-methyl-5-quinolinyl)-1-piperidinyl]ethyl}-imidazo[1,5-a]quinoline-3-carboxamide dihydrochloride (E90)

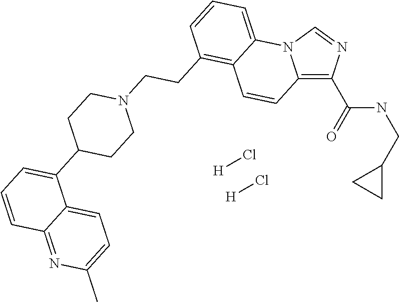

A solution of trimethylaluminium (0.15 ml of a 2.0M sol. in hexanes, 0.3 mmol) and cyclopropanemethylamine (0.021 ml, 0.3 mmol) in dry DCM (0.5 ml) was stirred at room temperature for 15 min. Ethyl 6-{2-[4-(2-methyl-5-quinolinyl)-1-piperidinyl]ethyl}imidazo[1,5-a]quinoline-3-carboxylate (free base of E86) (30 mg, 0.061 mmol) in dry DCM (1 ml), was added and stirring was continued for another 8 hours at 60° C. After reaction was completed, water was added dropwise at 0° C. followed by 1M aqueous NaOH until the organic and aqueous phase separated. The aqueous mixture was extracted with DCM (3×10 ml) and the combined organic phases dried (Na₂SO₄) and then evaporated in vacuo. The residue was triturated with diethyl ether affording the free base of the title compound as a white solid (31 mg, 100%). The free base (30 mg, 0.061 mmol) was dissolved in dry MeOH (1 ml) and treated with HCl (0.107 ml of a 1.25M solution in MeOH) at 0° C. The resulting suspension was stirred at room temperature for 1 hour. Evaporation of the volatiles and trituration with diethyl ether gave the title compound (30 mg, 83%) as a pale yellow solid; ¹H-NMR (500 MHz, DMSO-d⁶) δ: 11.04 (bs, 1H), 9.25 (s, 1H), 8.74 (bs, 1H), 8.46 (d, 1H), 8.25 (t, 1H), 8.1 (d, 1H), 7.87 (d, 1H), 8.0-7.4 (bm, 4H), 7.72 (t, 1H), 7.54 (d, 1H), 3.82 (d, 2H), 3.75 (t, 1H), 3.59 (m, 2H), 3.5-3.1 (bm+water, 4H), 3.17 (t, 2H), 2.73 (s, 3H), 2.25 (q, 2H), 2.11 (d, 2H), 1.08 (m, 1H), 0.42 (d, 2H), 0.26 (d, 2H).

### Example 91

### 6-{2-[4-(2-Methyl-5-quinolinyl)-1-piperidinyl]ethyl}imidazo[1,5-a]quinoline-3-carboxamide dihydrochloride (E91)

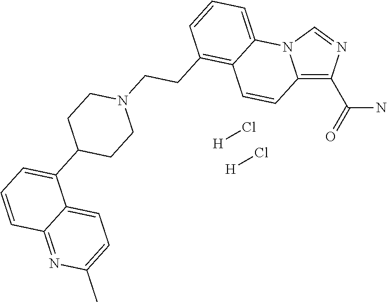

A mixture of ethyl-6-{2-[4-(2-methyl-5-quinolinyl)-1-piperidinyl]ethyl}imidazo-[1,5-a]quinoline-3-carboxylate (free base of E86) (40 mg, 0.081 mmol) and potassium hydroxide (0.49 ml of 1M solution in MeOH) was stirred at 80° C. for 2 hours. After SCX purification the ammonium 6-{2-[4-(2-methyl-5-quinolinyl)-1-piperidinyl]ethyl}imidazo[1,5-a]quinoline-3-carboxylate was isolated and used, without further purification, to prepare the free base of the title compound following the general procedure for amide formation using hexamethyldisilazane (0.021 ml, 1.1 eq). The reaction mixture was then evaporated in vacuo and purified by SCX to afford the title compound as a free base (20 mg, 0.043 mmol) which was dissolved in dry MeOH (0.5 ml) and treated with HCl (0.076 ml of a 1.25 M solution in ethanol) at 0° C. The resulting suspension was stirred at room temperature for 1 hour. Evaporation of the volatiles and trituration with diethyl ether gave the title compound as a pale yellow solid (20 mg, overall yield 46%); MS (ES) m/z: 464.2 [MH⁺], C29H29N5O requires 463.6; ¹H-NMR (500 MHz, DMSO-d⁶) δ: 11.43 (bs, 1H), 9.3 (bs, 1H), 9.22 (s, 1H), 8.45 (d, 1H), 8.26 (bd, 1H), 8.08 (d, 2H), 7.96 (bd, 1H), 7.91 (d, 1H), 7.73 (m, 2H), 7.58 (bs, 1H), 7.55 (d, 1H), 7.25 (bs, 1H), 3.82 (d, 2H), 3.61 (dd, 2H), 3.6-3.2 (m+water, 5H), 2.96 (s, 3H), 2.32 (q, 2H), 2.11 (d, 2H).

### Example 92

### 6-{2-[4-(2-Methyl-5-quinolinyl)-1-piperazinyl]ethyl}tetrazolo[1,5-a]quinoline dihydrochloride (E92)

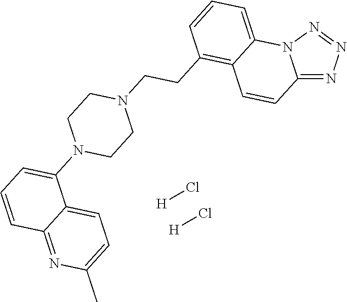

A mixture of tetrazolo[1,5-a]quinolin-6-ylacetaldehyde (D93) (50 mg, 0.24 mmol), 2-methyl-5-piperazin-1-yl-quinoline (WO2004/046124) (60 mg, 0.29 mmol) in 1,2-dichloroethane (3 ml) was stirred at room temperature for 30 min. Sodium triacetoxyborohydride (61 mg, 0.29 mmol) was then added and the resulting reaction mixture was stirred overnight and then concentrated in vacuo. The residue was purified by SPE-Si cartridge eluting with DCM to 4% MeOH in DCM to afford the free base of the title compound as a white solid (83 mg, 81%). The free base (80 mg, 0.19 mmol) was dissolved in dry MeOH (1.5 ml) and treated with HCl (0.33 ml of a 1.25 M solution in ethanol) at 0° C. The resulting suspension was stirred at room temperature for 4 hours. Evaporation of the volatiles and trituration with diethyl ether gave the title compound (82 mg, 87%) as a yellow solid; MS (ES) m/z: 424.0 [MH⁺], C25H25N7 requires 423.5; ¹H-NMR (500 MHz, DMSO-d⁶) δ: 11.88 (bs, 1H), 8.92 (bs, 1H), 8.71 (d, 1H), 8.63 (d, 1H), 8.2o (d, 1H), 8.0 (t, 1H), 8.0 (m, 2H), 7.8 (d, 1H), 7.8 (m, 1H), 7.47 (bd, 1H), 3.83 (d, 2H), 3.77 (dd, 1H), 3.7-3.3 (bm+water, 8H), 2.91 (s, 3H).

### Example 93

### 6-{2-[4-(2-Methyl-5-quinolinyl)-1-piperidinyl]ethyl}tetrazolo[1,5-a]quinoline (E93)

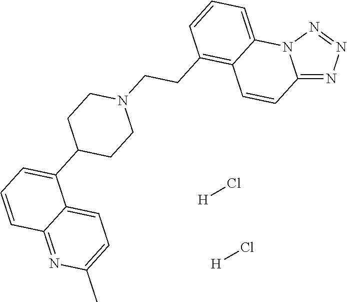

A mixture of tetrazolo[1,5-a]quinolin-6-ylacetaldehyde (D93) (50 mg, 0.24 mmol), 2-methyl-5-(4-piperidinyl)quinoline (WO2004/046124) (66 mg, 0.29 mmol) in 1,2-dichloroethane (3 ml) was stirred at room temperature for 30 min. Sodium triacetoxyborohydride (61 mg, 0.29 mmol) was then added and the resulting reaction mixture was stirred overnight and then concentrated in vacuo. The residue was purified by SPE-Si cartridge eluting with DCM to 4% MeOH in DCM to afford the free base of the title compound as a white solid (66 mg, 65%). The free base (60 mg, 0.14 mmol) was dissolved in dry WON (1.5 ml) and treated with HCl (0.25 ml of a 1.25 M solution in ethanol) at 0° C. The resulting suspension was stirred at room temperature for 4 hours. Evaporation of the volatiles and trituration with diethyl ether gave the title compound (62 mg, 89%) as a pale yellow solid; MS (ES) m/z: 423.0 [MH]⁺, C26H26N6 requires 422.6; ¹H-NMR (500 MHz, DMSO-d⁶) δ: 11.14 (bs, 1H), 8.84 (bs, 1H), 8.68 (d, 1H), 8.63 (d, 1H), 8.20 (d, 1H), 8.0 (t, 1H), 8.1-7.8 (bm, 2H), 7.82 (d, 1H), 7.69 (bs, 1H), 7.56 (bs, 1H), 3.83 (d, 2H), 3.74 (dd, 2H), 3.5-3.2 (bm+water, 5H), 2.78 (s, 3H), 2.26 (q, 2H), 2.12 (d, 2H).

### Example 94

### 6-{2-[(2R)-2-Methyl-4-(2-methyl-5-quinolinyl)-1-piperazinyl]ethyl}tetrazolo[1,5-a]quinoline dihydrochloride (E94)

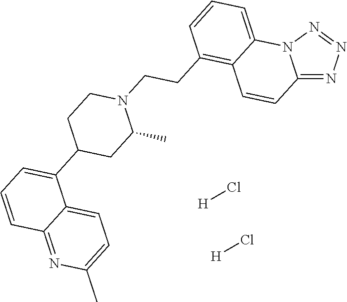

A mixture of tetrazolo[1,5-a]quinolin-6-ylacetaldehyde (D93) (50 mg, 0.24 mmol), 2-methyl-5-[(3R)-3-methyl-1-piperazinyl]quinoline (WO2004/046124) (70 mg, 0.29 mmol) in 1,2-dichloroethane (3 ml) was stirred at room temperature for 30 min. Sodium triacetoxyborohydride (61 mg, 0.29 mmol) was then added and the resulting reaction mixture was stirred overnight and then concentrated in vacuo. The residue was purified by SPE-Si cartridge eluting with DCM to 4% MeOH in DCM to afford the free base of the title compound as a white solid (64 mg, 61%). The free base (60 mg, 0.14 mmol) was dissolved in dry MeOH (1.5 ml) and treated with HCl (0.24 ml of a 1.25 M solution in ethanol) at 0° C. The resulting suspension was stirred at room temperature for 4 hours. Evaporation of the volatiles and trituration with diethyl ether gave the title compound (64 mg, 90%) as a yellow solid; MS (ES) m/z: 438.0 [MH]⁺, C26H27N7 requires 437.6; ¹H-NMR (500 MHz, DMSO-d⁶) δ: 11.70 (bs, 1H), 8.8 (bs, 1H), 8.69 (d, 1H), 8.63 (d, 1H), 8.20 (d, 1H), 8.0 (t, 1H), 7.9-7.8 (bs, 2H), 7.85 (d, 1H), 7.69 (bs, 1H), 7.38 (bs, 1H), 3.96 (d, 2H), 3.9-3.1 (m+water, 8H), 3.82 (m, 1H), 2.82 (s, 3H), 1.47 (d, 3H).

### Example 95

### 6-(2-{4-[2-(Difluoromethyl)-5-quinolinyl]-1-piperazinyl}ethyl)tetrazolo[1,5-a]quinoline dihydrochloride (E95)

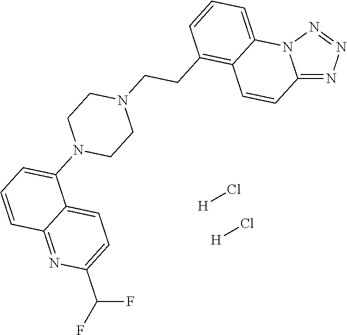

A mixture of tetrazolo[1,5-a]quinolin-6-ylacetaldehyde (D93) (50 mg, 0.24 mmol), 2-(difluoromethyl)-5-(1-piperazinyl)quinoline (D143) (76 mg, 0.29 mmol) in 1,2-dichloroethane (3 ml) was stirred at room temperature for 30 min. Sodium triacetoxyborohydride (61 mg, 0.29 mmol) was then added and the resulting reaction mixture was stirred overnight and then concentrated in vacuo. The residue was purified by SPE-Si cartridge eluting with DCM to 4% MeOH in DCM to afford the free base of the title compound as a white solid (48 mg, 44%). The free base (45 mg, 0.098 mmol) was dissolved in dry MeOH (1.5 ml) and treated with HCl (0.172 ml of a 1.25 M solution in ethanol) at 0° C. The resulting suspension was stirred at room temperature for 4 h. Evaporation of the volatiles and trituration with diethyl ether gave the title compound (46 mg, 83%) as a white solid; MS (ES) m/z: 460.0 [MH⁺], C25H23F2N7 requires 459.5; ¹H-NMR (500 MHz, DMSO-d⁶) δ: 10.88 (bs, 1H), 8.75 (d, 1H), 8.64 (m, 2H), 8.24 (d, 1H), 8.01 (t, 1H), 7.9-7.6 (m, 4H), 7.43 (d, 1H), 7.13 (t, 1H), 3.84 (d, 2H), 3.71 (m, 2H), 3.55 (m, 6H), 3.3 (m+water, 2H).

### Example 96

### 1-Methyl-6-{2-[4-(2-methyl-5-quinolinyl)-1-piperazinyl]ethyl}-[1,2,4]triazolo[4,3-a]quinoline dihydrochloride (E96)

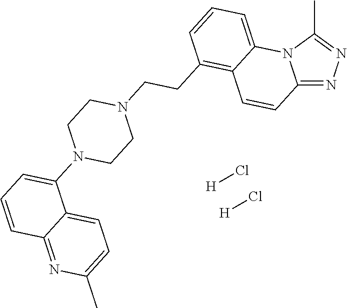

A mixture of (1-methyl[1,2,4]triazolo[4,3-a]quinolin-6-yl)acetaldehyde and 1-(methyloxy)-2-(1-methyl[1,2,4]triazolo[4,3-a]quinolin-6-yl)ethanol (see D97) (30 mg, 0.13 mmol), 2-methyl-5-piperazin-1-yl-quinoline (WO2004/046124) (36 mg, 0.16 mmol) and a drop of glacial acetic acid in a 3:1 mixture of 1,2-dichloroethane/acetonitrile (4 ml) was stirred at room temperature under nitrogen for 30 min. Sodium triacetoxyborohydride (34 mg, 0.16 mmol) was then added and the resulting reaction mixture was stirred for 6 hours, quenched with a saturated aqueous solution of NaHCO₃ (10 ml) and extracted with DCM (3×10 ml). The combined organics were dried (Na₂SO₄) and concentrated in vacuo. The crude product was purified by SPE cartridge (silica gel) eluting with 5% MeOH in DCM to afford the free base of the title compound as a white solid (32 mg, 55%). The free base (30 mg, 0.07 mmol) was dissolved in dry MeOH (2 ml) and treated with HCl (0.123 ml of a 1.25 M solution in MeOH, 0.15 mmol) at 0° C. The resulting suspension was stirred at room temperature for 1 hour. Evaporation of the volatiles and trituration with diethyl ether gave the title compound (30 mg, 87%) as a yellow solid; ¹H-NMR (500 MHz, DMSO-d⁶) δ: 11.4 (bs, 1H), 8.53 (bs, 1H), 8.36 (d, 1H), 8.11 (d, 1H), 7.75 (m, 4H), 7.59 (d, 1H), 7.54 (bs, 1H), 7.29 (bs, 1H), 3.82 (m, 2H), 3.64 (m, 2H), 3.52 (m, 6H), 3.2 (m, 2H), 3.09 (s, 3H), 2.72 (bs, 3H).

### Example 97

### Ethyl 6-{2-[4-(2-methyl-5-quinolinyl)-1-piperazinyl]ethyl}[1,2,3]triazolo[1,5-a]quinoline-3-carboxylate dihydrochloride (E97)

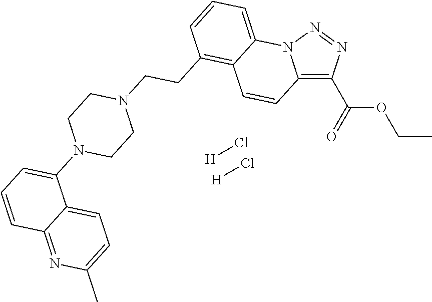

A mixture of ethyl 6-(2-oxoethyl)[1,2,3]triazolo[1,5-a]quinoline-3-carboxylate (D99) (36 mg, 0.13 mmol), 2-methyl-5-piperazin-1-yl-quinoline (36 mg, 0.16 mmol) in 1,2-dichloroethane (2 ml) was stirred at room temperature for 30 min. Sodium triacetoxyborohydride (34 mg, 0.16 mmol) was then added and the resulting reaction mixture was stirred overnight and then concentrated in vacuo. The crude product was purified by SPE-Si cartridge eluting with 30% cyclohexane in ethyl acetate followed by 2% MeOH in DCM to afford the free base of the title compound (60 mg, 93%). A portion of this material (15 mg) was dissolved in dry MeOH (1 ml) and treated with HCl (0.055 ml of a 1.25 M solution in ethanol) at 0° C. The resulting suspension was stirred at room temperature for 1 hour. Evaporation of the volatiles and trituration with diethyl ether gave the title compound as a yellow solid; ¹H-NMR (500 MHz, DMSO-d⁶) δ: 11.64 (bs, 1H), 8.92 (bs, 1H), 8.77 (d, 1H), 8.58 (d, 1H), 8.09 (d, 1H), 7.98 (t, 1H), 7.95 (bs, 2H), 7.82 (d, 1H), 7.47 (bs, 1H), 3.83 (d, 2H), 3.74 (dd, 1H), 3.7-3.5 (bm+water, 6H), 3.39 (t, 2H), 2.90 (s, 3H), 1.41 (t, 3H).

### Example 98

### 6-{2-[4-(2-Methyl-5-quinolinyl)-1-piperazinyl]ethyl}[1,2,3]triazolo[1,5-a]quinoline-3-Carboxamide dihydrochloride (E98)

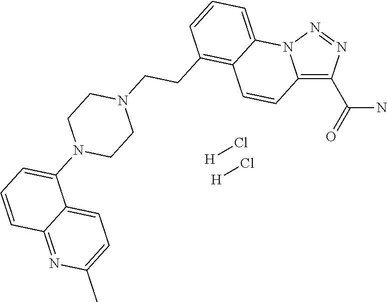

A mixture of ethyl 6-{2-[4-(2-methyl-5-quinolinyl)-1-piperazinyl]ethyl}-[1,2,3]triazolo[1,5-a]quinoline-3-carboxylate (free base of E97) (25 mg, 0.081 mmol) and potassium hydroxide (0.3 ml of a 1M sol. in MeOH) was stirred at 80° C. for 2 hours. After SCX purification the ammonium 6-{2-[4-(2-methyl-5-quinolinyl)-1-piperazinyl]ethyl}[1,2,3]triazolo[1,5-a]quinoline-3-carboxylate was isolated and used, without further purification, to prepare the free base of the title compound following the general procedure for amide formation using hexamethyldisilazane (0.009 ml, 1.1 eq). The reaction mixture was then evaporated in vacuo and purified by SCX followed by SPE-Si eluting with 2% MeOH in DCM. The free base (9 mg, 0.019 mmol) was dissolved in dry MeOH (0.5 ml) and treated with HCl (0.034 ml of a 1.25 M solution in ethanol) at 0° C. The resulting suspension was stirred at room temperature for 1 hour. Evaporation of the volatiles and trituration with diethyl ether gave the title compound as yellow solid (10 mg, overall yield 23%); ¹H-NMR (500 MHz, DMSO-d⁶) δ: 11.76 (bs, 1H), 8.75 (d, 1H), 8.55 (bs, 1H), 8.36 (d, 1H), 8.21 (d, 1H), 8.2 (bs, 2H), 7.96 (t, 1H), 7.77 (d, 1H), 7.74 (bs, 2H), 7.69 (s, 1H), 7.56 (bs, 1H), 7.31 (bs, 1H), 3.84 (d, 2H), 3.68 (m, 2H), 3.56 (m+water, 5H), 3.3 (m, 2H), 2.73 (s, 3H).

### Example 99

### Ethyl 6-{2-[4-(2-methyl-5-quinolinyl)-1-piperidinyl]ethyl}[1,2,3]triazolo[1,5-a]quinoline-3-carboxylate dihydrochloride (E99)

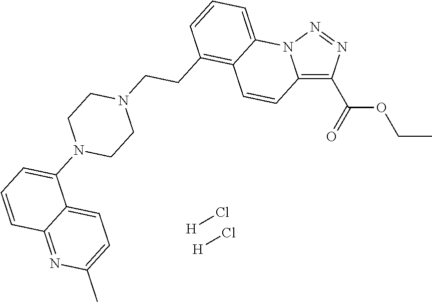

A mixture of ethyl 6-(2-oxoethyl)[1,2,3]triazolo[1,5-a]quinoline-3-carboxylate (D99) (41 mg, 0.14 mmol) and 2-methyl-5-(4-piperidinyl)quinoline (39 mg, 0.17 mmol) (WO2004/046124) in 1,2-dichloroethane (2 ml) was stirred at room temperature under nitrogen for 30 minutes. Sodium triacetoxyborohydride (36 mg, 0.17 mmol) was then added, the resulting reaction mixture was stirred for 6 hours and then concentrated in vacuo. The crude product was purified by SPE-Si cartridge eluting with 20% cyclohexane in ethyl acetate to afford the free base of the title compound (65 mg, 94%). Free base (15 mg) was dissolved in dry methanol (0.5 ml) and treated with HCl (0.053 ml of a 1.25 M solution in methanol) at 0° C. The resulting suspension was stirred at room temperature for 2 hours. Evaporation of the volatiles and trituration with diethyl ether gave the title compound (16 mg, 93%); ¹H-NMR (500 MHz, DMSO-d⁶) δ: 10.58 (bs, 1H), 8.76 (d, 1H), 8.58 (d, 1H), 8.49 (d, 1H), 8.10 (d, 1H), 7.97 (t, 1H), 7.84 (d, 1H), 7.80 (d, 1H), 7.72 (t, 1H), 7.49 (d, 1H), 7.44 (d, 1H), 4.43 (q, 2H), 3.84 (d, 2H), 3.71 (m, 3H), 3.45 (m, 2H), 3.33 (m, 2H), 2.67 (s, 3H), 2.18 (m, 4H), 1.41 (t, 3H).

### Example 100

### Ethyl 6-{2-[(2R)-2-methyl-4-(2-methyl-5-quinolinyl-1-piperazinyl]ethyl}-[1,2,3]triazolo[1,5-a]quinoline-3-carboxylate dihydrochloride (E100)

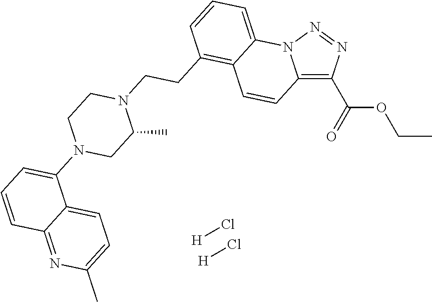

A mixture of ethyl 6-(2-oxoethyl)[1,2,3]triazolo[1,5-a]quinoline-3-carboxylate (D99) (41 mg, 0.14 mmol), 2-methyl-5-[(3R)-3-methyl-1-piperazinyl]quinoline (WO2004/046124) (41 mg, 0.17 mmol) in 1,2-dichloroethane (2 ml) was stirred at room temperature under nitrogen for 30 minutes. Sodium triacetoxyborohydride (36 mg, 0.17 mmol) was then added and the resulting reaction mixture was stirred 6 hours and then concentrated in vacuo. The crude product was purified by SPE-Si cartridge eluting with 20% cyclohexane in ethyl acetate to afford the free base of the title compound (55 mg, 77%). Free base (15 mg) was dissolved in dry methanol (0.5 ml) and treated with HCl (0.052 ml of a 1.25 M solution in methanol) at 0° C. The resulting suspension was stirred at room temperature for 2 hours. Evaporation of the volatiles and trituration with diethyl ether gave the title compound (15 mg, 90%); ¹H-NMR (500 MHz, DMSO-d⁶) δ: 10.94 (bs, 1H), 8.77 (d, 1H), 8.52 (d, 1H), 8.45 (d, 1H), 8.12 (d, 1H), 7.98 (t, 1H), 7.83 (d, 1H), 7.67 (m, 2H), 7.45 (d, 1H), 7.23 (bs, 1H), 4.45 (q, 2H), 3.83 (m, 1H), 3.69 (m, 4H), 3.49 (m, 3H), 3.30 (m, 2H), 3.08 (m, 1H), 2.66 (s, 3H), 1.46 (d, 3H), 1.41 (t, 3H).

### Example 101

### Ethyl 6-{2-[4-(2-methyl-5-quinolinyl)-1-piperidinyl]ethyl}-[1,2,3]triazolo[1,5-a]quinoline-3-carboxylate dihydrochloride (E101)

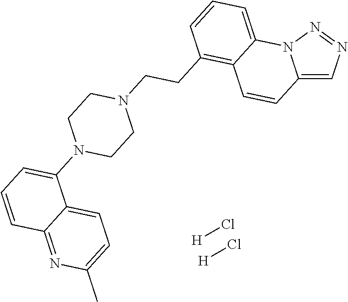

A mixture of ammonium 6-{2-[4-(2-methyl-5-quinolinyl)-1-piperidinyl]ethyl}-[1,2,3]triazolo[1,5-a]quinoline-3-carboxylate (E181) (20 mg) in 1,2-dichlorobenzene (1 ml) was irradiated in a microwave reactor (PersonalChemistry Emrys™ Optimiser, 300W, 250° C., 10 minutes). The solvent was removed by SPE-SCX cartridge (eluting with methanol followed by 2N ammonia solution in methanol) to afford the free base of title compound as a white solid (12.3 mg). The free base was dissolved in dry methanol (0.5 ml) and treated with HCl (0.073 ml of a 1.25 M solution in methanol) at 0° C. The resulting suspension was stirred at room temperature for 2 hours. Evaporation of the volatiles and trituration with diethyl ether gave the title compound (14 mg, 96%); ¹H-NMR (500 MHz, DMSO-d⁶) δ: 10.52 (bs, 1H), 8.69 (d, 1H), 8.6 (bs, 1H), 8.34 (s, 1H), 8.13 (d, 1H), 7.95 (d, 1H), 7.87 (m, 2H), 7.74 (m, 2H), 7.52 (bs, 1H), 7.46 (d, 1H), 3.83 (d, 1H), 3.74 (d, 2H), 3.64 (m, 2H), 3.43 (m, 2H), 3.35 (m, 2H), 2.69 (s, 3H), 2.16 (m, 4H).

### Example 102

### Methyl 6-{2-[4-(2-methyl-5-quinolinyl)-1-piperidinyl]ethyl}-[1,2,3]triazolo-[1,5-a]quinoline-3-carboxylate (E102)

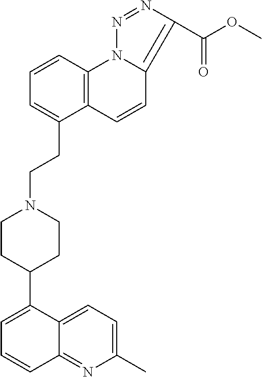

Methyl 6-(2-oxoethyl)[1,2,3]triazolo[1,5-a]quinoline-3-carboxylate (D104) (355 mg, 1.32 mmol) and 2-methyl-5-(4-piperidinyl)quinoline (328 mg, 1.45 mmol) (WO2004/046124) in 1,2-dichloroethane (15 ml) was stirred at room temperature under nitrogen for 30 minutes. Sodium triacetoxyborohydride (306 mg, 1.45 mmol) was then added, the resulting reaction mixture was stirred over night and then it was quenched with NaHCO₃ (100 ml) and extracted with DCM (3×100 ml). The organic layers were combined, dried (Na₂SO₄) and concentrated in vacuo. The crude material was purified by SPE-Si cartridge eluting with 2% MeOH in DCM to afford the title compound as a white solid (501 mg, 79%); MS (ES) m/z: 480.3 [MH⁺], C29H29N5O2 requires 479.6.

### Example 103

### 6-{2-[(2R)-2-Methyl-4-(2-methyl-5-quinolinyl)-1-piperazinyl]ethyl}[1,2,3]-triazolo[1,5-a]quinoline-3-carboxamide dihydrochloride (E103)

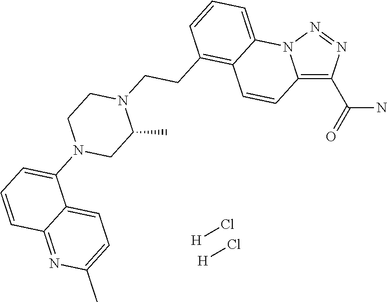

A mixture of ethyl 6-(2-[(2R)-2-methyl-4-(2-methyl-5-quinolinyl)-1-piperazinyl]-ethyl)[1,2,3]triazolo[1,5-a]quinoline-3-carboxylate (free base of E100) (40 mg, 0.078 mmol) and KOH (1 M solution in MeOH, 0.5 ml) was stirred at reflux for 2 hours. The solution was cooled to room temperature and the precipitate was dissolved adding H₂O and then the solution was purified by SPE-SCX cartridge (eluting with methanol followed by 2N ammonia solution in methanol) affording 38 mg of ammonium 6-{2-[(2R)-2-methyl-4-(2-methyl-5-quinolinyl)-1-piperazinyl]ethyl}[1,2,3]triazolo[1,5-a]quinoline-3-carboxylate. 20 mg (0.04 mmol) of this intermediate were used, without further purification, to prepare the free base of the title compound following the general procedure for amide formation using hexamethyldisilazane (0.01 ml, 1.1 eq) (see Example 14). The reaction mixture was then evaporated in vacuo and purified by SCX. The free base (18 mg, 0.038 mmol) was dissolved in dry MeOH (0.5 ml) and treated with HCl (0.083 ml of a 1.25 M solution in ethanol) at 0° C. The resulting suspension was stirred at room temperature for 1 hour. Evaporation of the volatiles and trituration with diethyl ether gave the title compound as a yellow solid (20 mg, 90%); ¹H-NMR (500 MHz, DMSO-d⁶) δ: 10.56 (bs, 1H), 8.74 (d, 1H), 8.48 (bs, 1H), 8.36 (m, 2H), 8.2 (m, 2H), 7.95 (t, 1H), 7.8 (d, 1H), 7.68 (m, 2H), 7.48 (d, 1H), 7.25 (d, 1H), 3.86 (m, 1H), 3.61 (m, 9H), 3.06 (t, 1H), 2.68 (s, 3H), 1.44 (d, 3H).

### Example 104

### N-Methyl-6-{2-[(2R)-2-methyl-4-(2-methyl-5-quinolinyl)-1-piperazinyl]-ethyl}imidazo[1,5-a]quinoline-3-carboxamide dihydrochloride (E104)

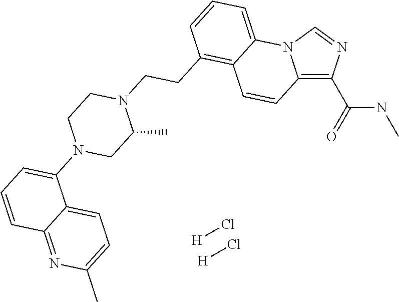

A solution of trimethylaluminium (0.113 ml of 2.0M sol. in hexanes, 0.226 mmol) and methylamine (0.113 ml of 2.0M sol. in THF, 0.226 mmol) in dry DCM (0.5 ml) was stirred at room temperature for 15 minutes. Ethyl 6-{2-[(2R)-2-methyl-4-(2-methyl-5-quinolinyl)-1-piperazinyl]ethyl}imidazo[1,5-a]quinoline-3-carboxylate (free base of E55) (20.7 mg, 0.041 mmol) was added and stirring was continued for 8 hours at 56° C. After reaction was completed, water was added dropwise at 0° C. followed by 1M NaOH until the organic and aqueous phase separated. The mixture was extracted with DCM (3×10 ml) and the combined organic phases were dried (Na₂SO₄) and then evaporated in vacuo. The residue was purified by chromatography eluting with 0.25% of NH₃ (2 M in MeOH) in DCM affording the free base of the title compound as a white solid (13 mg, 64%). This material was dissolved in a mixture MeOH:DCM (1:1, 0.5 ml) and treated with HCl (0.047 ml of a 1.25 M solution in MeOH) at 0° C. The resulting suspension was stirred at room temperature for 1 hour. Evaporation of the volatiles and titration with diethyl ether gave the title compound as a yellow solid (13 mg); ¹H-NMR (500 MHz, DMSO-d⁶) δ: 10.8 (bs, 1H), 9.25 (s, 1H), 8.55 (m, 1H), 8.46 (d, 1H), 8.22 (d, 1H), 8.12 (d, 1H), 7.82 (d, 1H), 7.72 (bm, 3H), 7.58 (d, 1H), 7.51 (bs, 1H), 7.27 (bs, 1H), 3.94 (d, 1H), 3.83 (m, 1H), 3.73 (m, 1H), 3.42 (m, 7H), 3.08 (m, 1H), 2.81 (s, 3H), 2.7 (s, 3H), 1.45 (d, 3H).

### Example 105

### 6-{2-[(2R)-2-Methyl-4-(2-methyl-5-quinolinyl)-1-piperazinyl]ethyl}-3-(3-methyl-1,2,4-oxadiazol-5-yl)imidazo[1,5-a]quinoline dihydrochloride (E105)

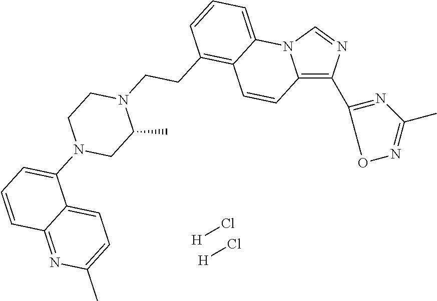

Methyl carboxyamide oxime (4 mg, 0.054 mmol) was added to a suspension of sodium hydride (2.1 mg of 60% suspension in oil, 0.054 mmol) in dry THF (1 ml) followed after 10 minutes by the addition of ethyl 6-{2-[(2R)-2-methyl-4-(2-methyl-5-quinolinyl)-1-piperazinyl]ethyl}imidazo[1,5-a]quinoline-3-carboxylate (free base of E55) (25 mg, 0.049 mmol). After 30 minutes DMF (0.2 ml) was added and the reaction was stirred at room temperature overnight. The conversion of the starting material was not complete so NaH (4.2 mg, 0.108 mmol) and methyl carboxyamide oxime (8 mg, 0.108 mmol) were added and the reaction was stirred for additional 3 hours. The reaction mixture was quenched with water (5 ml) and extracted with ethyl acetate (3×5 ml). After drying and evaporation of the organic solvents, the crude material was purified by chromatography eluting with 0.25% of NH₃ (2 M in MeOH) in DCM affording the free base of the title compound as a white solid (11 mg). This material was dissolved in a mixture 1:1=MeOH:DCM (0.5 ml) and treated with HCl (0.038 ml of a 1.25 M solution in MeOH) at 0° C. The resulting suspension was stirred at room temperature for 1 hour. Evaporation of the volatiles and trituration with diethyl ether gave the title compound as a yellow solid (11 mg); 1H NMR (500 MHz, DMSO-d₆) δ ppm 1.44 (d, 3H) 2.69 (s, 3H) 3.08 (t, 1H) 3.19-3.49 (m, 5H) 3.52 (d, 2H) 3.61 (s, 3H) 3.69-3.79 (m, 1H) 3.79-3.88 (m, 1H) 3.95 (d, 1H) 7.26 (br. s., 1H) 7.49 (br. s., 1H) 7.64 (d, 1H) 7.67-7.74 (m, 2H) 7.82 (t, 1H) 8.08 (d, 1H) 8.14 (d, 1H) 8.49 (br. s., 1H) 8.55 (d, 1H) 9.43-9.51 (m, 1H) 10.75 (br. s., 1H).

### Example 106

### 6-{2-[(2R)-2-Methyl-4-(2-methyl-5-quinolinyl)-1-piperazinyl]ethyl}-imidazo[1,5-a]quinoline-3-carboxamide dihydrochloride (E106)

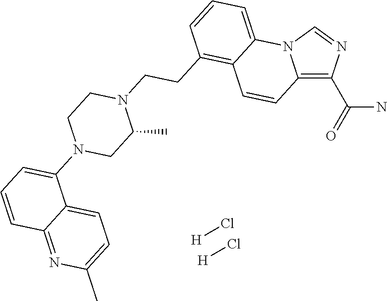

A mixture of ethyl 6-{2-[(2R)-2-methyl-4-(2-methyl-5-quinolinyl)-1-piperazinyl]ethyl}imidazo[1,5-a]quinoline-3-carboxylate (free base of E55) (28 mg, 0.055 mmol) and potassium hydroxide (0.33 ml of a 1M sol. in MeOH) was stirred at 80° C. for 3 hours. After SCX purification the ammonium 6-{2-[(2R)-2-methyl-4-(2-methyl-5-quinolinyl)-1-piperazinyl]ethyl}imidazo[1,5-a]quinoline-3-carboxylate was isolated and used, without further purification, to prepare the free base of the title compound following the general procedure for amide formation using hexamethyldisilazane (0.014 ml, 1.1 eq) (see Example 14). The reaction mixture was then evaporated in vacuo and purified by SCX followed by SPE-Si eluting with 2% MeOH in DCM. The free base (10 mg, 0.021 mmol) was dissolved in dry 1:1 MeOH:DCM (0.5 ml) and treated with HCl (0.037 ml of a 1.25 M solution in ethanol) at 0° C. The resulting suspension was stirred at room temperature for 1 hour. Evaporation of the volatiles and trituration with diethyl ether gave the title compound as yellow solid; ¹H NMR (500 MHz, DMSO-d₆) δ ppm 1.44 (d, 3H) 2.77 (br. s., 3H) 3.00-3.16 (m, 2H) 3.19-3.66 (m, 6H) 3.69-3.80 (m, 1H) 3.80-3.91 (m, 1H) 3.91-4.00 (m, 1H) 7.26 (br. s., 1H) 7.33 (br. s., 1H) 7.48-7.65 (m, 3H) 7.80 (br. s., 2H) 7.71-7.79 (m, 1H) 7.82 (d, 1H) 8.12 (d, 1H) 8.64 (br. s., 1H) 8.47 (d, 1H) 9.15-9.33 (m, 1H) 10.71 (br. s., 1H).

### Example 107

### Ethyl 1-methyl-6-{2-[4-(2-methyl-5-quinolinyl)-1-piperidinyl]ethyl}imidazo[1,5-a]quinoline-3-carboxylate dihydrochloride (E107)

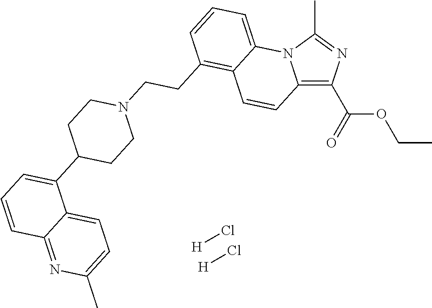

A mixture ethyl 1-methyl-6-(2-oxoethyl)imidazo[1,5-a]quinoline-3-carboxylate (D106) (85 mg, 0.29 mmol), 2-methyl-5-(4-piperidinyl)quinoline (77 mg, 0.34 mmol) (WO2004/046124) in 1,2-dichloroethane (5 ml) was stirred at room temperature for 30 minutes. Sodium triacetoxyborohydride (34 mg, 0.16 mmol) was then added and the resulting reaction mixture was stirred overnight and then concentrated in vacuo. The crude product was purified by SPE-Si cartridge eluting with 2% MeOH in DCM to afford the free base of the title compound (70 mg, 48%). A portion of this material (10 mg) was dissolved in dry MeOH (0.3 ml) and treated with HCl (0.035 ml of a 1.25 M solution in ethanol) at 0° C. The resulting suspension was stirred at room temperature for 1 hour. Evaporation of the volatiles and trituration with diethyl ether gave the title compound E107 as a white solid (10 mg, 91%); ¹H NMR (500 MHz, DMSO-d₆) δ ppm 1.36 (t, 81-1) 2.06-2.17 (m, 2H) 2.16-2.33 (m, 2H) 2.81 (s, 3H) 3.07 (s, 3H) 3.24-3.34 (m, 2H) 3.32-3.41 (m, 2H) 3.56-3.67 (m, 2H) 3.73-3.89 (m, 3H) 4.27-4.39 (m, 2H) 7.54-7.63 (m, 2H) 7.73 (t, 1H) 7.77-7.94 (m, 2H) 7.93-8.00 (m, 1H) 7.99-8.07 (m, 2H) 8.43 (d, 1H) 8.91 (br. s., 1H) 10.93 (br. s., 1H).

### Example 108

### 1-Methyl-6-{2-[4-(2-methyl-5-quinolinyl)-1-piperidinyl]ethyl}imidazo[1,5-a]quinoline-3-carboxamide dihydrochloride (E108)

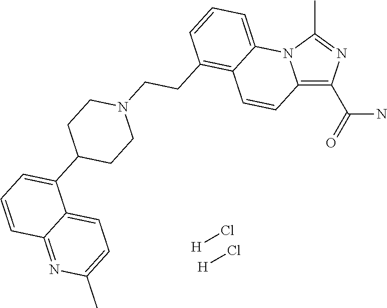

A mixture of Ethyl 1-methyl-6-{2-[4-(2-methyl-5-quinolinyl)-1-piperidinyl]ethyl}imidazo[1,5-a]quinoline-3-carboxylate (free base of E107) (55 mg, 0.108 mmol) and potassium hydroxide (0.65 ml of a 1M sol. in MeOH) was stirred at 80° C. for 3 hours. After SPE-SCX purification the ammonium 6-{2-[(2R)-2-methyl-4-(2-methyl-5-quinolinyl)-1-piperazinyl]ethyl}imidazo[1,5-a]quinoline-3-carboxylate was isolated and used, without further purification, to prepare the free base of the title compound following the general procedure for amide formation using hexamethyldisilazane (0.097 ml, 1.2 eq). The reaction mixture was then evaporated in vacuo and purified by SCX followed by SPE-Si eluting with 2% MeOH in DCM to 5% MeOH in DCM (20 mg, 39%). This material was dissolved in dry MeOH (0.5 ml) and treated with HCl (0.075 ml of a 1.25 M solution in ethanol) at 0° C. The resulting suspension was stirred at room temperature for 1 hour. Evaporation of the volatiles and trituration with diethyl ether gave the title compound as white solid (18 mg, 82%); ¹H NMR (500 MHz, DMSO-d₆) δ ppm 2.05-2.18 (m, 2H) 2.20-2.32 (m, 2H) 2.84 (s, 3H) 3.06 (s, 3H) 3.24-3.51 (m, 4H) 3.53-3.64 (m, 2H) 3.70-3.90 (m, 3H) 7.14-7.25 (m, 1H) 7.43-7.52 (m, 1H) 7.54 (d, 1H) 7.57-7.66 (m, 1H) 7.69 (t, 1H) 7.72-7.86 (m, 2H) 7.84-8.00 (m, 1H) 8.00-8.12 (m, 1H) 8.15 (d, 1H) 8.38 (d, 1H) 8.63-9.35 (m, 1H) 11.07 (br. s., 1H)

### Example 109

### 6-{2-[4-(2-Methyl-5-quinolinyl)-1-piperidinyl]ethyl}[1,2,3]triazolo[1,5-a]quinoline-3-carboxamide dihydrochloride (E109)

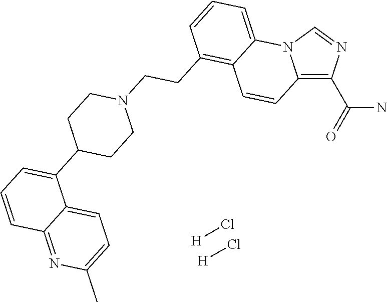

Methyl 6-{2-[4-(2-methyl-5-quinolinyl)-1-piperidinyl]ethyl}[1,2,3]triazolo[1,5-a]quinoline-3-carboxylate (free base of E102) (100 mg, 0.42 mmol) was treated with 2.5 ml of NH₃ (7M in MeOH) and reacted in a microwave reactor (PersonalChemistry Emrys™ Optimiser, 300W) at 145° C. for 1 hour. To improve the conversion of the starting material, further 7M NH₃ in MeOH (2 ml) was added and the reaction mixture was reacted in the microwave reactor at 145° C. for 1 hour. The crude product was evaporated in vacuo and then purified by silica gel chromatography to give an amount of the title product in low yield. The reaction was repeated on the same scale and under the same conditions. The crude material from this second reaction was combined with the impure fractions from the first reaction, and was purified by silica gel chromatography eluting with 2% MeOH in DCM to 4% MeOH in DCM to afford the free base of the title compound (80 mg, 41%). The free base (77 mg, 0.166 mmol) was dissolved in dry MeOH (3 ml) and treated with HCl (0.292 ml of a 1.25 M solution in methanol) at 0° C. The resulting suspension was stirred at room temperature for 1 hour. Evaporation of the volatiles and trituration with diethyl ether gave the title compound as a yellow solid (79 mg, 88.5%); ¹H NMR (400 MHz, DMSO-d₆) δ ppm 2.1-2.2 (m, 4H) 2.67 (s, 3H) 3.32-3.40 (m, 2H) 3.42-3.52 (m, 2H) 3.61-3.69 (m, 2H) 3.69-3.77 (m, 1H) 3.85 (d, 2H) 7.4-8.0 (m, 7H) 8.34 (d, 1H) 8.7 (br.s., 1H) 8.75 (d, 1H) 10.4 (br. s., 1H).

### Example 110

### Ethyl 7-methyl-6-{2-[4-(2-methyl-5-quinolinyl)-1-piperidinyl]ethyl}imidazo-[1,5-a]quinoline-3-carboxylate (E110)

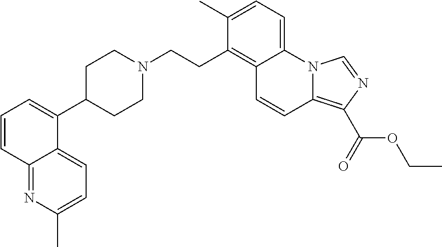

A mixture of ethyl 7-methyl-6-(2-oxoethyl)imidazo[1,5-a]quinoline-3-carboxylate (D112) (75 mg, 0.25 mmol) and 2-methyl-5-(4-piperidinyl)quinoline (WO2064/046124) (67 mg, 0.30 mmol) in 1,2-dichloroethane (2.5 ml) was stirred at room temperature for 1 hour. Sodium triacetoxyborohydride (63 mg, 0.30 mmol) was then added and after stirring at room temperature for 18 hours the reaction mixture was quenched with water (10 ml), extracted with ethyl acetate (3×15 ml) and then concentrated in vacuo. The crude product was purified by SPE-Si cartridge eluting with 5% methanol in DCM to afford the title compound as a white solid (60 mg, 47%); MS (ES/+) m/z: 507.3 [MH⁺], C₃₂H₃₅N₄O₂ requires 506.65.

### Example 111

### 7-Methyl-6-{2-[4-(2-methyl-5-quinolinyl)-1-piperidinyl]ethyl}imidazo[1,5-a]quinoline-3-carboxamide dihydrochloride (E111)

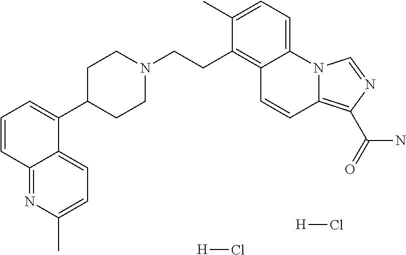

A mixture of ethyl 7-methyl-6-{2-[4-(2-methyl-5-quinolinyl)-1-piperidinyl]ethyl}imidazo[1,5-a]quinoline-3-carboxylate (E110) (60 mg, 0.12 mmol) and potassium hydroxide (4 ml of 1M solution in MeOH) was stirred at 80° C. for 2 hours. After SCX purification the ammonium 7-methyl-6-{2-[4-(2-methyl-5-quinolinyl)-1-piperidinyl]ethyl}imidazo[1,5-a]quinoline-3-carboxylate was isolated and used, without further purification, to prepare the free base of the title compound following the general procedure for amide formation using hexamethyldisilazane (0.002 ml, 1.1 eq). A white solid precipitated from the reaction mixture, was collected by filtration and triturated with CH₃OH/CH₂Cl₂ 9/1 to afford 16 mg of the title free base compound as a white solid which was dissolved in dry CH₃OH (0.5 ml) and treated with HCl (0.059 ml of a 1.25 M solution in ethanol). The resulting suspension was stirred at room temperature for 1 hour. Evaporation of the volatiles and trituration with diethyl ether gave the title compound as a white solid (13 mg, overall yield 27%); ¹H NMR (500 MHz, DMSO-d₅) δ ppm 10.23 (br. s., 1H), 9.17 (s, 1H), 8.55 (d, 1H), 8.36 (d, 1H), 8.08 (d, 1H), 7.84 (d, 1H), 7.78 (d, 1H), 7.72 (t, 1H), 7.62 (d, 1H), 7.57 (s, 1H), 7.49 (d, 1H), 7.45 (d, 1H), 7.22 (s, 1H), 3.18-3.39 (m, 4H), 3.17-3.95 (m, 5H), 2.66 (s, 3H), 2.55 (s, 3H), 2.00-2.26 (m, 4H).

### Example 112

### 6-{2-[4-(2-Methyl-5-quinolinyl)-1-piperidinyl]ethyl}-4H-imidazo[5,1-c][1,4]-benzoxazine-3-carboxamide dihydrochloride (E112)

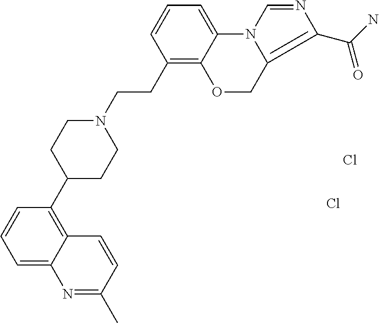

A mixture of ethyl 6-{2-[4-(2-methyl-5-quinolinyl)-1-piperidinyl]ethyl}-4H-imidazo[5,1-c][1,4]benzoxazine-3-carboxylate (free base of E37) (89 mg, 0.18 mmol) and potassium hydroxide (4 ml of a 1M solution in MeOH) was stirred at 90° C. for 2 hours. After SCX purification the ammonium 6-{2-[4-(2-methyl-5-quinolinyl)-1-piperidinyl]ethyl}-4H-imidazo[5,1-c][1,4]benzoxazine-3-carboxylate was isolated and used, without further purification, to prepare the free base of the title compound following the general procedure for amide formation using hexamethyldisilazane (0.042 ml, 1.10 eq) (see Example 14). The free base of the title compound precipitated and was filtered off from the reaction mixture and triturated with diethyl ether. The free base (38 mg, 0.082 mmol, 45% yield) was then suspended in dry DCM (2 ml) and treated with HCl (0.165 ml of a 1.25M solution in methanol) at 0° C. The resulting suspension was stirred at 0° C. for 2 hours. Evaporation of the volatiles and trituration with diethyl ether gave the title compound (40 mg, 0.074 mmol) as white solid; MS (ES) m/z 468.2 [MH⁺], C₂₈H₂₉N₅O₂ requires 467.57; ¹H NMR (500 MHz, DMSO-d₆) δ ppm 2.05-2.15 (m, 2H) 2.19-2.34 (m, 2H) 2.95 (s, 3H) 3.15-3.22 (m, 2H) 3.24-3.32 (m, 2H) 3.30-3.37 (m, 2H) 3.70-3.79 (m, 1H) 3.79-3.89 (m, 1H) 5.58 (s, 2H) 7.16 (t, 1H) 7.26 (d, 1H) 7.36 (br. s., 1H) 7.54 (br. s., 1H) 7.74 (d, 1H) 7.84 (d, 1H) 7.99 (d, 1H) 8.04-8.13 (m, 1H) 8.28 (d, 1H) 8.57 (s, 1H) 9.31 (br. s., 1H) 10.89 (br. s., 1H).

### Example 113

### 3-(3-Methyl-1,2,4-oxadiazol-5-yl)-6-{2-[4-(2-methyl-5-quinolinyl)-1-piperidinyl]ethyl}-4H-imidazo[5,1-c][1,4]-benzoxazine dihydrochloride (E113)

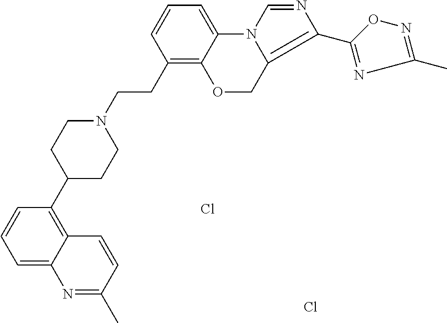

Methyl carboxyamide oxime (10.6 mg, 0.14 mmol) was added to a suspension of sodium hydride (5.60 mg of 60% suspension in oil, 0.14 mmol) in dry THF (3.50 ml) followed after 10 minutes by the addition of ethyl 6-{2-[4-(2-methyl-5-quinolinyl)-1-piperidinyl]ethyl}-4H-imidazo[5,1-c][1,4]benzoxazine-3-carboxylate (free base of E37) (66 mg, 0.13 mmol). After 10 minutes DMF (0.50 ml) was added and the reaction was stirred at room temperature overnight. The reaction mixture was purified by SCX followed by SPE-Si eluting with 3% MeOH in DCM to afford the free base of the title compound as white foam (13.8 mg, 0.027 mmol, 19% yield). The free base was then suspended in dry DCM (2 ml) and treated with HCl (0.054 ml of a 1.25 M solution in methanol) at 0° C. The resulting suspension was stirred at 0° C. for 2 hours. Evaporation of the volatiles and trituration with diethyl ether gave the title compound (13 mg, 0.022 mmol) as white solid; MS (ES) m/z: 507.3 [MH⁺], C₃₀H₃₀N₆O₂ requires 506.61; ¹H NMR (500 MHz, DMSO-d₆) δ ppm 2.04-2.13 (m, 2H) 2.13-2.28 (m, 2H) 2.40 (s, 3H) 2.79 (s, 3H) 3.13-3.25 (m, 2H) 3.22-3.47 (m, 4H) 3.66-3.85 (m, 3H) 5.68 (s, 2H) 7.20 (t, 1H) 7.31 (d, 1H) 7.47-7.61 (m, 1H) 7.61-7.78 (m, 1H) 7.78-7.89 (m, 1H) 7.91 (d, 1H) 7.93-8.08 (m, 1H) 8.85 (br. s., 1H) 8.80 (s, 1H) 10.53 (br. s., 1H).

### Example 114

### 1-(6-{2-[4-(2-Methyl-5-quinolinyl)-1-piperidinyl]ethyl}-4H-imidazo[5,1-c][1,4]-benzoxazin-3-yl)ethanone dihydrochloride (E114)

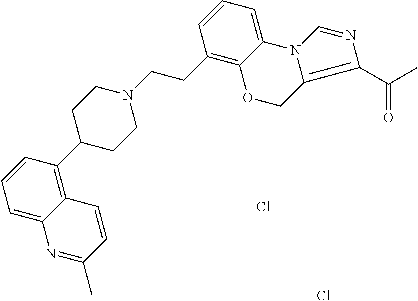

To an ice cooled stirred solution of N-methyl-N-(methyloxy)-6-{2-[4-(2-methyl-5-quinolinyl)-1-piperidinyl]ethyl}-4H-imidazo[5,1-c][1,4]benzoxazine-3-carboxamide (E182) (0.39 g, 0.76 mmol) in anhydrous THF (5 ml) was added methyl magnesium bromide (0.30 ml of a 3M solution in diethyl ether, 0.90 mmol) and the resulting solution was stirred for 1 hour at 0° C. The reaction mixture was poured into cold 1N aqueous hydrochloric acid (5 ml), then treated with NaHCO₃ (15 ml) and extracted with EtOAc (3×15 ml). The combined organic layers were dried (Na₂SO₄), evaporated in vacuo and the residue purified by SPE-Si eluting with 3% methanol to afford the free base of the title compound (175 mg, 0.38 mmol, 50% yield) as white solid. The free base (19.8 mg, 0.042 mmol) was then suspended in dry DCM (2 ml) and treated with HCl (0.085 ml of a 1.25 M solution in methanol) at 0° C. The resulting suspension was stirred at 0° C. for 1 hour. Evaporation of the volatiles and trituration with diethyl ether gave the title compound (17.5 mg, 0.032 mmol) as white solid; MS (ES) m/z: 467.3 [MH⁺], C₂₉H₃₀N₄O₂ requires 466.58; ¹H NMR (500 MHz, DMSO-d₆) δ ppm 2.08 (d, 2H) 2.16-2.31 (m, 2H) 2.48 (s, 3H) 2.85 (s, 3H) 3.11-3.24 (m, 2H) 3.24-3.50 (m, 2H) 3.27-3.35 (m, 2H) 3.63-3.87 (m, 3H) 5.59 (s, 2H) 7.16 (t, 1H) 7.28 (d, 1H) 7.50-7.65 (m, 1H) 7.72-7.84 (m, 1H) 7.86 (d, 1H) 7.88-7.99 (m, 1H) 8.01-8.17 (m, 1H) 8.63 (s, 1H) 8.72-9.24 (m, 1H) 10.78 (br. s., 1H).

### Example 115

### 3(3-Methyl-5-isoxazolyl)-6-{2-[4(2-methyl-5-quinolinyl)-1-piperidinyl]ethyl}-4H-imidazo[5,1-c][1,4]benzoxazine dihydrochloride (E115)

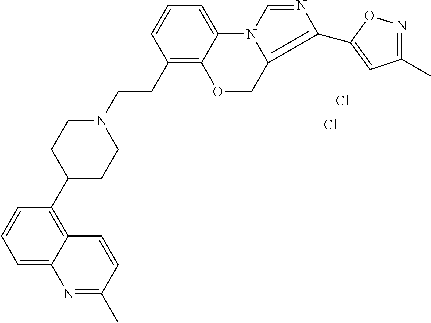

A mixture of (2Z)-3-(dimethylamino)-1-(6-{2-[4(2-methyl-5-quinolinyl)-1-piperidinyl]ethyl}-4H-imidazo[5,1-c][1,4]benzoxazin-3-yl)-2-buten-1-one (D113) (57 mg, 0.16 mmol) and hydroxylamine hydrochloride (16.68 mg, 0.24 mmol) in EtOH (1.50 ml) was heated with stirring at 150° C. under microwave irradiation for 5 min. The precipitate was filtered off from the reaction mixture and washed with diethyl ether to afford the title compound (54 mg, 0.10 mmol, 65% yield) as white solid; MS (ES) m/z: 506.3 [MH⁺], C₃₁H₃₁N₅O₂ requires 505.62; ¹H NMR (300 MHz, DMSO-d₅) δ ppm 2.02-2.20 (m, 2H) 2.18-2.40 (m, 2H) 2.31 (s, 3H) 2.99 (s, 3H) 3.13-3.51 (m, 4H) 3.41-3.89 (m, 5H) 5.63 (s, 2H) 6.62 (s, 1H) 7.20 (t, 1H) 7.31 (d, 1H) 7.75 (d, 1H) 7.90 (d, 1H) 8.00 (d, 1H) 8.10 (t, 1H) 8.28 (d, 1H) 8.73 (s, 1H) 9.31 (d, 1H) 10.87 (br. s., 1H).

### Example 116

### 3(3-Methyl-1H-pyrazol-5-yl)-6-{2-[4-(2-methyl-5-quinolinyl)-1-piperidinyl]ethyl}-4H-imidazo[5,1-c][1,4]-benzoxazine dihydrochloride (E116)

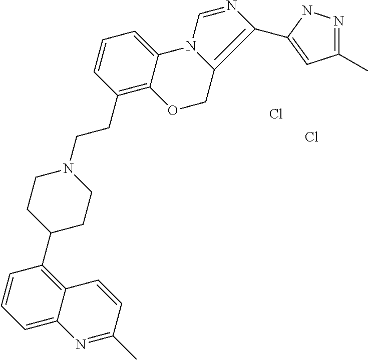

A mixture of (2Z)-3-(dimethylamino)-1-(6-{2-[4-(2-methyl-5-quinolinyl)-1-piperidinyl]ethyl}-4H-imidazo[5,1-c][1,4]benzoxazin-3-yl)-2-buten-1-one (D113) (33 mg, 0.062 mmol) and hydrazine monohydrate (0.003 ml, 0.096 mmol) in EtOH (1.5 ml) was heated with stirring at 150° C. under microwave irradiation for 5 minutes. The free base of the title compound precipitated and was filtered off from the reaction mixture and triturated with diethyl ether. The free base (29 mg, 0.058 mmol, 60% yield) was then suspended in dry DCM (2 ml) and treated with HCl (0.116 ml of a 1.25 M solution in methanol) at 0° C. The resulting suspension was stirred at 0° C. for 1 hour. Evaporation of the volatiles and trituration with diethyl ether gave the title compound (28 mg, 0.048 mmol) as pale yellow solid; MS (ES) m/z: 505.3 [MH⁺], C₃₁H₃₂N₆O requires 504.63; ¹H NMR (300 MHz, DMSO-d₆) δ ppm 2.01-2.36 (m, 4H) 2.30 (s, 3H) 2.92 (s, 3H) 3.12-4.01 (m, 9H) 5.62 (s, 2H) 6.43 (s, 1H) 7.20 (t, 1H) 7.33 (d, 1H) 7.61-7.77 (m, 1H) 7.80-7.96 (m, 2H) 7.96-8.08 (m, 1H) 8.07-8.22 (m, 1H) 8.96 (s, 1H) 9.04-9.24 (m, 1H) 10.52 (br. s., 1H).

### Example 117

### Ethyl 6-(2-{4-[2-(5-methyl-1,3,4-oxadiazol-2-yl)-1-benzofuran-4-yl]-1-piperazinyl}ethyl)imidazo[1,5-a]quinoline-3-carboxylate hydrochloride (E117)

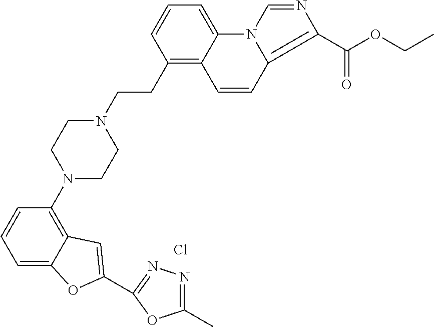

The title compound was prepared in 20% yield following the general procedure described in Example 1 starting from ethyl 6-(2-oxoethyl)imidazo[1,5-a]quinoline-3-carboxylate (D91) (44 mg, 0.15 mmol) and 1-[2-(5-methyl-1,3,4-oxadiazol-2-yl)-1-benzofuran-4-yl]piperazine (D116) (55 mg, 0.19 mmol); MS (ES) m/z: 551.2 [MH⁺]. C₃₁H₃₀N₆O₄ requires 550.62; ¹H NMR (500 MHz, DMSO-d₆) δ ppm 1.37 (t, J=6.83 Hz, 3H) 2.63 (s, 3H) 3.24-3.52 (m, 6H) 3.60 (dd, 2H) 3.80 (d, J=10.74 Hz, 2H) 3.86 (d, J=12.69 Hz, 2H) 4.36 (q, 2H) 6.92 (d, J=7.81 Hz, 1H) 7.37-7.47 (m, 2H) 7.58 (d, J=6.83 Hz, 1H) 7.79 (t, 1H) 8.01-8.04 (m, 3H) 8.52 (d, J=7.81 Hz, 1H) 9.32 (s, 1H) 11.01 (br. s., 1H).

### Example 118

### Ethyl 6-{2-[(2R)-4-(7-fluoro-2-methyl-5-quinolinyl)-2-methyl-1-piperazinyl]ethyl}-4H-imidazo[5,1-c][1,4]benzoxazine-3-carboxylate (E118)

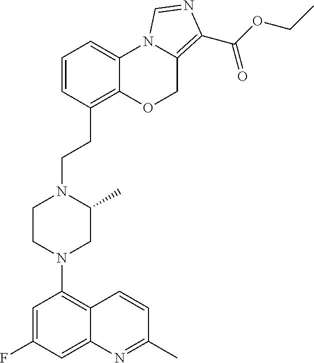

A mixture of ethyl 6-(2-oxoethyl)-4H-imidazo[5,1-c][1,4]benzoxazine-3-carboxylate (D6) (83 mg, 0.28 mmol) and 7-fluoro-2-methyl-5-[(3R)-3-methyl-1-piperazinyl]quinoline (90 mg, 0.35 mmol) (made by a similar procedure to procedures described in WO2004/046124) in dry 1,2-dichloroethane (10 ml) was stirred at room temperature under nitrogen for 40 minutes. Sodium triacetoxyborohydride (74 mg, 0.35 mmol) was then added and the resulting reaction mixture was stirred for 3 hours, quenched with a saturated aqueous solution of NaHCO₃ (10 ml) and extracted with DCM (3×10 ml). The organic layers were combined, dried (Na₂SO₄) and concentrated in vacuo. The crude product was purified by column chromatography on silica, eluting with 1% methanol in DCM to afford the title compound as a white foam (63 mg, 34%); MS (ES) m/z 530.1 [MH⁺], C30H32FN5O3 requires 529.61; ¹H-NMR (500 MHz, CDCl₃) δ: 8.34 (d, 1H), 8.03 (s, 1H), 7.76 (d, 1H), 7.4 (d, 1H), 7.3 (m, 1H), 7.2 (d, 1H), 7.1 (m, 2H), 5.59 (s, 2H), 4.45 (quart., 2H), 3.-2.7 (bm, 11H), 2.77 (s, 3H), 1.46 (t, 3H), 1.2 (m, 3H).

### Example 119

### 6-{2-[(2R)-4-(7-fluoro-2-methyl-5-quinolinyl)-2-methyl-1-piperazinyl]ethyl}-4H-imidazo[5,1-c][1,4]-benzoxazine-3-carboxamide dihydrochloride (E119)

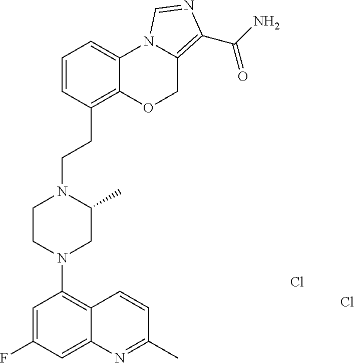

The title compound was prepared in 45% yield according to the general amide formation procedure (see Example 14) starting from 6-{2-[(2R)-4-(7-fluoro-2-methyl-5-quinolinyl)-2-methyl-1-piperazinyl]ethyl}-4H-imidazo[5,1-c][1,4]benzoxazine-3-carboxylic acid (E183) (38 mg, 0.07 mmol) and hexamethyldisilazane (1.1 eq); MS (ES) m/z: 501.5 [MH⁺], C₂₆H₂₉N₆O₂ requires 500.58; ¹H-NMR (500 MHz, DMSO-d⁶) δ: 11.20 (bs, 1H), 9.1 (d, 1H), 8.58 (s, 1H), 8.16 (m, 1H),), 8.11 (d, 1H), 7.92 (d, 1H), 78.4 (d, 1H), 7.5 (d, 1H), 7.3 (d, 1H), 7.15 (t, 1H), 5.62 (s, 2H), 4.0-2.9 (m., 11H), 2.99 (s, 3H), 2.74 (d, 3H).

### Example 120

### 2-Methyl-6-{2-[4-(2-methyl-5-quinolinyl)-1-piperazinyl]ethyl}-2,4-dihydro-1H-[1,2,4]triazolo[3,4-c][1,4]benzoxazin-1-one dihydrochloride (E120)

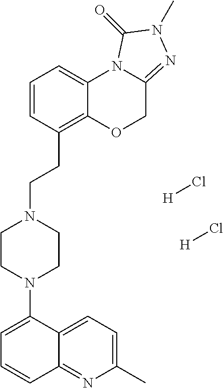

The title compound was prepared in 61% yield following the general reductive amination procedure of Example 1 starting from (2-methyl-1-oxo-2,4-dihydro-1H-[1,2,4]triazolo[3,4-c][1,4]benzoxazin-6-yl)acetaldehyde (D120) (64 mg, 0.261 mmol). The crude product was purified by flash chromatography on silica gel eluting with a gradient of methanol in DCM (1 to 2%) to afford the free base of the title compound (72 mg, 61%). Treatment with HCl (2.2 eq of 1.25M solution in MeOH) in 1:1 methanol/DCM (4 ml) at 0° C. gave the title compound as a solid; MS (ES) m/z: 457.00 [MH⁺], C26H28N6O2 requires 456.55; ¹H NMR (500 MHz, DMSO-d₆) δ ppm 2.69 (s, 3H) 3.11-3.17 (m, 2H) 3.22 (t, 2H) 3.26-3.39 (m, 4H) 3.40 (s, 3H) 3.41-3.54 (m, 2H) 3.66-3.76 (m, 2H) 5.24 (s, 2H) 7.08-7.34 (m, 3H) 7.37-7.58 (m, 1H) 7.58-7.84 (m, 2H) 8.06 (d, 1H) 8.26-8.61 (m, 1H) 10.45 (br. s., 1H).

### Example 121

### 1-(6-{2-[4-(2-Methyl-5-quinolinyl)-1-piperidinyl]ethyl}-4,5-dihydroimidazo[1,5-a]quinolin-3-yl)ethanone dihydrochloride (E121)

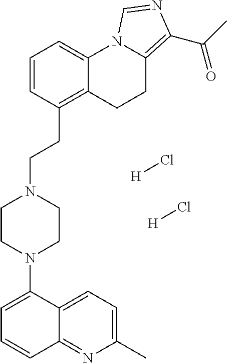

The title compound was prepared following the procedure of Example 35 using methyl magnesium bromide (0.081 ml of a 3M solution in diethyl ether, 0.243 mmol) and N-methyl-N-(methyloxy)-6-{2-[4-(2-methyl-5-quinolinyl)-1-piperidinyl]ethyl}-4,5-dihydroimidazo[1,5-a]quinoline-3-carboxamide (E184) (105 mg, 0.206 mmol). The crude product was purified by flash chromatography on silica gel eluting with a gradient of methanol in DCM (1 to 5%) to afford the free base of the title compound (24 mg, 25%). Treatment with HCl (2.2 eq of 1.25M solution in MeOH) in 1:1 methanol/DCM (2 ml) at 0° C. gave the title compound as a white solid; MS (ES) m/z: 465.00 [MH⁺], C30H32N4O requires 464.61; ¹H NMR (500 MHz, DMSO-d₆) δ ppm 2.05-2.23 (m, 4H) 2.46 (s, 3H) 2.77 (s, 3H) 3.00 (t, 2H) 3.07-3.61 (m, 6H) 3.28 (t, 2H) 3.66-3.83 (m, 3H) 7.29 (d, 1H) 7.41 (t, 1H) 7.47-7.59 (m, 1H) 7.60-7.76 (m, 1H) 7.80 (d, 1H) 7.82-7.90 (m, 1H) 7.90-8.03 (m, 1H) 8.53 (s, 1H) 8.66-8.99 (m, 1H) 10.39 (br. s., 1H).

### Example 122

### 6-{2-[4-(2-Methyl-5-quinolinyl)-1-piperidinyl]ethyl}-4,5-dihydroimidazo[1,5-a]quinoline-3-carboxamide dihydrochloride (E122)

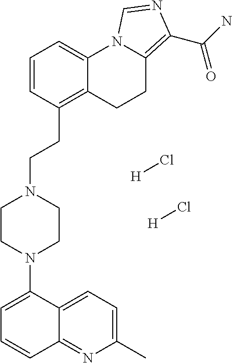

A mixture of the free base of ethyl 6-{2-[4-(2-methyl-5-quinolinyl)-1-piperidinyl]ethyl}-4,5-dihydroimidazo[1,5-a]quinoline-3-carboxylate (E82) (32 mg, 0.065 mmol) and potassium hydroxide (5 ml, 1M solution in MeOH) was stirred at reflux for 2.5 hours. After SCX purification 6-{2-[4-(2-methyl-5-quinolinyl)-1-piperidinyl]ethyl}-4,5-dihydroimidazo[1,5-a]quinoline-3-carboxylic acid was isolated (0.065 mmol) and used without further purification according to the general procedure for amide formation using hexamethyldisilazane (0.015 ml, 0.071 mmol) (see Example 14). The precipitated free base was filtered and triturated with ethyl ether (12 mg, 40%) and then treated with HCl (2.2 eq of 1.25M solution in MeOH) in 1:1 methanol/DCM (2 ml) at 0° C. to afford the title compound as a white solid; MS (ES) m/z: 466.00 [MH⁺], C29H31N5O requires 465.60; ¹H NMR (500 MHz, DMSO-d₆) δ ppm 2.08-2.24 (m, 4H) 2.75 (br. s., 3H) 2.99 (t, 2H) 3.18-3.49 (m, 8H) 3.71-3.77 (m, 1H) 3.78 (d, 2H) 7.15 (br. s., 1H) 7.27 (d, 1H) 7.36 (br. s., 1H) 7.40 (t, 1H) 7.51 (br. s., 1H) 7.62 (br. s., 1H) 7.78 (d, 1H) 7.81 (br. s., 1H) 7.93 (br. s., 1H) 8.47 (s, 1H) 8.72 (br. s., 1H) 10.38 (br. s., 1H).

### Example 123

### Ethyl 7-methyl-6-{2-[4-(2-methyl-5-quinolinyl)-1-piperidinyl]ethyl}-4H-imidazo[5,1-c][1,4]-benzoxazine-3-carboxylate dihydrochloride (E123)

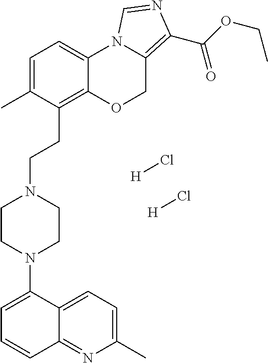

The title compound was prepared following the procedure of Example 80 starting from 7-methyl-8-{2-[4-(2-methyl-5-quinolinyl)-1-piperidinyl]ethyl}-2H-1,4-benzoxazin-3(4H)-one (D121) (173 mg, 0.417 mmol). The crude product was purified by flash chromatography on silica gel eluting with a gradient of methanol in DCM (1 to 3%) to afford the free base of the title compound (87 mg, 41%). Treatment with HCl (2.2 eq of 1.25M solution in MeOH) in 1:1 methanol/DCM (2 ml) at 0° C. gave the title compound as a white solid; MS (ES) m/z: 511.00 [MH⁺]; C31H34N4O3 requires 510.63; ¹H NMR (400 MHz, DMSO-d₆) δ ppm 1.31 (t, 3H) 2.03-2.24 (m, 4H) 2.40 (s, 3H) 2.69 (s, 3H) 3.03-3.25 (m, 4H) 3.25-3.51° (m, 2H) 3.64-3.77 (m, 1H) 3.80 (d, J=11.35 Hz, 2H) 4.27 (q, 2H) 5.57 (s, 2H) 7.06 (d, J=8.42 Hz, 1H) 7.49 (br. s., 1H) 7.61 (br. s., 1H) 7.76 (d, J=8.06 Hz, 1H) 7.81 (br. s., 1H) 7.90 (br. s., 1H) 8.58 (s, 1H) 8.80 (br. s., 1H) 10.17 (br. s., 1H).

### Example 124

### N,7-Dimethyl-6-{2-[4-(2-methyl-5-quinolinyl)-1-piperidinyl]ethyl}-4H-imidazo[5,1-c][1,4]-benzoxazine-3-carboxamide dihydrochloride (E124)

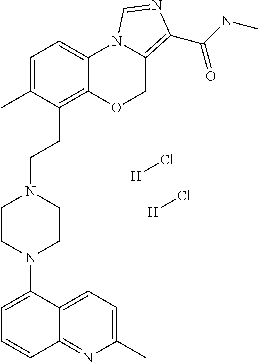

The title compound was prepared following the procedure of Example 38 using trimethylaluminium (2.0M in hexanes, 392 μl, 784 mmol), methylamine (2.0M in THF, 392 μl, 0.784 mmol) and the free base of ethyl 7-methyl-6-{2-[4-(2-methyl-5-quinolinyl)-1-piperidinyl]ethyl}-4H-imidazo[5,1-c][1,4]benzoxazine-3-carboxylate (E123) (40 mg, 0.078 mmol). The crude product was purified by flash chromatography on silica gel eluting with a gradient of methanol in DCM (1 to 3%) to afford the free base of the title compound (25 mg, 64%). Treatment with HCl (2.2 eq of 1.25M solution in MeOH) in 1:1 methanol/DCM (2 ml) at 0° C. gave the title compound as a white solid; MS (ES) m/z: 496.00 [MH⁺], C30H33N5O2 requires 495.62; ¹H NMR (400 MHz, DMSO-d₅) δ ppm 2.10-2.18 (m, 4H) 2.40 (s, 3H) 2.71 (s, 3H) 2.76 (d, 3H) 3.11-3.24 (m, 2H) 3.24-3.62 (m, 4H) 3.67-3.75 (m, 1H) 3.81 (d, 2H) 5.60 (s, 2H) 7.05 (d, 1H) 7.47 (br. s., 1H) 7.58 (br. s., 1H) 7.72 (d, 1H) 7.75-7.84 (m, 1H) 7.83-7.98 (m, 1H) 8.13 (q, 1H) 8.55 (s, 1H) 8.68 (br. s., 1H) 10.21 (br. s., 1H).

### Example 125

### 7-Methyl-6-{2-[4-(2-methyl-5-quinolinyl)-1-piperidinyl]ethyl}-4H-imidazo[5,1-c][1,4]-benzoxazine-3-carboxamide dihydrochloride (E125)

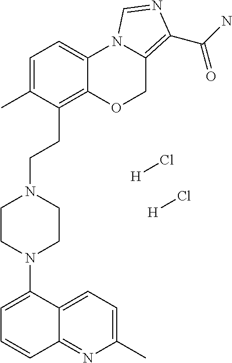

A mixture of the free base of ethyl 7-methyl-6-{2-[4-(2-methyl-5-quinolinyl)-1-piperidinyl]ethyl}-4H-imidazo[5,1-c][1,4]benzoxazine-3-carboxylate (E123) (36 mg, 0.071 mmol) and potassium hydroxide (4 ml, 1M solution in MeOH) was stirred at reflux for 2 hours. After SCX purification the 7-methyl-6-{2-[4-(2-methyl-5-quinolinyl)-1-piperidinyl]ethyl}-4H-imidazo[5,1-c][1,4]benzoxazine-3-carboxylic acid was isolated (24 mg, 0.050 mmol, 71%) and used without further purification, to prepare the free base of the title compound following the general procedure for amide formation using hexamethyldisilazane (0.023 ml, 0.108 mmol). The free base of the title compound precipitate during the reaction and it was filtered and triturated with ethyl ether (10.3 mg, 26%). The free base was then treated with HCl (2.2 eq of 1.25M solution in MeOH) in 1:1 methanol/DCM (2 ml) at 0° C. gave the title compound as a white solid; MS (ES) m/z: 482.00 [MH⁺], C29H31N5O2 requires 481.60; ¹H NMR (500 MHz, DMSO-d₆) δ ppm 2.07-2.18 (m, 4H) 2.38 (s, 3H) 2.73 (s, 3H) 3.10-3.24 (m, 4H) 3.24-3.52 (m, 2H) 3.69-3.77 (m, 1H) 3.80 (d, 2H) 5.57 (s, 2H) 7.03 (d, 1H) 7.31 (s, 1H) 7.45-7.56 (m, 2H) 7.60 (br. s., 1H) 7.73 (d, J=8.18 Hz, 1H) 7.79 (br. s., 1H) 7.91 (br. s., 1H) 8.52 (s, 1H) 8.69 (br. s., 1H) 10.03 (br. s., 1H).

### Example 126

### Ethyl 6-{2-[(2R)-2-methyl-4-(2-methyl-5-quinolinyl)-1-piperazinyl]ethyl}-4,5-dihydroimidazo[1,5-a]quinoline-3-carboxylate dihydrochloride (E126)

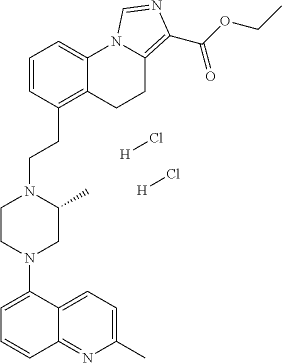

The title compound was prepared in 72% yield following the general reductive amination procedure of Example 1 starting from ethyl 6-(2-oxoethyl)-4,5-dihydroimidazo[1,5-a]quinoline-3-carboxylate (D86) (103 mg, 0.363 mmol) and 2-methyl-5-[(3R)-3-methyl-1-piperazinyl]quinoline (131 mg, 0.544 mmol) (WO2004/046124). The crude product was purified by flash chromatography on silica gel eluting with a gradient of methanol in DCM (1 to 3%) to afford the free base of the title compound (134 mg, 72%). Treatment with HCl (2.2 eq of 1.25M solution in MeOH) in 1:1 methanol/DCM (2 ml) at 0° C. gave the title compound as a yellow solid; MS (ES) m/z: 510.10 [MH⁺], C31H35N5O2 requires 509.65; ¹H NMR (500 MHz, DMSO-d₆) δ ppm 1.30 (t, 3H) 1.43 (s, 3H) 2.74 (s, 3H) 3.03 (t, 2H) 3.17-3.91 (m, 6H) 3.20-3.37 (m, 2H) 3.26 (t, 2H) 3.40-3.56 (m, 2H) 3.85 (d, 1H) 4.26 (q, 2H) 7.23-7.30 (m, 1H) 7.32 (d, 1H) 7.40 (t, 1H) 7.46-7.65 (m, 1H) 7.69-7.78 (m, 2H) 7.80 (d, 1H) 8.52 (s, 1H) 8.53-8.69 (m, 1H) 10.94 (br. s., 1H).

### Example 127

### 6-{2-[(2R)-2-Methyl-4-(2-methyl-5-quinolinyl)-1-piperazinyl]ethyl}-4,5-dihydroimidazo[1,5-a]quinoline-3-carboxamide dihydrochloride (E127)

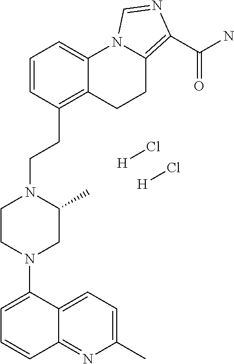

A mixture of the free base of ethyl 6-{2-[(2R)-2-methyl-4-(2-methyl-5-quinolinyl)-1-piperazinyl]ethyl}-4,5-dihydroimidazo[1,5-a]quinoline-3-carboxylate (E126) (122 mg, 0.24 mmol) and potassium hydroxide (5 ml, 1M solution in MeOH) was stirred at reflux for 4 hours. After SCX purification the 6-{2-[(2R)-2-methyl-4-(2-methyl-5-quinolinyl)-1-piperazinyl]ethyl}-4,5-dihydroimidazo[1,5-a]quinoline-3-carboxylic acid was isolated (114 mg, 0.24 mmol, 100%) and used without further purification, to prepare the free base of the title compound following the general procedure for amide formation using hexamethyldisilazane (0.055 ml, 0.261 mmol) (see Example 14). The free base of the title compound precipitate during the reaction and it was filtered and triturated with ethyl ether (89 mg, 78%). The free base was then treated with HCl (2.2 eq of 1.25M solution in MeOH) in 1:1 methanol/DCM (4 ml) at 0° C. gave the title compound as a yellow solid; MS (ES) m/z: 481.30 [MH⁺], C29H32N6O requires 480.61; ¹H NMR (500 MHz, DMSO-d₆) δ ppm 1.43 (s, 3H) 2.70 (s, 3H) 2.99 (t, 2H) 3.16-3.82 (m, 6H) 3.27 (t, 2H) 3.26-3.36 (m, 2H) 3.43-3.53 (m, 2H) 3.86 (d, 1H) 7.14 (s, 1H) 7.20-7.27 (m, 1H) 7.30 (d, 1H) 7.35 (s; 1H) 7.40 (t, 1H) 7.43-7.56 (m, 1H) 7.65-7.75 (m, 2H) 7.78 (d, 1H) 8.46 (s, 1H) 8.46-8.60 (m, 1H) 10.41 (br. s., 1H).

### Example 128

### Ethyl 7-methyl-6-{2-[4-(2-methyl-5-quinolinyl)-1-piperazinyl]ethyl}-4,5-dihydroimidazo[1,5-a]quinoline-3-carboxylate dihydrochloride (E128)

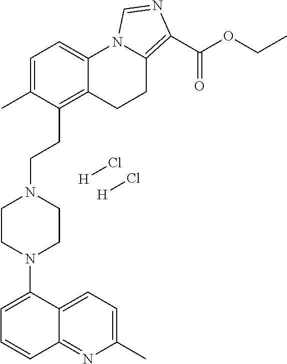

The title compound was prepared in 62% yield following the general reductive amination procedure of Example 1 starting from ethyl 7-methyl-6-(2-oxoethyl)-4,5-dihydroimidazo[1,5-a]quinoline-3-carboxylate (D125) (110 mg, 0.369 mmol) and 2-methyl-5-(1-piperazinyl)quinoline (101 mg, 0.443 mmol). The crude product was purified by flash chromatography on silica gel eluting with methanol in DCM (2%) to afford the free base of the title compound (117 mg, 62%). Treatment with HCl (2.2 eq of 1.25M solution in MeOH) in methanol (1 ml) at 0° C. gave the title compound as a yellow solid; MS (ES) m/z: 510.10 [MH⁺], C31H35N5O2 requires 509.65; ¹H NMR (500 MHz, DMSO-d₆) δ ppm 1.31 (t, 3H) 2.41-2.46 (m, 3H) 2.78 (br. s., 3H) 2.99-3.09 (m, 2H) 3.21-3.43 (m, 8H) 3.44-3.61 (m, 4H) 3.80 (d, 2H) 4.25 (q, 2H) 7.28 (d, 1H) 7.35 (br. s., 1H) 7.58 (br. s., 1H) 7.70 (d, 1H) 7.84 (br. s., 2H) 8.47-8.53 (m, 1H) 8.68 (br. s., 1H) 10.89 (br. s., 1H)

### Example 129

### Ethyl 7-methyl-6-{2-[4-(2-methyl-5-quinolinyl)-1-piperidinyl]ethyl}-4,5-dihydroimidazo[1,5-a]quinoline-3-carboxylate dihydrochloride (E129)

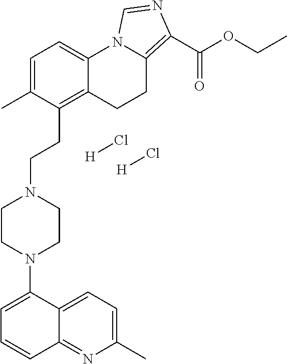

The title compound was prepared in 88% yield following the general reductive amination procedure of Example 1 starting from ethyl 7-methyl-6-(2-oxoethyl)-4,5-dihydroimidazo[1,5-a]quinoline-3-carboxylate (D125) (110 mg, 0.369 mmol) and 2-methyl-5-(4-piperidinyl)quinoline (100 mg, 0.443 mmol). The crude product was purified by flash chromatography on silica gel eluting with methanol in DCM (2%) to afford the free base of the title compound (166 mg, 88%). Treatment with HCl (2.2 eq of 1.25M solution in MeOH) in methanol (1 ml) at 0° C. gave the title compound as a white solid; MS (ES) m/z: 509.10 [MH⁺], C32H36N4O2 requires 508.66; ¹H NMR (500 MHz, DMSO-d₆) δ ppm 1.30 (t, 3H) 2.03-2.29 (m, 4H) 2.39-2.46 (m, 3H) 2.81 (br. s., 3H) 2.97-3.09 (m, 2H) 3.11-3.54 (m, 8H) 3.69-3.78 (m, 1H) 3.80-3.92 (m, 2H) 4.26 (q, 2H) 7.27 (d, 1H) 7.46-8.10 (m, 4H) 7.70 (d, 1H) 8.47-8.54 (m, 1H) 8.95 (br. s., 1H) 10.65 (br. s., 1H)

### Example 130

### Ethyl 7-methyl-6-{2-[(2R)-2-methyl-4-(2-methyl-5-quinolinyl)-1-piperazinyl]-ethyl}-4,5-dihydroimidazo[1,5-a]quinoline-3-carboxylate dihydrochloride (E130)

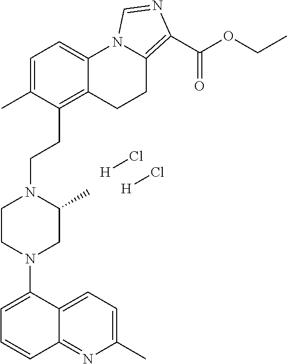

The title compound was prepared in 89% yield following the general reductive amination procedure of Example 1 starting from ethyl 7-methyl-6-(2-oxoethyl)-4,5-dihydroimidazo[1,5-a]quinoline-3-carboxylate (D125) (110 mg, 0.369 mmol) and 2-methyl-5-[(3R)-3-methyl-1-piperazinyl]quinoline (107 mg, 0.443 mmol) (WO2004/046124). The crude product was purified by flash chromatography on silica gel eluting with a gradient of methanol in DCM (2 to 3%) to afford the free base of the title compound (172 mg, 89%). Treatment with HCl (2.2 eq of 1.25M solution in MeOH) in methanol (1 ml) at 0° C. gave the title compound as a yellow solid; MS (ES) m/z: 524.10 [MH⁺], C32H37N5O2 requires 523.68; ¹H NMR (500 MHz, DMSO-d₆) δ ppm 1.31 (t, 3H) 1.45 (d, 3H) 2.41-2.46 (m, 3H) 280 (br. s.; 3H) 2.97-3.12 (m, 2H) 3.19-3.44 (m, 8H) 3.47-3.60 (m, 2H) 3.68-3.85 (m, 2H) 3.87-3.99 (m, 1H) 4.25 (q, 2H) 7.26-7.30 (m, 1H) 7.36 (br. s., 1H) 7.61 (br. s., 1H) 7.68-7.73 (m, 1H) 7.86 (br. s., 2H) 8.46-8.54 (m, 1H) 8.76 (br. s., 1H) 10.99 (br. s., 1H)

### Example 131

### N,7-Dimethyl-6-{2-[4-(2-methyl-5-quinolinyl)-1-piperazinyl]ethyl}-4,5-dihydroimidazo[1,5-a]quinoline-3-carboxamide dihydrochloride (E131)

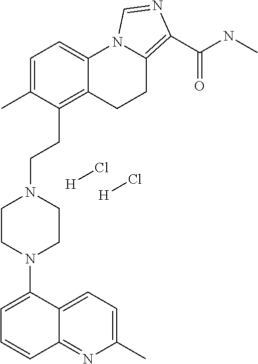

The title compound was prepared following the procedure of Example 38 using the free base of ethyl 7-methyl-6-{2-[4-(2-methyl-5-quinolinyl)-1-piperazinyl]ethyl}-4,5-dihydroimidazo[1,5-a]quinoline-3-carboxylate (E128) (50 mg, 0.098 mmol). The crude product was purified by SCX cartridge to afford the free base of the title compound (30 mg, 61%). Treatment with HCl (2.2 eq of 1.25M solution in MeOH) in 2:1 methanol/DCM (3 ml) at 0° C. gave the title compound as a yellow solid; MS (ES) m/z: 495.10 [MH⁺], C30H34N6O requires 494.64; ¹H NMR (400 MHz, DMSO-d₆) δ ppm 2.44 (s, 3H) 2.72 (s, 3H) 2.76 (d, 3H) 3.02 (t, 2H) 3.17-3.44 (m, 8H) 3.45-3.63 (m, 4H) 3.74-3.90 (m, 2H) 7.21-7.33 (m, 2H) 7.54 (br. s., 1H) 7.68 (d, 1H) 7.71-7.80 (m, 2H) 7.93-8.02 (m, 1H) 8.45 (s, 1H) 8.53 (br. s., 1H) 11.12 (br. s., 1H)

### Example 132

### N,7-Dimethyl-6-{2-[4-(2-methyl-5-quinolinyl)-1-piperidinyl]ethyl}-4,5-dihydroimidazo[1,5-a]quinoline-3-carboxamide dihydrochloride (E132)

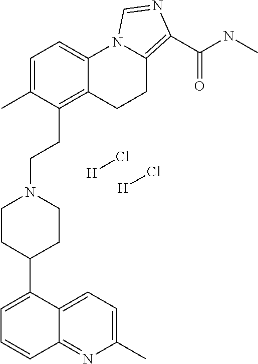

The title compound was prepared following the procedure of Example 38 using the free base of ethyl 7-methyl-6-{2-[4-(2-methyl-5-quinolinyl)-1-piperidinyl]ethyl}-4,5-dihydroimidazo[1,5-a]quinoline-3-carboxylate (E129) (60 mg, 0.118 mmol). The crude product was purified by flash chromatography on silica gel eluting with a gradient of methanol in DCM (1 to 2%) to afford the free base of the title compound (40 mg, 69%). Treatment with HCl (2.2 eq of 1.25M solution in MeOH) in 2:1 methanol/DCM (3 ml) at 0° C. gave the title compound as a white solid; MS (ES) m/z: 494.10 [MH⁺], C31H35N5O requires 493.65; ¹H NMR (400 MHz, DMSO-d₆) δ ppm 2.07-2.20 (m, 2H) 2.18-2.36 (m, 2H) 2.43 (s, 3H) 2.76 (d, 3H) 2.76 (s, 3H) 3.02 (t, 2H) 3.11-3.52 (m, 8H) 3.66-3.79 (m, 1H) 3.77-3.91 (m, 2H) 7.25 (d, 1H) 7.45-7.59 (m, 1H) 7.59-7.74 (m, 2H) 7.76-7.89 (m, 1H) 7.92-8.05 (m, 2H) 8.39-8.49 (m, 1H) 8.78 (br. s., 1H) 11.02 (br. s., 1H)

### Example 133

### N,7-Dimethyl-6-{2-[(2R)-2-methyl-4-(2-methyl-5-quinolinyl)-1-piperazinyl]-ethyl}-4,5-dihydroimidazo[1,5-a]quinoline-3-carboxamide dihydrochloride (E133)

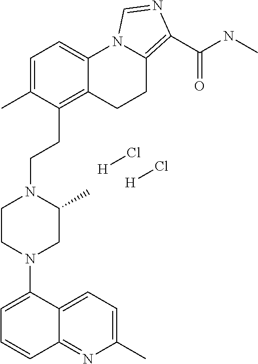

The title compound was prepared following the procedure of Example 38 using the free base of ethyl 7-methyl-6-{2-[(2R)-2-methyl-4-(2-methyl-5-quinolinyl)-1-piperazinyl]-ethyl}-4,5-dihydroimidazo[1,5-a]quinoline-3-carboxylate (E130) (70 mg, 0.134 mmol). The crude product was purified by flash chromatography on silica gel eluting with a gradient of methanol in DCM (1 to 2%) to afford the free base of the title compound (55 mg, 79%). Treatment with HCl (2.2 eq of 1.25M solution in MeOH) in 2:1 methanol/DCM (3 ml) at 0° C. gave the title compound as a yellow solid; MS (ES) m/z: 509.10 [MH⁺], C31H36N6O requires 508.67; ¹H NMR (400 MHz, DMSO-d₆) δ ppm 1.49 (d, 3H) 2.45 (s, 3H) 2.76 (s, 3H) 2.79 (s, 3H) 2.96-3.10 (m, 2H) 3.13-3.57 (m, 12H) 3.67-3.86 (m, 1H) 7.26 (d, 1H) 7.29-7.38 (m, 1H) 7.57-7.66 (m, 1H) 7.69 (d, 1H) 7.75-7.89 (m, 2H) 7.92-8.02 (m, 1H) 8.45 (s, 1H) 8.70 (br. s., 1H) 11.64 (br. s., 1H).

### Example 134

### 7-Methyl-6-{2-[4-(2-methyl-5-quinolinyl)-1-piperazinyl]ethyl}-4,5-dihydroimidazo[1,5-a]quinoline-3-carboxamide dihydrochloride (E134)

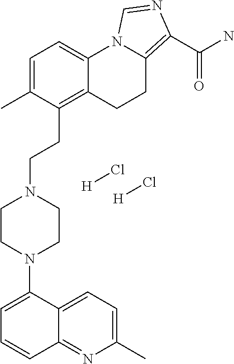

A mixture of free base of ethyl 7-methyl-6-{2-[4-(2-methyl-5-quinolinyl)-1-piperazinyl]ethyl}-4,5-dihydroimidazo[1,5-a]quinoline-3-carboxylate (E128) (57 mg, 0.112 mmol) and potassium hydroxide (4 ml, 1M solution in MeOH) was stirred at reflux for 4 hours. After SCX purification the 7-methyl-6-{2-[4-(2-methyl-5-quinolinyl)-1-piperazinyl]ethyl}-4,5-dihydroimidazo[1,5-a]quinoline-3-carboxylic acid was isolated (54 mg, 0.112 mmol, 100%) and used without further purification, to prepare the free base of the title compound following the general procedure for amide formation using hexamethyldisilazane (0.031 ml, 0.146 mmol). The precipitated free base was filtered and triturated with ethyl ether (24 mg, 50%). The free base was then treated with HCl (2.2 eq of 1.25M solution in MeOH) in 2:1 methanol/DCM (3 ml) at 0° C. to give the title compound as a yellow solid; MS (ES) m/z: 481.30 [MH⁺], C29H32N6O requires 480.61; ¹H NMR (300 MHz, DMSO-d₆) δ ppm 2.42 (s, 3H) 2.71 (s, 3H) 2.99 (t, 2H) 3.10-3.60 (m, 12H) 3.74-3.90 (m, 2H) 7.01 (s, 1H) 7.24 (d, 1H) 7.24-7.33 (m, 2H) 7.54 (br. s., 1H) 7.66 (d, 1H) 7.71-7.80 (br. m, 2H) 8.41 (s, 1H) 8.53 (br. s., 1H) 10.71 (br. s., 1H).

### Example 135

### 7-Methyl-6-{2-[4-(2-methyl-5-quinolinyl)-1-piperidinyl]ethyl}-4,5-dihydroimidazo[1,5-a]quinoline-3-carboxamide dihydrochloride (E135)

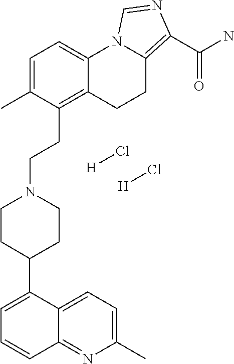

A mixture of the free base of ethyl 7-methyl-6-{2-[4-(2-methyl-5-quinolinyl)-1-piperidinyl]ethyl}-4,5-dihydroimidazo[1,5-a]quinoline-3-carboxylate (E129) (96 mg, 0.189 mmol) and potassium hydroxide (4 ml, 1M solution in MeOH) was stirred at reflux for 4 hours. After SCX purification the 7-methyl-6-{2-[4-(2-methyl-5-quinolinyl)-1-piperidinyl]ethyl}-4,5-dihydroimidazo[1,5-a]quinoline-3-carboxylic acid was isolated (91 mg, 0.189 mmol, 100%) and used without further purification, to prepare the free base of the title compound following the general procedure for amide formation using hexamethyldisilazane (0.052 ml, 0.246 mmol). The precipitated free base of the title compound was filtered and triturated with diethyl ether (46 mg, 50%). The free base was then treated with HCl (2.2 eq of 1.25M solution in MeOH) in 2:1 methanol/DCM (3 ml) at 0° C. to give the title compound as a white solid; MS (ES) m/z: 480.30 [MH⁺], C30H33N5O requires 479.62; ¹H NMR (400 MHz, DMSO-d₆) δ ppm 2.10-2.29 (m, 4H) 2.44 (s, 3H) 2.76 (s, 3H) 3.01 (t, 2H) 3.13-3.52 (m, 8H) 3.68-3.79 (m, 1H) 3.80-3.90 (m, 2H) 7.14 (s, 1H) 7.28 (d, 1H) 7.35 (s, 1H) 7.54 (br. m, 1H) 7.59-7.68 (br. m, 1H) 7.69 (d, 1H) 7.84 (br. m, 1H) 7.98 (br. m, 1H) 8.44 (s., 1H) 8.75 (br. s., 1H) 10.59 (br. s., 1H).

### Example 136

### 7-Methyl-6-{2-[(2R)-2-methyl-4-(2-methyl-5-quinolinyl)-1-piperazinyl]ethyl}-4,5-dihydroimidazo[1,5-a]quinoline-3-carboxamide dihydrochloride (E136)

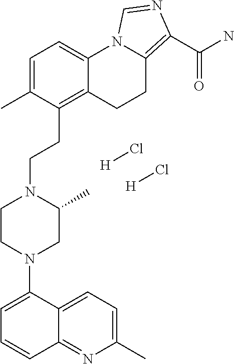

A mixture of ethyl 7-methyl-6-{2-[(2R)-2-methyl-4-(2-methyl-5-quinolinyl)-1-piperazinyl]-ethyl}-4,5-dihydroimidazo[1,5-a]quinoline-3-carboxylate (free base of E130) (92 mg, 0.176 mmol) and potassium hydroxide (4 ml, 1M solution in MeOH) was stirred at reflux for 4 hours. After SCX purification the 7-methyl-6-{2-[(2R)-2-methyl-4-(2-methyl-5-quinolinyl)-1-piperazinyl]ethyl}-4,5-dihydroimidazo[1,5-a]quinoline-3-carboxylic acid was isolated (87 mg, 0.176 mmol, 100%) and used without further purification to prepare the free base of the title compound following the general procedure for amide formation using hexamethyldisilazane (0.048 ml, 0.228 mmol) (see Example 14). The precipitated free base of the title compound was filtered and triturated with ethyl ether (26 mg, 30%). The free base was then treated with HCl (2.2 eq of 1.25M solution in MeOH) in 2:1 methanol/DCM (3 ml) at 0° C. to give the title compound as a yellow solid; MS (ES) m/z: 495.30 [MH⁺], C30H34N6O requires 494.64; ¹H NMR (400 MHz, DMSO-d₆) δ ppm 1.47 (d, 3H) 2.45 (s, 3H) 2.75 (s, 3H) 3.03 (t, 2H) 3.13-3.57 (m, 12H) 3.67-3.86 (m, 1H) 7.14 (s, 1H) 7.26-7.36 (m, 3H) 7.57 (br. m, 1H) 7.70 (d, 1H) 7.77 (s, 1H) 8.45 (s, 1H) 8.62 (br. s, 1H) 10.89 (br. s, 1H).

### Example 137

### 6-{2-[4-(2-Methyl-5-quinolinyl)-1-piperazinyl]ethyl}-4H-tetrazolo[5,1-c][1,4]-benzoxazine dihydrochloride (E137)

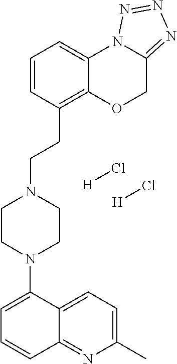

The title compound was prepared in 77% yield following the general reductive amination procedure of Example 1 starting from 4H-tetrazolo[5,1-c][1,4]benzoxazin-6-ylacetaldehyde (D129) (47 mg, 0.218 mmol) and 2-methyl-5-(1-piperazinyl)quinoline (74 mg, 0.326 mmol). The crude product was purified by flash chromatography on silica gel eluting with methanol in DCM (2%) to afford the free base of the title compound (72 mg, 77%). Treatment with HCl (2.2 eq of 1.25M solution in MeOH) in 2:1 methanol/DCM (3 ml) at 0° C. gave the title compound as a yellow solid; MS (ES) m/z: 428.00 [MH⁺], C24H25N7O requires 427.51; ¹H NMR (500 MHz, DMSO-d₆) δ ppm 2.72 (s, 3H) 3.18-3.56 (m, 10H) 3.66-3.78 (m, 2H) 5.84-5.97 (m, 2H) 7.22-7.32 (m, 2H) 7.43 (d, 1H) 7.47-7.63 (m, 1H) 7.67-7.83 (m, 2H) 7.89 (dd, 1H) 8.36-8.75 (m, 1H) 11.24 (br. s., 1H).

### Example 138

### 6-{2-[4-(2-Methyl-5-quinolinyl)-1-piperidinyl]ethyl}-4H-tetrazolo[5,1-c][1,4]-benzoxazine dihydrochloride (E138)

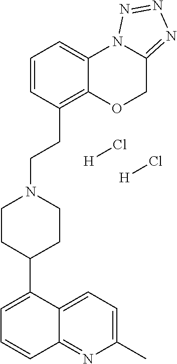

The title compound was prepared in 86% yield following the general reductive amination procedure of Example 1 starting from 4H-tetrazolo[5,1-c][1,4]benzoxazin-6-ylacetaldehyde (D129) (50 mg, 0.231 mmol) and 2-methyl-5-(4-piperidinyl)quinoline (78 mg, 0.347 mmol). The crude product was purified by flash chromatography on silica gel eluting with a gradient of methanol in DCM (2 to 3%) to afford the free base of the title compound (86 mg, 86%). Treatment with HCl (2.2 eq of 1.25M solution in MeOH) in 3:1 methanol/DCM (4 ml) at 0° C. gave the title compound as a solid; MS (ES) m/z: 427.10 [MH⁺], C25H26N6O requires 426.52; ¹H NMR (500 MHz, DMSO-d₆) δ ppm 2.02-2.24 (m, 4H) 2.71 (s, 3H) 3.15-3.25 (m, 2H) 3.23-3.41 (m, 4H) 3.65-3.82 (m, 3H) 5.91 (s, 2H) 7.27 (t, 1H) 7.37-7.49 (m, 2H) 7.49-7.63 (m, 1H) 7.68-7.82 (m, 1H) 7.83-7.95 (m, 2H) 8.44-8.83 (m, 1H) 10.32 (br. s., 1H).

### Example 139

### 6-{2-[(2R)-2-Methyl-4-(2-methyl-5-quinolinyl)-1-piperazinyl]ethyl}-4H-tetrazolo[5,1-c][1,4]-benzoxazine dihydrochloride (E139)

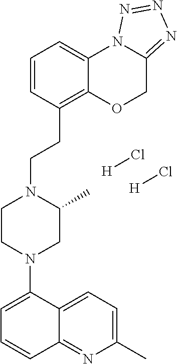

The title compound was prepared in 77% yield following the general reductive amination procedure of Example 1 starting from 4H-tetrazolo[5,1-c][1,4]benzoxazin-6-ylacetaldehyde (D129) (47 mg, 0.217 mmol) and 2-methyl-5-[(3R)-3-methyl-1-piperazinyl]quinoline (79 mg, 0.326 mmol). The crude product was purified by flash chromatography on silica gel eluting with methanol in DCM (2%) to afford the free base of the title compound (74 mg, 77%). Treatment with HCl (2.2 eq of 1.25M solution in MeOH) in 2:1 methanol/DCM (3 ml) at 0° C. gave the title compound as a yellow solid; MS (ES) m/z: 442.00 [MH⁺], C25H27N7O requires 441.54; ¹H NMR (500 MHz, DMSO-d₆) δ ppm 1.45 (d, 3H) 2.70 (s, 3H) 3.08-3.86 (m, 11H) 5.91 (s, 2H) 7.17-7.32 (m, 2H) 7.39-7.58 (m, 2H) 7.63-7.81 (m, 2H) 7.88 (d, 1H) 8.42-8.71 (m, 1H) 11.47 (br. s., 1H).

### Example 140

### 6-{2-[4-(2-Methyl-5-quinolinyl)-1-piperazinyl]ethyl}-4,5-dihydrotetrazolo[1,5-a]quinoline dihydrochloride (E140)

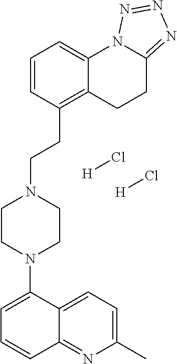

The title compound was prepared in 82% yield following the general reductive amination procedure of Example 1 starting from 4,5-dihydrotetrazolo[1,5-a]quinolin-6-ylacetaldehyde (D130) (41 mg, 0.192 mmol) and 2-methyl-5-(1-piperazinyl)quinoline (65 mg, 0.287 mmol). The crude product was purified by flash chromatography on silica gel eluting with a gradient of methanol in DCM (2 to 3%) to afford the free base of the title compound (67 mg, 82%). Treatment with HCl (2.2 eq of 1.25M solution in MeOH) in 2:1 methanol/DCM (3 ml) at 0° C. gave the title compound as a yellow solid; MS (ES) m/z: 426.10 [MH⁺], C25H27N7 requires 425.54; ¹H NMR (500 MHz, DMSO-d₆) δ ppm 2.72 (s, 3H) 3.19-3.43 (m, 10H) 3.42-3.58 (m, 4H) 3.67-3.82 (m, 2H) 7.21-7.31 (m, 1H) 7.41 (d, 1H) 7.45-7.59 (m, 1H) 7.51 (t, 1H) 7.67-7.82 (m, 2. H) 7.88 (d, 1H) 8.37-8.70 (m, 1H) 11.69 (br. s., 1H).

### Example 141

### 6-{2-[4-(2-Methyl-5-quinolinyl)-1-piperidinyl]ethyl}-4,5-dihydrotetrazolo[1,5-a]quinoline dihydrochloride (E141)

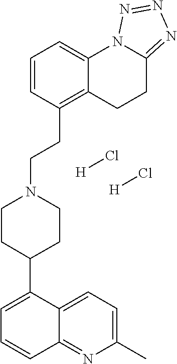

The title compound was prepared in 86% yield following the general reductive amination procedure of Example 1 starting from 4,5-dihydrotetrazolo[1,5-a]quinolin-6-ylacetaldehyde (D130) (50 mg, 0.234 mmol) and 2-methyl-5-(4-piperidinyl)quinoline (79 mg, 0.350 mmol). The crude product was purified by flash chromatography on silica gel eluting with a gradient of methanol in DCM (2 to 3%) to afford the free base of the title compound (86 mg, 86%). Treatment with HCl (2.2 eq of 1.25M solution in MeOH) in 3:1 methanol/DCM (4 ml) at 0° C. gave the title compound as a solid; MS (ES) m/z: 425.10 [MH⁺], C26H28N6 requires 424.55; ¹H NMR (500 MHz, DMSO-d₆) δ ppm 2.02-2.27 (m, 4H) 2.72 (s, 3H) 3.17-3.46 (m, 10H) 3.64-3.85 (m, 3H) 7.43 (d, 1H) 7.44-7.49 (m, 1H) 7.51 (t, 1H) 7.53-7.63 (m, 1H) 7.71-7.84 (m, 1H) 7.84-7.94 (m, 2H) 8.31-8.91 (m, 1H) 10.56 (br. s., 1H).

### Example 142

### 6-{2-[(2R)-2-Methyl-4-(2-methyl-5-quinolinyl)-1-piperazinyl]ethyl}-4,5-dihydrotetrazolo[1,5-a]quinoline dihydrochloride (E142)

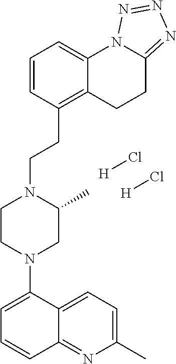

The title compound was prepared in 90% yield following the general reductive amination procedure of Example 1 starting from 4,5-dihydrotetrazolo[1,5-a]quinolin-6-ylacetaldehyde (D130) (41 mg, 0.192 mmol) and 2-methyl-5-[(3R)-3-methyl-1-piperazinyl]quinoline (69 mg, 0.287 mmol) (WO2004/046124). The crude product was purified by flash chromatography on silica gel eluting with a gradient of methanol in DCM (2 to 3%) to afford the free base of the title compound (76 mg, 90%). Treatment with HCl (2.2 eq of 1.25M solution in MeOH) in 2:1 methanol/DCM (3 ml) at 0° C. gave the title compound as a yellow solid; MS (ES) m/z: 440.10 [MH⁺], C26H29N7 requires 439.56; ¹H NMR (500 MHz, DMSO-d₆) δ ppm 1.46 (d, 3H) 2.71 (s, 3H) 3.10-3.60 (m, 12H) 3.60-3.73 (m, 1H) 3.73-3.81 (m, 1H) 3.81-3.89 (m, 1H) 7.20-7.35 (m, 1H) 7.46 (d, 1H) 7.51 (t, 1H) 7.53-7.62 (m, 1H) 7.68-7.83 (m, 2H) 7.90 (d, 1H) 8.41-8.77 (m, 1H) 11.53 (br. s., 1H).

### Example 143

### Ethyl 6-{2-[4-(2-methyl-5-quinolinyl)-1-piperidinyl]ethyl}-4H-[1,2,3]triazolo-[5,1-c][1,4]-benzoxazine-3-carboxylate dihydrochloride (E143)

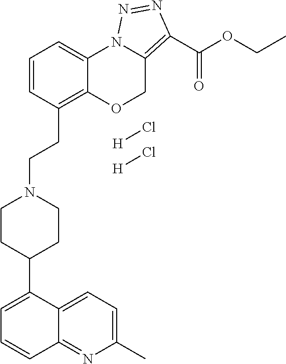

The title compound was prepared in 88% yield following the general reductive amination procedure of Example 1 starting from ethyl 6-(2-oxoethyl)-4H-[1,2,3]triazolo[5,1-c][1,4]benzoxazine-3-carboxylate (D136) (96 mg, 0.33 mmol) and 2-methyl-5-(4-piperidinyl)quinoline (113 mg, 0.50 mmol). The crude product was purified by flash chromatography on silica gel eluting with a gradient of methanol in DCM (2 to 3%) to afford the free base of the title compound (144 mg, 88%). Treatment with HCl (2.2 eq of 1.25M solution in MeOH) in 1:1 methanol/DCM (2 ml) at 0° C. gave the title compound as a solid; MS (ES) m/z: 498.30 [MH⁺], C29H31N5O3 requires 497.60; ¹H NMR (500 MHz, DMSO-d₆) δ ppm 1.34 (t, 3H) 2.03-2.13 (m, 2H) 2.12-2.28 (m, 2H) 2.75 (s, 3H) 3.16-3.23 (m, 2H) 3.23-3.48 (m, 4H) 3.66-3.82 (m, 3H) 4.35 (q, 2H) 5.78 (s, 2H) 7.24 (t, 1H) 7.42 (d, 1H) 7.45-7.55 (m, 1H) 7.54-7.72 (m, 1H) 7.73-7.87 (m, 1H) 7.87-7.97 (m, 1H) 7.99 (dd, 1H) 8.43-9.08 (m, 1H) 10.49 (br. s., 1H).

### Example 144

### N-Methyl-6-{2-[4-(2-methyl-5-quinolinyl-1-piperidinyl]ethyl}-4H-[1,2,3]triazolo[5,1-c][1,4]-benzoxazine-3-carboxamide dihydrochloride (E144)

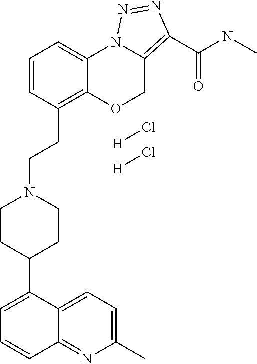

The title compound was prepared following the procedure of Example 38 using the free base of ethyl 6-{2-[4-(2-methyl-5-quinolinyl)-1-piperidinyl]ethyl}-4H-[1,2,3]triazolo[5,1-c][1,4]benzoxazine-3-carboxylate (E143) (44 mg, 0.089 mmol).

The crude product was purified by flash chromatography on silica gel eluting with a gradient of methanol in DCM (2 to 3%) to afford the free base of the title compound (34 mg, 79%). Treatment with HCl (2.2 eq of 1.25M solution in MeOH) in 2:1 methanol/DCM (3 ml) at 0° C. gave the title compound as a solid; MS (ES) m/z: 509.10 [MH⁺], C28H30N6O2 requires 482.58; ¹H NMR (400 MHz, DMSO-d₆) δ ppm 1.98-2.15 (m, 4H) 2.67 (s, 3H) 2.76 (d, 3H) 3.08-3.18 (m, 2H) 3.18-3.38 (m, 4H) 3.58-3.78 (m, 3H) 5.74 (s, 2H) 7.20 (t, 1H) 7.35 (d, 1H) 7.39-7.46 (m, 1H) 7.45-7.62 (m, 1H) 7.66-7.78 (m, 1H) 7.78-7.89 (m, 1H) 7.93 (d, 1H) 8.51-8.67 (m, 1H) 8.71 (d, 1H) 10.05 (br. s., 1H).

### Example 145

### 6-{2-[4-(2-Methyl-5-quinolinyl)-1-piperidinyl]ethyl}-4H-[1,2,3]triazolo[5,1-c][1,4]benzoxazine-3-carboxamide dihydrochloride (E145)

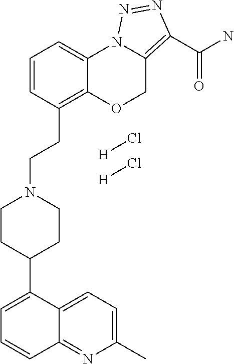

A mixture of ethyl 6-{2-[4-(2-methyl-5-quinolinyl)-1-piperidinyl]ethyl}-4H-[1,2,3]triazolo-[5,1-c][1,4]benzoxazine-3-carboxylate (free base of E143) (90 mg, 0.181 mmol) and lithium hydroxide (30 mg, 0.724 mmol) in THF/H₂O 3/1 (8 ml) was stirred at room temperature for 2 hours. After SCX purification the 6-{2-[4-(2-methyl-5-quinolinyl)-1-piperidinyl]ethyl}-4H-[1,2,3]triazolo[5,1-c][1,4]benzoxazine-3-carboxylic acid was isolated (80 mg, 0.171 mmol, 94%) and used without further purification to prepare the free base of the title compound following the general procedure for amide formation using hexamethyldisilazane (0.040 ml, 0.188 mmol). The precipitated free base was filtered and triturated with ethyl ether (31 mg, 39%) and then treated with HCl (2.2 eq of 1.25M solution in MeOH) in 1:1 methanol/DCM (4 ml) at 0° C. to give the title compound as a white solid; MS (ES) m/z: 469.00 [MH⁺], C27H28N6O2 requires 468.56; ¹H NMR (500 MHz, DMSO-d₆) δ ppm 2.03-2.15 (m, 2H) 2.14-2.31 (m, 2H) 2.78 (s, 3H) 3.03-3.59 (m, 6H) 3.65-3.86 (m, 3H) 5.77 (s, 2H) 7.21 (t, 1H) 7.40 (d, 1H) 7.47-7.58 (m, 1H) 7.58-7.72 (m, 1H) 7.74 (s, 1H) 7.78-7.91 (m, 1H) 7.91-8.07 (m, 1H) 7.96 (d, 1H) 8.15 (s, 1H) 8.56-9.08 (m, 1H) 10.64 (br. s., 1H).

### Example 146

### Ethyl 6-{2-[4-(2-methyl-5-quinolinyl)-1-piperidinyl]ethyl}-4,5-dihydro[1,2,3]-triazolo[1,5-a]quinoline-3-carboxylate dihydrochloride (E146)

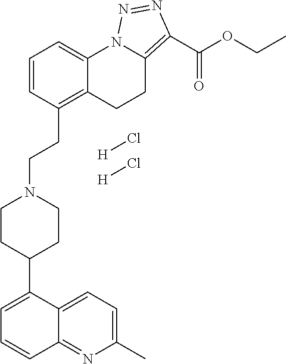

The title compound was prepared in 92% yield following the general reductive amination procedure of Example 1 starting from ethyl 6-(2-oxoethyl)-4,5-dihydro[1,2,3]triazolo[1,5-a]quinoline-3-carboxylate (D137) (71 mg, 0.249 mmol) and 2-methyl-5-(4-piperidinyl)quinoline (84 mg, 0.374 mmol). The crude product was purified by flash chromatography on silica gel eluting with a gradient of methanol in DCM (2 to 3%) to afford the free base of the title compound (113 mg, 92%). Treatment with HCl (2.2 eq of 1.25M solution in MeOH) in 1:1 methanol/DCM (2 ml) at 0° C. gave the title compound as a solid; MS (ES) m/z: 496.10 [MH⁺], C30H33N5O2 requires 495.62; ¹H NMR (500 MHz, DMSO-d₆) δ ppm 1.35 (t, 3H) 2.04-2.29 (m, 4H) 2.75 (s, 3H) 3.14 (t, 2H) 3.21-3.49 (m, 8H) 3.68-3.84 (m, 3H) 4.36 (q, 2H) 7.42 (d, 1H) 7.45-7.55 (m, 1H) 7.49 (t, 1H) 7.55-7.69 (m, 1H) 7.74-7.87 (m, 1H) 7.87-7.99 (m, 1H) 8.01 (d, 1H) 8.45-9.05 (m, 1H) 10.55 (br. s., 1H).

### Example 147

### 6-{2-[4-(2-Methyl-5-quinolinyl)-1-piperidinyl]ethyl}-4,5-dihydro[1,2,3]-triazolo[1,5-a]quinoline-3-carboxamide dihydrochloride (E147)

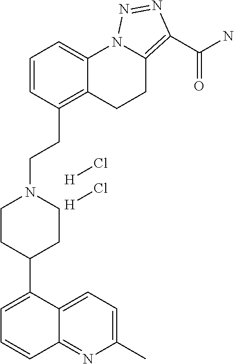

A mixture of ethyl 6-{2-[4-(2-methyl-5-quinolinyl)-1-piperidinyl]ethyl}-4,5-dihydro[1,2,3]-triazolo[1,5-a]quinoline-3-carboxylate (free base of E146) (103 mg, 0.208 mmol) and lithium hydroxide (35 mg, 0.832 mmol) in THF/H₂O 3/1 (8 ml) was stirred at room temperature for 5 hours. After SCX purification the 6-{2-[4-(2-methyl-5-quinolinyl)-1-piperidinyl]ethyl}-4,5-dihydro[1,2,3]triazolo[1,5-a]quinoline-3-carboxylic acid was isolated (88 mg, 0.188 mmol, 91%) and used without further purification to prepare the free base of the title compound following the general procedure for amide formation using hexamethyldisilazane (0.044 ml, 0.207 mmol) (see Example 14). The precipitated free base was filtered and triturated with ethyl ether and methanol (30.9 mg, 35%) and then treated with HCl (2.2 eq of 1.25M solution in MeOH) in 1:1 methanol/DCM (2 ml) at 0° C. to give the title compound as a white solid; MS (ES) m/z: 467.30 [MH⁺], C28H30N6O requires 466.59; ¹H NMR (500 MHz, DMSO-d₆) δ ppm 2.04-2.24 (m, 4H) 2.71 (s, 3H) 3.10 (t, 2H) 3.20-3.47 (m, 8H) 3.64-3.87 (m, 3H) 7.40 (d, 1H) 7.43-7.51 (m, 2H) 7.52-7.59 (m, 2H) 7.68-7.82 (m, 1H) 7.82-7.93 (m, 1H) 7.94 (s, 1H) 8.00 (d, 1H) 8.45-8.80 (m, 1H) 10.34 (br. s., 1H).

### Example 148

### Cyclopropyl(6-{2-[4-(2-methyl-5-quinolinyl)-1-piperazinyl]ethyl}-4H-imidazo[5,1-c][1,4]-benzoxazin-3-yl)methanone dihydrochloride (E148)

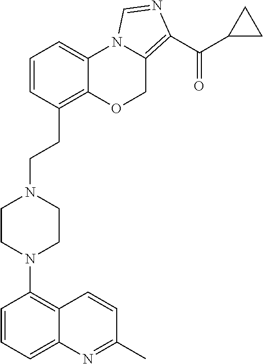

To an ice cooled stirred solution of N-methyl-N-(methyloxy)-6-{2-[4-(2-methyl-5-quinolinyl)-1-piperazinyl]ethyl}-4H-imidazo[5,1-c][1,4]benzoxazine-3-carboxamide (E25) (150 mg, 0.292 mmol) in THF (2.5 ml) was added cyclopropyl magnesium bromide (0.72 ml of a 0.5M solution in THF, 0.351 mmol) and the resulting solution was stirred first for 1 hour at 0° C., then for 2 days at room temperature. The reaction mixture was poured into cold aqueous hydrochloric acid (4 ml of 2.5 M solution), then treated with NaHCO₃ saturated solution (15 ml) and extracted with DCM (3×15 ml). The combined organic layers were dried (Na₂SO₄) and evaporated in vacuo and the resulting crude brown oil was purified by SPE-Si cartridge (2 g) eluting with diethyl ether to afford the free base of the title compound (22 mg, 60%) as a white solid. The free base was treated with HCl (2.1 eq. of 1 M solution in diethyl ether) in dry methanol at 0° C. Evaporation of solvent and trituration with diethyl ether gave the title compound as a yellow solid; MS (ES) m/z: 494.4 [MH⁺], C₃₀H₃₁N₆O₂ requires 493.61; ¹H-NMR (500 MHz, DMSO-d⁶) δ: 11.05 (bs, 1H), 9.02 (bd, 1H), 8.70 (s, 1H), 8.02 (m, 2H), 7.9 (m, 2H), 7.51 (m, 1H), 7.3 (d, 1H), 7.2 (t, 1H), 5.62 (s, 2H), 3.8-3.2 (m, 12H), 3.09 (m, 1H), 2.96 (s, 3H), 1.00 (m, 4H).

### Example 149

### (Mixture of E/Z Isomers): 1-(6-{2-[4-(2-Methyl-5-quinolinyl)-1-piperazinyl]ethyl}-4H-imidazo[5,1-c][1,4]-benzoxazin-3-yl)ethanone O-methyloxime dihydrochloride (E149)

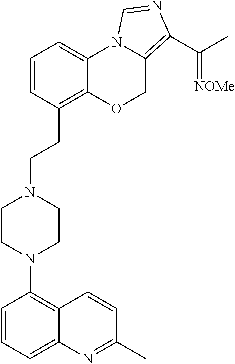

A solution of 1-(6-{2-[4-(2-methyl-5-quinolinyl)-1-piperazinyl]ethyl}-4H-imidazo[5,1-c][1,4]benzoxazin-3-yl)ethanone (free base of E35) (75 mg, 0.160 mmol), pyridine (4 ml) and methoxylamine hydrochloride (27 mg, 0.32 mmol) in 95% ethanol (4 ml) was stirred at reflux for 2 hours. The solvent was evaporated in vacuo and the residue dissolved in water and extracted with DCM (3×15 ml). The combined organic layers were dried (Na₂SO₄) and evaporated in vacuo and the resulting brown oil was purified by SPE-Sil cartridge (2 g) eluting with 4% methanol in DCM to afford the free base of the title compound as a white solid (80 mg, 100%). The free base was treated with HCl (2.1 eq. of 1 M solution in diethyl ether) in dry methanol at 0° C. Evaporation of solvent and trituration with diethyl ether gave the title compound as a yellow solid (mixture of E/Z isomers with 85/15 ratio); MS (ES) m/z: 497.4 [MH⁺], C₂₉H₃₂N₆O₂ requires 496.61; ¹H-NMR (500 MHz, DMSO-d⁶) δ: 11.29 (bs, 1H), 8.95 (bd, 1H), 8.58 (s, 1H), 7.99 (m, 2H), 7.84 (m, 2H), 7.46 (bd, 1H), 7.3-7.1 (m, 2H), 5.48 (s, 2H), 3.89 (s, 3H), 3.8-3.1 (m, 12H), 2.92 (s, 3H), 2.2 (s, 3H).

### Example 150

### (Mixture of E/Z Isomers): Cyclopropyl(6-{2-[4-(2-methyl-5-quinolinyl)-1-piperazinyl]ethyl}-4H-imidazo[5,1-c][1,4]-benzoxazin-3-yl)methanone O-methyloxime dihydrochloride (E1501

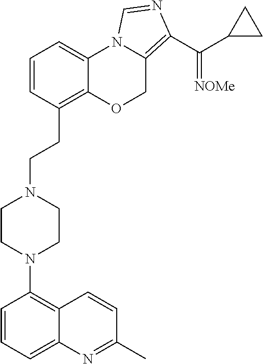

A solution of cyclopropyl(6-{2-[4-(2-methyl-5-quinolinyl)-1-piperazinyl]ethyl}-4H-imidazo[5,1-c][1,4]benzoxazin-3-yl)methanone (free base of E148) (80 mg, 0.162 mmol) pyridine (4 ml) and methoxylamine hydrochloride (27 mg, 0.32 mmol) in 95% ethanol (4 ml) was stirred at reflux for 2 hours. The solvent was evaporated in vacuo and the residue dissolved in water and extracted with DCM (3×15 ml). The combined organic layers were dried (Na₂SO₄) and evaporated in vacuo and the resulting brown oil was purified by SPE-Si cartridge (2 g) eluting with 4% methanol in DCM to afford the free base of the title compound E150 as a white solid (44 mg, 52%). The free base was treated with HCl (2.1 eq. of 1 M solution in diethyl ether) in dry methanol at 0° C. Evaporation of solvent and trituration with diethyl ether gave the title compound as a yellow solid (mixture of E/Z isomers with 65/15 ratio); MS (ES) m/z: 523.4 [MH⁺], C₃₁H₃₄N₆O₂ requires 522.6; ¹H-NMR (500 MHz, DMSO-d⁶) (assigned only the prevalent isomer) δ: 11.58 (bs, 1H), 9.10 (d, 1H), 8.58 (s, 1H), 8.14 (d, 1H), 8.05 (t, 1H), 7.94 (d, 1H), 7.83 (dd, 1H), 7.53 (d, 1H), 7.26 (m, 1H), 7.17 (m, 1H), 5.41 (s, 2H), 3.90 (s, 3H), 3.77 (d, 2H), 3.6-3.4 (m, 8H), 3.23 (m, 2H), 3.01 (s, 3H), 2.41 (m, 1H), 1.42 (m, 2H), 0.89 (m, 2H).

### Example 151

### 2,2,2-Trifluoroethyl 6-{2-[4-(2-methyl-5-quinolinyl)-1-piperazinyl]ethyl}-4H-imidazo[5,1-c][1,4]-benzoxazine-3-carboxylate dihydrochloride (E151)

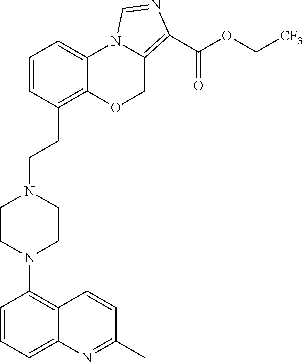

To a mixture of 6-{2-[4-(2-methyl-5-quinolinyl)-1-piperazinyl]ethyl}-4H-imidazo[5,1-c][1,4]benzoxazine-3-carboxylic acid (E2) (50 mg, 0.106 mmol) in DMF (1 ml) was added DIPEA (0.018 ml, 0.116 mmol) and the suspension slowly turned a clear solution. TBTU (38 mg, 0.116 mmol) was added to the solution and the reaction stirred at room temperature for 1 hour. 2,2,2-Trifluoro-1-ethanol (0.05 ml) was added and the final reaction stirred for one night at room temperature. The crude solution was purified by SPE-SCX cartridge (eluting with methanol followed by 2N ammonia solution in methanol) and then by SPE-Si cartridge (2 g) eluting with 3% methanol in DCM to afford the free base of the title compound as a white solid (15 mg, 26%). The free base was treated with HCl (2.1 eq. of 1 M solution in diethyl ether) in dry methanol at 0° C. Evaporation of solvent and trituration with diethyl ether gave the title compound as a yellow solid; MS (ES) m/z: 552.2 [MH⁺], C₂₉H₂₈F₃N₅O₃ requires 551.57; ¹H-NMR (500 MHz, DMSO-d⁶) δ: 11.35 (bs, 1H), 9.05 (d, 1H), 8.72 (s, 1H), 8.06 (d, 1H), 8.01 (t, 1H), 7.91 (d, 1H), 7.89 (d, 1H), 7.5 (d, 1H), 7.29 (d, 1H), 7.19 (t, 1H), 5.6 (s, 2H), 4.97 (q, 2H), 3.73 (bd, 2H), 3.5 (bm, 4H), 3.39 (bm, 4H), 3.21 (bt, 2H), 2.97 (s, 3H).

### Example 152

### 2,22-Trifluoro-1-methylethyl 6-{2-[4-(2-methyl-5-quinolinyl)-1-piperazinyl]-ethyl}-4H-imidazo[5,1-c][1,4]benzoxazine-3-carboxylate dihydrochloride (E152)

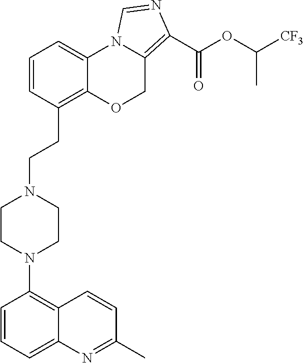

To a mixture of 6-{2-[4-(2-methyl-5-quinolinyl)-1-piperazinyl]ethyl}-4H-imidazo[5,1-c][1,4]benzoxazine-3-carboxylic acid (E2) (40 mg, 0.0852 mmol) in DMF (1 ml) was added DIPEA (0.017 ml, 0.0938 mmol) and the suspension slowly turned to a clear solution. TBTU (30 mg, 0.0938 mmol) was added to the solution and the reaction stirred at room temperature for 1 hour before 1,1,1-trifluoro-2-Propanol (0.05 ml) was added and the reaction mixture stirred for one night at room temperature. The crude solution was purified by SPE-SCX cartridge (eluting with methanol followed by 2N ammonia solution in methanol) and then by SPE-Si cartridge (2 g) eluting with 3% methanol in DCM to afford the free base of the title compound as a white solid (40 mg, 83%). The free base was treated with HCl (2.1 eq. of 1 M solution in diethyl ether) in dry methanol at 0° C. Evaporation of solvent and trituration with diethyl ether gave the title compound as a yellow solid. MS (ES) m/z 566.7 [MH⁺], C₃₀H₃₀F₃N₅O₃ requires 565.59; ¹H NMR (500 MHz, DMSO-d₆) δ ppm 1.49 (d, 3H) 2.97 (s, 3H) 3.22 (dd, 2H) 3.35-3.47 (m, 4H) 3.47-3.57 (m, 4H) 3.75 (d, 2H) 5.61 (s, 2H) 5.66-5.74 (m, 1H) 7.20 (t, 1H) 7.30 (d, 1H) 7.51 (d, 1H) 7.87-7.95 (m, 2H) 7.98-8.11 (m, 2H) 8.72 (s, 1H) 9.06 (d, 1H) 11.34 (br. s., 1H)

### Example 153

### Cyclopropylmethyl 6-{2-[4-(2-methyl-5-quinolinyl)-1-piperazinyl]ethyl}-4H-imidazo[5,1-c][1,4]-benzoxazine-3-carboxylate dihydrochloride (E153)

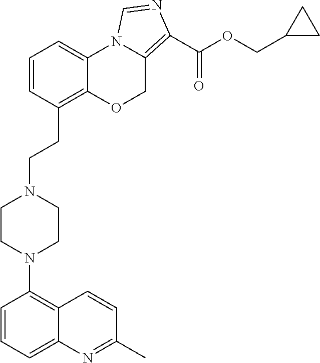

To a mixture of 6-{2-[4-(2-methyl-5-quinolinyl)-1-piperazinyl]ethyl}-4H-imidazo[5,1-c][1,4]benzoxazine-3-carboxylic acid (E2) (40 mg, 0.0852 mmol) in DMF (1 ml) was added DIPEA (0.017 ml, 0.0938 mmol) and the suspension slowly turned to a clear solution. TBTU (30 mg, 0.0938 mmol) was added to the solution and the reaction stirred at room temperature for 1 hour before cyclopropylmethanol (0.06 ml) was added and the final reaction stirred for one night at room temperature. The crude solution was purified by SPE-SCX cartridge (eluting with methanol followed by 2N ammonia solution in methanol) and then by SPE-Si cartridge (2 g) eluting with 3% Methanol in DCM to afford the free base of the title compound as a white solid (40 mg, 83%). The free base was treated with HCl (2.1 eq. of 1 M solution in diethyl ether) in dry methanol at 0° C. Evaporation of solvent and trituration with diethyl ether gave the title compound as a yellow solid; MS (ES) m/z: 524.3 [MH⁺], C₃₁H₃₃N₆O₃ requires 523.63; ¹H NMR (500 MHz, DMSO-d₆) δ ppm 0.35 (q, J=5.86 Hz, 2H) 0.57 (q, J=7.81 Hz, 2H) 1.15-1.27 (m, 1H) 2.99 (s, 3H) 3.22 (dd, J=7.81 Hz, 2H) 3.41-3.44 (m, J=10.74 Hz, 4H) 3.49-3.54 (m, J=10.74 Hz, 4H) 3.74 (d, J=10.74 Hz, 2H) 4.09 (d, J=7.81 Hz, 2H) 5.62 (s, 2H) 7.18 (t, J=7.81 Hz, 1H) 7.29 (d, J=6.83 Hz, 1H) 7.52 (d, J=7.81 Hz, 1H) 7.88 (d, J=7.81 Hz, 1H) 7.93 (d, J=8.79 Hz, 1H) 8.03 (t, J=8.30 Hz, 1H) 8.10 (d, 1H) 8.67 (s, 1H) 9.08 (d, J=8.79 Hz, 1H) 11.48 (br. s., 1H).

### Example 154

### 1-Methylethyl 6-{2-[4-(2-methyl-5-quinolinyl)-1-piperazinyl]ethyl}-4H-imidazo[5,1-c][1,4]-benzoxazine-3-carboxylate dihydrochloride (E154)

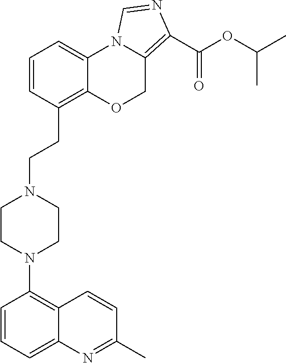

To a mixture of 6-{2-[4-(2-methyl-5-quinolinyl)-1-piperazinyl]ethyl}-4H-imidazo[5,1-c][1,4]benzoxazine-3-carboxylic acid (E2) (40 mg, 0.0852 mmol) in DMF (1 ml) was added DIPEA (0.017 ml, 0.0938 mmol) and the suspension slowly turned to a clear solution. TBTU (30 mg, 0.0938 mmol) was added to the solution and the reaction stirred at room temperature for 1 hour before isopropanol (0.10 ml) was added and the final reaction stirred for one night at room temperature. The crude solution was purified by SPE-SCX cartridge (eluting with methanol followed by 2N ammonia solution in methanol) and then by SPE-Si cartridge (2 g) eluting with 3% methanol in DCM to afford the free base of the title compound as a white solid (40 mg, 92%). The free base was treated with HCl (2.1 eq. of 1 M solution in diethyl ether) in dry methanol at 0° C. Evaporation of the solvent and trituration with diethyl ether gave the title compound as a yellow solid; MS (ES) m/z 512.3 [MH⁺], C₃₀H₃₃N₆O₃ requires 511.62; ¹H NMR (500 MHz, DMSO-d₆) δ ppm 1.31 (d, J=6.83 Hz, 6H) 2.99 (s, 3H) 3.22 (t, 2H) 3.42-3.44 (m, 4H) 3.51-3.54 (m, J=9.76 Hz, 4H) 3.74 (d, J=9.76 Hz, 2H) 5.06-5.19 (m, 1H) 5.60 (s, 2H) 7.17 (t, J=7.81 Hz, 1H) 7.28 (d, J=7.81 Hz, 1H) 7.52 (d, 1H) 7.87 (d, J=8.79 Hz, 1H) 7.93 (d, J=8.79 Hz, 1H) 8.03 (t, J=8.30 Hz, 1H) 8.09 (d, 1H) 8.65 (s, 1H) 9.07 (d, J=8.79 Hz, 1H) 11.48 (br. s., 1H)

### Example 155

### Cyclopentyl 6-{2-[4-(2-methyl-5-quinolinyl)-1-piperazinyl]ethyl}-4H-imidazo[5,1-c][1,4]-benzoxazine-3-carboxylate dihydrochloride (E155)

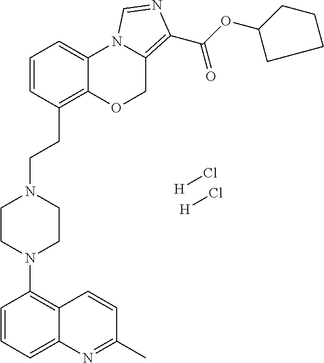

To a mixture of 6-{2-[4-(2-methyl-5-quinolinyl)-1-piperazinyl]ethyl}-4H-imidazo[5,1-c][1,4]benzoxazine-3-carboxylic acid (E2) (40 mg, 0.0852 mmol) in DMF (1 ml) was added DIPEA (0.017 ml, 0.0938 mmol) and the suspension slowly turned to a clear solution. TBTU (30 mg, 0.0938 mmol) was added to the solution and the reaction stirred at room temperature for 1 hour before cyclopentanol (0.10 ml) was added and the final reaction stirred one night at room temperature. The crude solution was purified by SPE-SCX cartridge (eluting with methanol followed by 2N ammonia solution in methanol) and then by SPE-Si cartridge (2 g) eluting with 3% methanol in DCM to afford the free base of the title compound as a white solid (40 mg, 87%). The free base was treated with HCl (2.1 eq. of 1 M solution in diethyl ether) in dry methanol at 0° C. Evaporation of solvent and trituration with diethyl ether gave the title compound as a yellow solid; MS (ES) m/z: 538.3 [MH⁺], C₃₂H₃₅N₅O₃ requires 537.66; ¹H-NMR (500 MHz, DMSO-d⁶) δ: 11.41 (bs, 1H), 9.07 (d, 1H), 8.65 (s, 1H), 8.08 (d, 1H), 8.02 (t, 1H), 7.92 (d, 1H), 7.87 (d, 1H), 7.5 (d, 1H), 7.27 (d, 1H), 7.16 (t, 1H), 5.58 (s, 2H), 5.28 (m, 1H), 3.74 (bd, 2H), 3.47 (m, 4H), 3.38 (m, 4H), 3.19 (m, 2H), 2.97 (s, 3H), 1.91 (m, 2H), 1.74 (m, 4H), 1.61 (bm, 2H).

### Example 156

### N′-Acetyl-6-{2-[4-(2-methyl-5-quinolinyl)-1-piperazinyl]ethyl}-4H-imidazo[5,1-c][1,4]-benzoxazine-3-carbohydrazide dihydrochloride (E156)

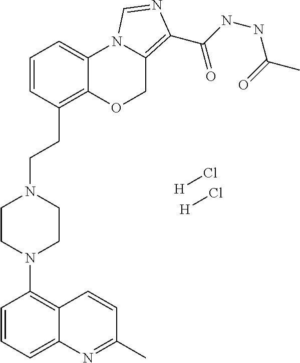

To a solution of trimethylaluminium (0.9 ml of a 2 M sol. in hexane, 1.8 mmol) in dry DCM (1.5 ml) at 0° C. was added acetohydrazide (134 mg, 1.8 mmol) and the resulting mixture was stirred at room temperature for 30 minutes. Ethyl 6-{2-[4-(2-methyl-5-quinolinyl)-1-piperazinyl]ethyl}-4H-imidazo[5,1-c][1,4]-benzoxazine-3-carboxylate dihydrochloride (E1) (150 mg, 0.3 mmol) in dry DCM (1.5 ml) was added and the resulting solution was stirred at 54° C. for 4 hours. The reaction mixture was quenched with water (caution, very exotermic reaction) then NaOH 1 M (15 ml) was added and the reaction extracted with DCM (3×10 ml). The combined organic layers were dried (Na₂SO₄) and evaporated in vacuo to give a pale yellow solid that was triturated with ethyl ether to afford the free base of the title compound (116 mg, 74%) as a white solid. The free base was treated with HCl (2.1 eq. of 1 M solution in diethyl ether) in dry methanol at 0° C. Evaporation of solvent and trituration with diethyl ether gave the title compound as a yellow solid; MS (ES) m/z: 527.2 [MH⁺], C₂₃H₃₃H₇O₃ requires 526.6; ¹H NMR (500 MHz, DMSO-d₆) d ppm 1.88 (s, 3H) 2.94 (s, 3H) 3.16-3.25 (m, 2H) 3.32-3.40 (m, 2H) 3.40-3.47 (m, 2H) 3.46-3.56 (m, 4H) 3.73 (d, 2H) 5.58 (s, 2H) 7.17 (t, 1H) 7.27 (d, 1H) 7.49 (d, 1H) 7.80-7.94 (m, 2H) 7.93-8.08 (m, 2H) 8.62 (s, 1H) 9.02 (br. s., 1H) 9.77 (s, 1H) 9.89 (s, 1H) 11.11 (br. s., 1H).

### Example 157

### 3-(5-Methyl-1,3,4-oxadiazol-2-yl)-6-{2-[4-(2-methyl-5-quinolinyl)-1-piperazinyl]ethyl}-4H-imidazo[5,1-c][1,4]-benzoxazine dihydrochloride (E157)

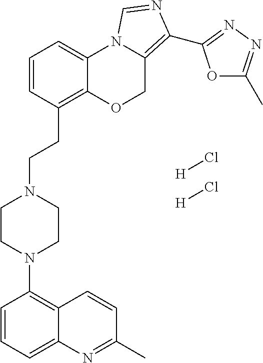

To a stirred suspension of N′-acetyl-6-{2-[4-(2-methyl-5-quinolinyl)-1-piperazinyl]ethyl}-4H-imidazo[5,1-c][1,4]benzoxazine-3-carbohydrazide (free base of E156) (50 mg, 0.095 mmol) and dry pyridine (0.015 ml, 0.19 mmol) in dry DCM at 0° C. was added triflic anhydride (0.029 ml, 1.71 mmol) and the resulting mixture was stirred for 1 hour at 0° C. and for 1 night at room temperature. The reaction mixture was basified to pH 8-9 with NaHCO₃ and then extracted with DCM (3×10 ml). The combined organic layers were dried (Na₂SO₄) and evaporated in vacuo and the resulting crude oil was purified by SPE-Si cartridge (2 g) eluting with 4% methanol in DCM to afford the free base of the title compound as a white solid (24 mg, 50%). The free base was treated with HCl (2.1 eq. of 1 M solution in diethyl ether) in dry methanol at 0° C. Evaporation of solvent and trituration with diethyl ether gave the title compound as a yellow solid; MS (ES) m/z: 508.2 [MH⁺], C₂₃H₂₉N₇O₂ requires 507.6; ¹H NMR (500 MHz, DMSO-d₆) d ppm 2.58 (s, 3H) 2.92 (s, 3H) 3.22 (t, 2H) 3.32-3.41 (m, 2H) 3.41-3.48 (m, 2H) 3.48-3.58 (m, 2H) 3.58-3.83 (m, 4H) 5.66 (s, 2H) 7.20 (t, 1H) 7.30 (d, 1H) 7.47 (d, 1H) 7.81-7.87 (m, 1H) 7.91 (d, 1H) 7.99 (br. s., 2H) 8.77 (s, 1H) 8.97 (br. s., 1H) 11.13 (br. s., 1H).

### Example 158

### 6-{2-[4-(2-Methyl-5-quinolinyl)-1-piperazinyl]ethyl}-3-(1,3-oxazol-5-yl)-4H-imidazo[5,1-c][1,4]-benzoxazine dihydrochloride (E158)

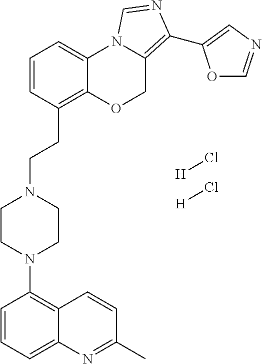

A mixture of 6-{2-[4-(2-methyl-5-quinolinyl)-1-piperazinyl]ethyl}-4H-imidazo[5,1-c][1,4]benzoxazine-3-carbaldehyde (D138) (50 mg, 0.11 mmol), K₂CO₃ (31 mg, 0.22 mmol), p-toluenesulfonylmethyl isocyanide (21 mg, 0.11 mmol) in MeOH (1 ml) was stirred overnight at room temperature. The crude solution was purified by SPE-SCX cartridge (eluting with methanol followed by 2N ammonia solution in methanol) and then triturated with diethyl ether to afford the corresponding free base of the title compound as a solid (51 mg, 100%). The free base was treated with HCl (2.1 eq. of 1 M solution in diethyl ether) in dry methanol at 0° C. Evaporation of solvent and trituration with diethyl ether gave the title compound as a yellow solid; MS (ES) m/z: 493.4 [MH⁺], C₂₉H₂₈N₆O₂ requires 492.58; ¹H NMR (500 MHz, DMSO-d₆) d ppm 2.99 (s, 3H) 3.19-3.28 (m, 2H) 3.37-3.48 (m, 4H) 3.47-3.60 (m, 4H) 3.74 (d, 2H) 5.56 (s, 2H) 7.17 (t, 1H) 7.27 (d, 1H) 7.42 (s, 1H) 7.51 (d, 1H) 7.86 (d, 1H) 7.93 (d, 1H) 8.03 (t, 1H) 8.13 (d, 1H) 8.44 (s, 1H) 8.73 (s, 1H) 9.08 (d, 1H) 11.61 (br. s., 1H).

### Example 159

### 3-(3-Methyl-5-isoxazolyl)-6-{2-[4-(2-methyl-5-quinolinyl)-1-piperazinyl]ethyl}-4H-imidazo[5,1-c][1,4]-benzoxazine dihydrochloride (E159)

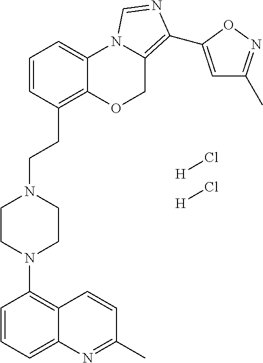

(2Z)-3-(Dimethylamino)-1-(6-{2-[4-(2-methyl-5-quinolinyl)-1-piperazinyl]-ethyl}-4H-imidazo[5,1-c][1,4]benzoxazin-3-yl)-2-buten-1-one (D139) (33 mg, 0.0614 mmol), was dissolved in ethanol (2 ml) and hydroxylamine hydrochloride (6.5 mg, 0.0922 mmol) was added. The reaction mixture was irradiated in a microwave reactor (PersonalChemistry Emrys™ Optimiser, 300W, 150° C., 5 minutes). The solvent was evaporated, then saturated aqueous NH₄Cl solution (10 ml) was added and the mixture extracted with ethyl acetate (3×10 ml). The combined organic layers were dried (Na₂SO₄) and evaporated in vacuo to afford the free base of the title compound as a cream solid (15 mg, 48%). The free base was treated with HCl (2.1 eq. of 1 M solution in diethyl ether) in dry methanol at 0° C. Evaporation of solvent and trituration with diethyl ether gave the title compound as a yellow solid; MS (ES) m/z: 507.3 [MH⁺], C₃₀H₃₀N₆O₂ requires 506.61; ¹H NMR (500 MHz, DMSO-d₆) d ppm 2.28 (s, 3H) 2.98 (s, 3H) 3.18-3.27 (m, 2H) 3.36-3.47 (m, 4H) 3.47-3.57 (m, 4H) 3.74 (d, 2H) 5.61 (s, 2H) 6.60 (s, 1H) 7.18 (t, 1H) 7.27 (d, 1H) 7.51 (d, 1H) 7.87 (d, 1H) 7.93 (d, 1H) 8.02 (t, 1H) 8.10 (d, 1H) 8.72 (s, 1H) 9.07 (d, 1H) 11.48 (br. s., 1H).

### Example 160

### 3(3-Methyl-1H-pyrazol-5-yl)-6-{2-[4(2-methyl-5-quinolinyl)-1-piperazinyl]ethyl}-4H-imidazo[5,1-c][1,4]-benzoxazine dihydrochloride (E160)

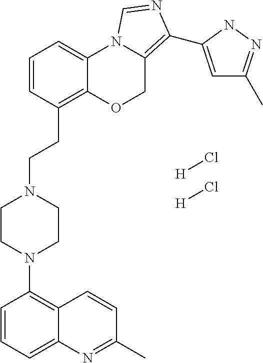

(2Z)-3-(Dimethylamino)-1-(6-{2-[4-(2-methyl-5-quinolinyl)-1-piperazinyl]-ethyl}-4H-imidazo[5,1-c][1,4]benzoxazin-3-yl)-2-buten-1-one (D139) (33 mg, 0.0614 mmol) was dissolved in ethanol (2 ml) and hydrazine hydrate (0.003 mg, 0.0922 mmol) was added. The reaction mixture was irradiated in a microwave reactor (PersonalChemistry Emrys™ Optimiser, 300W, 150° C., 5 min). The solvent was evaporated then saturated aqueous NH₄Cl solution (5 ml) was added and the mixture extracted with ethyl acetate (3×10 ml). The combined organic layers were dried (Na₂SO₄) and evaporated in vacuo to afford the free base of the title compound as a cream solid (16 mg, 48%). The free base was treated with HCl (2.1 eq. of 1 M solution in diethyl ether) in dry methanol at 0° C. Evaporation of solvent and trituration with diethyl ether gave the title compound as a yellow solid; MS (ES) m/z: 506.4 [MH⁺] C₃₀H₃₁N₇O requires 505.62.

### Example 161

### Ethyl 6-{2-[4-fluoro-4-(5-quinolinyl)-1-piperidinyl]ethyl}-4H-imidazo[5,1-c][1,4]benzoxazine-3-carboxylate dihydrochloride (E161)

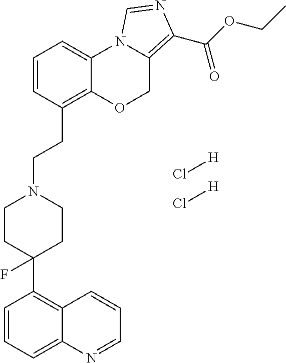

A solution of 5-(4-fluoro-4-piperidinyl)quinoline (D142) (90 mg, 0.391 mmol) and ethyl 6-(2-oxoethyl)-4H-imidazo[5,1-c][1,4]benzoxazine-3-carboxylate (D6) (100 mg, 0.355 mmol) dissolved in 1,2 DCE (2 ml) was stirred at room temperature for 30 minutes then sodium triacetoxyborohydride (75 mg, 0.355 mmol) was added and the resulting mixture stirred at the same temperature for 1 night. The reaction mixture was quenched with water (10 ml) and extracted with DCM (3×10 ml). The combined organic layers were dried (Na₂SO₄) and evaporated in vacuo and the resulting mixture was purified by SPE-Si cartridge (2 g) eluting with 50% ethyl acetate in cyclohexane to afford the free base of the title compound as a white foam (122 mg, 62%); MS (ES) m/z: 501.3 [MH⁺], C₂₉H₂₉FN₄O₃ requires 500.57; ¹H-NMR (400 MHz, CDCl₃-d⁶) δ: 9.07 (bd, 2H), 8.15 (d, 1H), 8.05 (s, 1H), 7.7 (t, 1H), 75 (bd, 1H), 7.4 (bd, 2H), 7.27 (bd, 1H), 7.16 (t, 1H), 5.58 (s, 2H), 4.4 (q, 2H), 3.3-2.0 (bm, 12H), 1.4 (t, 3H). The free base was treated with HCl (2.1 eq. of 1 M solution in diethyl ether) in dry methanol at 0° C. Evaporation of solvent and trituration with diethyl ether gave the title compound as a yellow solid; MS (ES) m/z: 501.3 [MH⁺], C₂₉H₂₉FN₄O₃ requires 500.57.

### Example 162

### Ethyl 6-(2-{4-[2-(fluoromethyl)-5-quinolinyl]-1-piperazinyl}ethyl)-4H-imidazo[5,1-c][1,4]-benzoxazine-3-carboxylate (E162)

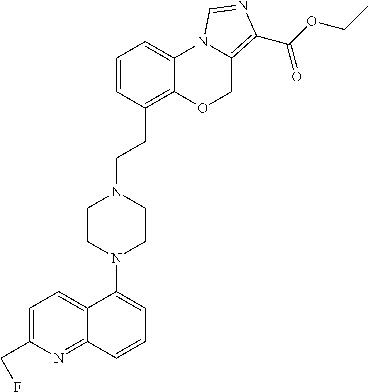

The title compound (118 mg, 54%) was obtained by the procedure described for Example 56 using 2-(fluoromethyl)-5-(1-piperazinyl)quinoline (D146) (104 mg, 0.424 mmol); MS (ES; m/z): 516 [MH⁺], C₂₉H₃₀FN₅O₃ requires 515.59; NMR (¹H, 300 MHz, CDCl₃) δ: 8.56 (d, 1H), 7.97 (s, 1H), 7.7 (d, 1H), 7.56 (m, 2H), 7.34 (d, 1H), 7.14 (m, 2H), 7.11 (t, 1H), 5.62 (d, 2H), 5.53 (s, 2H), 4.38 (q, 2H), 3.1 (m, 4H), 2.94 (m, 2H), 2.82 (m, 4H), 2.72 (m, 2H), 1.40 (t, 3H).

### Example 163

### Ethyl 6-(2-{4-[2-(difluoromethyl)-5-quinolinyl]-1-piperazinyl}ethyl)-4H-imidazo[5,1-c][1,4]-benzoxazine-3-carboxylate (E163)

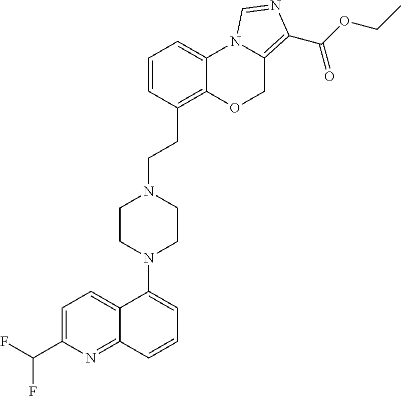

The title compound (211 mg, 95%) was obtained by the procedure of Example 56 using 2-(difluoromethyl)-5-(1-piperazinyl)quinoline (D143) (109 mg, 0.414 mmol); MS (ES; m/z): 534 [MH⁺], C₂₉H₂₉F₂N₅O₃ requires 533.58; NMR (¹H, 300 MHz, CDCl₃) δ: 8.64 (d, 1H), 7.98 (s, 1H), 7.82 (d, 1H), 7.67 (m, 2H), 7.34 (m, 1H), 7.18 (m, 2H), 7.14 (t, 1H), 6.75 (t, 1H), 5.53 (s, 2H), 4.39 (q, 2H), 3.15 (m, 4H), 2.95 (m, 2H), 2.83 (bm, 4H), 2.72 (m, 2H), 1.41 (t, 3H).

### Example 164

### Ethyl 6-[2-(4-{2-[(dimethylamino)carbonyl]-5-quinolinyl}-1-piperazinyl)-ethyl]-4H-imidazo[5,1-c][1,4]-benzoxazine-3-carboxylate (E164)

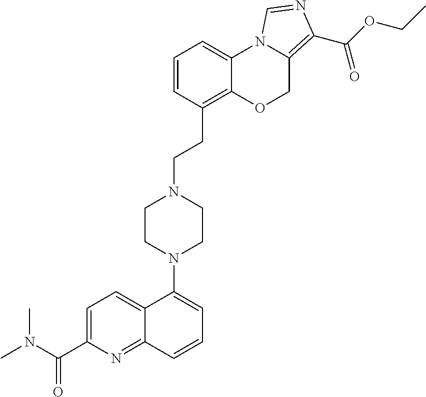

The title compound (152 mg, 76%) was obtained by the procedure of Example 56 using N,N-dimethyl-5-(1-piperazinyl)-2-quinolinecarboxamide (D150) (102 mg, 0.359 mmol); MS (ES; m/z): 555 [MH⁺], C₃₁H₃₄N₈O₄ requires 554.66; NMR (¹H, 300 MHz, CDCl₃) δ: 8.58 (s, 1H), 7.97 (s, 1H), 7.76 (d, 1H), 7.64 (m, 2H), 7.34 (d, 1H), 7.15 (m, 2H), 7.05 (t, 1H), 5.53 (s, 2H), 4.39 (q, 2H), 3.1 (m, 10H), 2.95 (m, 2H), 2.83 (bs, 4H), 2.70 (m, 2H), 1.40 (t, 3H).

### Example 165

### Ethyl 6-[2-(4-{2-[(methylamino)carbonyl]-5-quinolinyl}-1-piperazinyl)ethyl]-4H-imidazo[5,1-c][1,4]benzoxazine-3-carboxylate (E165)

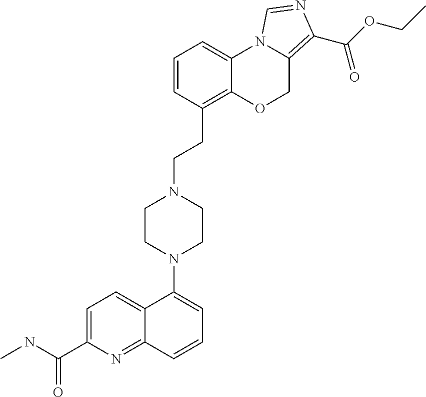

The title compound (133 mg, 69%) was obtained by the procedure of Example 56 using N-methyl-5-(1-piperazinyl)-2-quinolinecarboxamide (D151) (96 mg, 0.355 mmol); MS (ES; m/z): 541 [MH⁺], C₃₀H₃₂N₆O₄ requires 540.62; NMR (¹H, 300 MHz, CDCl₃) δ: 8.62 (d, 1H), 8.25 (d, 1H), 8.2 (d, 1H), 7.97 (s, 1H), 7.74 (d, 1H), 7.67 (t, 1H), 7.34 (d, 1H), 7.15 (m, 2H), 7.04 (t, 1H), 5.53 (s, 2H), 4.39 (q, 2H), 3.1 (m, 7H), 2.95 (m, 2H), 2.90 (m, 4H), 2.72 (m, 2H), 1.40 (t, 3H).

### Example 166

### 6-(2-{4-[2-(Fluoromethyl)-5-quinolinyl]-1-piperazinyl}ethyl)-N-methyl-4H-imidazo[5,1-c][1,4]-benzoxazine-3-carboxamide (E166)

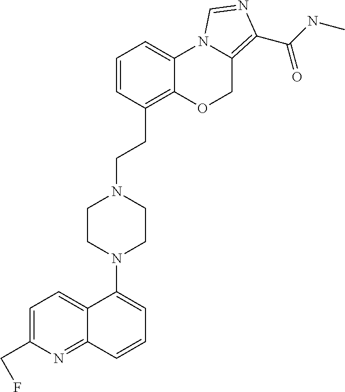

The title compound (21 mg, 74%) was obtained by the procedure of Example 57 using ethyl 6-(2-{4-[2-(fluoromethyl)-5-quinolinyl]-1-piperazinyl}ethyl)-4H-imidazo[5,1-c][1,4]-benzoxazine-3-carboxylate (E162) (29 mg, 0.057 mmol). Stirring was carried out at room temperature overnight; MS (ES; m/z): 501 [MH⁺], C₂₈H₂₆FN₆O₂ requires 500.58; NMR (¹H, 400 MHz, DMSO-d₆) δ: 8.58 (d, 1H), 8.55 (s, 1H), 8.14 (q, 1H), 7.76 (dd, 1H), 7.7 (m, 2H), 7.64 (d, 1H), 7.22 (m, 2H), 7.10 (t, 1H), 5.66 (d, 2H), 5.55 (s, 2H), 3.07 (bs, 4H), 2.89 (t, 2H), 2.8 (bs, 4H), 2.77 (d, 3H), 2.65 (t, 2H).

### Example 167

### 6-(2-{4-[2-(Fluoromethyl)-5-quinolinyl]-1-piperazinyl}ethyl)-N,N-dimethyl-4H-imidazo[5,1-c][1,4]-benzoxazine-3-carboxamide (E167)

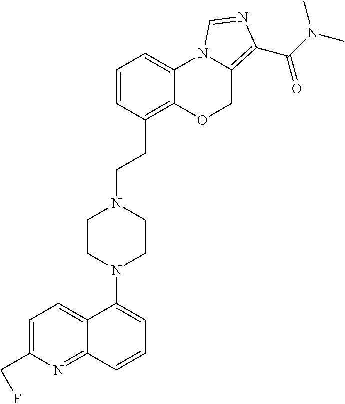

The title compound (24 mg, 82%) was obtained by the procedure of Example 58 using ethyl 6-(2-{4-[2-(fluoromethyl)-5-quinolinyl]-1-piperazinyl}ethyl)-4H-imidazo[5,1-c][1,4]benzoxazine-3-carboxylate (E162) (29 mg, 0.057 mmol) and dimethylamine (2.0M/THF). Stirring was carried out at room temperature overnight; MS (ES; m/z): 515 [MH⁺], C₂₉H₃₁FN₆O₂ requires 514.61; NMR (¹H, 400 MHz, DMSO-d₆) δ: 8.58 (d, 1H), 8.58 (s, 1H), 7.76 (dd, 1H), 7.7 (m, 2H), 7.64 (d, 1H), 7.22 (m, 2H), 7.10 (t, 1H), 5.66 (d, 2H), 5.48 (s, 2H), 3.49 (bs, 3H), 3.07 (bs, 4H), 2.98 (bs, 3H), 2.89 (t, 2H), 2.78 (bs, 4H), 2.66 (t, 2H).

### Example 168

### 6-(2-{4-[2-(Fluoromethyl)-5-quinolinyl]-1-piperazinyl}ethyl)-3-(4-morpholinylcarbonyl)-4H-imidazo[5,1-c][1,4]-benzoxazine (E168)

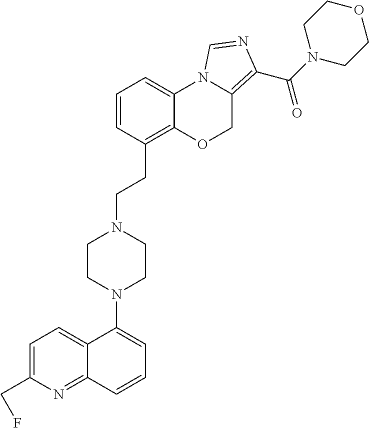

The title compound (25 mg, 80%) was obtained by the procedure of Example 59 using ethyl 6-(2-{4-[2-(fluoromethyl)-5-quinolinyl]-1-piperazinyl}ethyl)-4H-imidazo[5,1-c][1,4]benzoxazine-3-carboxylate (E162) (29 mg, 0.057 mmol). Stirring was carried out at room temperature overnight followed by 4 hours at 50° C.; MS (ES; m/z): 557 [MH⁺], C₃₁H₃₃FN₆O₃ requires 556.65; NMR (¹H, 400 MHz, DMSO-d₆) δ: 8.58 (d, 1H), 8.56 (s, 1H), 7.77 (dd, 1H), 7.7 (m, 2H), 7.64 (d, 1H), 7.22 (m, 2H), 7.11 (t, 1H), 5.66 (d, 2H), 5.51 (s, 2H), 4.3 (bs, 2H), 3.66 (m, 6H), 3.08 (bs, 4H), 2.89 (bt, 2H), 2.78 (bs, 4H), 2.66 (bt, 2H).

### Example 169

### 6-(2-{4-[2-(Difluoromethyl)-5-quinolinyl]-1-piperazinyl}ethyl)-N-methyl-4H-imidazo[5,1-c][1,4]benzoxazine-3-carboxamide (E169)

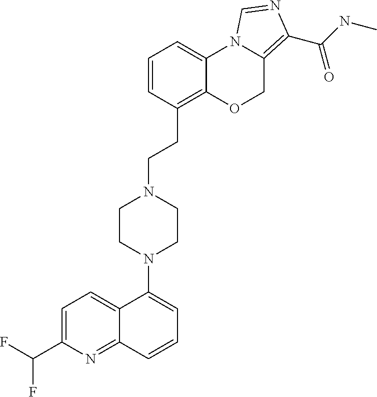

The title compound (40 mg, 78%) was obtained by the procedure of Example 57 using ethyl 6-(2-{4-[2-(difluoromethyl)-5-quinolinyl]-1-piperazinyl}ethyl)-4H-imidazo[5,1-c][1,4]benzoxazine-3-carboxylate (E163) (53 mg, 0.099 mmol). Stirring was carried out at room temperature overnight; MS (ES; m/z): 519 [MH⁺], C₂₈H₂₈F₂N₆O₂ requires 518.57; NMR (¹H, 400 MHz, DMSO-d₆) δ: 8.71 (d, 1H), 8.55 (s, 1H), 8.13 (q, 1H), 7.78 (m, 4H), 7.30 (dd, 1H), 7.24 (dd, 1H), 7.1 (t, 1H), 7.12 (t, 1H), 5.55 (s, 2H), 3.09 (bs, 4H), 2.89 (t, 2H), 2.8 (bs, 4H), 2.77 (d, 3H), 2.66 (t, 2H).

### Example 170

### 6-(2-{4-[2-(Difluoromethyl)-5-quinolinyl]-1-piperazinyl}ethyl)-N,N-dimethyl-4H-imidazo[5,1-c][1,4]-benzoxazine-3-carboxamide (E170)

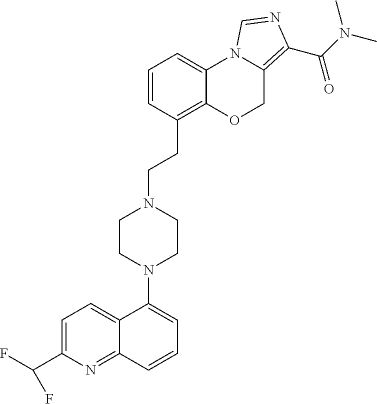

The title compound (47 mg, 90%) was obtained by the procedure of Example 58 using ethyl 6-(2-{4-[2-(difluoromethyl)-5-quinolinyl]-1-piperazinyl}ethyl)-4H-imidazo[5,1-c][1,4]benzoxazine-3-carboxylate (E163) (53 mg, 0.099 mmol) and dimethylamine (2.0M/THF). Stirring was carried out at room temperature overnight; MS (ES; m/z): 533 [MH⁺], C₂₉H₃₀F₂N₆O₂ requires 532.60; NMR (¹H, 400 MHz, DMSO-d₈) δ: 8.71 (d, 1H), 8.55 (s, 1H), 7.78 (m, 4H), 7.30 (dd, 1H), 7.25 (dd, 1H), 7.1 (t, 1H), 7.1 (t, 1H), 5.48 (s, 2H), 3.49 (bs, 3H), 3.09 (bs, 4H), 2.98 (bs, 3H), 2.89 (t, 2H), 2.78 (bs, 4H), 2.66 (t, 2H).

### Example 171

### 6-(2-{4-[2-(Difluoromethyl)-5-quinolinyl]-1-piperazinyl}ethyl)-3-(4-morpholinylcarbonyl)-4H-imidazo[5,1-c][1,4]benzoxazine (E171)

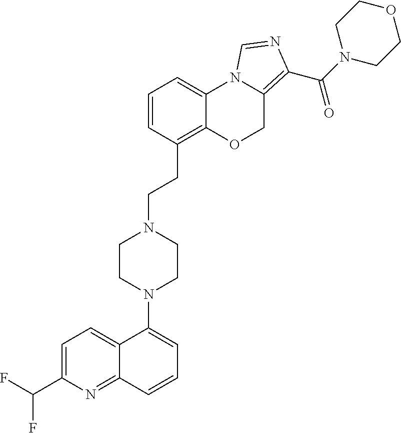

The title compound (39 mg, 68%) was obtained by the procedure of Example 59 using ethyl 6-(2-{4-[2-(difluoromethyl)-5-quinolinyl]-1-piperazinyl}ethyl)-4H-imidazo[5,1-c][1,4]benzoxazine-3-carboxylate (E163) (29 mg, 0.057 mmol). Stirring was carried out at room temperature overnight followed by 4 hours at 50° C.; MS (ES; m/z): 575 [MH⁺], C₃₁H₃₂F₂N₆O₃ requires 574.64; NMR (¹H, 400 MHz, DMSO-d₆) δ: 8.71 (d, 1H), 8.56 (s, 1H), 7.8 (m, 4H), 7.31 (quint., 1H), 7.24 (d, 1H), 7.1 (m, 2H), 5.51 (s, 2H), 4.3 (vbs, 2H), 3.7 (m, 6H), 3.09 (bs, 4H), 2.89 (t, 2H), 2.78 (bs, 4H), 2.66 (m, 2H).

### Example 172

### N-Methyl-6-[2-(4-{2-[(methylamino)carbonyl]-5-quinolinyl}-1-piperazinyl)ethyl]-4H-imidazo[5,1-c][1,4]benzoxazine-3-carboxamide (E172)

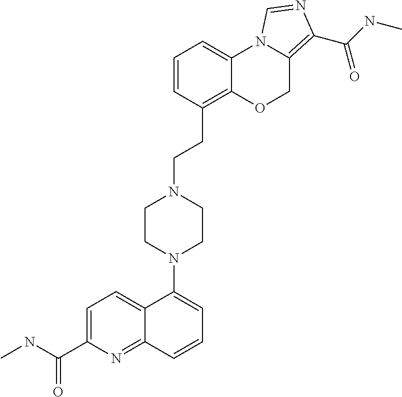

The title compound (14 mg, 43%) was obtained by the procedure of Example 57 using ethyl 6-[2-(4-{2-[(methylamino)carbonyl]-5-quinolinyl}-1-piperazinyl)ethyl]-4H-imidazo[5,1-c][1,4]benzoxazine-3-carboxylate (E165) (33 mg, 0.061 mmol). Stirring was carried out at room temperature overnight; MS (ES; m/z): 526 [MH⁺], C₂₆H₃₁N₇O₃ requires 525.62; NMR (¹H, 400 MHz, DMSO-d₆) δ: 8.88 (q, 1H), 8.66 (d, 1H), 8.55 (s, 1H), 8.13 (dd, 1H), 8.13 (q, 1H), 7.78 (m, 3H), 7.29 (dd, 1H), 7.25 (dd, 1H), 7.1 (t, 1H), 5.55 (s, 2H), 3.09 (bs, 4H), 2.9 (d, 3H), 2.88 (t, 2H), 2.8 (bs, 4H), 2.77 (d, 3H), 2.66 (t, 2H).

### Example 173

### N,N-Dimethyl-6-[2-(4-{2-[(methylamino)carbonyl]-5-quinolinyl}-1-piperazinyl)ethyl]-4H-imidazol[5,1-c][1,4]-benzoxazine-3-carboxamide (E173)

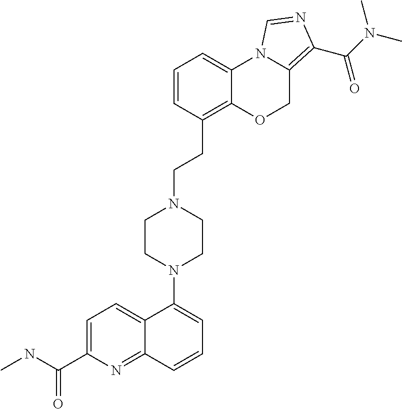

The title compound (29 mg, 88%) was obtained by the procedure of Example 58 using ethyl 6-[2-(4-{2-[(methylamino)carbonyl]-5-quinolinyl}-1-piperazinyl)ethyl]-4H-imidazo[5,1-c][1,4]benzoxazine-3-carboxylate (E165) (33 mg, 0.061 mmol) and dimethylamine (2.0M/THF). Stirring was carried out at room temperature overnight; MS (ES; m/z): 540 [MH⁺], C₃₀H₃₃N₇O₃ requires 539.64; NMR (¹H, 400 MHz, DMSO-d₆) δ: 8.88 (q, 1H), 8.66 (d, 1H), 8.55 (s, 1H), 8.13 (d, 1H), 7.78 (m, 3H), 7.28 (dd, 1H), 7.24 (dd, 1H), 7.1 (t, 1H), 5.48 (s, 2H), 3.49 (bs, 3H), 3.09 (bs, 4H), 2.98 (bs, 3H), 2.86 (t, 2H), 2.9 (d, 3H), 2.78 (bs, 4H), 2.66 (t, 2H).

### Example 174

### N-Methyl-5-(4-{2-[3-(4-morpholinylcarbonyl)-4H-imidazo[5,1-c][1,4]-benzoxazin-6-yl]ethyl}-1-piperazinyl)-2-quinolinecarboximide (E174)

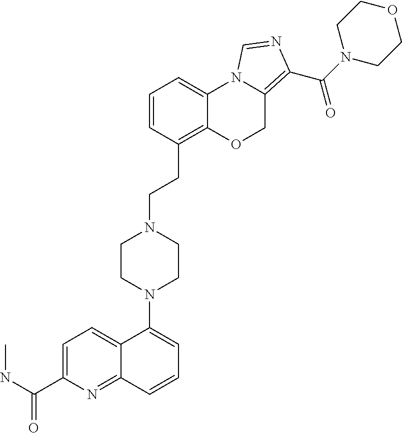

The title compound (24 mg, 70%) was obtained by the procedure of Example 59 using ethyl 6-[2-(4-{2-[(methylamino)carbonyl]-5-quinolinyl}-1-piperazinyl)ethyl]-4H-imidazo[5,1-c][1,4]benzoxazine-3-carboxylate (E165) (33 mg, 0.061 mmol). Stirring was carried out at room temperature overnight followed by 4 h at 50° C.; MS (ES; m/z): 582 [MH⁺], C₃₂H₃₆N₇O₄ requires 581.68; NMR (¹H, 400 MHz, DMSO-d₆) δ: 8.88 (quart., 1H), 8.66 (dd, 1H), 8.56 (s, 1H), 8.13 (d, 1H), 7.8 (m, 3H), 7.28 (dd, 1H), 7.24 (dd, 1H), 7.11 (t, 1H), 5.51 (s, 2H), 4.3 (bs, 2H), 3.7 (m, 6H), 3.09 (bs, 4H), 2.9 (d, 3H), 2.9 (m, 2H), 2.78 (bs, 4H), 2.66 (m, 2H).

### Example 175

### 6-[2-(4-{2-[(Dimethylamino)carbonyl]-5-quinolinyl}-1-piperazinyl)ethyl]-N,N-dimethyl-4H-imidazo[5,1-c][1,4]-benzoxazine-3-carboxamide (E175)

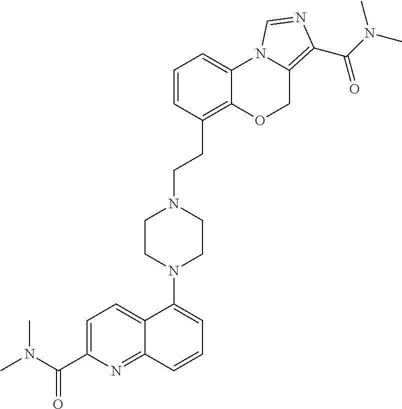

The title compound (35 mg, 92%) was obtained by the procedure of Example 58 using ethyl 6-[2-(4-{2-[(dimethylamino)carbonyl]-5-quinolinyl}-1-piperazinyl)-ethyl]-4H-imidazo[5,1-c][1,4]benzoxazine-3-carboxylate (E164) (38 mg, 0.068 mmol) and dimethylamine (2.0M/THF). Stirring was carried out at room temperature overnight; MS (ES; m/z): 554 [MH⁺], C₃₁H₃₅N₇O₃ requires 553.67; NMR (¹H, 400 MHz, DMSO-d₆) δ: 8.58 (d, 1H), 8.55 (s, 1H), 7.76 (dd, 1H), 7.73 (m, 2H), 7.63 (d, 1H), 7.25 (m, 2H), 7.1 (t, 1H), 5.48 (s, 2H), 3.49 (bs, 3H), 3.09 (bs, 4H), 3.08 (s, 3H), 3.0 (s, 3H), 2.98 (bs, 3H), 2.86 (t, 2H), 2.69 (bs, 4H), 2.66 (t, 2H).

### Example 176

### N,N-Dimethyl-5-(4-{2-[3-(4-morpholinylcarbonyl)-4H-imidazo[5,1-c][1,4]-benzoxazin-6-yl]ethyl}-1-piperazinyl)-2-quinolinecarboxamide (E176)

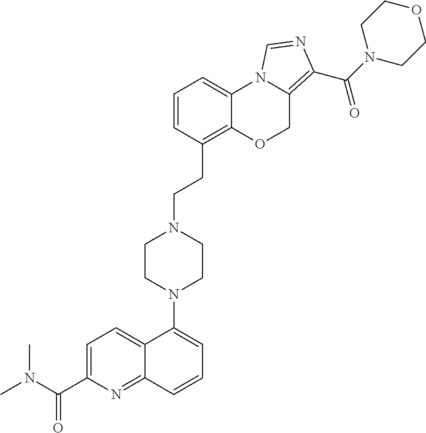

The title compound (37 mg, 92%) was obtained by the procedure of Example 59 using ethyl 6-[2-(4-{2-[(dimethylamino)carbonyl]-5-quinolinyl}-1-piperazinyl)-ethyl]-4H-imidazo[5,1-c][1,4]benzoxazine-3-carboxylate (E164) (38 mg, 0.068 mmol). Stirring was carried out at room temperature overnight followed by 4 hours at 50° C.; MS (ES; m/z): 596 [MH⁺], C₃₃H₃₇N₇O₄ requires 595.71; NMR (¹H, 400 MHz, DMSO-d₆) δ: 8.59 (d, 1H), 8.56 (s, 1H), 7.77 (dd, 1H), 7.7 (m, 2H), 7.63 (d, 1H), 7.25 (m, 2H), 7.11 (t, 1H), 5.51 (s, 2H), 4.3 (bs, 2H), 3.7 (m, 6H), 3.08 (bs, 4H), 3.08 (s, 3H), 3.0 (s, 3H), 2.89 (t, 2H), 2.77 (bs, 4H), 2.66 (m, 2H).

### Examples 177-179 (Pure Enantiomers)

### Ethyl 6-{2-[2-methyl-4-(2-methyl-5-quinolinyl)-1-piperidinyl]ethyl}-4,5-dihydroimidazo[1,5-a]quinoline-3-carboxylate (E177-179)

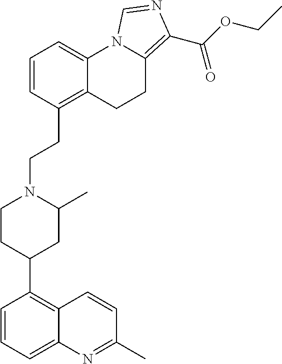

A solution of 2-methyl-5-(2-methyl-4-piperidinyl)quinoline (D163) (212 mg, 0.883 mmol) and ethyl 6-(2-oxoethyl)-4,5-dihydroimidazo[1,5-a]quinoline-3-carboxylate (D86) (228 mg, 0.803 mmol) in 1,2 DCE (2 ml) was stirred at room temperature for 30 minutes. Sodium triacetoxyborohydride (170 mg, 0.803 mmol) was added and the resulting mixture stirred at the same temperature overnight. The reaction mixture was quenched with water (10 ml) and extracted with DCM (3×10 ml). The combined organic layers were dried (Na₂SO₄) and evaporated in vacuo. The residue was purified on a Horizon column (25M) eluting with 3% methanol in DCM to afford a racemic mixture of the title compounds as a white foam (340 mg, 77%); MS (ES) m/z: 509.3 [MH⁺], C₃₂H₃₆N₄O₂ requires 508.66.

The racemic mixture was separated by semi-preparative SFC (Gilson) chromatography [CHIRALCEL AD-H, 25×2.1 cm; modifier 27% (Ethanol+0.1% isopropylamine), flow rate=22 ml/min; pressure 195 bar, T=36° C.; UV wavelength: 220 nm; loop=1 ml to obtain enantiomer 1 (E177) (21 mg), enantiomer 4 (E178) (13 mg) enantiomer 2+3 (E179) (110 mg). The enantiomeric excess of enantiomers 1 and 4 were verified by analytical SFC (Berger) conditions: Chiral column: CHIRALPAK AD-H, 25×0.46 cm; modifier: 27%% (Ethanol+0.1% isopropylamine), flow rate=2.5 ml/min; pressure 180 bar, T=35° C.; UV wavelength: 220 nm; loop=10 microl

Enantiomer 1 E177 (100% a/a by UV, retention time min, e.e=100%)
Enantiomer 4 E178 (100% a/a by UV, retention time min, e.e=100%)

### Enantiomer 2+3 E179

### Example 180

### 6-{2-[4-(2-Methyl-5-quinolinyl)-1-piperazinyl]ethyl}-4,5-dihydroimidazo[1,5-a]quinoline-3-carboxylate (E180)

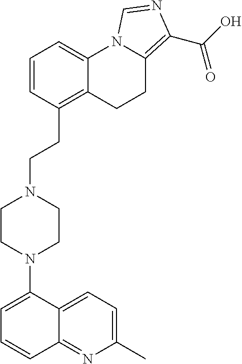

To ethyl 6-{2-[4-(2-methyl-5-quinolinyl)-1-piperazinyl]ethyl}-4,5-dihydroimidazo[1,5-a]quinoline-3-carboxylate (free base of E77) (138 mg, 0.379 mmol) was added KOH (1M solution in MeOH, 10 ml) and the mixture was left to stir at reflux. After 3 hours the solution was cooled down to room temperature, the solvent was removed in vacuo and the crude product was purified by SCX cartridge. The title compound was obtained as the ammonium salt in 94% yield (122 mg); MS (ES) m/z: 468.20 [MH⁺], C28H29N5O2 requires 467.57; ¹H-NMR (300 MHz, DMSO-d6) δ: 8.40 (s, 1H), 8.33 (d, 1H), 7.66 (d, 1H), 7.57 (m, 2H), 7.36 (d, 1H), 7.29 (t, 1H), 7.20 (d, 1H), 7.09 (q, 1H), 3.22 (bt, 2H), 3.03 (vbm, 4H), 2.91 (bm, 4H), 2.76 (vbm, 4H), 2.62 (s, 3H), 2.62-2.45 (m, 2H).

### Example 181

### Ammonium 6-{2-[4-(2-methyl-5-quinolinyl)-1-piperidinyl]ethyl}[1,2,3]-triazolo[1,5-a]quinoline-3-carboxylate (D181)

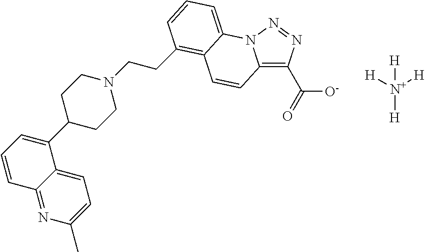

To ethyl 6-{2-[4-(2-methyl-5-quinolinyl)-1-piperidinyl]ethyl}[1,2,3]triazolo[1,5-a]quinoline-3-carboxylate (free base of E99) (50 mg, 0.1 mmol) was added KOH (1M solution in MeOH, 0.6 ml) and the mixture was left to stir at reflux. After 2 hours the solution was cooled down to room temperature, the precipitate was completely dissolved adding H₂O and then the solution was purified by SPE-SCX cartridge (eluting with methanol followed by 2N ammonia solution in methanol) affording the title compound; MS (ES) m/z: 466.2 [MH⁺], C28H27N5O2 requires 465.5.

### Example 182

### N-Methyl-N-(methyloxy)-6-{2-[4-(2-methyl-5-quinolinyl)-1-piperidinyl]ethyl}-4H-imidazo[5,1-c][1,4]-benzoxazine-3-carboxamide (E182)

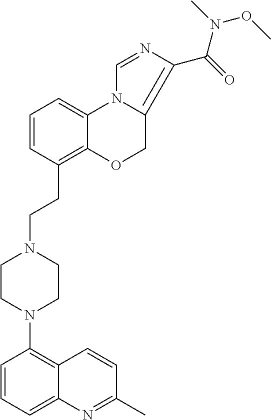

A solution of trimethylaiuminium (2.0M in hexanes, 2.60 ml, 5.20 mmol) and N,O-dimethylhydroxylamine hydrochloride (0.51 g, 5.20 mmol) in dry DCM (20 ml) was stirred at room temperature for 30 minutes. Ethyl 6-{2-[4-(2-methyl-5-quinolinyl)-1-piperidinyl]ethyl}-4H-imidazo[5,1-c][1,4]benzoxazine-3-carboxylate (free base of E37) (0.43 g, 0.87 mmol) was then slowly added and the resulting reaction mixture was stirred at 40° C. for a further 2 hours. After the reaction was completed, 1M NaOH aqueous solution (20 ml) was added dropwise until no more gas evolved. The aqueous solution was extracted with DCM (3×20 ml). The combined organic phases were dried (Na₂SO₄) and evaporated in vacuo. The reaction crude was triturated with diethyl ether to afford the title compound (0.39 g, 0.76 mmol, 87% yield) as a pale yellow solid; MS (ES; m/z): 512.3 [MH⁺]. C₃₀H₃₃N₅O₃ requires 511.62; ¹H NMR (400 MHz, CDCl₃) δ ppm 1.95-2.05 (bm, 4H) 2.34-2.42 (bm, 2H) 2.63-2.70 (bm, 1H) 2.75 (s, 3H) 2.95-3.08 (bm, 2H) 3.30-3.37 (bm, 4H) 3.61 (bs, 3H) 3.89 (s, 3H) 5.58 (s, 2H) 7.09 (t, 1H) 7.21 (d, 1H) 7.40-7.32 (m, 2H) 7.48 (d, 1H) 7.64 (t, 1H) 7.92 (d, 1H) 8.04 (s, 1H) 8.31 (d, 1H).

### Example 183

### 6-{2-[(2R)-4-(7-Fluoro-2-methyl-5-quinolinyl)-2-methyl-1-piperazinyl]ethyl}-4H-imidazo[5,1-c][1,4]benzoxazine-3-carboxylic acid (E184)

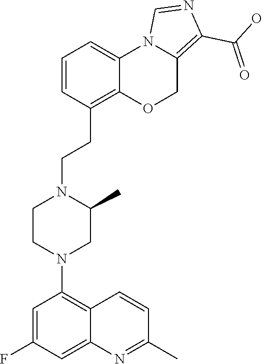

To a solution of ethyl 6-{2-[(2R)-4-(7-fluoro-2-methyl-5-quinolinyl)-2-methyl-1-piperazinyl]ethyl}-4H-imidazo[5,1-c][1,4]benzoxazine-3-carboxylate (E118) (58 mg, 0.1 mmol) in MeOH (3 ml) was added NaOH (1 ml of a 10% aqueous solution) and the resulting white suspension was heated for 2 hours at 80 C. The mixture was evaporated and the crude product purified by SCX column eluting with ammonia in methanol, recovering 0.038 g of the title compound E183 as a pale yellow foam; MS (ES) m/z: 502.5 [MH⁺], C₂₈H₂₈FN₅O₃ requires 501.56.

### Example 184

### N-Methyl-N-(methyloxy)-6-{2-[4-(2-methyl-5-quinolinyl)-1-piperidinyl]ethyl}-4,5-dihydroimidazo[1,5-a]quinoline-3-carboxamide (E184)

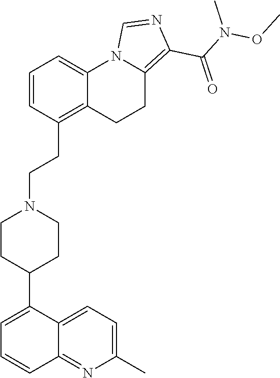

The title compound was prepared following the procedure of Example 41 using methoxylamine hydrochloride (118 mg, 1.21 mmol) and the free base of ethyl 6-{2-[4-(2-methyl-5-quinolinyl)-1-piperidinyl]ethyl}-4,5-dihydroimidazo-[1,5-a]quinoline-3-carboxylate (E82) (100 mg, 0.202 mmol); MS (ES) m/z: 510.1 [MH⁺], C31H35N5O2 requires 509.65; ¹H NMR (300 MHz, CDCl₃) δ ppm 2.06 (br. m, 3H) 2.40 (br. m, 2H) 2.60-2.85 (br. m, 2H) 2.77 (s, 3H) 2.90-3.60 (m, 13H) 3.89 (s, 3H) 7.20-7.40 (m, 4H) 7.47 (d, 1H) 7.68 (t, 1H) 7.93 (d, 1H) 7.99 (s, 1H) 8.32 (d, 1H).

### Example 185

### Ethyl 6-{2-[(2R)-4-(7-fluoro-2-methyl-5-quinolinyl)-2-methyl-1-piperazinyl]ethyl}-4,5-dihydroimidazo[1,5-a]quinoline-3-carboxylate

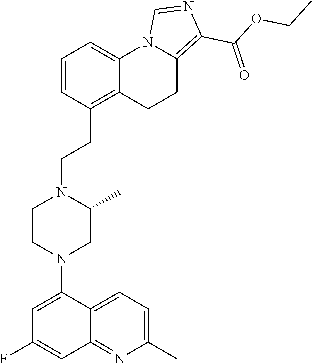

The title compound was prepared in 49% yield following the general reductive amination procedure of Example 1 starting from ethyl 6-(2-oxoethyl)-4,5-dihydroimidazo[1,5-a]quinoline-3-carboxylate (D86) (91 mg, 0.32 mmol) and 7-fluoro-2-methyl-5-[(3R)-3-methyl-1-piperazinyl]quinoline (made by a procedure similar to those described in WO2004/046124) (100 mg, 0.39 mmol). The crude product was purified by flash chromatography on silica gel eluting with methanol in DCM (1%) to afford the free base of the title compound; MS (ES) m/z: 528.3.1 [MH⁺], C13H34FN5O2 requires 527.64; ¹H-NMR (400 MHz, CDCl₃) δ: 8.39 (d, 1H), 8.00 (s, 1H), 7.37-7.56 (m, 4H) 7.3 (m, 1H), 6.9 (d, 1H), 4.45 (quart., 2H), 3.-2.7 (bm, 11H), 2.77 (s, 3H), 1.46 (t, 3H), 1.25 (m, 3H).

### Example 186

### 6-{2-[(2R)-4-(7-fluoro-2-methyl-5-quinolinyl)-2-methyl-1-piperazinyl]ethyl}-4,5-dihydroimidazo[1,5-a]quinoline-3-carboxylic acid (E186)

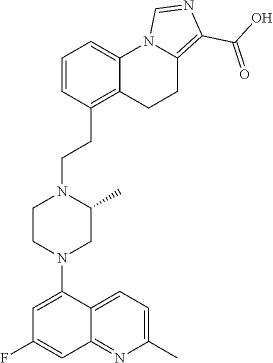

A mixture of free base of ethyl 6-{2-[(2R)-4-(7-fluoro-2-methyl-5-quinolinyl)-2-methyl-1-piperazinyl]ethyl}-4,5-dihydroimidazo[1,5-a]quinoline-3-carboxylate (E185) (100 mg, 0.19 mmol) and KOH (1 M sol in MeOH, 1.2 ml) was stirred at reflux for 2 hrs. The yellow solid was filtered, collected and suspended in water (5 mL). Acetic acid was added until pH=7 and the pale yellow solid formed was filtered, washed with diethylether and dried under vacuum. The title compound (80 mg, 0.14 mmol, 84%) was recovered as a pale yellow foam; MS (ES) m/z: 500.3 [MH⁺]. C₂₉H₃₀FN₅O₂ requires 501.5.

### Example 187

### Ethyl 6-{2-[(2R)-4-(7-fluoro-2-methyl-5-quinolinyl)-2-methyl-1-piperazinyl]ethyl}-4,5-dihydro[1,2,3]triazolo[1,5-a]quinoline-3-carboxylate dihydrochloride (E187)

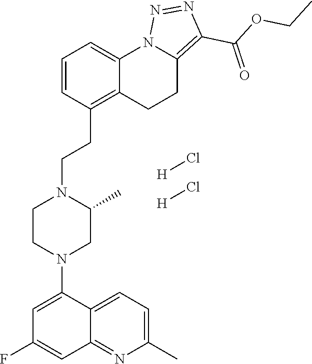

The title compound was prepared in 92% yield following the general reductive amination procedure of Example 1 starting from ethyl 6-(2-oxoethyl)-4,5-dihydro[1,2,3]triazolo[1,5-a]quinoline-3-carboxylate (D137) (50 mg, 0.175 mmol) and 7-fluoro-2-methyl-5-[(3R)-3-methyl-1-piperazinyl]quinoline (50 mg, 0.193 mmol). The crude product was purified by flash chromatography on silica gel eluting with methanol in DCM (2%) to afford the free base of the title compound (85 mg, 92%). Treatment with HCl (2.2 eq of 1.25M solution in MeOH) in methanol (1 ml) at 0° C. gave the title compound as a yellow solid; MS (ES) m/z: 529.10 [MH⁺]. C30H33FN6O2 requires 528.63; ¹H NMR (500 MHz, DMSO-d₆) δ ppm 1.34 (t, 3H) 1.45 (d; 3H) 2.70 (s, 3H) 2.97-3.67 (m, 4H) 3.15 (t, 2H) 3.20-3.54 (m, 4H) 3.39 (t, 2H) 3.48-3.94 (m, 3H) 4.26-4.45 (m, 2H) 7.20 (d, 1H) 7.37-7.56 (m, 4H) 8.02 (d, 1H) 8.39-8.62 (m, 1H) 10.96 (br. s., 1. H).

### Example 188

### 6-{2-[(2R)-4-(7-fluoro-2-methyl-5-quinolinyl)-2-methyl-1-piperazinyl]ethyl}-4,5-dihydro[1,2,3]triazolo[1,5-a]quinoline-3-carboxylic acid (E188)

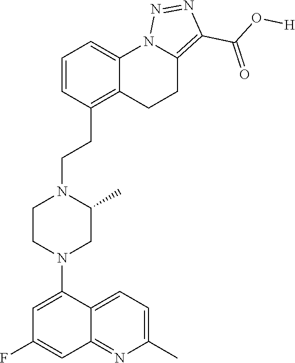

A mixture of ethyl 6-{2-[(2R)-4-(7-fluoro-2-methyl-5-quinolinyl)-2-methyl-1-piperazinyl]ethyl}-4,5-dihydro[1,2,3]triazolo[1,5-a]quinoline-3-carboxylate (free base of E187) (75 mg, 0.142 mmol) and KOH (1M sol in MeOH, 2 ml) was stirred at reflux for 45 minutes. The mixture was purified by SCX column eluting with NH3 in methanol, affording the title compound (70 mg, 0.14 mmol, 99%) as a pale yellow foam; MS (ES) m/z: 501.3 [MH⁺]. C₂₈H₂₈FN₆O₂ requires 500.5.

### Example 189

### 6-{2-[(2R)-4-(7-fluoro-2-methyl-5-quinolinol)-2-methyl-1-piperazinyl]ethyl}-4,5-dihydro[1,2,3]triazolo[1,5-a]quinoline-3-carboxamide dihydrochloride (E189)

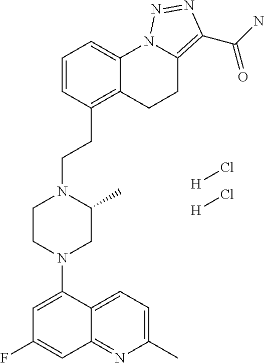

The title compound was prepared following the general procedure for amide formation (see Example 14) starting from 6-{2-[(2R)-4-(7-fluoro-2-methyl-5-quinolinyl)-2-methyl-1-piperazinyl]ethyl}-4,5-dihydro[1,2,3]triazolo[1,5-a]quinoline-3-carboxylic acid (E188) (70 mg, 0.14 mmol) using hexamethyldisilazane (0.033 ml, 0.154 mmol). The crude product was purified by SCX cartridge and flash chromatography on silica gel eluting with a gradient of methanol in DCM (2 to 3%) to afford the free base of the title compound. Treatment with HCl (2.2 eq of 1.25M solution in MeOH) in 1:2 methanol/DCM (3 ml) at 0° C. gave the title compound as a yellow solid; MS (ES) m/z: 500.10 [MH⁺]. C28H30FN7O requires 499.59; ¹H NMR (500 MHz, DMSO-d₆) δ ppm 1.45 (d, 3H) 2.71 (s, 3H) 3.05-3.21 (m, 2H) 3.19-3.52 (m, 4H) 3.22-3.33 (m, 2H) 3.23-3.63 (m, 6H) 3.58-3.84 (m, 1H) 7.22 (d, 1H) 7.43 (d, 1H) 7.47 (t, 1H) 7.45-7.54 (m, 1H) 7.45-7.53 (m, 1H) 7.57 (s, 1H) 7.96 (s, 1H) 8.00 (d, 1H) 8.36-8.69 (m, 1H) 11.19 (br. s., 1H)

### Example 190

### N-methyl-N-(methyloxy)-6-{2-[4-(2-methyl-5-quinolinyl)-1-piperidinyl]ethyl}-4,5-dihydro[1,2,3]triazolo[1,5-a]quinoline-3-carboxamide (E190)

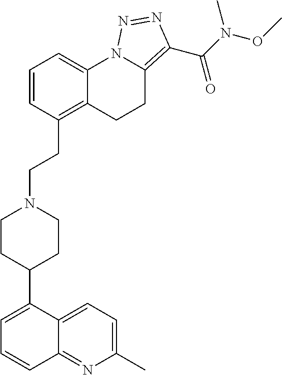

The title compound was prepared following the procedure of Example 184 using N,O-dimethylhydroxylamine hydrochloride (178 mg, 1.82 mmol) and ethyl 6-{2-[4-(2-methyl-5-quinolinyl)-1-piperidinyl]ethyl}-4,5-dihydro[1,2,3]-triazolo[1,5-a]quinoline-3-carboxylate (free base of E146) (150 mg, 0.303 mmol). The crude product was purified by flash chromatography on silica gel eluting with a gradient of methanol in DCM (2 to 3%) to afford the title compound (106 mg, 69%); MS (ES) m/z: 511.3 [MH⁺]. C30H34N6O2 requires 510.64; ¹H NMR (300 MHz, CDCl₃) δ ppm 2.03 (br. m, 3H) 2.40 (br. m, 2H) 2.61-2.82 (br. m, 2H) 2.75 (s, 3H) 2.95-3.65 (m, 13H) 3.95 (s, 3H) 7.22-7.48 (m, 4H) 7.65 (t, 1H) 7.90 (d, 1H) 8.06 (d, 1H) 8.28 (d, 1H).

### Example 191

### 1-(6-{2-[4-(2-methyl-5-quinolinyl)-1-piperidinyl]ethyl}-4,5-dihydro[1,2,3]triazolo[1,5-a]quinolin-3-yl)ethanone dihydrochloride (E191)

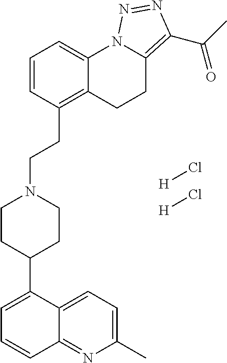

The title compound was prepared following the procedure of Example 121 using methyl magnesium bromide (0.082 ml of a 3M solution in diethyl ether, 0.245 mmol) and E190 (106 mg, 0.208 mmol). The crude product was purified by flash chromatography on silica gel eluting with methanol in DCM (2%) to afford the free base of the title compound (44 mg, 45%). Treatment with HCl (2.2 eq of 1.25M solution in MeOH) in 1:1 methanol/DCM (2 ml) at 0° C. gave the title compound as a white solid; MS (ES) m/z: 466.00 [MH⁺]. C29H31N5O requires 465.60; ¹H NMR (500 MHz, DMSO-d₆) δ ppm 2.03-2.15 (m, 2H) 2.13-2.34 (m, 2H) 2.63 (s, 3H) 2.77 (s, 3H) 3.12 (t, 2H) 3.14-3.49 (m, 6H) 3.19-3.47 (m, 2H) 3.64-3.88 (m, 3H) 7.42 (d, 1H) 7.49 (t, 1H) 7.48-7.58 (m, 1H) 7.57-7.76 (m, 1H) 7.76-7.90 (m, 1H) 7.90-8.01 (m, 1H) 8.01 (d, 1H) 8.35-9.27 (m, 1H) 10.87 (br. s., 1H).

### Example 192

### 6-{2-[2-methyl-4-(2-methyl-5-quinolinyl)-1-piperidinyl]ethyl}-4,5-dihydroimidazo[1,5-a]quinoline-3-carboxylic acid (E192)

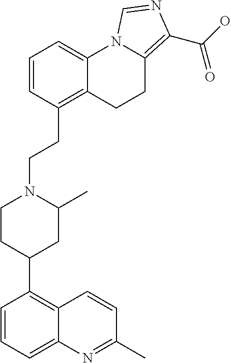

Ethyl 6-{2-[2-methyl-4-(2-methyl-5-quinolinyl)-1-piperidinyl]ethyl}-4,5-dihydroimidazo[1,5-a]quinoline-3-carboxylate (E177—enantiomer 1) (45 mg, 0.08 mmol) and KOH (1M sol in MeOH, 1.5 ml) was stirred at reflux for 1 h. The mixture was then loaded on SCX column eluting with NH3 in methanol to affording the title compound (43 mg, 0.089 mmol, 100%); MS (ES) m/z: 481.3 [MH⁺]. C₃₀H₃₂N₄O₂ requires 480.61.

### Example 193

### 6-{2-[2-methyl-4-(2-methyl-5-quinolinyl)-1-piperidinyl]ethyl}-4,5-dihydroimidazo[1,5-a]quinoline-3-carboxamide

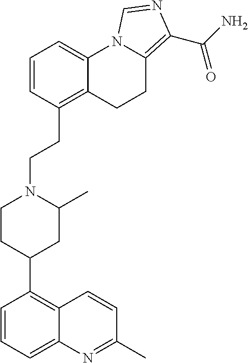

The title compound was prepared following the general procedure for amide formation (See example 14) starting from 6-{2-[2-methyl-4-(2-methyl-5-quinolinyl)-1-piperidinyl]ethyl}-4,5-dihydroimidazo[1,5-a]quinoline-3-carboxylic acid (E192) (43 mg, 0.089 mmol) using hexamethyldisilazane (0.02 ml, 0.098 mmol). The crude product was purified by SCX cartridge and flash chromatography on silica gel eluting with a gradient of methanol in DCM (2 to 3%) to afford the free base of the title compound. Treatment with HCl (2.2 eq of 1.25M solution in MeOH) in 1:2 methanol/DCM (3 ml) at 0° C. gave the title compound as a white solid (39 mg, 79%); MS (ES) m/z: 480.1 [MH⁺]. C₃₀H₃₃N₆O requires 479.62: ¹H NMR (400 MHz, DMSO-d₆) δ ppm: 11.19 (b. s., 1H) 9.33 (b. s., 1H) 8.64 (b. s., 1H) 8.27 (b. s., 1H) 8.09 (t, 1H) 7.9 (d, 1H) 7.8 (d, 1H) 7.75 (d, 1H) 7.46-7.41 (m, 2H) 7.37 (d, 1H) 7.26 (b. s., 1H) 3.93-3.24 (b. m., 8H) 3.02-2.97 (m, 7H) 2.3 (m, 1H) 2.1 (b. m. 3H) 1.45 (d, 3H).

### Example 194

### 6-{2-[2-Methyl-4-(2-methyl-5-quinolinyl)-1-piperidinyl]ethyl}-4,5-dihydroimidazo[1,5-a]quinoline-3-carboxylic acid (E194)

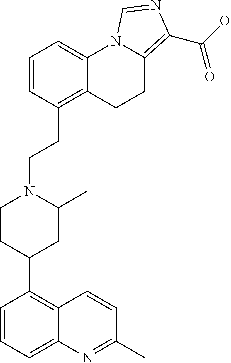

Ethyl 6-{2-[2-methyl-4-(2-methyl-5-quinolinyl)-1-piperidinyl]ethyl}-4,5-dihydroimidazo[1,5-a]quinoline-3-carboxylate (E178, enantiomer 4) (20 mg, 0.039 mmol) and KOH (1M sol in MeOH, 1.5 ml) was stirred at reflux for 1 h. The mixture was then loaded on SCX column eluting with NH3 in methanol, affording the title compound (20 mg, 100%); MS (ES) m/z: 481.3 [MH⁺]. C₃₀H₃₂N₄O₂ requires 480.61.

### Example 195

### 6-{2-[2-methyl-4-(2-methyl-5-quinolinyl)-1-piperidinyl]ethyl}-4,5-dihydroimidazo[1,5-a]quinoline-3-carboxamide

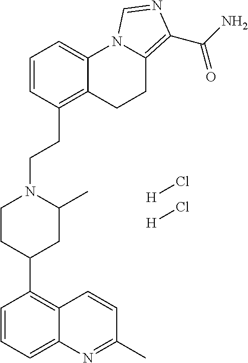

The title compound was prepared following the general procedure for amide formation (see Example 14) starting from 6-{2-[2-methyl-4-(2-methyl-5-quinolinyl)-1-piperidinyl]ethyl}-4,5-dihydroimidazo[1,5-a]quinoline-3-carboxylic acid (E194) (20 mg, 0.039 mmol) using hexamethyldisilazane (0.015 ml, 0.068 mmol). The crude product was purified by SCX cartridge and flash chromatography on silica gel eluting with a gradient of methanol in DCM (2 to 3%) to afford the free base of the title compound. Treatment with HCl (2.2 eq of 1.25M solution in MeOH) in 1:2 methanol/DCM (3 ml) at 0° C. gave the title compound as a white solid (22 mg, 100%); MS (ES) m/z: 480.1 [MH⁺]. C₃₀H₃₃N₅O requires 479.62; ¹H NMR (400 MHz, DMSO-d₆) δ ppm: 11.51 (b. s., 1H) 9.41 (d, 1H) 8.82 (b. s., 1H) 8.3 (d, 1H) 8.1 (t, 1H) 8.0 (d, 1H) 7.7 (t, 2H) 7.5 (d, 1H) 7.4 (m, 2H) 7.37 (d, 1H) 4.0 (m, 3H) 3.58 (b. d., 1H) 3.4 (b. d., 1H) 3.2 (b. m., 3H) 3.07-3.00 (m, 6H) 2.75 (t, 1H) 2.25 (m, 1H) 1.98 (t, 2H) 1.74 (d, 1H) 1.55 (d, 3H)

### Biological Assays

### a) Functional Potency—Primary Screen

The functional potency may be determined by the following GTPγS binding protocol. Cells used in the study are CHO Cells and Human Embryo Kidney (HEK293). Cells were transfected with DNA coding for human receptors as follows: HEK293_{—}5-HT_{1A}; CHO_{—}5-HT_{1B}; and CH_{—}5-HT_{1D}. Test compounds were initially dissolved in 100% dimethyl sulfoxide to a concentration of 10 mM. Serial dilution of the test compounds in 100% dimethyl sulphoxide was carried out using a Biomek FX in 384 well assay plates, so that the final top concentration of test compound is 3 μM in the assay. Add the test compound at 1.0% total assay volume (TAV) to a solid, white, 384 well assay plate (Costar). Add 50% TAV of precoupled (for 90 mins @ RT) membranes (5 ug/well), Wheatgerm Agglutinin Polystyrene Scintillation Proximity Assay beads (RPNQ0260 Amersham International) (0.25 mg/well) in 20 mM HEPES pH 7.4, 100 mM NaCl, 3 mM MgCl₂ and 10 μM GDP. The third addition was a 20% TAV addition of either buffer, agonist format, or EC₈₀ final assay concentration (FAC) of agonist, 5HT antagonist format, prepared in assay buffer. The assay was started by the addition of 29% TAV of GTPγS 0.38 nM FAC. After all additions assay plates were incubated at RT for 2-3 hours. Assay plates were counted on a Viewlux, 613/55 filter for 5 mins. Assay plates were read between 2-6 hours after the final addition.

Using assay a), typically the Examples give an fpKi against 5-HT_{1A} of greater than 6.0. Using assay a) the compounds of Examples 14, 15, 24, 83, 89, 91, 94, 97, 105, 109, 122, 124 and 125 gave fpKi values greater than 8.0 at the 5-HT_{1A} receptor. A number of these examples gave fpKi values for 5-HT_{1B} and 5-HT_{1D} receptors that were similar to the 5-HT_{1A} receptor. Using assay a) Example 24 gave a fpKi of 9.7.

### b) Receptor Affinity

The affinities of the compounds of the invention for the 5-HT_{1A}, 5-HT_{1B} and 5-HT_{1D} receptors may be determined by the following assay.

Homogenise chinese hamster overy (CHO) cells expressing 5-HT_{1A} receptors (4×10⁷ cells/ml) in Tris buffer and store in 1 ml aliquots. Homogenise CHO cells expressing 5-HT_{1B} receptors (4×10⁷ cells/ml) in Tris buffer and store in 1.5 ml aliquots. Homogenise CHO cells expressing 5-HT_{1D} receptors (1×10⁸/ml) in Tris buffer and stored in 1 ml aliquots. The binding assays are carried out in a total volume of 500 μl. For each compound to be tested make up seven solutions ranging in concentration from 0.3 mM to 0.3 nM (100× final concentrations). Dispense 5 μl of solution containing the test compound per well and add 100 μl of radioligand at 5× final desired assay concentration, i.e. [³H]-5-HT 15 nM (final assay concentration: 3 nM) in Tris Mg HCl buffer (pH 7.7) for 5-HT_{1B/1D} receptors and [³H]WAY100635 2.5 nM (final assay concentration: 0.5 nM) in Tris Mg HCl buffer (pH 7.7) containing 150 μM GPP(NH)p (final assay concentration: 30 μM) for 5-HT_{1A} receptors. Add 400 μl/well of a cell membrane suspension in Tris Mg HCl buffer (pH 7.7) to make a total volume of 505 μl. Incubate at 37° C. for 45 minutes. Determine non-specific binding using 0.01 mM 5-HT for 5-HT_{1B/1D} receptors and 0.01 mM WAY100635 for 5-HT_{1A} receptors. Terminate incubation by rapid filtration using a Packard Filtermate. Measure radioactivity using Topcount scintillation counting. Calculate pKi values from the IC₅₀ generated by an iterative least squares curve fitting programme.

c) [³H]citalopram Binding Assay for Human SERT

The affinity of the compounds to bind the re-uptake site of serotonin transporter (SERT) may be assessed using [³H]citalopram binding assay performed in recombinant epithelial pig kidney cells stably transfected with human SERT (hSERT/LLCPK). Grow cells in Petri dishes of 500 cm² and use for membrane preparation at 80% of confluence. Harvest cells in phosphate buffered saline (PBS) containing 5 mM EDTA and centrifuge at 900 g for 8 min at 4° C. Homogenize the pellet in 30-50 vols of assay buffer (50 mM Tris, 120 mM NaCl, 5 mM KCl, 10 μM pargyline, 0.1% ascorbate (pH=7.7)) and centrifuge at 48000 g for 20 min at 4° C. Resuspend the pellet in the same volume and after incubation at 37° C. for 20 min, centrifuge as before and finally aliquot at ˜0.2 mg protein/ml in cold assay buffer. For [³H]citalopram binding assay, add 4 μl of test compound (100 times in neat DMSO) (to define total binding) or a final concentration of 10 μM fluoxetine in DMSO (to define non-specific binding), 200 μl of [³H]citalopram at final concentration of 0.25 nM in assay buffer and 200 μl of membranes diluted in assay buffer at concentration of 2 μg/well of protein (final assay volume 400 μl). Add membranes to initiate the reaction and incubate at room temperature for 2 h. Stop the reaction by rapid filtration through GF/B 96-filterplate pre-soaked in 0.5% polyethylenimmine (PEI) using a Packard cell harvester. Wash 96-filterplate 3 times with 1 ml/well cold 0.9% NaCl solution and count the radioactivity in Packard TopCount.

1. A compound of formula (I), a salt or prodrug thereof 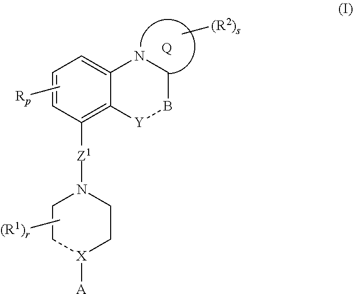
wherein
— represents independently a single or double bond;
ring Q is a 5-membered heteroaromatic ring or a 5-membered heterocyclic ring either of which contains at least one ring-nitrogen atom as shown in formula (I) and optionally 1 to 3 additional ring-heteroatoms independently selected from oxygen, nitrogen and sulphur;
B is C(R⁷)(R⁸) or C(R⁷), wherein where the bond connecting B and Y is a single bond B is C(R⁷)(R⁸) and when the bond connecting B and Y is a double bond B is C(R⁷);
Y is C(R⁷), C(R⁷)(R⁸), O or S(O)ₜ, wherein where the bond connecting B and Y is a single bond, Y is C(R⁷)(R⁸), O or S(O)ₜ and when the bond connecting B and Y is a double bond, B is C(R⁷);
Z¹ is a linking group of formula (A) 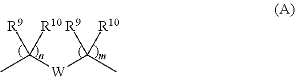
wherein
W is —(CH═CH)—; —C(═O)—; —C(═CH₂)—; —C(R⁷)(R⁸)—; —C(R⁷)(R⁸)—S(O)ₜ—; —C(R⁷)(R⁸)—O—; or a 3 to 7-membered cycloalkylene group or 3 to 7-membered cycloalkenylene group either of which groups are optionally substituted by 1 to 3 substituents which may be the same or different, selected from halogen hydroxy, cyano, C₁₋₆alkyl, haloC₁₋₆alkyl, C₁₋₆alkanoyl and C₁₋₆alkoxy; and
n and m are independently 0, 1 or 2;
X is C(R¹), N or C; wherein where the dotted bond attached to X is a single bond, X is C(R¹) or N, and when the dotted bond attached to X is a double bond, X is C;
A is indolyl, quinolyl, quinazolinyl, benzofuranyl or benzothienyl, any of which are optionally substituted by 1-4 substituents, which substituents may be the same or different, and are selected from the group consisting of halogen, hydroxy, cyano, nitro, C₁₋₆alkyl, haloC₁₋₆alkyl, C₃₋₇cycloalkyl, arylC₁₋₆alkoxy, C₁₋₆alkylthio, C₁₋₆alkoxy, haloC₁₋₆alkoxy, C₁₋₆alkoxyC₁₋₆alkyl, C₁₋₆alkoxyC₁₋₄alkoxy, C₂₋₆alkenyl, C₃₋₆alkynyl, haloC₂₋₆alkenyl, —C(O)N(R³)(R⁴), —C(O)N(R³)C₁₋₆alkoxy, —S(O)₂N(R³)(R⁴), —N(R³)(R⁴), —C(NOR⁵)R⁶, —N(R³)C(O)(R⁶), —N(R³)S(O)₂(R⁶), —C(O)R⁶, C(O)OR⁷, a heterocyclic ring, aroyl and aryl; wherein the heterocyclic ring, aroyl and aryl moieties are optionally substituted by one, two or three substituents independently selected from the group consisting of halogen, hydroxy, cyano, nitro, amino, C₁₋₆alkyl, haloC₁₋₆alkyl, C₁₋₆alkoxy and haloC₁₋₆alkoxy;
where present, each R is independently halogen, C₁₋₆alkyl, cyano, haloC₁₋₆alkyl, C₁₋₆alkanoyl, C₁₋₆alkoxy, hydroxy or trifluoromethoxy;
each R¹ is hydrogen, halogen, C₁₋₆alkyl, cyano, haloC₁₋₆alkyl, C₁₋₆alkanoyl, C₁₋₆alkoxy, hydroxy or C₁₋₆alkoxyC₁₋₆alkyl;
each R² is hydrogen, halogen, hydroxy, cyano, nitro, C₁₋₆alkyl, haloC₁₋₆alkyl, C₃₋₇cycloalkyl, arylC₁₋₆alkoxy, C₁₋₆alkylthio, C₁₋₆alkoxy, haloC₁₋₆alkoxy, C₁₋₆alkoxyC₁₋₆alkyl, C₁₋₆alkoxyC₁₋₄alkoxy, C₂₋₆alkenyl, C₃₋₆alkynyl, haloC₂₋₆alkenyl, ═O, —C(O)N(R³)(R⁴), —C(O)N(R³)C₁₋₆alkoxy, —S(O)₂N(R³)(R⁴), —N(R³)(R⁴), —C(NOR⁵)R⁶, —N(R³)C(O)(R⁶), —N(R³)S(O)₂(R⁶), —C(O)R⁶, —C(O)OR⁷, —C(O)NHNHC(O)R⁶, a heterocyclic ring, aroyl or aryl; wherein the heterocyclic ring, aroyl or aryl moieties are optionally substituted by one, two or three substituents independently selected from the group consisting of halogen, hydroxy, cyano, nitro, amino, C₁₋₆alkyl, haloC₁₋₆alkyl, C₁₋₆alkoxy and haloC₁₋₆alkoxy;
R³ and R⁴ are independently hydrogen; C₁₋₆alkyl; aryl; C₃₋₇cycloalkyl; C₃₋₇cycloalkylC₁₋₆alkyl; or where R³ and R⁴ are connected to the same nitrogen atom, together with the nitrogen, they form a 4-, 5-, 6- or 7-membered ring optionally containing one additional O, N or S ring-atom;
R⁵ is C₁₋₄alkyl, C₃₋₇cycloalkylC₁₋₆alkyl or C₃₋₇cycloalkyl;
R⁶ is hydrogen, halogen, cyano, C₃₋₇cycloalkylC₁₋₆alkyl, C₃₋₇cycloalkyl or C₁₋₆alkyl;
R⁷ and R⁸ are independently hydrogen, C₁₋₆alkyl, C₃₋₇cycloalkyl, C₃₋₇cycloalkylC₁₋₆alkyl or haloC₁₋₆alkyl;
R⁹ and R¹⁰ are independently hydrogen, C₁₋₆alkyl, cyano, haloC₁₋₆alkyl, C₁₋₆alkanoyl, C₁₋₆alkoxy, hydroxyl, halogen or C₁₋₆alkoxyC₁₋₆alkyl;
p is 0, 1 or 2;
r is 0, 1, 2 or 3;
s is 0, 1, 2 or 3; and
t is 0, 1 or 2.

2. A compound according to claim 1, a salt or prodrug thereof, wherein ring Q is an imidazole, triazole.

3. A compound according to claim 1, a salt or prodrug thereof, wherein Y is C(R⁷), C(R⁷)(R⁸) or O wherein where the bond connecting B and Y is a single bond, Y is C(R⁷)(R⁸) or O and where the bond connecting B and Y is a double bond, B is C(R⁷).

4. A compound according to claim 1, a salt or prodrug thereof, wherein Z¹ is —CH₂—, —(CH₂)₂—, —CH₂CH(CH₃)— (wherein the left hand side is attached to the nitrogen atom) or —(CH₂)₃—.

5. A compound according to claim 1, a salt or prodrug thereof, wherein X is C(R¹) or N.

6. A compound according to claim 1, a salt or prodrug thereof, wherein A is quinolyl or quinazolinyl, either of which are optionally substituted by 1-4 substituents, which substituents may be the same or different, and are selected from the group consisting of halogen, hydroxy, cyano, nitro, C₁₋₆alkyl, haloC₁₋₆alkyl, C₃₋₇cycloalkyl, arylC₁₋₆alkoxy, C₁₋₆alkylthio, C₁₋₆alkoxy, haloC₁₋₆alkoxy, C₁₋₆alkoxyC₁₋₆alkyl, C₁₋₆alkoxyC₁₋₄alkoxy, C₂₋₆alkenyl, C₃₋₆alkynyl, haloC₂₋₆alkenyl, —C(O)N(R³)(R⁴), —C(O)N(R³)C₁₋₆alkoxy, —S(O)₂N(R³)(R⁴), —N(R³)(R⁴), —C(NOR⁵)R⁶, —N(R³)C(O)(R⁶), —N(R³)S(O)₂(R⁶), —C(O)R⁶, C(O)OR⁷, a heterocyclic ring, aroyl and aryl; wherein the heterocyclic ring, aroyl and aryl moieties are optionally substituted by one, two or three substituents independently selected from the group consisting of halogen, hydroxy, cyano, nitro, amino, C₁₋₆alkyl, haloC₁₋₆alkyl, C₁₋₆alkoxy and haloC₁₋₆alkoxy.

7. A compound according to claim 6, a salt or prodrug thereof, wherein the substituents on A are selected from the group consisting of halogen, cyano, C₁₋₆alkyl, haloC₁₋₆alkyl, C₁₋₆alkoxy, haloC₁₋₆alkoxy, —C(O)N(R³)(R⁴) and —C(O)R⁶

8. A compound according to claim 1, a salt or prodrug thereof, wherein when present R is halogen or C₁₋₆alkyl.

9. A compound according to claim 1, a salt or prodrug thereof, wherein, when present R¹ is hydrogen or C₁₋₆alkyl.

10. A compound according to claim 1, a salt or prodrug thereof, wherein each R² is hydrogen, cyano, C₁₋₆alkyl, C₁₋₆alkoxy, haloC₁₋₆alkyl, ═O, —C(O)N(R³)(R⁴), —C(O)N(R³)C₁₋₆alkoxy, —S(O)₂N(R³)(R⁴), —N(R³), R⁴, —C(NOR⁵)R⁶, —N(R³)C(O)(R⁶), —N(R³)S(O)₂(R⁶), —C(O)R⁶, —C(O)OR⁷, —C(O)NHNHC(O)R⁶, a heterocyclic ring or aryl; wherein the heterocyclic ring or aryl moieties are optionally substituted by one, two or three substituents independently selected from the group consisting of halogen, cyano, nitro, amino, C₁₋₆alkyl, haloC₁₋₆alkyl, C₁₋₆alkoxy and haloC₁₋₆alkoxy.

11. A compound according to claim 1, a salt or prodrug thereof, wherein each R² is hydrogen, cyano, C₁₋₆alkyl, ═O, —C(O)N(R³)(R⁴), —C(O)N(R³)C₁₋₆alkoxy, —C(NOR⁵)R⁶, —N(R³)C(O)(R⁶), —C(O)R⁶, —C(O)OR⁷, —C(O)NHNHC(O)R⁶, a heterocyclic ring or aryl; wherein the heterocyclic ring or aryl moieties are optionally substituted by one, two or three substituents independently selected from the group consisting of halogen, cyano, nitro, amino, C₁₋₆alkyl, haloC₁₋₆alkyl, C₁₋₆alkoxy and haloC₁₋₆alkoxy.

12. A compound according to claim 1, a salt or prodrug thereof, wherein each R² is C₁₋₆alkyl, —C(O)N(R³)(R⁴) or —C(O)R⁶.

13. A compound according to claim 1, a salt or prodrug thereof, wherein s is 1 or 2.

14. A compound according to claim 1, a salt or prodrug thereof wherein
— represents independently a single or double bond;
ring Q is an imidazole, triazole or tetrazole;
B is C(R⁷)(R⁸) or C(R⁷), wherein where the bond connecting B and Y is a single bond B is C(R⁷)(R⁸) and when the bond connecting B and Y is a double bond B is C(R⁷);
Y is C(R⁷), C(R⁷)(R⁸) or O wherein where the bond connecting B and Y is a single bond, Y is C(R⁷)(R⁸) or O and where the bond connecting B and Y is a double bond, B is C(R⁷);
Z¹ is —(CH₂)₂—;
X is CH or N;
A is quinolyl or quinazolinyl, either of which are optionally substituted by 1-4 substituents, which substituents may be the same or different, and are selected from the group consisting of halogen, cyano, C₁₋₆alkyl, haloC₁₋₆alkyl, C₁₋₆alkoxy, haloC₁₋₆alkoxy, —C(O)N(R³)(R⁴) and —C(O)R⁶;
when present R is halogen (such as fluoro or chloro) or C₁₋₆alkyl (such as methyl or ethyl);
when present R¹ is hydrogen or C₁₋₆alkyl (such as methyl or ethyl);
each R² is hydrogen, cyano, C₁₋₆alkyl, ═O, —C(O)N(R³)(R⁴), —C(O)N(R³)C₁₋₆alkoxy, —C(NOR⁵)R⁶, —N(R³)C(O)(R⁶), —C(O)R⁶, —C(O)OR⁷, —C(O)NHNHC(O)R⁶, a heterocyclic ring or aryl; wherein the heterocyclic ring or aryl moieties are optionally substituted by one, two or three substituents independently selected from the group consisting of halogen, cyano, nitro, amino, C₁₋₆alkyl, haloC₁₋₆alkyl, haloC₁₋₆alkyl, C₁₋₆alkoxy and haloC₁₋₆alkoxy;
R³ and R⁴ are independently hydrogen; C₁₋₆alkyl; aryl; C₃₋₇cycloalkyl; C₃₋₇cycloalkylC₁₋₆alkyl; or where R³ and R⁴ are connected to the same nitrogen atom, together with the nitrogen, they form a 4-, 5-, 6- or 7-membered ring optionally containing one additional O, N or S ring-atom;
R⁵ is C₁₋₄alkyl, C₃₋₇cycloalkylC₁₋₆alkyl or C₃₋₇cycloalkyl;
R⁶ is hydrogen, halogen, cyano, C₃₋₇cycloalkylC₁₋₆alkyl, C₃₋₇cycloalkyl or C₁₋₆alkyl;
R⁷ and R⁸ are independently hydrogen, C₁₋₆alkyl, C₃₋₇cycloalkyl, C₃₋₇cycloalkylC₁₋₆alkyl or haloC₁₋₆alkyl;
p is 0, 1 or 2;
r is 0, 1, 2 or 3; and
s is 0, 1 or 2.

15. A compound according to claim 1, a salt or prodrug thereof, selected from the list consisting of:
6-{2-[4-(2-methyl-5-quinolinyl)-1-piperazinyl]ethyl}-4H-imidazo[5,1-c][1,4]benzoxazine-3-carboxamide dihydrochloride;
N-methyl-6-{2-[4-(2-methyl-5-quinolinyl)-1-piperazinyl]ethyl}-4H-imidazo[5,1-c][1,4]benzoxazine-3-carboxamide dihydrochloride;
6-{2-[4-(2-methyl-5-quinolinyl)-1-piperazinyl]ethyl}-3-(4-morpholinyl carbonyl)-4H-imidazo[5,1-c][1,4]benzoxazine dihydrochloride;
N-methyl-6-{2-[4-(2-methyl-5-quinolinyl)-1-piperidinyl]ethyl}-4,5-dihydroimidazo[1,5-a]quinoline-3-carboxamide dihydrochloride;
N-methyl-6-{2-[4-(2-methyl-5-quinolinyl)-1-piperidinyl]ethyl}imidazo[1,5-a]quinoline-3-carboxamide dihydrochloride;
6-{2-[4-(2-methyl-5-quinolinyl)-1-piperidinyl]ethyl}imidazo[1,5-a]quinoline-3-carboxamide dihydrochloride;
6-{2-[(2R)-2-methyl-4-(2-methyl-5-quinolinyl)-1-piperazinyl]ethyl}tetrazolo[1,5-a]quinoline dihydrochloride;
ethyl 6-{2-[4-(2-methyl-5-quinolinyl)-1-piperazinyl]ethyl}[1,2,3]triazolo[1,5-a]quinoline-3-carboxylate dihydrochloride;
6-{2-[(2R)-2-methyl-4-(2-methyl-5-quinolinyl)-1-piperazinyl]ethyl}-3-(3-methyl-1,2,4-oxadiazol-5-yl)imidazo[1,5-a]quinoline dihydrochloride;
6-{2-[4-(2-methyl-5-quinolinyl)-1-piperidinyl]ethyl}[1,2,3]triazolo[1,5-a]quinoline-3-carboxamide dihydrochloride;
6-{2-[4-(2-methyl-5-quinolinyl)-1-piperidinyl]ethyl}-4,5-dihydroimidazo[1,5-a]quinoline-3-carboxamide dihydrochloride;
N,7-dimethyl-6-{2-[4-(2-methyl-5-quinolinyl)-1-piperidinyl]ethyl}-4H-imidazo[5,1-c][1,4]benzoxazine-3-carboxamide dihydrochloride; and
7-methyl-6-{2-[4-(2-methyl-5-quinolinyl)-1-piperidinyl]ethyl}-4H-imidazo[5,1-c][1,4]benzoxazine-3-carboxamide dihydrochloride;
or other pharmaceutical acceptable salts or free bases thereof.

16-23. (canceled)

24. A compound of claim 15, which is
6-{2-[4-(2-Methyl-5-quinolinyl)-1-piperazinyl]ethyl}-4H-imidazo[5,1-c][1,4]benzoxazine-3-carboxamide, which has the following chemical structure:

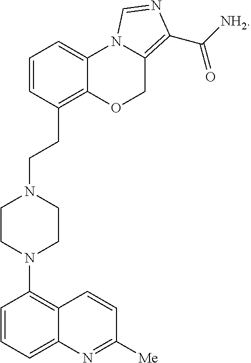

25. A compound of claim 15, which is
6-{2-[4-(2-Methyl-5-quinolinyl)-1-piperidinyl]ethyl}imidazo[1,5-a]quinoline-3-carboxamide, which has the following chemical structure:

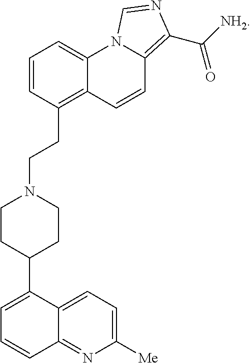

26. A compound of claim 15, which is
6-{2-[4-(2-Methyl-5-quinolinyl)-1-piperidinyl]ethyl}[1,2,3]triazolo[1,5-a]quinoline-3-carboxamide, which has the following chemical structure:

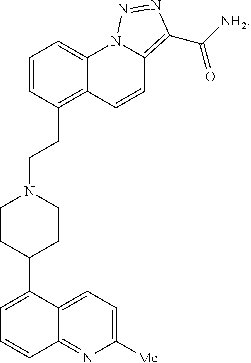

27. A compound of claim 15, which is
7-Methyl-6-{2-[4-(2-methyl-5-quinolinyl)-1-piperazinyl]ethyl}-4,5-dihydroimidazo[1,5-a]quinoline-3-carboxamide, which has the following chemical structure:

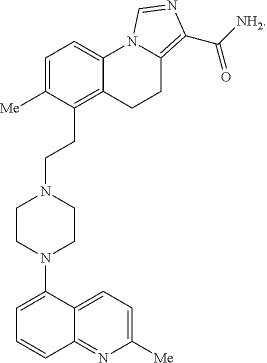

28. A compound of claim 15, which is
7-Methyl-6-{2-[4-(2-methyl-5-quinolinyl)-1-piperazinyl]ethyl}-4,5-dihydroimidazo[1,5-a]quinoline-3-carboxamide dihydrochloride.

29. A compound of formula (I) or a pharmaceutically acceptable salt thereof: 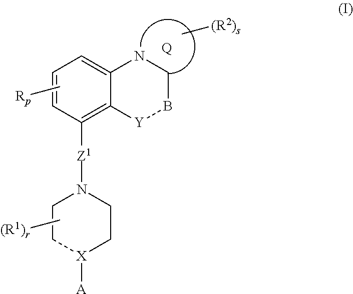
wherein
A is indolyl, quinolyl, quinazolinyl, benzofuranyl or benzothienyl, which groups are optionally substituted by 1-4 substituents, which substituents may be the same or different, and which are selected from the group consisting of halogen, hydroxy, cyano, nitro, C₁₋₆alkyl, haloC₁₋₆alkyl, C₃₋₇cycloalkyl, arylC₁₋₆alkoxy, C₁₋₆alkanoyl, C₁₋₆alkylthio, C₁₋₆alkoxy, haloC₁₋₆alkoxy, C₁₋₆alkoxyC₁₋₆alkyl, C₁₋₆alkoxyC₁₋₄alkoxy, C₁₋₆alkenyl, C₃₋₆alkynyl, haloC₁₋₆alkenyl, C(O)N(R³)(R⁴), C(O)N(R³)C₁₋₆alkoxy, S(O)₂N(R³)(R⁴), N(R³)(R⁴); C(NOR⁵)R⁶, N(R³)C(O)(R⁴), N(R³)S(O)₂(R⁴), C(O)R⁷, C(O)OR⁷, heterocyclic, aroyl or aryl wherein the aroyl or heterocyclic moiety is optionally substituted by one, two or three substituents selected from the group consisting of halogen, hydroxy, cyano, nitro, amino, C₁₋₆alkyl, haloC₁₋₆alkyl, C₁₋₆alkoxy, haloC₁₋₆alkoxy;
B is C(R⁷)(R⁸) or C(R⁷);
X is C(R¹), N or C;
Y is C(R⁷), C(R⁷)(R⁸), O or S(O)ₜ;
Z¹ is a group 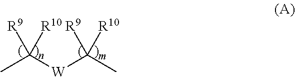
wherein n and m are independently 0, 1 or 2 and W is 3 to 7 membered cycloalkylene group or 3 to 7 membered cycloalkenylene group which groups are optionally substituted by 1 to 3 substituents which may be the same or different, and which are selected from halogen hydroxy, cyano, C₁₋₆alkyl, haloC₁₋₆alkyl, C₁₋₆alkanoyl, or C₁₋₆alkoxy or W is —(CH═CH)—, —C(═O)—, —C(═CH₂)—, —C(R⁷)(R⁸)—, C(R⁷)(R⁸)—S(O)ₜ— or —C(R⁷)(R⁸)—O—;
ring Q is a 5 membered heteroaromatic ring or a 5 membered heterocyclic ring containing at least one nitrogen and optionally containing 1 to 3 additional heteroatoms selected from oxygen, nitrogen and sulphur;
— represents independently a single or double bond provided that when:
(a) X is N or C(R¹), the bond — linked to X is a single bond
(b) Y is C(R²)(R³), N(R²), O or S(O)ₜ, the bond — linked to Y is a single bond;
R is independently halogen, C₁₋₆alkyl, cyano, haloC₁₋₆alkyl, C₁₋₆alkanoyl, C₁₋₆alkoxy, hydroxy or trifluoromethoxy;
R¹ (a) is hydrogen, C₁₋₆alkyl, cyano, haloC₁₋₆alkyl, C₁₋₆alkanoyl, C₁₋₆alkoxy hydroxy or C₁₋₆alkoxyC₁₋₆alkyl;
R² is hydrogen, halogen, hydroxy, cyano, nitro, C₁₋₆alkyl, haloC₁₋₆alkyl, C₃₋₇cycloalkyl, arylC₁₋₆alkoxy, C₁₋₆alkanoyl, C₁₋₆alkylthio, C₁₋₆alkoxy, haloC₁₋₆alkoxy, C₁₋₆alkoxyC₁₋₆alkyl, C₁₋₆alkoxyC₁₋₄alkoxy, C₁₋₆alkenyl, C₃₋₆alkynyl, haloC₁₋₆alkenyl, ═O, C(O)N(R³)(R⁴), C(O)N(R³)C₁₋₆alkoxy, S(O)₂N(R³)(R⁴), N(R³)(R⁴); C(NOR⁵)R⁶, N(R³)C(O)(R⁴), N(R³)S(O)₂(R⁴), C(O)R⁷, C(O)OR⁷, heterocyclic, aroyl or aryl wherein the aroyl or heterocyclic moiety is optionally substituted by one, two or three substituents selected from the group consisting of halogen, hydroxy, cyano, nitro, amino, C₁₋₆alkyl, haloC₁₋₆alkyl, C₁₋₆alkoxy, haloC₁₋₆alkoxy;
R³ and R⁴ are independently hydrogen, C₁₋₆alkyl, aryl, C₃₋₇cycloalkyl, C₃₋₇cycloalkylC₁₋₆alkyl or NR³R⁴ together N or form a 4-, 5-, 6- or 7-membered azacyclic group optionally containing one additional O, N or S atom in the azacycle and having 3-8 carbon atoms (including the carbon atoms contained in any optional substituent(s) of the azacycle);
R⁵ is C₁₋₄alkyl, C₃₋₇cycloalkylC₁₋₆alkyl or C₃₋₇cycloalkyl;
R⁶ is hydrogen, halogen, cyano, C₃₋₇cycloalkylC₁₋₆alkyl or C₃₋₇cycloalkyl or C₁₋₆alkyl;
R⁷ and R⁸ are independently hydrogen, C₁₋₆alkyl, or haloC₁₋₆alkyl;
R⁹ and R¹⁰ are independently hydrogen, C₁₋₆alkyl, cyano, haloC₁₋₆alkyl, C₁₋₆alkanoyl, C₁₋₆alkoxy, hydroxyl, halogen or C₁₋₆alkoxyC₁₋₆alkyl;
p is 0, 1 or 2;
r is 1, 2 or 3;
s is 1, 2 or 3;
t is 0, 1 or 2.

30. The method of treating or preventing a disease or condition mediated by modulation of the 5-HT₁ receptor and/or the serotonin re-uptake receptor which comprises administering a compound according to claim 1.

31. The method of claim 30, wherein the disease or condition is selected from:
Psychotic disorders (for example Schizophrenia (including the subtypes Paranoid Type (295.30), Disorganised Type (295.10), Catatonic Type (295.20), Undifferentiated Type (295.90) and Residual Type (295.60)); Schizophreniform Disorder (295.40); Schizoaffective Disorder (295.70) (including the subtypes Bipolar Type and Depressive Type); Delusional Disorder (297.1) (including the subtypes Erotomanic Type, Grandiose Type, Jealous Type, Persecutory Type, Somatic Type, Mixed Type and Unspecified Type); Brief Psychotic Disorder (298.8); Shared Psychotic Disorder (297.3); Psychotic Disorder due to a General Medical Condition (including the subtypes with Delusions and with Hallucinations); Substance-Induced Psychotic Disorder (including the subtypes with Delusions (293.81) and with Hallucinations (293.82)); and Psychotic Disorder Not Otherwise Specified (298.9));
Depression and mood disorders (for example Depressive Episodes (including Major Depressive Episode, Manic Episode, Mixed Episode and Hypomanic Episode); Depressive Disorders (including Major Depressive Disorder, Dysthymic Disorder (300.4), Depressive Disorder Not Otherwise Specified (311)); Bipolar Disorders (including Bipolar I Disorder, Bipolar II Disorder (i.e. Recurrent Major Depressive Episodes with Hypomanic Episodes) (296.89), Cyclothymic Disorder (301.13) and Bipolar Disorder Not Otherwise Specified (296.80)); Other Mood Disorders (including Mood Disorder due to a General Medical Condition (293.83) which includes the subtypes With Depressive Features, With Major Depressive-like Episode, With Manic Features and With Mixed Features); Substance-Induced Mood Disorder (including the subtypes With Depressive Features, With Manic Features and With Mixed Features); and Mood Disorder Not Otherwise Specified (296.90));
Anxiety disorders (for example Social Anxiety Disorder; Panic Attack; Agoraphobia, Panic Disorder; Agoraphobia Without History of Panic Disorder (300.22); Specific Phobia (300.29) (including the subtypes Animal Type, Natural Environment Type, Blood-Injection-Injury Type, Situational Type and Other Type); Social Phobia (300.23); Obsessive-Compulsive Disorder (300.3); Posttraumatic Stress Disorder (309.81); Acute Stress Disorder (308.3); Generalized Anxiety Disorder (300.02); Anxiety Disorder Due to a General Medical Condition (293.84); Substance-Induced Anxiety Disorder; and Anxiety Disorder Not Otherwise Specified (300.00)); and
Sexual dysfunctions (such as Sexual Desire Disorders (including Hypoactive Sexual Desire Disorder (302.71) and Sexual Aversion Disorder (302.79)); sexual arousal disorders (including Female Sexual Arousal Disorder (302.72) and Male Erectile Disorder (302.72)); orgasmic disorders (including Female Orgasmic Disorder (302.73), Male Orgasmic Disorder (302.74) and Premature Ejaculation (302.75)); sexual pain disorder (including Dyspareunia (302.76) and Vaginismus (306.51)); Sexual Dysfunction Not Otherwise Specified (302.70); paraphilias (including Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9)); gender identity disorders (including Gender Identity Disorder in Children (302.6) and Gender Identity Disorder in Adolescents or Adults (302.85)); and Sexual Disorder Not Otherwise Specified (302.9)).

32. The method of claim 30, wherein the disease or condition is selected from:
Depression and mood disorders (for example Depressive Episodes (including Major Depressive Episode, Manic Episode, Mixed Episode and Hypomanic Episode); Depressive Disorders (including Major Depressive Disorder, Dysthymic Disorder (300.4), Depressive Disorder Not Otherwise Specified (311)); Bipolar Disorders (including Bipolar I Disorder, Bipolar II Disorder (i.e. Recurrent Major Depressive Episodes with Hypomanic Episodes) (296.89), Cyclothymic Disorder (301.13) and Bipolar Disorder Not Otherwise Specified (296.80)); Other Mood Disorders (including Mood Disorder due to a General Medical Condition (293.83) which includes the subtypes With Depressive Features, With Major Depressive-like Episode, With Manic Features and With Mixed Features); Substance-Induced Mood Disorder (including the subtypes With Depressive Features, With Manic Features and With Mixed Features); and Mood Disorder Not Otherwise Specified (296.90));
Anxiety disorders (for example Social Anxiety Disorder; Panic Attack; Agoraphobia, Panic Disorder; Agoraphobia Without History of Panic Disorder (300.22); Specific Phobia (300.29) (including the subtypes Animal Type, Natural Environment Type, Blood-Injection-Injury Type, Situational Type and Other Type); Social Phobia (300.23); Obsessive-Compulsive Disorder (300.3); Posttraumatic Stress Disorder (309.81); Acute Stress Disorder (308.3); Generalized Anxiety Disorder (300.02); Anxiety Disorder Due to a General Medical Condition (293.84); Substance-Induced Anxiety Disorder; and Anxiety Disorder Not Otherwise Specified (300.00)); and
Sexual dysfunctions (such as Sexual Desire Disorders (including Hypoactive Sexual Desire Disorder (302.71) and Sexual Aversion Disorder (302.79)); sexual arousal disorders (including Female Sexual Arousal Disorder (302.72) and Male Erectile Disorder (302.72)); orgasmic disorders (including Female Orgasmic Disorder (302.73), Male Orgasmic Disorder (302.74) and Premature Ejaculation (302.75)); sexual pain disorder (including Dyspareunia (302.76) and Vaginismus (306.51)); Sexual Dysfunction Not Otherwise Specified (302.70); paraphilias (including Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9)); gender identity disorders (including Gender Identity Disorder in Children (302.6) and Gender Identity Disorder in Adolescents or Adults (302.85)); and Sexual Disorder Not Otherwise Specified (302.9)).

33. The method of claim 30, wherein the disease or condition is premature ejaculation.

34. A pharmaceutical composition comprising a compound, a salt or prodrug according to claim 1, in association with one or more pharmaceutically acceptable carrier(s), diluents(s) and/or excipient(s).
